(19) United States
(12) Patent Application Publication
(10) Pub. No.: US 2015/0320754 A1
(43) Pub. Date: Nov. 12, 2015

(54) COMBINATION THERAPIES

(71) Applicant: INFINITY PHARMACEUTICALS, INC., Cambridge, MA (US)

(72) Inventors: Jeffery L. Kutok, Natick, MA (US); Howard M. Stern, Waban, MA (US)

(21) Appl. No.: 14/687,714

(22) Filed: Apr. 15, 2015

Related U.S. Application Data
(60) Provisional application No. 62/110,278, filed on Jan. 30, 2015.
(60) Provisional application No. 62/042,756, filed on Aug. 27, 2014.
(60) Provisional application No. 62/042,681, filed on Aug. 27, 2014.
(60) Provisional application No. 61/980,540, filed on Apr. 16, 2014.

(51) Int. Cl. A61K 31/52; A61K 45/06; A61K 31/573

(52) U.S. Cl. CPC A61K 31/52; A61K 31/573; A61K 45/06

(57) ABSTRACT

Provided herein are pharmaceutical compositions comprising a phosphatidylinositol 3-kinase inhibitor, or pharmaceutically acceptable form thereof, in combination with a second agent, or a pharmaceutically acceptable form thereof, wherein the second agent is chosen from one or more of 1) a CDK4/6 inhibitor, 2) an HDAC inhibitor, 3) a MEK inhibitor, 4) a mTOR inhibitor, 5) an AKT inhibitor, 6) a proteasome inhibitor, 7) an immunomodulator, 8) a glucocorticosteroid, 9) a BET inhibitor, 10) an epigenetic inhibitor, 11) a PI3K alpha inhibitor, 12) a topoisomerase inhibitor, or 13) an ERK inhibitor. Also provided herein are methods of treatment comprising administration of the compositions, and uses of the compositions, e.g., for treatment of cancer.

This application claims priority to U.S. Ser. No. 61/980,540, filed Apr. 16, 2014, U.S. Ser. No. 62/042,756 filed Aug. 27, 2014, U.S. Ser. No. 62/110,278, filed Jan. 30, 2015, and U.S. Ser. No. 62/042,681 filed Aug. 27, 2014, the contents of which are incorporated herein by reference in their entireties.

### BACKGROUND

The phosphoinositide 3-kinases (PI3Ks) signaling pathway is one of the most highly mutated systems in human cancers. PI3Ks are members of a unique and conserved family of intracellular lipid kinases that phosphorylate the 3′—OH group on phosphatidylinositols or phosphoinositides. The PI3K family comprises 15 kinases with distinct substrate specificities, expression patterns, and modes of regulation. The class I PI3Ks (p110α, p110β, p110δ, and p110γ) are typically activated by tyrosine kinases or G-protein coupled receptors to generate phosphatidylinositol (3,4,5)-trisphosphate (PIP3), which engages downstream effectors such as those in the AKT/PDK1 pathway, mTOR, the Tec family kinases, and the Rho family GTPases. The class II and III PI3Ks play a key role in intracellular trafficking through the synthesis of phosphatidylinositol 3-bisphosphate (PI(3)P) and phosphatidylinositol (3,4)-bisphosphate (PI(3,4)P2). The PI3Ks are protein kinases that control cell growth (mTORC1) or monitor genomic integrity (ATM, ATR, DNA-PK, and hSmg-1).

There are four mammalian isoforms of class I PI3Ks: PI3K-α, β, δ (class Ia PI3Ks) and PI3K-γ (a class Ib PI3K). These enzymes catalyze the production of PIP3, leading to activation of downstream effector pathways important for cellular survival, differentiation, and function. PI3K-α and PI3K-β are widely expressed and are important mediators of signaling from cell surface receptors. PI3K-α is the isoform most often found mutated in cancers and has a role in insulin signaling and glucose homeostasis (Knight et al. Cell (2006) 125(4):733-47; Vanhaesebroeck et al. Current Topic Microbiol. Immunol. (2010) 347:1-19). PI3K-β is activated in cancers where phosphatase and tensin homolog (PTEN) is deleted. Both isoforms are targets of small molecule therapeutics in development for cancer.

PI3K-δ and -γ are preferentially expressed in leukocytes and are important in leukocyte function. These isoforms also contribute to the development and maintenance of hematologic malignancies (Vanhaesebroeck et al. Current Topic Microbiol. Immunol. (2010) 347:1-19; Clayton et al. J Exp Med. (2002) 196(6):753-63; Fung-Leung Cell Signal. (2011) 23(4):603-8; Okkenhaug et al. Science (2002) 297(5583):1031-34). PI3K-δ is activated by cellular receptors (e.g., receptor tyrosine kinases) through interaction with the Sarc homology 2 (SH2) domains of the PI3K regulatory subunit (p85), or through direct interaction with RAS.

### SUMMARY

The present invention provides, at least in part, compositions and methods comprising a PI3K inhibitor in combination with a selected second therapeutic agent. In one embodiment, it has been discovered that combinations of a PI3K inhibitor with a second therapeutic agent chosen from one or more of: 1) a MEK inhibitor, 2) an mTOR inhibitor, 3) an AKT inhibitor, 4) a proteasome inhibitor, 5) immunomodulator, 6) a glucocorticosteroid, 7) a CDK4/6 inhibitor, 8) an histone deacetylase (HDAC) inhibitor, 9) a BET inhibitor, 10) an epigenetic inhibitor, 11) a PI3K alpha inhibitor, 12) a topoisomerase inhibitor, or 13) an ERK inhibitor have a synergistic effect in treating a cancer (e.g., in reducing cancer cell growth or viability, or both). The combinations of PI3K inhibitors and selected second therapeutic agents can allow the PI3K inhibitor, the second therapeutic agent, or both, to be administered at a lower dosage than would be required to achieve the same therapeutic effect compared to a monotherapy dose. In some embodiments, the combination can allow the PI3K inhibitor, second therapeutic agent, or both, to be administered at a lower frequency than if the PI3K inhibitor or second therapeutic agent were administered as a monotherapy. Such combinations provide advantageous effects, e.g., in reducing, preventing, delaying, and/or decreasing in the occurrence of one or more of: a side effect, toxicity, or resistance that would otherwise be associated with administration of a higher dose of the agents.

Accordingly, in one aspect, the invention features a composition (e.g., one or more pharmaceutical compositions or dosage forms), comprising a PI3K inhibitor (e.g., one or more PI3K inhibitors), or a pharmaceutically acceptable form thereof, in combination with a second agent (e.g., one or more second therapeutic agents), or a pharmaceutically acceptable form thereof. In certain embodiments, the second therapeutic agent is chosen from one or more of: 1) a MEK inhibitor, 2) an mTOR inhibitor, 3) an AKT inhibitor, 4) a proteasome inhibitor, 5) immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor. The PI3K inhibitor and the second agent can be present in a single composition or as two or more different compositions. The PI3K inhibitor and the second agent can be administered via the same administration route or via different administration routes.

In some embodiments, the composition (e.g., one or more compositions or dosage forms) comprising the combination of PI3K inhibitor and the second agent) is synergistic, e.g., has a synergistic effect in treating a cancer (e.g., in reducing cancer cell growth or viability, or both). In certain embodiments, the amount or dosage of the PI3K inhibitor, the second agent, or both, present in the composition(s) does not exceed the level at which each agent is used individually, e.g., as a monotherapy. In certain embodiments, the amount or dosage of the PI3K inhibitor, the second agent, or both, present in the composition(s) is lower (e.g., at least 20%, at least 30%, at least 40%, or at least 50%) than the amount or dosage of each agent used individually, e.g., as a monotherapy. In other embodiments, the amount or dosage of the PI3K inhibitor, the second agent, or both, present in the composition(s) that results in a desired effect (e.g., treatment of cancer, achieve inhibition e.g., 50% inhibition, achieve growth inhibition e.g., 50% growth inhibition, achieve a therapeutic effect) is lower (e.g., at least 20%, at least 30%, at least 40%, or at least 50% lower) than the amount or dosage of each agent used individually, e.g., as a monotherapy. In certain embodiments, the frequency of administration of the PI3K inhibitor that achieves a therapeutic effect is lower (e.g., at least 20%, 30%, 40%, or 50% lower), when the PI3K inhibitor is administered in combination with the second agent than when the PI3K inhibitor is administered alone. In some embodiments, the frequency of administration of the second agent that achieves a therapeutic effect is lower (e.g., at least 20%, 30%, 40%, or 50% lower), when the second agent is administered in combination with PI3K inhibitor than when the second agent is administered alone.

In another aspect, the invention features a method of treating (e.g., inhibiting, reducing, ameliorating, managing, or preventing) a cancer in a subject. The method includes administering to the subject a PI3K inhibitor (e.g., one or more PI3K inhibitors), or a pharmaceutically acceptable form thereof, in combination with a second agent (e.g., one or more second therapeutic agents), or pharmaceutically acceptable form thereof. In certain embodiments, the second agent is chosen from one or more of: 1) a MEK inhibitor, 2) a mTOR inhibitor, 3) an AKT inhibitor, 4) a proteasome inhibitor, 5) immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor. In a related aspect, the invention features a composition for use in the treatment of a cancer. The composition for use comprises a PI3K inhibitor (e.g., one or more PI3K inhibitors), or a pharmaceutically acceptable form thereof, in combination with a second agent (e.g., one or more second therapeutic agents), or pharmaceutically acceptable form thereof. The PI3K inhibitor and the second therapeutic agent can be present in a single dose form, or as two or more dose forms.

The combination of the PI3K inhibitor and the second agent can be administered together in a single composition or administered separately in two or more different compositions, e.g., pharmaceutical compositions or dosage forms as described herein. The administration of the PI3K inhibitor and the second agent can be in any order. For example, the PI3K inhibitor can be administered concurrently with, prior to, or subsequent to, the second agent. In one embodiment, the second agent is administered to a subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the second agent is administered concurrently with the PI3K inhibitor (e.g., Compound 1), or a pharmaceutically acceptable form thereof, e.g., in a single dosage form or separate dosage forms. In yet another embodiment, the second agent is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered. In some embodiments, the PI3K inhibitor and the second agent are administered with a timing that results in both agents being present at therapeutic levels at the same time in the patient. In some embodiments, the PI3K inhibitor and the second agent are administered sequentially. In some embodiments, administration of the PI3K inhibitor and the second agent overlaps in part with each other. In some embodiments, initiation of administration of the PI3K inhibitor and the second agent occurs at the same time. In some embodiments, the PI3K inhibitor is administered before initiating treatment with the second agent. In some embodiments, the second agent is administered before initiating treatment with the PI3K inhibitor. In some embodiments, the PI3K inhibitor continues after cessation of administration of administration of the second agent. In some embodiments, the second agent continues after cessation of administration of administration of the PI3K inhibitor.

In some embodiments, the combination of the PI3K inhibitor and the second agent is additive, e.g., the effect of the combination is similar to their individual effects added together. In certain embodiments, the combination of the PI3K inhibitor and the second agent is synergistic, e.g., has a synergistic effect in treating the cancer (e.g., in reducing cancer cell growth or viability, or both). In some embodiments, the amount or dosage of the PI3K inhibitor, the second agent, or both, used in combination does not exceed the level at which each agent is used individually, e.g., as a monotherapy. In certain embodiments, the amount or dosage of the PI3K inhibitor, the second agent, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the second agent, or both, used in combination that results in treatment of cancer is lower (e.g., at least 20%, at least 30%, at least 40%, or at least 50% lower) than the amount or dosage of each agent used individually, e.g., as a monotherapy. In certain embodiments, the frequency of administration of the PI3K inhibitor, the second agent, or both, used in combination that results in treatment of cancer is lower (e.g., at least 20%, 30%, 40%, or 50% lower), than the frequency of administration of each agent used individually, e.g., as a monotherapy.

The combination of PI3K inhibitor and the second agent can be administered during periods of active disorder, or during a period of remission or less active disease. The combination can be administered before a third treatment (e.g., a third therapeutic agent) or procedure (e.g., radiation or surgery), concurrently with the third treatment, post-treatment, or during remission of the disorder.

In another aspect, the invention features a method of inhibiting the growth, the viability, or both, of a cancer cell. The method includes contacting the cancer cell with a PI3K inhibitor (e.g., one or more PI3K inhibitors), or a pharmaceutically acceptable form thereof, in combination with a second agent (e.g., one or more second therapeutic agents), or pharmaceutically acceptable form thereof. In certain embodiments, the second agent is chosen from one or more of: 1) a MEK inhibitor, 2) a mTOR inhibitor, 3) an AKT inhibitor, 4) a proteasome inhibitor, 5) immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor. The methods described herein can be used in vitro or in vivo, e.g., in an animal subject or as part of a therapeutic protocol.

The contacting of the cell with the PI3K inhibitor and the second agent can be in any order. In certain embodiments, the cell is contacted with the PI3K inhibitor concurrently, prior to, or subsequent to, the second agent. In certain embodiments, the combination of the PI3K inhibitor and the second agent is synergistic, e.g., has a synergistic effect in reducing cancer cell growth or viability, or both. In some embodiments, the amount or dosage of the PI3K inhibitor, the second agent, or both, used in combination does not exceed the level at which each agent is used individually, e.g., as a monotherapy. In certain embodiments, the amount or dosage of the PI3K inhibitor, the second agent, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the second agent, or both, used in combination that results in a reducing cancer cell growth or viability, or both is lower (e.g., at least 20%, at least 30%, at least 40%, or at least 50% lower) than the amount or dosage of each agent used individually, e.g., as a monotherapy.

In another aspect, the present disclosure provides synergistic combination of a PI3K inhibitor or a pharmaceutically acceptable form thereof, and a second therapeutic agent, or a pharmaceutically acceptable form thereof, wherein the second agent is selected from one or more of 1) a MEK inhibitor, 2) a mTOR inhibitor, 3) an AKT inhibitor, 4) a proteasome inhibitor, 5) an immunomodulator, 6) a glucocorticosteroid, 7) a CDK 4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor, or a combination thereof, for use in treating cancer. In another aspect, the present disclosure provides a synergistic combination of a PI3K inhibitor or a pharmaceutically acceptable form thereof, and a second therapeutic agent, or a pharmaceutically acceptable form thereof, wherein the second agent is selected from one or more of 1) a MEK inhibitor, 2) a mTOR inhibitor, 3) an AKT inhibitor, 4) a proteasome inhibitor, 5) an immunomodulator, 6) a glucocorticosteroid, 7) a CDK 4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor, or a combination thereof, for use in a medicament. In another aspect, the present disclosure provides a use of a synergistic combination of a PI3K inhibitor or a pharmaceutically acceptable form thereof, and a second therapeutic agent, or a pharmaceutically acceptable form thereof, wherein the second agent is selected from one or more of 1) a MEK inhibitor, 2) a mTOR inhibitor, 3) an AKT inhibitor, 4) a proteasome inhibitor, 5) an immunomodulator, 6) a glucocorticosteroid, 7) a CDK 4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor, or a combination thereof, for treating cancer. In another aspect, the present disclosure provides a use of a synergistic combination of a PI3K inhibitor or a pharmaceutically acceptable form thereof, and a second therapeutic agent, or a pharmaceutically acceptable form thereof, wherein the second agent is selected from one or more of 1) a MEK inhibitor, 2) a mTOR inhibitor, 3) an AKT inhibitor, 4) a proteasome inhibitor, 5) an immunomodulator, 6) a glucocorticosteroid, 7) a CDK 4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor, or a combination thereof for the manufacture of a medicament for treating cancer.

Additional features or embodiments of the compositions or methods described herein include one or more of the following:

In certain embodiments, the combination of the PI3K inhibitor and the second agent used in the compositions and methods described herein is synergistic, e.g., as indicated by a combination index value that is less than 1 for the combination of the PI3K inhibitor and the second agent. In certain embodiments, the combination is synergistic as indicated by a combination index value that is less than 0.7 for the combination of the PI3K inhibitor and the second agent. In certain embodiments, the combination is synergistic as indicated by a combination index value that is less than 0.5 for the combination of the PI3K inhibitor and the second agent. In certain embodiments, the combination is synergistic as indicated by a combination index value that is less than 0.7, 0.6, 0.5, 0.4, 0.3, 0.2, or 0.1 for the combination of the PI3K inhibitor and the second agent. In some embodiments, the combination of the PI3K inhibitor and the second agent used in the compositions and methods described herein is additive, e.g., as indicated by a combination index value that is equal to about 1 for the combination of the PI3K inhibitor and the second agent. In certain embodiments, the combination index value is assessed at 50% inhibition, e.g., as described herein in the Examples. In certain embodiments, the combination index value is assessed at 50% growth inhibition, e.g., as described herein in the Examples. In certain embodiments, the combination index value is assessed at 10%, 20%, 30%, 40%, 50%, 60%, 60%, 70%, 80%, or 90% inhibition or growth inhibition. In certain embodiments, the combination index value is calculated as described herein in the Examples.

In other embodiments, the combination of the PI3K inhibitor and the second agent used in the compositions and methods described herein is synergistic, e.g., as indicated by a synergy score value of greater than 1, 2, or 3. In certain embodiments, the combination is synergistic as indicated by a synergy score value of greater than 1. In certain embodiments, the combination is synergistic as indicated by a synergy score value of greater than 3. In some embodiments, the combination of the PI3K inhibitor and the second agent used in the compositions and methods described herein is additive, e.g., as indicated by a synergy score value of zero. In certain embodiments, the synergy score is calculated as described herein in the Examples.

In some embodiments, the anti-cancer effect provided by the combination of the PI3K inhibitor and the second agent used in the compositions and methods described herein is greater than the anti-cancer effect provided by an agent (e.g., the PI3K inhibitor or the second agent) used individually, e.g., as a monotherapy. In one embodiment, the anti-cancer effect provided by the combination of the PI3K inhibitor and the second agent is greater than the anti-cancer effect provided monotherapy with the same dose of the PI3K inhibitor. In certain embodiments, the anti-cancer effect provided by the combination of the PI3K inhibitor and the second agent is at least 2 fold greater, at least 3 fold greater, at least 5 fold greater, or at least 10 fold greater than the anti-cancer effect provided by an agent used individually, e.g., as a monotherapy (e.g., by a monotherapy with the same dose of the PI3K inhibitor, or by a monotherapy with the same dose of the second agent).

In some embodiments, the anti-cancer effect provided by the combination of the PI3K inhibitor and the second agent used in the compositions and methods described herein is greater than the anti-cancer effect provided by a monotherapy with the same dose of the PI3K inhibitor. In certain embodiments, the anti-cancer effect provided by the combination is at least 2 fold greater, at least 3 fold greater, at least 5 fold greater, or at least 10 fold greater than the anti-cancer effect provided by the monotherapy with the same dose of the PI3K inhibitor.

In some embodiments, the anti-cancer effect of the combination of the PI3K inhibitor and the second agent used in the compositions and methods described herein is greater than the anti-cancer effect provided by a monotherapy with the same dose of the second agent. In certain embodiments, the anti-cancer effect of the combination of the PI3K inhibitor and the second agent is at least 2 fold greater, at least 3 fold greater, at least 5 fold greater, or at least 10 fold greater than the anti-cancer effect provided by the monotherapy with the same dose of the second agent.

In some embodiments, one or more side effects of the PI3K inhibitor, the second agent, or both, is reduced compared with the side effects of each agent when used individually, e.g., as a monotherapy (e.g., a monotherapy comprising the PI3K inhibitor without the second agent at a dose that achieves the same therapeutic effect; or a monotherapy comprising the second agent without the PI3K inhibitor). For example, a reduction, prevention, delay, or decrease in the occurrence or the likelihood of occurrence of one or more side effects, toxicity, or resistance, that would otherwise be associated with administration of at least one of the agents, e.g., the PI3K inhibitor.

In some embodiments, one or more side effects of the compositions or methods described herein is reduced compared with the side effects of a monotherapy comprising either the second agent (or pharmaceutically acceptable form thereof) or the PI3K inhibitor (or pharmaceutically acceptable form thereof) at a dose that achieves the same therapeutic effect.

In some embodiments, said one or more side effects includes a liver enzyme level, e.g., a liver enzyme level indicative of toxicity.

In some embodiments, the combination of the PI3K inhibitor and the second agent used in the compositions and methods described herein results in a reduction in resistance (e.g., a decrease in a measure of resistance or a decreased likelihood of developing resistance), or a delay in the development of resistance, to at least one of the agents, e.g., resistance (e.g., acquired resistance) to the PI3K inhibitor.

In some embodiments, the combination of the PI3K inhibitor and the second agent used in the compositions and methods described herein results in a reduction in minimal residual disease (MRD). In certain embodiments, the combination of a PI3K inhibitor (e.g. a PI3K inhibitor described herein) and a second agent (e.g., a second agent described herein) is effective to reduce the MRD in the subject, e.g., below a level previously measured in the subject (e.g., the level measured before the combination was administered). In certain embodiments, the combination of a PI3K inhibitor and a second agent is effective to reduce the MRD in the subject below the level observed during or after treatment with a monotherapy, e.g., a monotherapy comprising either the PI3K inhibitor or the second agent. In certain embodiments, the MRD is decreased below the level observed during treatment with a monotherapy comprising the PI3K inhibitor. In certain embodiments, the MRD is decreased below the level observed during treatment with a monotherapy comprising the second agent. In certain embodiments, the combination is effective to reduce the level of MRD below a preselected cutoff value (e.g., 1 malignant cell in 100 normal cells, 1 malignant cell in 1000 normal cells, or 1 malignant cell in 10,000 normal cells). In certain embodiments, the preselected cutoff value is 1 malignant cell in 1000 or 10,000 normal cells. In some embodiments, a subject exhibits MRD negativity (or is MRD-negative) if the MRD is below a preselected cutoff value (e.g., a preselected cutoff value as described herein). In some embodiments, the level of MRD is not detectable by standard laboratory methodologies.

In another aspect, the invention features a method of treating a cancer in a subject, or a method of decreasing the level of MRD in a subject having a cancer. The method comprises:

(a) administering to the subject a PI3K inhibitor (e.g., Compound 1), or a pharmaceutically acceptable form thereof, in combination with a second agent (e.g., a second agent chosen from one or more of a MEK inhibitor, a mTOR inhibitor, an AKT inhibitor, a proteasome inhibitor, an immunomodulator, a glucocorticosteroid, a CDK 4/6 inhibitor, an HDAC inhibitor, a BET inhibitor, an epigenetic inhibitor, a PI3K alpha inhibitor, a topoisomerase inhibitor, or an ERK inhibitor as described herein) (also referred to as “a first treatment”);

(b) monitoring the level of MRD in the subject, e.g., by one or more methods described herein or known in the art (e.g., flow cytometry, sequencing, or PCR); and

(c) if the subject has a level of MRD below a preselected cutoff value ((e.g., 1 malignant cell in 100 normal cells, 1 malignant cell in 1000 normal cells, or 1 malignant cell in 10,000 normal cells), e.g., for a time period after therapy (e.g., at least 1, 2, 3, 6, 9, 12 months)), alter the combination treatment (e.g., reduce the dose or cease the first treatment).

In some embodiments, the method further includes monitoring the subject after altering the combination treatment (e.g., after reducing the dose or ceasing the first treatment), (e.g., for a period of at least 6 months, 9 months or 12 months), and if the level of MRD increases, e.g., increases above a preselected cutoff value (e.g., a preselected cutoff value as described herein (e.g., 1 malignant cell in 100 normal cells, 1 malignant cell in 1000 normal cells, or 1 malignant cell in 10,000 normal cells)), a second treatment is administered. In one embodiment, the second treatment is a PI3K inhibitor monotherapy. In another embodiment, the second treatment comprises a PI3K inhibitor in combination with a second agent (e.g., a second agent as described herein, e.g., one or more of a MEK inhibitor, an mTOR inhibitor, an AKT inhibitor, a proteasome inhibitor, an immunomodulator, a glucocorticosteroid, a CDK 4/6 inhibitor, an HDAC inhibitor, a BET inhibitor, an epigenetic inhibitor, a PI3K alpha inhibitor, a topoisomerase inhibitor, or an ERK inhibitor as described herein). In one embodiment, the second treatment includes the same second agent as the first treatment. In another embodiment, the second treatment includes a different second agent as the first treatment. In yet another embodiment, the second treatment comprises a PI3K inhibitor in combination with a third agent (e.g., an anti-CD20 antibody or a BTK inhibitor such as ibrutinib). In yet another embodiment, the second treatment comprises a PI3K inhibitor, a second agent (e.g., a second agent as described herein, e.g., one or more of a MEK inhibitor, an mTOR inhibitor, an AKT inhibitor, a proteasome inhibitor, an immunomodulator, a glucocorticosteroid, a CDK 4/6 inhibitor, an HDAC inhibitor, a BET inhibitor, an epigenetic inhibitor, a PI3K alpha inhibitor, a topoisomerase inhibitor, or an ERK inhibitor as described herein) and a third agent (e.g., an anti-CD20 antibody or a BTK inhibitor such as ibrutinib).

In another aspect, the invention features a method of treating a cancer in a subject, or a method of decreasing the level of MRD detected in a subject having a cancer. The method comprises:

(a) administering to the subject a PI3K inhibitor (e.g., Compound 1), or a pharmaceutically acceptable form thereof, in combination with a second agent (e.g., a second agent chosen from one or more of a MEK inhibitor, a mTOR inhibitor, an AKT inhibitor, a proteasome inhibitor, an immunomodulator, a glucocorticosteroid, a CDK 4/6 inhibitor, an HDAC inhibitor, a BET inhibitor, an epigenetic inhibitor, a PI3K alpha inhibitor, a topoisomerase inhibitor, or an ERK inhibitor as described herein) (also referred to as “a first treatment”);

(b) monitoring the level of MRD in the subject, e.g., by one or more methods described herein or known in the art (e.g., flow cytometry, sequencing, or PCR); and

(c) stop administering the first treatment (e.g., the combination) if the level of MRD in the subject decreases below a preselected cutoff value (e.g., 1 malignant cell in 100 normal cells, 1 malignant cell in 1000 normal cells, or 1 malignant cell in 10,000 normal cells).

In some embodiments, the method further comprises (d) monitoring the level of MRD in the subject, e.g., by one or more of the methods described herein or known in the art (e.g., flow cytometry, sequencing, or PCR) and (e) administering a second treatment (e.g., a monotherapy comprising a PI3K inhibitor, or administering a further combination comprising the PI3K inhibitor, or a pharmaceutically acceptable form thereof), if the level of MRD increases, e.g., increase above a preselected cutoff value (e.g., 1 malignant cell in 100 normal cells, 1 malignant cell in 1000 normal cells, or 1 malignant cell in 10,000 normal cells). Optionally, the method comprises repeating steps (b), (c), (d) and (e). In one embodiment the second treatment is a PI3K inhibitor monotherapy. In another embodiment, the second treatment comprises a PI3K inhibitor in combination with a second agent (e.g., a second agent as described herein, e.g., one or more of a MEK inhibitor, an mTOR inhibitor, an AKT inhibitor, a proteasome inhibitor, an immunomodulator, a glucocorticosteroid, a CDK 4/6 inhibitor, an HDAC inhibitor, a BET inhibitor, an epigenetic inhibitor, a PI3K alpha inhibitor, a topoisomerase inhibitor, or an ERK inhibitor as described herein). In one embodiment, the second treatment includes the same second agent as the first treatment. In another embodiment, the second treatment includes a different second agent as the first treatment. In yet another embodiment, the second treatment comprises a PI3K inhibitor in combination with a third agent (e.g., an anti-CD20 antibody or a BTK inhibitor such as ibrutinib). In yet another embodiment, the second treatment comprises a PI3K inhibitor, a second agent (e.g., a second agent as described herein, e.g., one or more of a MEK inhibitor, an mTOR inhibitor, an AKT inhibitor, a proteasome inhibitor, an immunomodulator, a glucocorticosteroid, a CDK 4/6 inhibitor, an HDAC inhibitor, a BET inhibitor, an epigenetic inhibitor, a PI3K alpha inhibitor, a topoisomerase inhibitor, or an ERK inhibitor as described herein) and a third agent (e.g., an anti-CD20 antibody or a BTK inhibitor such as ibrutinib).

The aforesaid compositions and methods can be used in combination with a monotherapy (e.g., a monotherapeutic administration or dose of the PI3K inhibitor, the second agent or a third agent). In one embodiment, the subject is administered a monotherapy with a PI3K inhibitor, which can be followed with a combination composition or method described herein. For example, if the subject is developing, or is identified as developing, a decreased responsiveness to a first monotherapy, (e.g., with a PI3K inhibitor, a second agent, or third agent), any of the combination compositions or methods described herein can be administered. In certain embodiments, the combination compositions or methods described herein improve responsiveness (e.g., as indicated by a decrease in the level of MRD, e.g., a decrease below the level of MRD observed during treatment with the first monotherapy). Alternatively, administration of any of the combination compositions or methods described herein can be followed by administration of a monotherapy, e.g., with a PI3K inhibitor, the second agent, or third agent.

In other embodiments, the composition and methods described herein can include further agents or therapies, including but not limited to, chemotherapeutics, radiation or surgery.

In some embodiments, the PI3K inhibitor is chosen from one or more of Compound 1, AMG-319, GSK 2126458, GSK 1059615, GDC-0032, GDC-0980, GDC-0941, XL147, XL499, XL765, BKM 120, GS1101, CAL 263, SF1126, PX-866, BEZ235, CAL-120, BYL719, RP6503, RP6530, TGR1202, INK1117, PX-886, BAY 80-6946, IC87114, Palomid 529, ZSTK474, PWT33597, TG100-115, GNE-477, CUDC-907, AEZS-136, BGT-226, PF-05212384, LY3023414, PI-103, LY294002, INCB-040093, CAL-130 and wortmannin. In some embodiments, the PI3K inhibitor is Compound 1 ((S)-3-(1-((9H-purin-6-yl)amino)ethyl)-8-chloro-2-phenylisoquinolin-1(2H)-one) or GS1101 (CAL-101, (S)-2-(1-(9H-purin-6-ylamino)propyl)-5-fluoro-3-phenylquinazolin-4(3H)-one),

In one embodiment, the PI3K inhibitor is Compound 1, or a pharmaceutically acceptable form thereof. Compound 1 has the following structure:

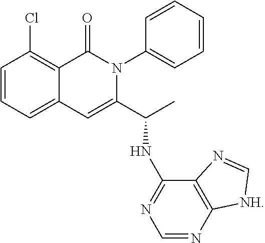 [SMILES: C[C@H](Nc1ncnc2[nH]cnc12)c1cc2cccc(Cl)c2c(=O)n1-c1ccccc1]

In one embodiment, the PI3K inhibitor is GS1101 (CAL-101), or a pharmaceutically acceptable form thereof. GS1101 (CAL-101) has the following structure:

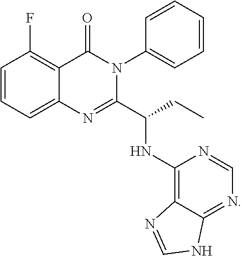 [SMILES: CC[C@H](Nc1ncnc2[nH]cnc12)c1nc2cccc(F)c2c(=O)n1-c1ccccc1]

In one embodiment, the PI3K inhibitor is Compound 1 or GS1101.

In certain embodiments of the compositions and methods described herein, the PI3K inhibitor is a PI3K delta inhibitor. In one embodiment, the PI3K inhibitor is a dual inhibitor of PI3K delta/gamma.

In some embodiments, the second agent is a chemotherapeutic. The chemotherapeutic agent can be, e.g., a cytotoxic agent (such as a DNA damaging agent) or a targeted agent. In some embodiments, the second agent is a HDAC inhibitor or a protesasome inhibitor. In some embodiments, the chemotherapeutic is administered at a lower dose (e.g., at least 20%, 30%, 40%, 50% lower) when the chemotherapeutic is administered in combination with the PI3K inhibitor than when the chemotherapeutic is administered as a monotherapy or in combination with an agent other than a PI3K inhibitor.

The combinations described herein can further comprise a third therapeutic agent which is a chemotherapeutic agent. The chemotherapeutic agent can be, for example, bendamustine, chlorambucil, cyclophosphamide, doxorubicin, vincristine, fludarabine, or any combination thereof such as CHOP (cyclophosphamide, doxorubicin, vincristine, prednisone) or FC (fludarabine, cyclophosphamide).

In some embodiments, the pharmaceutical composition further comprises a pharmaceutically acceptable excipient (e.g., one or more pharmaceutically acceptable excipients).

In some embodiments of the compositions and methods described herein, the combination of the PI3K inhibitor and the second agent is therapeutically effective (e.g., synergistically effective), in treating a cancer in the subject, e.g., for treatment of a cancer described herein.

In one embodiment, the cancer is of hematopoietic origin. In one embodiment, the cancer is lymphoma or leukemia. In one embodiment, the cancer is B-cell lymphoma, mantle cell lymphoma, non-Hodgkin's lymphoma (e.g., non-Hodgkin's B-cell lymphoma), T-cell lymphoma, cutaneous lymphoma, anaplastic large cell lymphoma, multiple myeloma, myeloma, or plasmacytoma. In one embodiment, the cancer is a multiple myeloma. In one embodiment, the cancer is a chronic lymphocytic leukemia (CLL).

In other embodiments, the cancer is a non-Hodgkin's lymphoma. In certain embodiments, the cancer is a B cell non-Hodgkin's lymphoma. In certain embodiments, the non-Hodgkin's lymphoma is a diffuse large B-cell lymphoma. In certain embodiments, the non-Hodgkin's lymphoma is a diffuse large B-cell lymphoma activated B-cell like or a diffuse large B-cell lymphoma germinal center B-cell-like. In certain embodiments, the cancer is an indolent non-Hodgkin's lymphoma, e.g., a follicular lymphoma. In certain embodiments, the cancer is a mantle cell lymphoma. In certain embodiments, the cancer is a T-cell non-Hodgkin's lymphoma.

In some embodiments, the cancer is a T cell lymphoma, e.g., a peripheral T cell lymphoma (PTCL) or a cutaneous T cell lymphoma (CTCL).

In one embodiment, the subject is a mammal, e.g., a human. In one embodiment, the subject is at risk or suffers from a cancer, e.g., a cancer described herein.

In one embodiment, the method delays resistance of the cancer, e.g., to a therapeutic agent, e.g., to the PI3K inhibitor such as Compound 1, or to the second agent. In one embodiment, the method reduces the risk that the cancer becomes resistant, e.g., to a therapeutic agent, e.g., to the PI3K inhibitor such as Compound 1, or to the second agent. In one embodiment, the cancer does not become resistant (e.g., to the PI3K inhibitor) for at least 1, 2, 3, 4, 5, 6, 7, 8, 9, 10, 11, 12, 18, 24, 30, or 36 months. In one embodiment, the method prolongs remission (e.g., complete remission or partial remission) in the subject. In one embodiment, the subject experiences remission (e.g., complete remission or partial remission) for at least 1, 2, 3, 4, 5, 6, 7, 8, 9, 10, 11, 12, 18, 24, 30, or 36 months. In one embodiment, the method increases the likelihood that the subject experiences complete remission. In one embodiment, the subject experiences complete remission. In one embodiment, the method results in a reduction in the level of minimal residual disease (MRD). In one embodiment, the subject has substantially no detectable MRD. In certain embodiments, the subject displays one or more of these characteristics (e.g., remission) after treatment with the PI3K inhibitor and the second agent for a therapeutically effective period of time, e.g., at least 1, 2, 3, or 4 weeks, or 1, 2, 4, 6, 9, or 12 months.

In one embodiment, the subject shows decreased responsiveness to a PI3K inhibitor (e.g., is resistant or refractive to treatment with a PI3K inhibitor, e.g., Compound 1). In one embodiment, the subject is identified as having a decreased susceptibility (e.g., resistance or acquired resistance) to a monotherapy treatment with a PI3K inhibitor (e.g., Compound 1 or GS1101), or a pharmaceutically acceptable form thereof. In one embodiment, the subject is identified as having a decreased susceptibility (e.g., resistance or acquired resistance) to a monotherapy treatment of a PI3K inhibitor (e.g., Compound 1), or a pharmaceutically acceptable form thereof. In one embodiment, the subject is identified as having an increased susceptibility to a combination therapy treatment provided herein.

In some embodiments of the compositions and methods described herein, the PI3K inhibitor and the second therapeutic agent are the only therapeutically active ingredients for treating a cancer.

Additional combinations of three or more agents are encompassed by the methods and compositions described herein.

In some embodiments of the compositions and methods described herein, the PI3K inhibitor and the second therapeutic agent are in a single dosage form. In other embodiments, the PI3K inhibitor and the second therapeutic agent are in separate dosage forms.

In some embodiments of the compositions and methods described herein, the combination of the PI3K inhibitor and the second agent is synergistic, e.g., in inhibiting tumor cell growth, viability or both, or in treating a cancer.

In some embodiments, the concentration, dose of the PI3K inhibitor, second therapeutic agent, or both, that achieves a therapeutic effect is lower (e.g., at least 20%, 30%, 40%, or 50% lower) when the PI3K inhibitor is administered in combination with the second therapeutic agent than when the PI3K inhibitor is administered individually or alone.

In certain embodiments, provided herein is a composition (e.g., a pharmaceutical composition) comprising a PI3K inhibitor, e.g., one or more PI3K inhibitors (e.g., Compound 1 or GS1101, or both), or a pharmaceutically acceptable form thereof, in combination with a MEK inhibitor (e.g., one or more MEK inhibitors), or a pharmaceutically acceptable form thereof. The PI3K inhibitor and the MEK inhibitor can be present in a single composition or as two or more different compositions. In some embodiments, the composition (e.g., one or more compositions comprising the combination of PI3K inhibitor and the MEK inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the MEK inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with a MEK inhibitor (e.g., one or more MEK inhibitors), or a pharmaceutically acceptable form thereof. In certain embodiments, the combination of the PI3K inhibitor and the MEK inhibitor is synergistic, e.g., has a synergistic effect in treating the cancer (e.g., in reducing cancer cell growth or viability, or both). In some embodiments, the amount or dosage of the PI3K inhibitor, the MEK inhibitor, or both, used in combination does not exceed the level at which each agent is used individually, e.g., as a monotherapy. In certain embodiments, the amount or dosage of the PI3K inhibitor, the MEK inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the MEK inhibitor, or both, used in combination that results in treatment of cancer is lower (e.g., at least 20%, at least 30%, at least 40%, or at least 50% lower) than the amount or dosage of each agent used individually, e.g., as a monotherapy.

In certain embodiments of the methods and compositions described herein, the MEK inhibitor is chosen from one or more of AZD8330, MEK162 (ARRY438162), PD-0325901, pimasertib (AS703026, MSC1935369), refametinib (BAY869766, RDEA119), R05126766, selumetinib, TAK733, trametinib (GSK1120212), WX-554, R04987655 (CH4987655), XL-518 (GDC-0973), PD184352 (CI-1040), AZD2644, or GDC0623, or a combination thereof. In one embodiment, the MEK inhibitor is trametinib or PD-0325901.

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with an mTOR inhibitor (e.g., one or more mTOR inhibitors), or a pharmaceutically acceptable form thereof. The PI3K inhibitor and the mTOR inhibitor can be present in a single composition or as two or more different compositions. In some embodiments, the composition (e.g., one or more compositions comprising the combination of PI3K inhibitor and the mTOR inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the mTOR inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with an mTOR inhibitor (e.g., one or more mTOR inhibitors), or a pharmaceutically acceptable form thereof. In certain embodiments, the combination of the PI3K inhibitor and the mTOR inhibitor is synergistic, e.g., has a synergistic effect in treating the cancer (e.g., in reducing cancer cell growth or viability, or both). In some embodiments, the amount or dosage of the PI3K inhibitor, the mTOR inhibitor, or both, used in combination does not exceed the level at which each agent is used individually, e.g., as a monotherapy. In certain embodiments, the amount or dosage of the PI3K inhibitor, the mTOR inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the mTOR inhibitor, or both, used in combination that results in treatment of cancer is lower (e.g., at least 20%, at least 30%, at least 40%, or at least 50% lower) than the amount or dosage of each agent used individually, e.g., as a monotherapy.

In one embodiment of the methods and compositions described herein, the mTOR inhibitor is chosen from one or more of AP23841, AZD8055, BEZ235, BGT226, deferolimus (AP23573/MK-8669), EM101/LY303511, everolimus (RAD001), EX2044, EX3855, EX7518, GDC0980, INK-128, KU-0063794, NV-128, OSI-027, PF-4691502, rapalogs, rapamycin, ridaforolimus, SAR543, SF1126, temsirolimus (CCI-779), WYE-125132, XL765, zotarolimus (ABT578), torin 1, GSK2126458, AZD2014, GDC-0349, or XL388, or a combination thereof. In one embodiment, the mTOR inhibitor is everolimus or AZD8055.

In certain embodiments, provided herein is a composition (e.g., a pharmaceutical composition) comprising a PI3K inhibitor (e.g., Compound 1 or GS1101), or a pharmaceutically acceptable form thereof, in combination with an AKT inhibitor (e.g., one or more AKT inhibitors), or a pharmaceutically acceptable form thereof. The PI3K inhibitor and the AKT inhibitor can be present in a single composition or as two or more different compositions. In some embodiments, the composition (e.g., one or more compositions comprising the combination of PI3K inhibitor and the AKT inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the AKT inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with an AKT inhibitor (e.g., one or more AKT inhibitors), or a pharmaceutically acceptable form thereof. In certain embodiments, the combination of the PI3K inhibitor and the AKT inhibitor is synergistic, e.g., has a synergistic effect in treating the cancer (e.g., in reducing cancer cell growth or viability, or both). In some embodiments, the amount or dosage of the PI3K inhibitor, the AKT inhibitor, or both, used in combination does not exceed the level at which each agent is used individually, e.g., as a monotherapy. In certain embodiments, the amount or dosage of the PI3K inhibitor, the AKT inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the AKT inhibitor, or both, used in combination that results in treatment of cancer is lower (e.g., at least 20%, at least 30%, at least 40%, or at least 50% lower) than the amount or dosage of each agent used individually, e.g., as a monotherapy.

In one embodiment, the AKT inhibitor is AZD5363, miltefosine, perifosine, VQD-002, MK-2206, GSK690693, GDC-0068, triciribine, CCT128930, PHT-427, or honokiol, or a combination thereof. In one embodiment, the AKT inhibitor is MK-2206 or perifosine.

In certain embodiments, provided herein is a composition, e.g., one or more pharmaceutical composition, comprising a PI3K inhibitor, e.g., one or more PI3K inhibitors (e.g., Compound 1 or GS1101), or a pharmaceutically acceptable form thereof, in combination with a proteasome inhibitor (e.g., one or more proteasome inhibitors), or a pharmaceutically acceptable form thereof. The PI3K inhibitor and the proteasome inhibitor can be present in a single composition or as two or more different compositions. In some embodiments, the composition (e.g., one or more compositions comprising the combination of PI3K inhibitor and the proteasome inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the proteasome inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject. The method includes administering to the subject a PI3K inhibitor, e.g., one or more PI3K inhibitors (e.g., Compound 1 or GS1101, or both) or a pharmaceutically acceptable form thereof, in combination with a proteasome inhibitor (e.g., one or more proteosome inhibitors), or a pharmaceutically acceptable form thereof. In certain embodiments, the combination of the PI3K inhibitor and the proteasome inhibitor is synergistic, e.g., has a synergistic effect in treating the cancer (e.g., in reducing cancer cell growth or viability, or both). In some embodiments, the amount or dosage of the PI3K inhibitor, the proteasome inhibitor, or both, used in combination does not exceed the level at which each agent is used individually, e.g., as a monotherapy. In certain embodiments, the amount or dosage of the PI3K inhibitor, the proteasome inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the proteasome inhibitor, or both, used in combination that results in treatment of cancer is lower (e.g., at least 20%, at least 30%, at least 40%, or at least 50% lower) than the amount or dosage of each agent used individually, e.g., as a monotherapy.

In one embodiment, the proteasome inhibitor is bortezomib, carfilzomib, CEP-18770, disulfiram, epigallocatechin-3-gallate, epoxomicin, lactacystin, MG132, MLN9708, ONX 0912, or salinosporamide A, or a combination thereof. In one embodiment, the proteasome inhibitor is bortezomib or carfilzomib.

In certain embodiments, provided herein is a composition, e.g., one or more pharmaceutical compositions, comprising a PI3K inhibitor (e.g., Compound 1 or GS1101), or a pharmaceutically acceptable form thereof, and an immunomodulator (e.g., one or more immunomodulators), or a pharmaceutically acceptable form thereof. The PI3K inhibitor and the immune modulator can be present in a single composition or as two or more different compositions. In some embodiments, the composition (e.g., one or more compositions comprising the combination of PI3K inhibitor and the immune modulator) is synergistic, e.g., has a synergistic effect in treating a cancer (e.g., in reducing cancer cell growth or viability, or both, e.g., as described herein). In certain embodiments, the amount or dosage of the PI3K inhibitor, the immune modulator, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, (e.g., inhibiting managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with a immunomodulator, or a pharmaceutically acceptable form thereof. In certain embodiments, the combination of the PI3K inhibitor and the immune modulator is synergistic, e.g., has a synergistic effect in treating the cancer (e.g., in reducing cancer cell growth or viability, or both). In some embodiments, the amount or dosage of the PI3K inhibitor, the immune modulator, or both, used in combination does not exceed the level at which each agent is used individually, e.g., as a monotherapy. In certain embodiments, the amount or dosage of the PI3K inhibitor, the immune modulator, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the immune modulator, or both, used in combination that results in treatment of cancer is lower (e.g., at least 20%, at least 30%, at least 40%, or at least 50% lower) than the amount or dosage of each agent used individually, e.g., as a monotherapy.

In one embodiment of the compositions or methods described herein, the immunomodulator is selected from thalidomide, lenalidomide (CC-5013), and pomalidomide (CC-4047, Pomalyst, ACTIMID). In certain embodiments, the immunomodulator is a thalidomide analog, e.g., lenalidomide or pomalidomide. In one embodiment, the immunomodulator is lenalidomide.

In certain embodiments, provided herein is a composition, e.g., one or more pharmaceutical composition, comprising a PI3K inhibitor, e.g., one or more PI3K inhibitors (e.g., Compound 1 or GS1101, or both), or a pharmaceutically acceptable form thereof, and a glucocorticosteroid, or a pharmaceutically acceptable form thereof. In some embodiments, the composition comprises Compound 1 and dexamethasone. In some embodiments, the composition comprises CAL-101 and dexamethasone. The PI3K inhibitor (e.g., Compound 1 or CAL-101) and the glucocorticoid (e.g., dexamethasone) can be present in a single composition or as two or more different compositions. In some embodiments, the composition (e.g., one or more compositions comprising the combination of PI3K inhibitor and the glucocorticoid) is synergistic, e.g., has a synergistic effect in treating a cancer (e.g., in reducing cancer cell growth or viability, or both, e.g., as described herein). In certain embodiments, the amount or dosage of the PI3K inhibitor, the glucocorticoid, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, (e.g., inhibiting managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with a glucocorticosteroid (e.g., one or more glucocorticoids), or a pharmaceutically acceptable form thereof. In some embodiments, the method comprises administering to the subject Compound 1 in combination with dexamethasone. In some embodiments, the method comprises administering CAL-101 in combination with dexamethasone. In certain embodiments, the combination of the PI3K inhibitor (e.g., Compound 1 or CAL-101) and the glucocorticoid (e.g., dexamethasone) is synergistic, e.g., has a synergistic effect in treating the cancer (e.g., in reducing cancer cell growth or viability, or both). In some embodiments, the amount or dosage of the PI3K inhibitor, the glucocorticoid, or both, used in combination does not exceed the level at which each agent is used individually, e.g., as a monotherapy. In certain embodiments, the amount or dosage of the PI3K inhibitor, the immunomodulator, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the immunomodulator, or both, used in combination that results in treatment of cancer is lower (e.g., at least 20%, at least 30%, at least 40%, or at least 50% lower) than the amount or dosage of each agent used individually, e.g., as a monotherapy. In some embodiments, the cancer is a hematological cancer, such as a lymphoma, e.g., diffuse large B cell lymphoma (DLBCL) (e.g., activated B-cell-like (ABC) DLBCL or germinal center B-cell-like (GCB) DLBCL) or follicular lymphoma. In some embodiments, the method comprises administering to the subject Compound 1 or CAL-101 in combination with dexamethasone to treat ABC DLBCL, GCB DLBCL, and/or follicular lymphoma.

In one embodiment, the glucocorticosteroid is chosen from one or more dexamethasone, aldosterone, beclomethasone, betamethasone, hydrocortisone, cortisone, deoxycorticosterone acetate (DOCA), fludrocortisone acetate, methylprednisolone, prednisolone, and prednisone, or a combination thereof. In certain embodiments, the glucocorticosteroid is dexamethasone.

In certain embodiments, provided herein is a composition, e.g., one or more pharmaceutical compositions, comprising a PI3K inhibitor, e.g., one or more PI3K inhibitors (e.g., Compound 1 or GS1101, or both) or a pharmaceutically acceptable form thereof, and a CDK4/6 inhibitor (e.g., one or more inhibitors of CDK4, CDK6 or both) or a pharmaceutically acceptable form thereof. The PI3K inhibitor and the CDK4/6 inhibitor can be present in a single composition or as two or more different compositions. In some embodiments, the composition comprises Compound 1 and LEE011. In some embodiments, the composition comprises CAL-101 and LEE011. In some embodiments, the composition comprises Compound 1 and PD-0332991. In some embodiments, the composition comprises CAL-101 and PD-0332991. In some embodiments, the composition (e.g., one or more compositions comprising the combination of PI3K inhibitor and the CDK4/6 inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the CDK4/6 inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, (e.g., inhibiting, reducing, ameliorating, managing, or preventing) a cancer in a subject. The method comprises administering to the subject a PI3K inhibitor, e.g., one or more PI3K inhibitors (e.g., Compound 1 or GS1101, or both) or a pharmaceutically acceptable form thereof, in combination with a CDK4/6 inhibitor (e.g., one or more inhibitors of CDK4, CDK6 or both), or a pharmaceutically acceptable form thereof. In some embodiments, the method comprises administering Compound 1 or CAL-101 to the subject in combination with LEE011 or PD-0332991. In some embodiments, the method comprises administering Compound 1 to the subject in combination with LEE011. In some embodiments, the method comprises administering Compound 1 to the subject in combination with PD-0332991. In some embodiments, the method comprises administering CAL-101 to the subject in combination with LEE011. In some embodiments, the method comprises administering CAL-101 to the subject in combination with PD-0332991. In certain embodiments, the combination of the PI3K inhibitor and the CDK4/6 inhibitor is synergistic, e.g., has a synergistic effect in treating the cancer (e.g., in reducing cancer cell growth or viability, or both). In some embodiments, the amount or dosage of the PI3K inhibitor, the CDK4/6 inhibitor, or both, used in combination does not exceed the level at which each agent is used individually, e.g., as a monotherapy. In certain embodiments, the amount or dosage of the PI3K inhibitor, the CDK4/6 inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the CDK4/6 inhibitor, or both, used in combination that results in treatment of cancer is lower (e.g., at least 20%, at least 30%, at least 40%, or at least 50% lower) than the amount or dosage of each agent used individually, e.g., as a monotherapy. In some embodiments, the cancer is a hematological cancer, such as a lymphoma, e.g., diffuse large B cell lymphoma (DLBCL) (e.g., activated B-cell-like (ABC) DLBCL or germinal center B-cell-like (GCB) DLBCL) or follicular lymphoma. In some embodiments, the method comprises administering to the subject Compound 1 or CAL-101 in combination with LEE011 or PD-0332991 to treat ABC DLBCL, GCB DLBCL, and/or follicular lymphoma.

Exemplary CDK4/6 inhibitors include, but are not limited to, e.g., LEE011 (Novartis), LY-2835219 (Eli Lilly), and PD 0332991 (Pfizer). In some embodiments, the CD4/6 inhibitor is selected from one or more of LEE011, PD0332991 (palbociclib), and LY2835219 (abemaciclib). In certain embodiments, the CD4/6 inhibitor is LEE011. In certain embodiments, the CD4/6 inhibitor is PD0332991 (palbociclib). In certain embodiments, the CD4/6 inhibitor is LY2835219 (abemaciclib). In one embodiment, the CDK4/6 inhibitor is LEE011 or PD0332991 or a mixture thereof. In one embodiment, the CDK4/6 inhibitor is LEE011 or LY2835219 or a mixture thereof. In one embodiment, the CDK4/6 inhibitor is LEE011 or LY2835219 or a mixture thereof. In one embodiment, the CDK4/6 inhibitor is PD0332991 or LY2835219 or a mixture thereof.

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with an HDAC (e.g., one or more HDAC inhibitors), or a pharmaceutically acceptable form thereof. The PI3K inhibitor and the HDAC inhibitor can be present in a single composition or as two or more different compositions. In some embodiments, the composition (e.g., one or more compositions comprising the combination of PI3K inhibitor and the HDAC inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the HDAC inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with an HDAC inhibitor (e.g., one or more HDAC inhibitors), or a pharmaceutically acceptable form thereof. In certain embodiments, the combination of the PI3K inhibitor and the HDAC inhibitor is synergistic, e.g., has a synergistic effect in treating the cancer (e.g., in reducing cancer cell growth or viability, or both). In some embodiments, the amount or dosage of the PI3K inhibitor, the HDAC inhibitor, or both, used in combination does not exceed the level at which each agent is used individually, e.g., as a monotherapy. In certain embodiments, the amount or dosage of the PI3K inhibitor, the HDAC inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the HDAC inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiment, the HDAC inhibitor is chosen from one or more of a hydroxamate, m-carboxycinnamic acid bis-hydroxamide (CBHA), a cyclic peptide, an aliphatic acid, a benzamide, or a sulphonamide anilide.

Exemplary HDAC inhibitors include, but are not limited to vorinostat (SAHA), romidepsin (depsipeptide or FK-228), panobinostat, valproic acid, belinostat (PXD101), mocetinostat (MGCD0103), abrexinostat, SB939, resminostat, givinostat (ITF2357), CUDC-101, AR-42, CHR-2845, CHR-3996, 4SC-202, CG200745, LAQ824, ACY-1215, kevetrin, sodium butyrate, trichostatin A, MS-275 (Entinostat), trapoxin, apicidin, chlamydocin, phenylbutyrate, AN-93, pimelic diphenylamide, N-acetyldinaline, N-2-aminophenyl-3-[4-(4-methylbenzenesulfonylamino)-phenyl]-2-propenamide, LBH-589, SK7041, SK7068, tubacin, depudecin, CI994, Quisinostat (JNJ-26481585), ME-344, sulforaphane, BML-210, PCI-3405, PCI-24781, luteolin, VAHA, chidamide, PTACH, Oxamflatin, biphenyl-4-sulfonyl chloride, HC toxin, (S)-HDAC-42, 4-iodo-SAHA, cambinol, splitomycin, SBHA, scriptaid, resveratrol, or a combination thereof. In one embodiment, the HDAC inhibitor is belinostat. In another embodiment, the HDAC inhibitor is romidepsin. In one embodiment, the HDAC inhibitor is tubastatin A hydrochloride.

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with a BET inhibitor (e.g., one or more BET inhibitors), or a pharmaceutically acceptable form thereof. The PI3K inhibitor and the BET inhibitor can be present in a single composition or as two or more different compositions. In some embodiments, the composition (e.g., one or more compositions comprising the combination of PI3K inhibitor and the BET inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the BET inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with a BET inhibitor (e.g., one or more BET inhibitors), or a pharmaceutically acceptable form thereof. In certain embodiments, the combination of the PI3K inhibitor and the BET inhibitor is synergistic, e.g., has a synergistic effect in treating the cancer (e.g., in reducing cancer cell growth or viability, or both). In some embodiments, the amount or dosage of the PI3K inhibitor, the BET inhibitor, or both, used in combination does not exceed the level at which each agent is used individually, e.g., as a monotherapy. In certain embodiments, the amount or dosage of the PI3K inhibitor, the BET inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the BET inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiments, the BET inhibitor is chosen from one or more of (+)-JQ1, GSK525762, I-BET151, PF-6405761, I-BET-762, RVX-208, OF-1, MS436, I-BET726, PFI-3, or CPI-203, or a combination thereof. In another embodiment, the BET inhibitor is (+)-JQ1.

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with an epigenetic inhibitor (e.g., one or more epigenetic inhibitors), or a pharmaceutically acceptable form thereof. The PI3K inhibitor and the epigenetic inhibitor can be present in a single composition or as two or more different compositions. In some embodiments, the composition (e.g., one or more compositions comprising the combination of PI3K inhibitor and the epigenetic inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the epigenetic inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with an epigenetic inhibitor (e.g., one or more epigenetic inhibitors), or a pharmaceutically acceptable form thereof. In certain embodiments, the combination of the PI3K inhibitor and the epigenetic inhibitor is synergistic, e.g., has a synergistic effect in treating the cancer (e.g., in reducing cancer cell growth or viability, or both). In some embodiments, the amount or dosage of the PI3K inhibitor, the epigenetic inhibitor, or both, used in combination does not exceed the level at which each agent is used individually, e.g., as a monotherapy. In certain embodiments, the amount or dosage of the PI3K inhibitor, the epigenetic inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the epigenetic inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiments, the epigenetic inhibitor is chosen from one or more of azacitidine, decitabine, RG108, thioguanine, zebularine, procainamide HCl, SGI-1027, or lomeguatrib or a combination thereof. In another embodiment, the epigenetic inhibitor is azacitidine.

In certain embodiments, provided herein is a composition (e.g., one or more pharmaceutical compositions or dosage forms), comprising a PI3K inhibitor, e.g., one or more PI3K inhibitors (e.g., a PI3K inhibitor that preferentially inhibits delta and gamma such as Compound 1, or a PI3K inhibitor that preferentially inhibits delta alone such as GS1101, or both) or a pharmaceutically acceptable form thereof, in combination with a PI3K alpha inhibitor (e.g., one or more PI3K alpha inhibitors such as GDC-0941 or GDC-0032), or a pharmaceutically acceptable form thereof. The PI3K inhibitor and the PI3K alpha inhibitor can be present in a single composition or as two or more different compositions. In some embodiments, the composition (e.g., one or more compositions comprising the combination of PI3K inhibitor and the PI3K alpha inhibitor) is synergistic, e.g., has a synergistic effect in treating a cancer (e.g., in reducing cancer cell growth or viability, or both, e.g., as described herein). The cancer can be, e.g., a cancer with a high expression level of PI3K alpha. In certain embodiments, the amount or dosage of the PI3K inhibitor, the PI3K alpha inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., a PI3K inhibitor that preferentially inhibits delta and gamma such as Compound 1 or a PI3K inhibitor that preferentially inhibits delta alone such as GS1101, or both) or a pharmaceutically acceptable form thereof, in combination with a PI3K alpha inhibitor (e.g., one or more PI3K alpha inhibitors such as GDC-0941 or GDC-0032), or a pharmaceutically acceptable form thereof. In certain embodiments, the combination of the PI3K inhibitor and the PI3K alpha inhibitor is synergistic, e.g., has a synergistic effect in treating the cancer (e.g., in reducing cancer cell growth or viability, or both). In some embodiments, the amount or dosage of the PI3K inhibitor, the PI3K alpha inhibitor, or both, used in combination does not exceed the level at which each agent is used individually, e.g., as a monotherapy. In certain embodiments, the amount or dosage of the PI3K inhibitor, the PI3K alpha inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the PI3K alpha inhibitor, or both, used in combination that results in treatment of cancer is lower (e.g., at least 20%, at least 30%, at least 40%, or at least 50% lower) than the amount or dosage of each agent used individually, e.g., as a monotherapy. The cancer can be, e.g., a cancer with a high expression level of PI3K alpha.

In certain embodiments, a PI3K inhibitor (e.g., Compound 1 or CAL-101) can be combined with a compound that inhibits PI3K alpha (e.g., GDC-0941 or GDC-0032). Certain diseases (e.g., cancer) can have a high expression level of PI3K alpha. A PI3K inhibitor that preferentially inhibits delta and gamma or delta alone can be combined with a PI3K alpha inhibitor in the treatment such diseases.

In some embodiments, the PI3K alpha inhibitor is chosen from one or more of GDC-0941, GDC-0032, HS-173, A66, PIK-75, Alpelisib, Gedatolisib, CH5132799, or Copanlisib, or a combination thereof. In some embodiments, the PI3K alpha inhibitor is GDC-0941.

In certain embodiments, provided herein is a composition (e.g., one or more pharmaceutical compositions or dosage forms), comprising a PI3K inhibitor, e.g., one or more PI3K inhibitors (e.g., a PI3K inhibitor that preferentially inhibits delta and gamma such as Compound 1, or a PI3K inhibitor that preferentially inhibits delta alone such as GS1101, or both) or a pharmaceutically acceptable form thereof, in combination with a PI3K beta inhibitor (e.g., one or more PI3K beta inhibitors such as GSK 2636771 or AZD8186), or a pharmaceutically acceptable form thereof. The PI3K inhibitor and the PI3K beta inhibitor can be present in a single composition or as two or more different compositions. In some embodiments, the composition (e.g., one or more compositions comprising the combination of PI3K inhibitor and the PI3K beta inhibitor) is synergistic, e.g., has a synergistic effect in treating a cancer (e.g., in reducing cancer cell growth or viability, or both, e.g., as described herein). The cancer can be, e.g., a cancer with a high expression level of PI3K beta. In certain embodiments, the amount or dosage of the PI3K inhibitor, the PI3K beta inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., a PI3K inhibitor that preferentially inhibits delta and gamma such as Compound 1 or a PI3K inhibitor that preferentially inhibits delta alone such as GS1101, or both) or a pharmaceutically acceptable form thereof, in combination with a PI3K beta inhibitor (e.g., one or more PI3K beta inhibitors such as GSK 2636771 or AZD8186), or a pharmaceutically acceptable form thereof. In certain embodiments, the combination of the PI3K inhibitor and the PI3K beta inhibitor is synergistic, e.g., has a synergistic effect in treating the cancer (e.g., in reducing cancer cell growth or viability, or both). In some embodiments, the amount or dosage of the PI3K inhibitor, the PI3K beta inhibitor, or both, used in combination does not exceed the level at which each agent is used individually, e.g., as a monotherapy. In certain embodiments, the amount or dosage of the PI3K inhibitor, the PI3K beta inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the PI3K beta inhibitor, or both, used in combination that results in treatment of cancer is lower (e.g., at least 20%, at least 30%, at least 40%, or at least 50% lower) than the amount or dosage of each agent used individually, e.g., as a monotherapy. The cancer can be, e.g., a cancer with a high expression level of PI3K beta.

In some aspects, provided herein is a composition (e.g., one or more pharmaceutical compositions or dosage forms), comprising two PI3K inhibitors, e.g., a PI3K alpha inhibitor and a PI3K beta inhibitor. The composition can optionally include one or more additional agents, such as one or more of: 1) a CDK 4/6 inhibitor, 2) an HDAC inhibitor, 3) a MEK inhibitor, 4) a mTOR inhibitor, 5) an AKT inhibitor, 6) a proteasome inhibitor, 7) an immunomodulator, 8) a glucocorticosteroid, 9) a BET inhibitor, 10) an epigenetic inhibitor, or 11) a topoisomerase inhibitor. The disclosure also provides methods of treating a disease, e.g., a cancer such as a hematological cancer, with the composition.

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with a topoisomerase inhibitor (e.g., one or more topoisomerase inhibitors), or a pharmaceutically acceptable form thereof. The PI3K inhibitor and the topoisomerase inhibitor can be present in a single composition or as two or more different compositions. In some embodiments, the composition (e.g., one or more compositions comprising the combination of PI3K inhibitor and the topoisomerase inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the topoisomerase inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with a topoisomerase inhibitor (e.g., one or more topoisomerase inhibitors), or a pharmaceutically acceptable form thereof. In certain embodiments, the combination of the PI3K inhibitor and the topoisomerase inhibitor is synergistic, e.g., has a synergistic effect in treating the cancer (e.g., in reducing cancer cell growth or viability, or both). In some embodiments, the amount or dosage of the PI3K inhibitor, the topoisomerase inhibitor, or both, used in combination does not exceed the level at which each agent is used individually, e.g., as a monotherapy. In certain embodiments, the amount or dosage of the PI3K inhibitor, the topoisomerase inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the topoisomerase inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiments, the topoisomerase inhibitor is chosen from one or more of doxorubicin HCl, Podophyllotoxin, Etoposide, Oxolinic Acid, Sedanolide, Mitoxantrone Dihydrochloride, 9-Hydroxyellipticine, or Amrubicin or a combination thereof. In some embodiments, the topoisomerase inhibitor is doxorubicin HCl.

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with an ERK inhibitor (e.g., one or more ERK inhibitors), or a pharmaceutically acceptable form thereof. The PI3K inhibitor and the ERK inhibitor can be present in a single composition or as two or more different compositions. In some embodiments, the composition (e.g., one or more compositions comprising the combination of PI3K inhibitor and the ERK inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the ERK inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with an ERK inhibitor (e.g., one or more topoisomerase inhibitors), or a pharmaceutically acceptable form thereof. In certain embodiments, the combination of the PI3K inhibitor and the ERK inhibitor is synergistic, e.g., has a synergistic effect in treating the cancer (e.g., in reducing cancer cell growth or viability, or both). In some embodiments, the amount or dosage of the PI3K inhibitor, the ERK inhibitor, or both, used in combination does not exceed the level at which each agent is used individually, e.g., as a monotherapy. In certain embodiments, the amount or dosage of the PI3K inhibitor, the ERK inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the ERK inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiments, the ERK inhibitor is chosen from one or more of SCH772984, BVD-523, MEK162, hypothemycin, or VX-11e, or a combination thereof.

Embodiments relating to dosages of the agents included in the compositions and methods described herein follow. In one embodiment, the PI3K inhibitor, e.g., Compound 1, is administered at a dosage of from about 0.01 mg to about 75 mg daily, and the second therapeutic agent is administered at a dosage of from about 0.01 to about 1100 mg daily.

In certain embodiments, the amount or dosage of the PI3K inhibitor, the second agent, or both, that is used in the method or composition is lower (e.g., at least 20%, at least 30%, at least 40%, at least 50%, at least 60%, at least 70%, or at least 80% lower) than the amount or dosage of each agent used individually, e.g., as a monotherapy. In other embodiments, the amount or dosage of the PI3K inhibitor, the second agent, or both, present in the composition(s) that results in a desired effect (e.g., treatment of cancer) is lower (e.g., at least 20%, at least 30%, at least 40%, at least 50%, at least 60%, at least 70%, or at least 80% lower) than the amount or dosage of each agent used individually, e.g., as a monotherapy.

In one embodiment, the molar ratio of the PI3K inhibitor, or the pharmaceutically acceptable form thereof, to the second therapeutic agent, or the pharmaceutically acceptable form thereof, is in the range of from about 10000:1 to about 1:10000.

In one embodiment, the composition comprises the PI3K inhibitor, or a pharmaceutically acceptable form thereof, at an amount of in the range of from about 0.01 mg to about 75 mg and the second therapeutic agent, or a pharmaceutically acceptable form thereof, at an amount of in the range of from about 0.01 mg to about 1100 mg.

In certain embodiments, the PI3K inhibitor is Compound 1 at a dosage of 25 mg (e.g., 25 mg BID). In certain embodiments, Compound 1 is effective as a monotherapy at a dosage of 25 mg (e.g., 25 mg BID). In certain embodiments, the combination of Compound 1 and the second agent is effective, e.g., in treating a cancer and/or in reducing cancer cell growth or viability, with Compound 1 at a dosage lower than 25 mg (e.g., 25 mg BID). In other embodiments, the dosage of Compound 1 included in the combination is 5 mg to 20 mg (e.g., 5 mg to 20 mg BID). In other embodiments, the dosage of Compound 1 included in the combination is 10 mg to 25 mg (e.g., 10 mg to 25 mg BID), 15 mg to 25 mg (e.g., 15 mg to 25 mg BID), 5 mg to 50 mg (e.g., 5 mg to 50 mg BID), 5 mg to 25 mg (e.g., 5 mg to 25 mg BID), 5 mg to 10 mg (e.g., 5 mg to 10 mg BID), 10 mg to 15 mg (e.g., 10 mg to 15 mg BID), 15 mg to 20 mg (e.g., 15 mg to 20 mg BID), 20 mg to 25 mg (e.g., 20 mg to 25 mg BID), 25 mg to 30 mg (e.g., 25 mg to 30 mg BID), 30 mg to 35 mg (e.g., 30 mg to 35 mg BID), 35 mg to 40 mg (e.g., 35 mg to 40 mg BID), 40 mg to 45 mg (e.g., 40 mg to 45 mg BID), or 45 mg to 50 mg (e.g., 45 mg to 50 mg BID). In certain embodiments, the dosage of Compound 1 is 22.5 mg (e.g., 22.5 mg BID), 20 mg (e.g., 20 mg BID), 17.5 mg (e.g., 17.5 mg BID), 15 mg (e.g., 15 mg BID), 12.5 mg (e.g., 12.5 mg BID), 10 mg (e.g., 10 mg BID), 7.5 mg (e.g., 7.5 mg BID), or 5 mg (e.g., 5 mg BID).

In some embodiments, the PI3K inhibitor, e.g., Compound 1, is administered at a dose frequency of twice per day (BID), once per day, once per two days, once per three days, once per four days, once per five days, once per six days, or once per week. In certain embodiments, the combination of the PI3K inhibitor (e.g., Compound 1) and the second agent is effective, e.g., in treating a cancer and/or in reducing cancer cell growth or viability, with the PI3K inhibitor (e.g., Compound 1) administered at a dose frequency of twice per day (BID), once per day, once per two days, once per three days, once per four days, once per five days, once per six days, or once per week.

In some embodiments, the PI3K inhibitor is GS1101 at a dosage of 150 mg (e.g., 150 mg BID). In certain embodiments, GS1101 is effective as a monotherapy at a dosage of 150 mg (e.g., 150 mg BID). In certain embodiments, the combination of GS1101 and the second agent is effective, e.g., in treating a cancer and/or in reducing cancer cell growth or viability, with GS1101 at a dosage lower than 150 mg (e.g., 150 mg BID). In some embodiments, the dosage of GS1101 included in the combination is 30 mg to 135 mg (e.g., 30 mg to 135 mg BID). In certain embodiments, the dosage of GS1101 is 135 mg (e.g., 135 mg BID), 120 mg (e.g., 120 mg BID), 105 mg (e.g., 105 mg BID), 90 mg (e.g., 90 mg BID), 75 mg (e.g., 75 mg BID), 60 mg (e.g., 60 mg BID), 45 mg (e.g., 45 mg BID), or 30 mg (e.g., 30 mg BID).

In some embodiments, the PI3K inhibitor is GS1101 and is administered at a dose frequency of twice per day, once per day, once per two days, once per three days, once per four days, once per five days, once per six days, or once per week. In certain embodiments, the combination of GS1101 and the second agent is effective, e.g., in treating a cancer and/or in reducing cancer cell growth or viability, with GS1101 administered at a dose frequency of twice per day (BID), once per day, once per two days, once per three days, once per four days, once per five days, once per six days, or once per week.

In one embodiment, the second agent is administered to a subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the second agent is administered concurrently with the PI3K inhibitor (e.g., Compound 1), or a pharmaceutically acceptable form thereof, e.g., in a single dosage form or separate dosage forms. In yet another embodiment, the second agent is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered.

In some embodiments, the second agent is a proteasome inhibitor, e.g., bortezomib. In certain embodiments, the second agent is bortezomib at a dosage of 1 mg/m². In certain embodiments, bortezomib is effective as a monotherapy at a dosage of 1 mg/m². In certain embodiments, the combination of a PI3K inhibitor (e.g., Compound 1) and bortezomib is effective, e.g., in treating a cancer and/or in reducing cancer cell growth or viability, with bortezomib at a dosage lower than 1 mg/m². In certain embodiments, the dosage of bortezomib is 0.9, 0.8, 0.7, 0.6, 0.5, 0.4, 0.3, or 0.2 mg/m².

In some embodiments, the the second agent is a proteasome inhibitor, e.g., bortezomib. In certain embodiments, the second agent is bortezomib at a dosage of 1.3 mg/m². In certain embodiments, bortezomib is effective as a monotherapy at a dosage of 1.3 mg/m². In some embodiments, the combination of a PI3K inhibitor (e.g., Compound 1) and the bortezomib is effective, e.g., in treating a cancer and/or in reducing cancer cell growth or viability, with bortezomib at a dosage lower than 1.3 mg/m². In some embodiments, the dosage of bortezomib included in the combination is 0.3 mg/m² to 1.2 mg/m². In some embodiments, the dosage of bortezomib included in the combination is 0.3 mg/m² to 1 mg/m². In some embodiments, the dosage of bortezomib is about 1.2, 1.1, 1, 0.9, 0.8, 0.7, 0.6, 0.5, 0.4, or 0.3 mg/m². In certain embodiments, the foregoing dosages of bortezomib are for daily administration.

In some embodiments, the the second agent is a proteasome inhibitor, e.g., carfilzomib. In certain embodiments, the second agent is carfilzomib at a dosage of 25 mg/m². In some embodiments, carfilzomib is effective as a monotherapy at a dosage of 25 mg/m². In some embodiments, the combination of a PI3K inhibitor (e.g., Compound 1) and the carfilzomib is effective, e.g., in treating a cancer and/or in reducing cancer cell growth or viability, with carfilzomib at a dosage lower than 25 mg/m². In some embodiments, the dosage of carfilzomib included in the combination is 5 mg/m² to 22.5 mg/m², e.g., 5 mg/m² to 20 mg/m². In certain embodiments, the dosage of carfilzomib is about 22.5, 20, 17.5, 15, 12.5, 10, 7.5, or 5 mg/m². In some embodiments, the foregoing dosages of carfilzomib are for daily administration.

In some embodiments, the second agent is a MEK inhibitor, e.g., GSK-1120212. In certain embodiments, the second agent is GSK-1120212 at a dosage of 2 mg (e.g., 2 mg QD). In some embodiments, GSK-1120212 is effective as a monotherapy at a dosage of 2 mg (e.g., 2 mg QD). In certain embodiments, the combination of a PI3K inhibitor (e.g., Compound 1) and GSK-1120212 is effective, e.g., in treating a cancer and/or in reducing cancer cell growth or viability, with GSK-1120212 at a dosage lower than 2 mg (e.g., 2 mg QD). In some embodiments, the dosage of GSK-1120212 included in the combination is 0.4 mg to 1.8 mg, e.g., 0.4 mg to 1.8 mg QD. In some embodiments, the dosage of bortezomib is 1.8 mg (e.g., 1.8 mg QD), 1.6 mg (e.g., 1.6 mg QD), 1.4 mg (e.g., 1.4 mg QD), 1.2 mg (e.g., 1.2 mg QD), 1 mg (e.g. 1 mg QD), 0.8 mg (e.g., 0.8 mg QD), 0.6 mg (e.g., 0.6 mg QD), or 0.4 mg (e.g., 0.4 mg QD).

In some embodiments, the second agent is an mTOR inhibitor, e.g., everolimus. In certain embodiments, the second agent is everolimus at a dosage of 0.75 mg (e.g., 0.75 mg BID). In certain embodiments, everolimus is effective as a monotherapy at a dosage of 0.75 mg (e.g., 0.75 mg BID). In certain embodiments, the combination of a PI3K inhibitor (e.g., Compound 1) and everolimus is effective, e.g., in treating a cancer and/or in reducing cancer cell growth or viability, with everolimus at a dosage lower than 0.75 mg (e.g., 0.75 mg BID). In some embodiments, the dosage of everolimus included in the combination is 0.15 mg to 0.675 mg (e.g., 0.15 mg to 0.675 mg). In certain embodiments, the dosage of everolimus included in the combination is 0.2 mg to 0.5 mg (e.g., 0.2 mg to 0.5 mg BID). In certain embodiments, the dosage of everolimus is about 0.675 mg (e.g., 0.675 mg BID), 0.6 mg (e.g., 0.6 mg BID), 0.525 mg (e.g., 0.525 mg BID), 0.45 mg (e.g., 0.45 mg BID), 0.375 mg (e.g. 0.375 mg BID), 0.3 mg (e.g., 0.3 mg BID), 0.225 mg (e.g., 0.225 mg BID), or 0.15 mg (e.g., 0.15 mg BID). In some embodiments, the second agent is an mTOR inhibitor, e.g., AZD8055. In certain embodiments, the second agent is AZD8055 at a dosage of 40 mg (e.g., 40 mg BID). In certain embodiments, AZD8055 is effective as a monotherapy at a dosage of 40 mg (e.g., 40 mg BID). In certain embodiments, the combination of a PI3K inhibitor (e.g., Compound 1) and AZD8055 is effective, e.g., in treating a cancer and/or in reducing cancer cell growth or viability, with AZD8055 at a dosage lower than 40 mg (e.g., 40 mg BID). In certain embodiments, the dosage of AZD8055 is about 35 mg (e.g., 35 mg BID), 30 mg (e.g., 30 mg BID), 25 mg (e.g., 25 mg BID), 20 mg (e.g., 20 mg BID), 15 mg (e.g. 15 mg BID), 10 mg (e.g., 10 mg BID), or 5 mg (e.g., 5 mg BID).

In some embodiments, the second agent is an immunomodulator, e.g., lenalidomide. In certain embodiments, the second agent is lenalidomide at a dosage of 10 mg. In some embodiments, lenalidomide is effective as a monotherapy at a dosage of 10 mg. In some embodiments, the combination of a PI3K inhibitor (e.g., Compound 1) and lenalidomide is effective, e.g., in treating a cancer and/or in reducing cancer cell growth or viability, with lenalidomide at a dosage lower than 10 mg. In some embodiments, the dosage of lenalidomide included in the combination is 2 mg to 9 mg. In some embodiments, the dosage of lenalidomide is 9, 8, 7, 6, 5, 4, 3, or 2 mg. In some embodiments, the foregoing dosages of lenalidomide are for daily administration.

In some embodiments, the second agent is an AKT inhibitor, e.g., perifosine. In some embodiments, the second agent is perifosine at a dosage of 100 mg. In certain embodiments, perifosine is effective as a monotherapy at a dosage of 100 mg. In certain embodiments, the combination of a PI3K inhibitor (e.g., Compound 1) and perifosine is effective, e.g., in treating a cancer and/or in reducing cancer cell growth or viability, with perifosine at a dosage lower than 100 mg. In certain embodiments, the dosage of perifosine included in the combination is 20 mg to 90 mg, or 20 mg to 50 mg. In certain embodiments, the dosage of perifosine is 90, 80, 70, 60, 50, 40, 30, or 20 mg. In certain embodiments, the foregoing dosages of perifosine are for daily administration.

In some embodiments, the second agent is an AKT inhibitor, e.g., MK-2206. In certain embodiments, the second agent is MK-2206 at a dosage of 60 mg. In certain embodiments, MK-2206 is effective as a monotherapy at a dosage of 60 mg. In certain embodiments, the combination of a PI3K inhibitor (e.g., Compound 1) and MK-2206 is effective, e.g., in treating a cancer and/or in reducing cancer cell growth or viability, with MK-2206 at a dosage lower than 60 mg. In certain embodiments, the dosage of MK-2206 is about 55, 50, 45, 40, 35, 30, 25, 20, 15, or 10 mg.

In some embodiments, the second agent is an MEK inhibitor, e.g., PD-0325901. In certain embodiments, the second agent is PD-0325901 at a dosage of 10 mg (e.g., 10 mg BID). In certain embodiments, PD-0325901 is effective as a monotherapy at a dosage of 10 mg (e.g., 10 mg BID). In certain embodiments, the combination of a PI3K inhibitor (e.g., Compound 1) and PD-0325901 is effective, e.g., in treating a cancer and/or in reducing cancer cell growth or viability, with PD-0325901 at a dosage lower than 10 mg (e.g., 10 mg BID). In certain embodiments, the dosage of PD-0325901 included in the combination is 2 mg to 9 mg (e.g., 2 mg to 9 mg BID) or 2 mg to 5 mg (e.g., 2 mg to 5 mg BID). In certain embodiments, the dosage of PD-0325901 is about 9 mg (e.g., 9 mg BID), 8 mg (e.g., 8 mg BID), 7 mg (e.g., 7 mg BID), 6 mg (e.g., 6 mg BID), 5 mg (e.g., 5 mg BID), 4 mg (e.g., 4 mg BID), 3 mg (e.g., 3 mg BID), or 2 mg (e.g., 2 mg BID).

In some embodiments, the second agent is a glucocorticosteroid, e.g., dexamethasone. In certain embodiments, the second agent is dexamethasone at a dosage of 1.5 mg. In certain embodiments, dexamethasone is effective as a monotherapy at a dosage of 1.5 mg. In certain embodiments, the combination of a PI3K inhibitor (e.g., Compound 1) and dexamethasone is effective, e.g., in treating a cancer and/or in reducing cancer cell growth or viability, with dexamethasone at a dosage lower than 1.5 mg. In certain embodiments, the dosage of dexamethasone included in the combination is 0.3 mg to 1.4 mg or about 0.3 mg to 1 mg. In certain embodiments, the dosage of dexamethasone is about 1.4, 1.3, 1.2, 1.1, 1.0, 0.9, 0.8, 0.7, 0.6, 0.5, 0.4, or 0.3 mg. In certain embodiments, the foregoing dosages of dexamethasone are for daily administration.

In certain embodiments, the the second agent is an HDAC inhibitor, e.g., romidepsin. In certain embodiments, the second agent is a HDAC inhibitor, e.g., romidepsin at a dosage of 14 mg/m². In certain embodiments, the HDAC inhibitor, e.g., romidepsin is effective as a monotherapy at a dosage of 14 mg/m². In certain embodiments, the combination of a PI3K inhibitor (e.g., Compound 1) and the HDAC inhibitor, e.g., romidepsin is effective, e.g., in treating a cancer and/or in reducing cancer cell growth or viability, with the HDAC inhibitor, e.g., romidepsin at a dosage lower than 14 mg/m². In certain embodiments, the dosage of HDAC inhibitor, e.g., romidepsin included in the combination is 1 mg/m² to 10 mg/m² or 1 mg/m² to 5 mg/m². In certain embodiments, the dosage of HDAC inhibitor, e.g., romidepsin is about 13.5, 12, 10, 8, 6, 5, 4, 3, 2, or 1 mg/m². In certain embodiments, the foregoing dosages of HDAC inhibitor, e.g., romidepsin are for daily administration.

In certain embodiments, the combination of a PI3K inhibitor (e.g., Compound 1) and the romidepsin is effective, e.g., in treating the cancer (e.g., in reducing cancer cell growth or viability, or both), with romidepsin at a dosage lower than 14 mg/m². In certain embodiments, the dosage of romidepsin included in the combination is 0.5 mg/m² to 10 mg/m² or 0.5 mg/m² to 5 mg/m². In certain embodiments, the dosage of romidepsin is about 13.5, 12, 10, 8, 6, 5, 4, 3, 2, 1, or 0.5 mg/m². In certain embodiments, the foregoing dosages of romidepsin are for daily administration.

In certain embodiments, the PI3K inhibitor is Compound 1 at a dosage of about 25 mg (e.g., 25 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of less than 50 mg (e.g., about 45 mg, 40 mg, 35 mg, 30 mg, 25 mg, about 22.5 mg, 20 mg, 17.5 mg, 15 mg, 12.5 mg, 10 mg, 7.5 mg, 5 mg or less) (e.g., less than 50 mg BID e.g., about 45 mg BID, 40 mg BID, 35 mg BID, 30 mg BID, 25 mg BID, 22.5 mg BID, 20 mg BID, 17.5 mg BID, 15 mg BID, 12.5 mg BID, 10 mg BID, 7.5 mg BID, 5 mg BID or less). In certain embodiments, the PI3K inhibitor is Compound 1 at a dosage of less than 25 mg (e.g., about 22.5 mg, 20 mg, 17.5 mg, 15 mg, 12.5 mg, 10 mg, 7.5 mg, 5 mg or less) (e.g., less than 25 mg BID e.g., about 22.5 mg BID, 20 mg BID, 17.5 mg BID, 15 mg BID, 12.5 mg BID, 10 mg BID, 7.5 mg BID, 5 mg BID or less) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of 10-25 mg (e.g., 10-25 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of 15-25 mg (e.g., 15-25 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of 5-20 mg (e.g., 5-20 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 22.5 mg (e.g., 22.5 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 20 mg (e.g., 20 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 17.5 mg (e.g., 17.5 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 15 mg (e.g., 15 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 12.5 mg (e.g., 12.5 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of about 10 mg (e.g., 10 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 7.5 mg (e.g., 7.5 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 5 mg (e.g., 5 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of about 5 mg to 50 mg (e.g., 5 mg to 50 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of about 10 mg to 15 mg (e.g., 10 mg to 15 mg BID), and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 5 mg to 25 mg (e.g., 5 mg to 25 mg BID), and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 5 mg to 10 mg (e.g., 5 mg to 10 mg BID), and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 15 mg to 20 mg (e.g., 15 mg to 20 mg BID), and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 20 mg to 25 mg (e.g., 20 mg to 25 mg BID), and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of about 25 mg to 30 mg (e.g., 25 mg to 30 mg BID), and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of about 30 mg to 35 mg (e.g., 30 mg to 35 mg BID), and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m²(e.g., daily). In certain embodiments, the PI3K inhibitor is Compound 1 at a dosage of about 35 mg to 40 mg (e.g., 35 mg to 40 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of about 40 mg to 45 mg (e.g., 40 mg to 45 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily). In certain embodiments, the PI3K inhibitor is Compound 1 at a dosage of about 45 mg to 50 mg (e.g., 45 mg to 50 mg BID) and the romidepsin dose is lower than 14 mg/m², e.g., 0.5 mg/m² to 10 mg/m² or 0.5 mg/m² to 5 mg/m², or about 13.5, 12, 10, 8, 6, 5, 4, 3, 2, 1, or 0.5 mg/m² (e.g., daily).

In certain embodiments, the the second agent is an HDAC inhibitor, e.g., romidepsin. In certain embodiments, the second agent is romidepsin at a dosage of 14 mg/m². In certain embodiments, romidepsin is effective as a monotherapy at a dosage of 14 mg/m². In certain embodiments, the combination of a PI3K inhibitor (e.g., Compound 1) and romidepsin is effective, e.g., in treating a cancer and/or in reducing cancer cell growth or viability, with romidepsin at a dosage lower than 14 mg/m². In certain embodiments, the dosage of romidepsin included in the combination is 1 mg/m² to 10 mg/m² or 1 mg/m² to 5 mg/m². In certain embodiments, the dosage of romidepsin is about 13.5, 12, 10, 8, 6, 5, 4, 3, 2, or 1 mg/m². In certain embodiments, the foregoing dosages of romidepsin are for daily administration. In one embodiment, the molar amount of romidepsin is 0.044 mmol. In one embodiment, the PI3K inhibitor is Compound 1 and the molar ratio of Compound 1 to romidepsin is about 2.6. In one embodiment, the molar amount of romidepsin is 0.044 mmol. In one embodiment, the PI3K inhibitor is GS1101 and the molar ratio of GS1101 to romidepsin is about 16.

In certain embodiments, the the second agent is an HDAC inhibitor, e.g., vorinostat. In certain embodiments, the second agent is vorinostat at a dosage of 14 mg/m². In certain embodiments, the second agent is vorinostat at a dosage of 400 mg or 300 mg. In certain embodiments, vorinostat is effective as a monotherapy at a dosage of 300 to 400 mg. In certain embodiments, the combination of a PI3K inhibitor (e.g., Compound 1) and vorinostat is effective, e.g., in treating a cancer and/or in reducing cancer cell growth or viability, with vorinostat at a dosage lower than 400 mg or 300 mg. In certain embodiments, the dosage of vorinostat included in the combination is 80 mg to 280 mg. In certain embodiments, the dosage of vorinostat is about 360, 320, 280, 240, 200, 160, 120, or 80 mg. In certain embodiments, the foregoing dosages of vorinostat are for daily administration. In one embodiment, the molar amount of vorinostat is about 1.1 to about 1.5 mmol. In one embodiment, the PI3K inhibitor is Compound 1 and the molar ratio of vorinostat to Compound 1 is in the range of about 10 to 13. In one embodiment, the PI3K inhibitor is GS1101 and the molar ratio of vorinostat to GS1101 is in the range of about 1.6 to 2.

In one embodiment, provided herein is a method of reducing the likelihood for a subject to develop resistance to a treatment with a PI3K inhibitor, comprising:

(a) administering to the subject a therapeutically effective amount of a monotherapy comprising the PI3K inhibitor, or a pharmaceutically acceptable form thereof, for a first period of time;

(b) after the first period of time, administering to the subject a therapeutically effective amount of a combination therapy comprising the PI3K inhibitor in combination with a second agent or a pharmaceutically acceptable form thereof, wherein the second agent is chosen from one or more of 1) a MEK inhibitor, 2) a mTOR inhibitor, 3) an AKT inhibitor, 4) a proteasome inhibitor, 5) an immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor, for a second period of time; and

(c) optionally repeating steps (a) and (b) one or more times.

In one embodiment, provided herein is a method of reducing the likelihood for a subject to develop resistance to a treatment with a PI3K inhibitor, comprising:

(a) administering to the subject a therapeutically effective amount of a monotherapy comprising the second agent, or a pharmaceutically acceptable form thereof, wherein the second agent is chosen from one or more of 1) a MEK inhibitor, 2) a mTOR inhibitor, 3) an AKT inhibitor, 4) a proteasome inhibitor, 5) an immunomodulator, 6) a glucocorticosteroid, 7) a CDK4/6 inhibitor, 08) an HDAC inhibitor, 9) a BET inhibitor, 10) an epigenetic inhibitor, 11) a PI3K alpha inhibitor, 12) a topoisomerase inhibitor, or 13) an ERK inhibitor, for a first period of time;

(b) after the first period of time, administering to the subject a therapeutically effective amount of a combination therapy comprising the PI3K inhibitor in combination with the second agent or a pharmaceutically acceptable form thereof; and

(c) optionally repeating steps (a) and (b) one or more times.

In certain embodiments, the subject is identified as developing resistance (e.g., acquired resistance) to the monotherapy.

In certain aspects, the disclosure provides a method of delaying or decreasing resistance of a subject having a cancer, comprising administering to the subject a synergistic amount of a PI3K inhibitor, or a pharmaceutically acceptable form thereof, and a second therapeutic agent selected from from 1) a MEK inhibitor, 2) a mTOR inhibitor, 3) an AKT inhibitor, 4) a proteasome inhibitor, 5) immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor, or a pharmaceutically acceptable form thereof. In a related aspect, the disclosure provides a composition for use in delaying or decreasing resistance of a subject having a cancer, said composition comprising a synergistic amount of a PI3K inhibitor, or a pharmaceutically acceptable form thereof, and a second therapeutic agent selected from from 1) a MEK inhibitor, 2) a mTOR inhibitor, 3) an AKT inhibitor, 4) a proteasome inhibitor, 5) immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor, or a pharmaceutically acceptable form thereof. In an embodiment, the resistance is resistance to the PI3K inhibitor. In an embodiment, the method comprises comprises administering the PI3K inhibitor before the second therapeutic agent.

In some aspects, this disclosure also provides a method of reducing the risk that a cancer becomes resistant to the PI3K inhibitor, comprising administering to a subject having a cancer a synergistic amount of a PI3K inhibitor, or a pharmaceutically acceptable form thereof, and a second therapeutic agent selected from 1) a MEK inhibitor, 2) a mTOR inhibitor, 3) an AKT inhibitor, 4) a proteasome inhibitor, 5) immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor.

In some aspects, this disclosure also provides a method of prolonging remission in a subject having a cancer, comprising administering to the subject a synergistic amount of a PI3K inhibitor, or a pharmaceutically acceptable form thereof, and a second therapeutic agent selected from 1) a MEK inhibitor, 2) a mTOR inhibitor, 3) an AKT inhibitor, 4) a proteasome inhibitor, 5) immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor.

In some aspects, this disclosure also provides a method of increasing the likelihood that a subject having a cancer experiences complete remission, comprising administering to the subject a synergistic amount of a PI3K inhibitor, or a pharmaceutically acceptable form thereof, and a second therapeutic agent selected from 1) a MEK inhibitor, 2) a mTOR inhibitor, 3) an AKT inhibitor, 4) a proteasome inhibitor, 5) immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor.

In some aspects, this disclosure also provides a method of reducing the level of minimal residual disease (MRD) compared to a reference value in a subject having a cancer, comprising administering to the subject a synergistic amount of a PI3K inhibitor, or a pharmaceutically acceptable form thereof, and a second therapeutic agent selected from 1) a MEK inhibitor, 2) a mTOR inhibitor, 3) an AKT inhibitor, 4) a proteasome inhibitor, 5) immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor. In a related aspect, this disclosure also provides a composition for use in reducing the level of minimal residual disease (MRD) compared to a reference value, said composition comprising a synergistic amount of a PI3K inhibitor, or a pharmaceutically acceptable form thereof, and a second therapeutic agent selected from from 1) a CDK 4/6 inhibitor, 2) an HDAC inhibitor, 3) a MEK inhibitor, 4) a mTOR inhibitor, 5) an AKT inhibitor, 6) a proteasome inhibitor, 7) an immunomodulator, 8) a glucocorticosteroid, 9) a BET inhibitor, 10) an epigenetic inhibitor, 11) a PI3K alpha inhibitor, 12) a topoisomerase inhibitor, or 13) an ERK inhibitor, or a pharmaceutically acceptable form thereof.

This disclosure also provides a method of treating a patient having a cancer, comprising administering to a patient who has, or who is identified as having, one or more of (e.g., 2, 3, 4, or all of): an elevated level of FOS, a reduced level of ATM, a reduced level of GADD45A, a reduced level of CCNG2, and a reduced level of CDKN1B, a therapeutically effective amount (e.g., a synergistic amount) of a PI3K inhibitor (e.g., Compound 1 or CAL-101) and a second therapeutic as described herein, wherein the second therapeutic is a chemotherapeutic such as a DNA-damaging agent. The chemotherapeutic agent can be, for example, bendamustine, chlorambucil, cyclophosphamide, doxorubicin, vincristine, fludarabine, or any combination thereof such as CHOP (cyclophosphamide, doxorubicin, vincristine, prednisone) or FC (fludarabine, cyclophosphamide).

The present invention also provides, at least in part, methods (e.g., diagnostic and prognostic methods) for evaluating, e.g., predicting, the responsiveness to a treatment of a cancer with a B-cell receptor (BCR) pathway inhibitor (e.g., a PI3K inhibitor, a BTK inhibitor, or a SYK inhibitor). In one embodiment, it is shown herein that STK11 copy number loss (with or without copy number loss of TSC1, TSC2, or both) is associated with, or is predictive of, decreased responsiveness (e.g., acquired resistance) of a cancer (e.g., chronic lymphocytic leukemia (CLL)) to a PI3K inhibitor (e.g., Compound 1). In other embodiments, it has been discovered that an alteration in the MAP kinase and p53 (MAPK/p53) pathway is associated with, or is predictive of, decreased responsiveness (e.g., acquired resistance) of a cancer (e.g., CLL) to a PI3K inhibitor (e.g., Compound 1). Thus, compositions, methods, and kits for the identification, assessment and/or treatment of a cancer or tumor responsive to a PI3K inhibitor treatment (e.g., a treatment that includes a PI3K inhibitor as a single agent or in combination) are disclosed herein.

Accordingly, in one aspect, the invention features a method of evaluating the responsiveness of a cancer or tumor, or a subject having a cancer or tumor, to a treatment with a BCR pathway inhibitor (e.g., a treatment with an inhibitor of PI3K, BTK or SYK, alone or in combination). In one embodiment, responsiveness to a PI3K inhibitor is evaluated. The method includes: acquiring a value (e.g., determining one or more of: the presence, absence, amount or level) of an alteration or biomarker chosen from one, two, three, four or all of: an STK11 copy number, TSC1 copy number, TSC2 copy number, a p53 pathway mutation (e.g., a mutation disclosed in Table 25), or MAPK pathway mutation (e.g., a mutation disclosed in Table 23), or any combination thereof (e.g., a dual MAPK/p53 pathway mutation, e.g., a mutation disclosed in Table 23 and a mutation disclosed in Table 25).

In another aspect, the invention features a method of monitoring a treatment of a subject with a BCR pathway inhibitor (e.g., a treatment with an inhibitor of PI3K, BTK or SYK, alone or in combination). In one embodiment, treatment with a PI3K inhibitor is monitored. The method includes: acquiring, at two or more time intervals, a value (e.g., determining one or more of: the presence, absence, amount or level) of an alteration or biomarker chosen from one, two, three, four or all of: an STK11 copy number, TSC1 copy number, TSC2 copy number, a p53 pathway mutation (e.g., a mutation disclosed in Table 25), or MAPK pathway mutation (e.g., a mutation disclosed in Table 23), or any combination thereof (e.g., a dual MAPK/p53 mutation, e.g., a mutation disclosed in Table 23 and a mutation disclosed in Table 25).

In another aspect, the invention features a method of treating (e.g., inhibiting, reducing, ameliorating, managing, or preventing) a cancer or tumor in a subject. The method includes: acquiring a value (e.g., determining one or more of: the presence, absence, amount or level) of an alteration or biomarker chosen from one, two, three, four or all of: an STK11 copy number, TSC1 copy number, TSC2 copy number, a p53 pathway mutation (e.g., a mutation disclosed in Table 25), or MAPK pathway mutation (e.g., a mutation disclosed in Table 23), or any combination thereof (e.g., a dual MAPK/p53 mutation, e.g., a mutation disclosed in Table 23 and a mutation disclosed in Table 25), and responsive to said value, administering to the subject a BCR pathway inhibitor, e.g., a PI3K inhibitor (e.g., one or more PI3K inhibitors).

In another aspect, the present disclosure provides a method of evaluating the responsiveness of a cancer or tumor, of a subject having a cancer or tumor, to a treatment with a BCR pathway inhibitor (e.g., a treatment with an inhibitor of PI3K, BTK or SYK, alone or in combination). In one embodiment, responsiveness to a PI3K inhibitor is evaluated. The method includes: acquiring a value (e.g., determining one or more of: the presence, absence, amount or level) of one or more of (e.g., 2, 3, 4, or all of): FOS, ATM, GADD45A, CCNG2, and CDKN1B.

In some embodiments, the methods that include acquiring a value of one or more of: FOS, ATM, GADD45A, CCNG2, CDKN1B include acquiring a value (e.g., determining one or more of: the presence, absence, amount or level) of an additional factor relevant to chemosensitization. In some embodiments, one or more of (e.g., 2, 3, 4, or all of) an elevated level of FOS, a reduced level of ATM, a reduced level of GADD45A, a reduced level of CCNG2, and a reduced level of CDKN1B indicate increased sensitization. In some embodiments, one or more of (e.g., 2, 3, 4, or all of) an elevated level of FOS, a reduced level of ATM, a reduced level of GADD45A, a reduced level of CCNG2, and a reduced level of CDKN1B indicate resistance to a PI3K inhibitor. In some embodiments, one or more of (e.g., 2, 3, 4, or all of) a normal or reduced level of FOS, a normal or elevated level of ATM, a normal or elevated level of GADD45A, a normal or elevated level of CCNG2, and a normal or elevated of CDKN1B indicate responsiveness to a PI3K inhibitor. In some embodiments, the methods involve administering a chemotherapeutic agent (e.g., a chemotherapeutic agent described herein such as a DNA-damaging agent), optionally in combination with a PI3K inhibitor, to a subject having one or more of (e.g., 2, 3, 4, or all of) an elevated level of FOS, a reduced level of ATM, a reduced level of GADD45A, a reduced level of CCNG2, and a reduced level of CDKN1B. In some embodiments, the methods involve administering a PI3K inhibitor as a monotherapy to a subject having a normal or reduced level of FOS, a normal or elevated level of ATM, a normal or elevated level of GADD45A, a normal or elevated level of CCNG2, and a normal or elevated level of CDKN1B. In some embodiments, the elevated, normal, or reduced levels of a biomarker are determined with reference to a non-cancerous control value.

The disclosure includes all combinations of any one or more of the foregoing aspects and/or embodiments, as well as combinations with any one or more of the embodiments set forth in the detailed description and examples.

### INCORPORATION BY REFERENCE

All publications, patents, and patent applications mentioned in this specification are herein incorporated by reference in their entirety and to the same extent as if each individual publication, patent, or patent application is specifically and individually indicated to be incorporated by reference.

### BRIEF DESCRIPTION OF THE DRAWINGS

FIG. 1 shows an isobologram depicting the synergistic effect of the combination of Compound 1 and trametinib in TMD8 cell line.

FIG. 2 shows an isobologram depicting the synergistic effect of the combination of Compound 1 and AZD8055 in TMD8 cell line.

FIG. 3 shows an isobologram depicting the synergistic effect of the combination of Compound 1 and everolimus in TMD8 cell line.

FIG. 4 shows an isobologram depicting the synergistic effect of the combination of Compound 1 and AZD8055 in Farage cell line.

FIG. 5 shows an isobologram depicting the synergistic effect of the combination of Compound 1 and everolimus in Farage cell line.

FIG. 6 shows an isobologram depicting the synergistic effect of the combination of Compound 1 and romidepsin in HH cutaneous T-cell lymphoma cell line.

FIG. 7 shows a matrix plot of percent growth inhibition of the combination of Compound 1 and romidepsin in HH cutaneous T-cell lymphoma cell line.

FIG. 8 is a graph showing the effects of Compound 1 in combination with dexamethasone (DEX) on tumor volume in the DoHH2 Follicular B cell lymphoma subcutaneous model.

FIG. 9 is a graph showing the effects of Compound 1 in combination with dexamethasone (DEX) on percent survival versus time for tumors to reach 3000 mm3 in the DoHH2 Follicular B cell lymphoma subcutaneous model.

FIG. 10 is a graph and table showing the IC50 of inhibition by Compound 1 in control cells (not resistant to Compound 1) and Compound 1-resistant cells.

FIG. 11 is a graph showing the synergy in growth inhibition between Compound 1 and dexamethasone in DOHH2 cells.

FIG. 12 is a graph showing the synergy in growth inhibition between Compound 1 and dexamethasone in SUDHL6 cells.

FIG. 13 is a graph showing the top upregulated and downregulated genes (≧2 fold change) in Compound 1-resistant cells (compared to non-resistant cells).

FIG. 14 is a graph showing the fold change in expression level of several genes in cells resistant to Compound 1 or ibrutinib.

FIG. 15 is a graphical representation of the relationship between mutations and responses to Compound 1. Each column represents a patient. Each row represents a mutation. The diagnosis is coded as 1: CLL/SLL (R/R), or 2: CLL/SLL (treatment-naïve). R/R refers to a patient that has relapsed or is refractory to treatment. Tx naïve refers to a patient that is treatment naïve, e.g., has not been previously administered Compound 1. The response is coded as 3: CR/PR, 4: PRwL, 5: SD/PD, or 6: SD/PD (nodal response). The ALC is coded as 7: high, 8: normal, or 9: low. PR refers to partial remission, SD refers to stable disease, PD refers to progressive disease, and CR refers to complete remission

FIG. 16 is a graphical representation of the relationship between mutations and responses to Compound 1. Each column represents a patient. Each row represents a mutation. The diagnosis is coded as 1: CLL/SLL (R/R), or 2: CLL/SLL (treatment-naïve). The response is coded as IWCLL complete remission or partial remission (CR/PR) or IWCLL stable disease or progressive disease (SD/PD). Nodal responses are indicated with an asterisk (*).

FIG. 17 is a graphical representation of the relationship between mutations and responses to Compound 1. The diagnosis and response is coded as in FIG. 16.

FIG. 18 is a graphical representation of the relationship between mutations and responses to Compound 1. The diagnosis and response is coded as in FIG. 16. Nodal responses are indicated with an asterisk (*). A non-assessable nodal response is indicated by a (#).

FIG. 19 is a graphical representation of the relationship between CLL common copy number variations (CNVs) and responses to Compound 1. The diagnosis and response is coded as in FIG. 16.

FIG. 20A is a graph depicting relative expression of TP53 (RNA levels) in patients with no loss or with a loss in TP53 copy number. FIG. 20B is a graph depicting relative expression of YWHAE (RNA levels) in patients with no loss or with a loss in YWHAE copy number. FIG. 20C is a graph depicting relative expression of STK11 (RNA levels) in patients with no loss or with a loss in STK11 copy number.

FIG. 21 is a graphical representation of the relationship between and responses to Compound 1 and alterations in various pathways. “Dual” in this figure refers to dual p53 and MAPK pathways. The diagnosis and response is coded as in FIG. 16.

FIG. 22 is a graph showing the PTEN RNA expression level in DMSO control treated cells or cells resistant to Compound 1. FPKM refers to fragments per kilobase of exon per million fragments mapped.

FIG. 23 is a bar chart showing the log (2) fold change of TYRO3 in Compound 1 resistant and ibrutinib resistant clones as compared to control.

### DETAILED DESCRIPTION

The present invention provides, at least in part, compositions and methods comprising a PI3K inhibitor in combination with a selected second therapeutic agent. In one embodiment, it has been discovered that combinations of a PI3K inhibitor with a second therapeutic agent chosen from one or more of: 1) a MEK inhibitor, 2) an mTOR inhibitor, 3) an AKT inhibitor, 4) a proteasome inhibitor, 5) immunomodulator, 6) a glucocorticosteroid, 7) a CDK4/6 inhibitor, 8) an histone deacetylase (HDAC) inhibitor, 9) a BET inhibitor, 10) an epigenetic inhibitor, 11) a PI3K alpha inhibitor, 12) a topoisomerase inhibitor, or 13) an ERK inhibitor have a synergistic effect in treating a cancer (e.g., in reducing cancer cell growth or viability, or both). The combinations of PI3K inhibitors and selected second therapeutic agents can allow the PI3K inhibitor, the second therapeutic agent, or both, to be administered at a lower dosage than would be required to achieve the same therapeutic effect compared to a monotherapy dose. In some embodiments, the combination can allow the PI3K inhibitor, second therapeutic agent, or both, to be administered at a lower frequency than if the PI3K inhibitor or second therapeutic agent were administered as a monotherapy. Such combinations provide advantageous effects, e.g., in reducing, preventing, delaying, and/or decreasing in the occurrence of one or more of: a side effect, toxicity, or resistance that would otherwise be associated with administration of a higher dose of the agents.

The present invention also provides, at least in part, methods (e.g., diagnostic and prognostic methods) for evaluating, e.g., predicting, the responsiveness to a treatment of a cancer with a B-cell receptor (BCR) pathway inhibitor (e.g., a PI3K inhibitor). In one embodiment, it is shown herein that STK11 copy number loss (with or without copy number loss of TSC1, TSC2, or both) is associated with, or is predictive of, decreased responsiveness (e.g., acquired resistance) of a cancer (e.g., chronic lymphocytic leukemia (CLL)) to a PI3K inhibitor (e.g., Compound 1). In other embodiments, it has been discovered that an alteration in the MAP kinase and p53 (MAPK/p53) pathway is associated with, or is predictive of, decreased responsiveness (e.g., acquired resistance) of a cancer (e.g., CLL) to a PI3K inhibitor (e.g., Compound 1). Thus, compositions, methods, and kits for evaluating responsiveness (e.g., acquisition of resistance) to, or monitor, therapy involving PI3K inhibition (including combination therapies); stratify patient populations; identify subjects likely to benefit from such agents, predict a time course of disease or a probability of a significant event in the disease for such subjects, and/or more effectively monitor, treat or prevent a cancer are disclosed.

Aspects of the invention disclosed herein are based, at least in part, on the following findings. Additional details are described herein in the Examples.

In experiments described herein, it was found that STK11 copy number loss is associated with or predictive of nonresponsiveness or resistance (e.g., acquired resistance) of a cancer (e.g., a CLL) to a PI3K inhibitor (e.g., Compound 1). Furthermore, in experiments described herein, it was found that a dual alteration in the MAPK/P53 pathway is associated with or predictive of nonresponsiveness or resistance (e.g., acquired resistance) of a cancer (e.g., a CLL) to a PI3K inhibitor (e.g., Compound 1).

In accordance with certain analyses described in the Examples, it was found that copy number loss of STK11 combined with copy number loss of TSC1, TSC2, or both is associated with or predictive of nonresponsiveness or resistance (e.g., acquired resistance) of a cancer (e.g., a CLL) to a PI3K inhibitor (e.g., Compound 1).

Also, in certain analyses described in the Examples, the following relationships were revealed. TSC2 copy number loss was associated with or predictive of nonresponsiveness or resistance (e.g., acquired resistance) of a cancer (e.g., a CLL) to a PI3K inhibitor (e.g., Compound 1). Copy number gain in each of BRAF, CTNNB1, FHIT, IRF4, MITF, MN1, and NF2 was associated with or predictive of nonresponsiveness or resistance (e.g., acquired resistance) of a cancer (e.g., a CLL) to a PI3K inhibitor (e.g., Compound 1). Copy number loss in each of NF2 and RET was associated with or predictive of nonresponsiveness or resistance (e.g., acquired resistance) of a cancer (e.g., a CLL) to a PI3K inhibitor (e.g., Compound 1). Loss of heterozygosity in RB1 was associated with or predictive of nonresponsiveness or resistance (e.g., acquired resistance) of a cancer (e.g., a CLL) to a PI3K inhibitor (e.g., Compound 1). Copy number gain in RANBP17 was associated with responsiveness or lack of resistance (e.g., acquired resistance) of a cancer (e.g., a CLL) to a PI3K inhibitor (e.g., Compound 1). Loss of heterozygosity in each of FGFR3, GMPS, and WHSC1 is associated with or predictive of responsiveness or lack of resistance (e.g., acquired resistance) of a cancer (e.g., a CLL) to a PI3K inhibitor (e.g., Compound 1).

### 1. DEFINITIONS

Unless defined otherwise, all technical and scientific terms used herein have the same meaning as is commonly understood by one of skill in the art to which this specification pertains.

As used in the specification and claims, the singular form “a”, “an” and “the” includes plural references unless the context clearly dictates otherwise.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “agonist” as used herein refers to a compound or agent having the ability to initiate or enhance a biological function of a target protein or polypeptide, such as increasing the activity or expression of the target protein or polypeptide. Accordingly, the term “agonist” is defined in the context of the biological role of the target protein or polypeptide. While some agonists herein specifically interact with (e.g., bind to) the target, compounds and/or agents that initiate or enhance a biological activity of the target protein or polypeptide by interacting with other members of the signal transduction pathway of which the target polypeptide is a member are also specifically included within this definition.

The terms “antagonist” and “inhibitor” are used interchangeably, and they refer to a compound or agent having the ability to reduce or inhibit a biological function of a target protein or polypeptide, such as by reducing or inhibiting the activity or expression of the target protein or polypeptide. Accordingly, the terms “antagonist” and “inhibitor” are defined in the context of the biological role of the target protein or polypeptide. An inhibitor need not completely abrogate the biological function of a target protein or polypeptide, and in some embodiments reduces the activity by at least 50%, 60%, 70%, 80%, 90%, 95%, or 99%. While some antagonists herein specifically interact with (e.g., bind to) the target, compounds that inhibit a biological activity of the target protein or polypeptide by interacting with other members of the signal transduction pathway of which the target protein or polypeptide are also specifically included within this definition. Non-limiting examples of biological activity inhibited by an antagonist include those associated with the development, growth, or spread of a tumor, or an undesired immune response as manifested in autoimmune disease.

The term “effective amount” or “therapeutically effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, a daily dosage can be achieved by a single administration of the targeted dosage amount or multiple administrations of smaller dosage amount(s). For example, a 150 mg daily dosage can be achieved by a single administration of 150 mg of the therapeutic agent per day, two administrations of 75 mg of the therapeutic agent per day, or three administrations of 50 mg of the therapeutic agent per day, or the like.

As used herein, the terms “treatment”, “treating”, “palliating” and “ameliorating” are used interchangeably herein. These terms refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

As used herein, the terms “prevention” and “preventing” are used herein to refer to an approach for obtaining beneficial or desired results including, but not limited, to prophylactic benefit. For prophylactic benefit, the pharmaceutical compositions may be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

As used herein, the “aggressiveness” of a tumor or cancer refers to the rate at which the tumor is growing. Thus, a tumor is more aggressive than another tumor or cancer if it is proliferating at a higher rate. Other determinants can be used to measure the level of aggressiveness of a tumor or cancer, for example, based on the appearance of tumor or cancer cells under a microscope to determine the extent to which tumors are differentiated. A well-differentiated tumor tends to be more aggressive than a poorly-differentiated tumor or cancer.

The term “selective inhibition” or “selectively inhibit” as applied to a biologically active agent refers to the agent's ability to selectively reduce the target signaling activity as compared to off-target signaling activity, via direct or indirect interaction with the target. For example, a compound that selectively inhibits one isoform of PI3K over another isoform of PI3K has an activity of at least greater than about 1× against a first isoform relative to the compound's activity against the second isoform (e.g., at least about 2×, 3×, 5×, 10×, 20×, 50×, 100×, 200×, 500×, or 1000×). In certain embodiments, these terms refer to (1) a compound described herein that selectively inhibits the gamma isoform over the alpha, beta, or delta isoform; or (2) a compound described herein that selectively inhibits the delta isoform over the alpha, beta, or gamma isoform. By way of non-limiting example, the ratio of selectivity can be greater than a factor of about 1, greater than a factor of about 2, greater than a factor of about 3, greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by IC₅₀. In certain embodiments, the IC₅₀ can be measured by in vitro or in vivo assays. In certain embodiments, the PI3K gamma isoform IC₅₀ activity of a compound provided herein can be less than about 1000 nM, less than about 500 nM, less than about 400 nM, less than about 300 nM, less than about 200 nM, less than about 100 nM, less than about 75 nM, less than about 50 nM, less than about 25 nM, less than about 20 nM, less than about 15 nM, less than about 10 nM, less than about 5 nM, or less than about 1 nM. In certain embodiments, the PI3K delta isoform IC₅₀ activity of a compound provided herein can be less than about 1000 nM, less than about 500 nM, less than about 400 nM, less than about 300 nM, less than about 200 nM, less than about 100 nM, less than about 75 nM, less than about 50 nM, less than about 25 nM, less than about 20 nM, less than about 15 nM, less than about 10 nM, less than about 5 nM, or less than about 1 nM.

“Subject” or “patient” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

The term “in vivo” refers to an event that takes place in a subject's body.

The term “in vitro” refers to an event that takes places outside of a subject's body. For example, an in vitro assay encompasses any assay conducted outside of a subject. In vitro assays encompass cell-based assays in which cells, alive or dead, are employed. In vitro assays also encompass a cell-free assay in which no intact cells are employed.

Combination therapy, or “in combination with” refer to the use of more than one compound or agent to treat a particular disorder or condition. For example, Compound 1 may be administered in combination with at least one additional therapeutic agent. By “in combination with,” it is not intended to imply that the other therapy and Compound 1 must be administered at the same time and/or formulated for delivery together, although these methods of delivery are within the scope of this disclosure. Compound 1 can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. In general, each therapeutic agent will be administered at a dose and/or on a time schedule determined for that particular agent. The other therapeutic agent can be administered with Compound 1 herein in a single composition or separately in a different composition. Higher combinations, e.g., triple therapy, are also contemplated herein.

The terms “co-administration of” and “co-administering” and their grammatical equivalents, as used herein, encompass administration of two or more agents to subject so that both agents and/or their metabolites are present in the subject at the same or substantially the same time. In one embodiment, co-administration of a PI3K inhibitor with an additional anti-cancer agent (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The additional agent can be administered prior to, at the same time as, or subsequent to administration of the PI3K inhibitor, or in some combination thereof. Where the PI3K inhibitor is administered to the patient at repeated intervals, e.g., during a standard course of treatment, the additional agent can be administered prior to, at the same time as, or subsequent to, each administration of the PI3K inhibitor, or some combination thereof, or at different intervals in relation to the PI3K inhibitor treatment, or in a single dose prior to, at any time during, or subsequent to the course of treatment with the PI3K inhibitor. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “monotherapy” refers to the use of an agent individually (also referred to herein as alone) (e.g., as a single compound or agent), e.g., without a second active ingredient to treat the same indication, e.g., cancer. For example, in this context, the term monotherapy includes the use of either the PI3K inhibitor or the second agent individually to treat the cancer.

The term “synergy” or “synergistic” encompasses a more than additive effect of a combination of two or more agents compared to their individual effects. In certain embodiments, synergy or synergistic effect refers to an advantageous effect of using two or more agents in combination, e.g., in a pharmaceutical composition, or in a method of treatment. In certain embodiments, one or more advantageous effects is achieved by using a PI3K inhibitor in combination with a second therapeutic agent (e.g., one or more second therapeutic agents) as described herein.

In some embodiments, the synergistic effect is that a lower dosage of one or both of the agents is needed to achieve an effect. For example, the combination can provide a selected effect, e.g., a therapeutic effect, when at least one of the agents is administered at a lower dosage than the dose of that agent that would be required to achieve the same therapeutic effect when the agent is administered as a monotherapy. In certain embodiments, the combination of a PI3K inhibitor (e.g., Compound 1) and a second agent (as described herein) allows the PI3K inhibitor to be administered at a lower dosage than would be required to achieve the same therapeutic effect if the PI3K inhibitor were administered as a monotherapy.

In some embodiments, the synergistic effect is a reduction, prevention, delay, or decrease in the occurrence or the likelihood of occurrence of one or more side effects, toxicity, resistance, that would otherwise be associated with administration of at least one of the agents.

In some embodiments, the synergistic effect is a reduction in resistance (e.g., a decrease in a measure of resistance or a decreased likelihood of developing resistance), or a delay in the development of resistance, to at least one of the agents.

In some embodiments, the synergistic effect is a reduction in MRD. In certain embodiments, the combination of a PI3K inhibitor (e.g. a PI3K inhibitor described herein) and a second agent (e.g., a second agent described herein) is effective to reduce the MRD in the subject, e.g., below a level previously measured in the subject (e.g., the level measured before the combination was administered). In certain embodiments, the combination of a PI3K inhibitor and a second agent is effective to reduce the MRD in the subject below the level observed during or after treatment with a monotherapy, e.g., a monotherapy comprising either the PI3K inhibitor or the second agent. In certain embodiments, the MRD is decreased below the level observed during treatment with a monotherapy comprising the PI3K inhibitor. In certain embodiments, the MRD is decreased below the level observed during treatment with a monotherapy comprising the second agent. In certain embodiments, the combination is effective to reduce the level of MRD below a preselected cutoff value (e.g., 1 malignant cell in 100 normal cells, 1 malignant cell in 1000 normal cells, or 1 malignant cell in 10,000 normal cells, or 1 malignant cell in 100,000 normal cells). In certain embodiments, the preselected cutoff value is 1 malignant cell in 1000 normal cells. In certain embodiments, the preselected cutoff value is 1 malignant cell in 100,000 normal cells.

In some embodiments, a synergistic effect refers to the combination of a PI3K inhibitor (e.g., Compound 1, or a pharmaceutically acceptable form thereof), and a second therapeutic agent (e.g., one or more additional therapeutic agent(s), or a pharmaceutically acceptable form thereof, as described herein), results in a therapeutic effect greater than the additive effect of the PI3K inhibitor and the second agent.

In some embodiments, a synergistic effect means that combination index value is less than a selected value, e.g., for a given effect, e.g., at a selected percentage (e.g., 50%) inhibition or growth inhibition, e.g., as described herein in the Examples. In certain embodiments, the selected value is 1. In certain embodiments, the selected value is 0.7. In certain embodiments, the selected value is 0.5.

In some embodiments, a synergistic effect means that the synergy score is 1 or more. In certain embodiments, the synergy score is greater than 1. In certain embodiments, the synergy score is greater than 3.

Combination index (CI) is a measure of potency shifting. The combination index is known in the art and is described, e.g., in Chou et al., Adv Enzyme Regul 1984; 22: 27-55 and in U.S. Patent Publication No. 2013/0295102, the contents of which are incorporated herein by reference. A CI value of greater than 1 indicates antagonistic effect; a CI value of 1.0 is indicative of an additive effect; and a CI value of less than 1 is indicative of a synergistic effect resulting from the combination. The CI value can be determined at various percentages of inhibition or growth inhibition.

The CI provides an estimate of the fraction of the original (monotherapy) doses of each of two drugs would be needed in combination relative to the single agent doses required to achieve a chosen effect level. For example, when the combination index has a value of 0.1, only about one tenth of the total fractional amounts of the individual agents (expressed as a fraction of the amount of that agent when administered as a monotherapy to achieve a chosen effect) are needed for the combination to reach the same chosen effect level. For example, if a dose of 100 mg/kg of drug A individually or a dose of 200 mg/kg of drug B individually is needed to achieve the chosen effect, and the combination index is 0.1, then approximately 5 mg/kg of drug A and 10 mg/kg of drug B would achieve the chosen effect (one twentieth of the original doses of each of the single agents adds up to a total of one tenth). The doses of the single agents need not be reduced by the same fractional value so long as the sum of their fractional values adds up to the combination index; thus, in this example, a dose of approximately 8 mg/kg of drug A and 4 mg/kg of drug B would also achieve the chosen effect (this is 0.08 times the original dose of drug A and 0.02 times the original dose of drug B; the sum of the fractional amounts (0.08+0.02) is equal to the combination index of 0.1.)

According to one embodiment, synergy score is a measure of the combination effects in excess of Loewe additivity. In one example, synergy score is a scalar measure to characterize the strength of synergistic interaction. The Synergy score can be calculated as:

Synergy Score=log f_{X} log f_{Y}Σmax(O,I_{data})(I_{data}−I_{Loewe})

In this example, the fractional inhibition for each component agent and combination point in the matrix is calculated relative to the median of all vehicle-treated control wells. The example Synergy Score equation integrates the experimentally-observed activity volume at each point in the matrix in excess of a model surface numerically derived from the activity of the component agents using the Loewe model for additivity. Additional terms in the Synergy Score equation (above) are used to normalize for various dilution factors used for individual agents and to allow for comparison of synergy scores across an entire experiment. The inclusion of positive inhibition gating or an I_{data} multiplier removes noise near the zero effect level, and biases results for synergistic interactions at that occur at high activity levels. According to other embodiments, a synergy score can be calculated based on a curve fitting approach where the curvature of the synergy score is extrapolated by introducing a median value and origin value (e.g., a dose zero value).

The synergy score measure can be used for the self-cross analysis. Synergy scores of self-crosses are expected to be additive by definition and, therefore, maintain a synergy score of zero. However, while some self-cross synergy scores are near zero, many are greater suggesting that experimental noise or non-optimal curve fitting of the single agent dose responses are contributing to the slight perturbations in the score. This strategy is cell line-centric, focusing on self-cross behavior in each cell line versus a global review of cell line panel activity. Combinations where the synergy score is greater than the mean self-cross plus two standard deviations or three standard deviations can be considered candidate synergies at 95% and 99% confidence levels, respectively. Additivity should maintain a synergy score of zero, and synergy score of two or three standard deviations indicate synergism at statistically significant levels of 95% and 99%.

Loewe Volume (Loewe Vol) is used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself. The calculation for Loewe additivity is:

I_{Loewe} that satisfies(X/X₁)+(Y/Y₁)=1

where X₁ and Y₁ are the single agent effective concentrations for the observed combination effect I. For example, if 50% inhibition is achieved separately by 1 μM of drug A or 1 μM of drug B, a combination of 0.5 μM of A and 0.5 μM of B should also inhibit by 50%.

As used herein, a daily dosage can be achieved by a single administration of the targeted dosage amount or multiple administrations of smaller dosage amount(s). For example, a 150 mg daily dosage can be achieved by a single administration of 150 mg of the therapeutic agent per day, two administrations of 75 mg of the therapeutic agent per day, or three administrations of 50 mg of the therapeutic agent per day, or the like.

The term “anti-cancer effect” refers to the effect a therapeutic agent has on cancer, e.g., a decrease in growth, viability, or both of a cancer cell. The IC₅₀ of cancer cells can be used as a measure the anti-cancer effect.

IC₅₀ refers to a measure of the effectiveness of a therapeutic agent in inhibiting cancer cells by 50%.

The term “tumor” refers to any neoplastic cell growth and proliferation, whether malignant or benign, and any pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus.

Hematopoietic origin refers to involving cells generated during hematopoiesis, a process by which cellular elements of blood, such as lymphocytes, leukocytes, platelets, erythrocytes and natural killer cells are generated. Cancers of hematopoietic origin includes lymphoma and leukemia.

Resistant or refractive refers to when a cancer that has a reduced responsiveness to a treatment, e.g., up to the point where the cancer does not respond to treatment. The cancer can be resistant at the beginning of treatment, or it may become resistant during treatment. The cancer subject may have one or more mutations that cause it to become resistant to the treatment, or the subject may have developed such mutations during treatment. The term “refractory” can refer to a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

“Copy number loss” as used herein refers to the loss of one or more copies of a DNA sequence from a genome. In some embodiments, the DNA sequence comprises a gene. In some embodiments, the DNA sequence comprises a portion of a gene, e.g., such that loss of the portion reduces or abrogates the gene function. In some embodiments, copy number loss is a result of a deletion, chromosome loss, or chromosome breakage event.

“Responsiveness,” to “respond” to treatment, and other forms of this term, as used herein, refer to the reaction of a subject to treatment with a therapeutic, e.g., a PI3K inhibitor, alone or in combination, e.g., monotherapy or combination therapy. In one embodiment, a response to a PI3K inhibitor is determined. Responsiveness to a therapy, e.g., treatment with a PI3K inhibitor alone or in combination, can be evaluated by using any of the alterations/biomarkers disclosed herein and/or comparing a subject's response to the therapy using one or more clinical criteria, such as IWCLL 2008 (for CLL) described in, e.g., Hallek, M. et al. (2008) Blood 111 (12): 5446-5456; RECIST criteria for solid tumors (Response Evaluation Criteria In Solid Tumors), and the like. Additional classifications of responsiveness are provided in Brown, J. R. (2014) Blood, 123(22):3390-3397 and Chesson, B. D. et al. Journal of Clinical Oncology, 30(23):2820-2822.

These criteria provide a set of published rules that define when cancer patients improve (“respond”), stay the same (“stable”) or worsen (“progression”) during treatments.

In one embodiment, a subject having CLL can be determined to be in complete remission (CR) or partial remission (PR). For example, according to IWCLL 2008, a subject is considered to be in CR if at least all of the following criteria as assessed after completion of therapy are met: (i) Peripheral blood lymphocytes (evaluated by blood and different count) below 4×10⁹/L (4000 μL); (ii) no hepatomegaly or splenomegaly by physical examination; (iii) absence of constitutional symptoms; and (iv) blood counts (e.g., neutrophils, platelets, hemoglobin) above the values set forth in Hallek, M. et al. supra at page 5451). Partial remission (PR) for CLL is defined according to IWCLL 2008 as including one of: (i) a decrease in number of blood lymphocytes by 50% or more from the value before therapy; (ii) a reduction in lymphadenopathy, as detected by CT scan or palpation; or (iii) a reduction in pretreatment enlargement of spleen or liver by 50% or more, as detected by CT scan or palpation; and blood counts (e.g., neutrophils, platelets, hemoglobin) according to the values set forth in Hallek, M. et al. supra at page 5451).

In other embodiments, a subject having CLL is determined to have progressive disease (PD) or stable disease (SD). For example, according to IWCLL 2008, a subject is considered to be in PD during therapy or after therapy if at least one of the following criteria is met: (i) progression on lymphadenopathy; (ii) an increase in pretreatment enlargement of spleen or liver by 50% or more, or de novo appearance of hepatomegaly or splenomegaly; (iii) an increase in the number of blood lymphocytes by 50% or more with at least 5000 B lymphocytes per microliter; (iv) transformation to a more aggressive histology (e.g., Richter syndrome); or (v) occurrence of cytopenia (neutropenia, anemia or thrombocytopenia) attributable to CLL, as described in Hallek, M. et al. supra at page 5452. Stable disease (SD) for CLL is defined according to IWCLL 2008 as a patient who has not achieved CR or a PR, and who has not exhibited progressive disease, see Hallek, M. et al. supra at page 5452.

In one embodiment, a subject with CLL responds to treatment with an PI3K inhibitor if at least one of the criteria for disease progression according to IWCLL is retarded or reduced, e.g., by about 10%, 20%, 30%, 40%, 50%, 60%, 70%, 80%, 90% or more. In another example, a subject responds to treatment with a PI3K inhibitor, if the subject experiences a life expectancy extension, e.g., extended by about 5%, 10%, 20%, 30%, 40%, 50% or more beyond the life expectancy predicted if no treatment is administered. In another example, a subject responds to treatment with a PI3K inhibitor, if the subject has one or more of: an increased progression-free survival, overall survival or increased time to progression (TTP), e.g., as described in Hallek, M. et al. supra at page 5452.

In another embodiment in solid tumors, a subject responds to treatment with a PI3K inhibitor if growth of a tumor in the subject is retarded about 10%, 20%, 30%, 40%, 50%, 60%, 70%, 80%, 90% or more. In another example, a subject responds to treatment with a PI3K inhibitor, if a tumor in the subject shrinks by about 5%, 10%, 20%, 30%, 40%, 50% or more as determined by any appropriate measure, e.g., by mass or volume. In another example, a subject responds to treatment with a PI3K inhibitor, if the subject experiences a life expectancy extended by about 5%, 10%, 20%, 30%, 40%, 50% or more beyond the life expectancy predicted if no treatment is administered. In another example, a subject responds to treatment with a PI3K inhibitor, if the subject has an increased disease-free survival, overall survival or increased time to progression. Several methods can be used to determine if a patient responds to a treatment including the RECIST criteria, as set forth above.

“Acquire” or “acquiring” as the terms are used herein, refer to obtaining possession of, determining, or evaluating, a value or information (e.g., one or more of: the presence, absence, amount or level) of an alteration or biomarker, by “directly acquiring” or “indirectly acquiring” the same. “Directly acquiring” means performing a process (e.g., performing a test) to obtain the value or information of the alteration or biomarker. “Indirectly acquiring” refers to receiving the value or information of the alteration or biomarker from another party or source (e.g., a diagnostic provider, a third party clinician or health professional).

“Alteration” of a gene or gene product (e.g., a biomarker gene or gene product) or an “altered gene” or “altered gene product” as used herein, refers to the presence of a mutation (e.g., one or more mutations) within a gene or gene product, which affects the structure, amount or activity of the gene or gene product, as compared to a reference gene or gene product, e.g., a normal or wild-type gene or gene product, or a responder gene or gene product (e.g., a gene or gene product in a responder subject (e.g., a subject in complete or partial cancer remission)). The alteration can be in amount, structure, and/or activity in a cancer tissue or cancer cell, as compared to its amount, structure, and/or activity, in a reference tissue or cell (e.g., a normal or healthy tissue or cell, or a responder tissue or cell (e.g., a tissue or cell from a subject in complete or partial cancer remission)). The alteration can be associated with, or be indicative of, a disease state, such as cancer (e.g., a hematologic malignancy as described herein, e.g., CLL). For example, an alteration which is associated with cancer, or is predictive of responsiveness or non-responsiveness to an anti-cancer therapeutic (e.g., a PI3K inhibitor disclosed herein), can have an altered nucleotide sequence (e.g., a mutation), amino acid sequence, chromosomal translocation, intra-chromosomal inversion, copy number, expression level, protein level, protein activity, or methylation status, in a cancer tissue or cancer cell, as compared to a reference tissue or cell. Exemplary mutations include, but are not limited to, point mutations (e.g., silent, missense, or nonsense), deletions, insertions, inversions, linking mutations, duplications, copy number changes, translocations, inter- and intra-chromosomal rearrangements. Mutations can be present in the coding or non-coding region of the gene (e.g., one or more exons, the 5′- and/or 3′-UTR).

In certain embodiments, the alteration(s) are associated (or not associated) with a phenotype, e.g., a cancerous phenotype (e.g., one or more of cancer risk; cancer progression; responsiveness to a cancer treatment (e.g., complete or partial remission); or decreased responsiveness or non-responsiveness to a cancer treatment (e.g., progressive or stable disease, or resistance, e.g., acquired resistance) to a cancer treatment). In one embodiment, the alteration is associated with, or is, a prognosis-positive predictor or a prognosis-negative predictor (also referred to herein as a “prognosis-positive alteration” or a “prognosis-negative alteration”). In another embodiment, the alteration is associated with, or is, a progression-positive predictor or a progression-negative predictor (also referred to herein as a “progression-positive alteration” or a “progression-negative alteration”).

As used herein, the term ‘prognosis-positive predictor’ refers to any alteration that indicates increased responsiveness (e.g., increased sensitivity) to a PI3K inhibitor. The prognosis-positive predictor can be evaluated relative to a reference value, e.g., a normal or wild-type gene or gene product, or a responder gene or gene product (e.g., a gene or gene product in a responder subject (e.g., a subject in complete or partial cancer remission)). Subjects in complete or partial cancer remission (e.g., CR or PR subjects as described herein) can have one or more prognosis-positive alterations.

The term ‘prognosis-negative predictor’ refers to any alteration that indicates decreased responsiveness (e.g., sensitivity) to a PI3K inhibitor. The prognosis-negative predictor can be evaluated relative to a reference value, e.g., a reference value disclosed herein. Subjects with progressive disease or stable disease (e.g., PD or SD subjects as described herein) can have one or more prognosis-negative alterations. This term can include a subject who has resistance (e.g., has developed or acquired resistance) to a PI3K inhibitor.

The term ‘progression-positive predictor’ refers to any alteration that indicates increased progression or increased likelihood of cancer progression. The progression-positive predictor can be evaluated relative to a reference value, e.g., a reference value disclosed herein. Subjects with progressive disease or stable disease (e.g., PD or SD subjects as described herein) can have one or more progression-positive alterations. This term can include a subject who has resistance (e.g., has developed or acquired resistance) to a PI3K inhibitor.

The term ‘progression-negative predictor’ refers to any alteration that indicates decreased progression or decreased likelihood of cancer progression. The progression-negative predictor can be evaluated relative to a reference value, e.g., a reference value disclosed herein. Subjects in complete or partial cancer remission (e.g., CR or PR subjects as described herein) can have one or more progression-negative alterations.

A “biomarker” or “marker” is a substance, e.g., a gene or gene product (e.g., mRNA or protein) which can be altered (e.g., having an alteration described herein), wherein said alteration is associated with, or is indicative of, a disease state, e.g., a cancer (e.g., a hematological malignancy described herein, e.g., CLL). The alteration can be in amount, structure, and/or activity of the substance (e.g., gene or gene product) in a cancer tissue or cancer cell, as compared to its amount, structure, and/or activity, in a reference sample, e.g., a normal or wild-type gene or gene product, or a responder gene or gene product (e.g., a gene or gene product in a responder subject (e.g., a subject in complete or partial cancer remission). For example, a biomarker described herein which is associated with cancer or predictive of responsiveness to anti-cancer therapeutics can have an altered nucleotide sequence, amino acid sequence, chromosomal translocation, intra-chromosomal inversion, copy number, expression level, protein level, protein activity, or methylation status, in a cancer tissue or cancer cell as compared to a normal, healthy tissue or cell. Furthermore, a “biomarker” includes a molecule whose structure is altered, e.g., mutated (contains an mutation), e.g., differs from the wild type sequence at the nucleotide or amino acid level, e.g., by substitution, deletion, or insertion, when present in a tissue or cell associated with a disease state, such as cancer. In some embodiments, a biomarker can be evaluated individually, or in combinations with one or more other biomarkers.

As used herein, the term ‘prognosis-positive biomarker’ refers to any biomarker that indicates increased responsiveness (e.g., increased sensitivity) to a PI3K inhibitor. The prognosis-positive biomarker can be evaluated relative to a reference value, e.g., a normal or wild-type gene or gene product, or a responder gene or gene product (e.g., a gene or gene product in a responder subject (e.g., a subject in complete or partial cancer remission)). Subjects in complete or partial cancer remission (e.g., CR or PR subjects as described herein) can have one or more prognosis-positive biomarkers.

The term ‘prognosis-negative biomarker’ refers to any biomarker that indicates decreased responsiveness (e.g., sensitivity) to a PI3K inhibitor. The prognosis-negative biomarker can be evaluated relative to a reference value, e.g., a reference value disclosed herein. Subjects with progressive disease or stable disease (e.g., PD or SD subjects as described herein) can have one or more prognosis-negative biomarkers. This term can include a subject who has resistance (e.g., has developed or acquired resistance) to a PI3K inhibitor.

The term ‘progression-positive biomarker’ refers to any biomarker that indicates increased progression or increased likelihood of cancer progression. The progression-positive biomarker can be evaluated relative to a reference value, e.g., a reference value disclosed herein. Subjects with progressive disease or stable disease (e.g., PD or SD subjects as described herein) can have one or more progression-positive biomarker. This term can include a subject who has resistance (e.g., has developed or acquired resistance) to a PI3K inhibitor.

The term ‘progression-negative biomarker’ refers to any biomarker that indicates decreased progression or decreased likelihood of cancer progression. The progression-negative biomarker can be evaluated relative to a reference value, e.g., a reference value disclosed herein. Subjects in complete or partial cancer remission (e.g., CR or PR subjects as described herein) can have one or more progression-negative biomarkers.

One skilled in the art can recognize that a prognostic biomarker may be used as a diagnostic biomarker or a predictive biomarker, and terms such as ‘prognosis-positive’, ‘prognosis-negative’, ‘progression-positive’ and progression-negative′ and the like may refer to biomarkers used in methods involving prediction or diagnosis.

### Chemical Definitions

As used herein, a “pharmaceutically acceptable form” of a disclosed compound includes, but is not limited to, pharmaceutically acceptable salts, hydrates, solvates, isomers, prodrugs, and isotopically labeled derivatives of disclosed compounds. In one embodiment, a “pharmaceutically acceptable form” includes, but is not limited to, pharmaceutically acceptable salts, isomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts may be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts may be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, the pharmaceutically acceptable form is a solvate (e.g., a hydrate). As used herein, the term “solvate” refers to compounds that further include a stoichiometric or non-stoichiometric amount of solvent bound by non-covalent intermolecular forces. The solvate may be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or one to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” refers to compounds that are transformed in vivo to yield a disclosed compound or a pharmaceutically acceptable form of the compound. A prodrug may be inactive when administered to a subject, but is converted in vivo to an active compound, for example, by hydrolysis (e.g., hydrolysis in blood). In certain cases, a prodrug has improved physical and/or delivery properties over the parent compound. Prodrugs are typically designed to enhance pharmaceutically and/or pharmacokinetically based properties associated with the parent compound. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, Chp 1, pp 1-12 and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it enhances absorption from the digestive tract, or it can enhance drug stability for long-term storage.

The term “prodrug” is also meant to include any covalently bonded carriers, which release the active compound in vivo when such prodrug is administered to a subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed., 1995), and Design of Prodrugs (H. Bundgaard ed., Elsevier, New York, 1985).

For example, if a disclosed compound or a pharmaceutically acceptable form of the compound contains a carboxylic acid functional group, a prodrug can comprise a pharmaceutically acceptable ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino (C₂-C₃)alkyl.

Similarly, if a disclosed compound or a pharmaceutically acceptable form of the compound contains an alcohol functional group, a prodrug may be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from naturally occurring L-amino acids, P(O)(OH)₂, —P(O)(O(C₁-C₆)alkyl)₂, and glycosyl (the radical resulting from the removal of a hydroxyl group of the hemiacetal form of a carbohydrate).

If a disclosed compound or a pharmaceutically acceptable form of the compound incorporates an amine functional group, a prodrug may be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, a natural α-aminoacyl or natural α-aminoacyl-natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino, morpholino, piperidin-1-yl or pyrrolidin-1-yl.

In certain embodiments, the pharmaceutically acceptable form is an isomer. “Isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown may be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that may be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which can potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)- and 20% (R)-, the enantiomeric purity of the compound with respect to the (S)-isomeric form is 80%. The enantiomeric purity of a compound may be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or the Pirkle alcohol, or derivatization of a compounds using a chiral compound such as Mosher's acid followed by chromatography or nuclear magnetic resonance spectroscopy.

In certain embodiments, the pharmaceutically acceptable form is a tautomer. As used herein, the term “tautomer” is a type of isomer that includes two or more interconvertable compounds resulting from at least one formal migration of a hydrogen atom and at least one change in valency (e.g., a single bond to a double bond, a triple bond to a double bond, or a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers may be reached. Tautomerizations (i.e., the reaction providing a tautomeric pair) may be catalyzed by acid or base, or can occur without the action or presence of an external agent. Exemplary tautomerizations include, but are not limited to, keto-enol; amide-imide; lactam-lactim; enamine-imine; and enamine-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Unless otherwise stated, structures depicted herein are also meant to include compounds which differ only in the presence of one or more isotopically enriched atoms. For example, compounds having the present structures except for the replacement or enrichment of a hydrogen by deuterium or tritium at one or more atoms in the molecule, or the replacement or enrichment of a carbon by ¹³C or ¹⁴C at one or more atoms in the molecule, are within the scope of this disclosure. In one embodiment, provided herein are isotopically labeled compounds having one or more hydrogen atoms replaced by or enriched by deuterium. In one embodiment, provided herein are isotopically labeled compounds having one or more hydrogen atoms replaced by or enriched by tritium. In one embodiment, provided herein are isotopically labeled compounds having one or more carbon atoms replaced or enriched by ¹³C. In one embodiment, provided herein are isotopically labeled compounds having one or more carbon atoms replaced or enriched by ¹⁴C.

The disclosure also embraces isotopically labeled compounds which are identical to those recited herein, except that one or more atoms are replaced by an atom having an atomic mass or mass number different from the atomic mass or mass number usually found in nature. Examples of isotopes that may be incorporated into disclosed compounds include isotopes of hydrogen, carbon, nitrogen, oxygen, phosphorus, sulfur, fluorine, and chlorine, such as, e.g., ²H, ³H, ¹³C, ¹⁴C, ¹⁵N, ¹⁸O, ¹⁷O, ³¹P, ³²P, ³⁵S, ¹⁸F, and ³⁶Cl, respectively. Certain isotopically-labeled disclosed compounds (e.g., those labeled with ³H and/or ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure.

As used herein, and unless otherwise specified, “polymorph” may be used herein to describe a crystalline material, e.g., a crystalline form. In certain embodiments, “polymorph” as used herein are also meant to include all crystalline and amorphous forms of a compound or a salt thereof, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms, as well as mixtures thereof, unless a particular crystalline or amorphous form is referred to. Compounds of the present disclosure include crystalline and amorphous forms of those compounds, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms of the compounds or a salt thereof, as well as mixtures thereof.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions as disclosed herein is contemplated. Supplementary active ingredients can also be incorporated into the pharmaceutical compositions.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

### 2. COMPOSITIONS AND METHODS

In the methods described herein, the PI3K inhibitor can be any PI3K inhibitor as described herein below, including pharmacologically acceptable salts or polymorphs thereof.

As used herein, a “phosphoinositide 3-kinase (PI3K) inhibitor” or “PI3K inhibitor” refers to an inhibitor of any PI3K. PI3Ks are members of a unique and conserved family of intracellular lipid kinases that phosphorylate the 3′—OH group on phosphatidylinositols or phosphoinositides. The PI3K family includes kinases with distinct substrate specificities, expression patterns, and modes of regulation (see, e.g., Katso et al., 2001, Anna. Rev. Cell Dev. Biol. 17, 615-675; Foster, F. M. et al., 2003, J Cell Sci 116, 3037-3040). The class I PI3Ks (e.g., p110 α, p110 β, p110 γ, and p110 δ) are typically activated by tyrosine kinases or G-protein coupled receptors to generate PIP3, which engages downstream mediators such as those in the Akt/PDK1 pathway, mTOR, the Tec family kinases, and the Rho family GTPases. The class II PI3Ks (e.g., PI3K-C2α, PI3K-C2β, PI3K-C2γ) and III PI3Ks (e.g., Vps34) play a key role in intracellular trafficking through the synthesis of PI(3)P and PI(3,4)P2. Specific exemplary PI3K inhibitors are disclosed herein.

The class I PI3Ks comprise a p110 catalytic subunit and a regulatory adapter subunit. See, e.g., Cantrell, D. A. (2001) Journal of Cell Science 114: 1439-1445. Four isoforms of the p110 subunit (including PI3K-α (alpha), PI3K-β (beta), PI3K-γ (gamma), and PI3K-δ (delta) isoforms) have been implicated in various biological functions. Class I PI3Kα is involved, for example, in insulin signaling, and has been found to be mutated in solid tumors. Class I PI3K-β is involved, for example, in platelet activation and insulin signaling. Class I PI3K-γ plays a role in mast cell activation, innate immune function, and immune cell trafficking (chemokines). Class I PI3K-δ is involved, for example, in B-cell and T-cell activation and function and in Fc receptor signaling in mast cells. In some embodiments provided herein, the PI3K inhibitor is a class I PI3K inhibitor. In some such embodiments, the PI3K inhibitor inhibits a PI3K-α (alpha), PI3K-β (beta), PI3K-γ (gamma), or PI3K-δ (delta) isoform, or a combination thereof.

Downstream mediators of the PI3K signal transduction pathway include Akt and mammalian target of rapamycin (mTOR). Manning et al., Cell 129, 1261-1274 Jun. 29, 2007. Akt possesses a plckstrin homology (PH) domain that binds PIP3, leading to Akt kinase activation. Akt phosphorylates many substrates and is a central downstream effector of PI3K for diverse cellular responses. One important function of Akt is to augment the activity of mTOR, through phosphorylation of TSC2 and other mechanisms. mTOR is a serine-threonine kinase related to the lipid kinases of the PI3K family. Laplante et al., Cell 149,274-293 Apr. 13, 2012 mTOR has been implicated in a wide range of biological processes including cell growth, cell proliferation, cell motility and survival. Disregulation of the mTOR pathway has been reported in various types of cancer. mTOR is a multifunctional kinase that integrates growth factor and nutrient signals to regulate protein translation, nutrient uptake, autophagy, and mitochondrial function.

MEK inhibitor is an agent that inhibits the mitogen-activated protein kinase kinase enzyme MEK1 and/or MEK2. Neuzillet et al., Pharmacology & Therapeutics 141 (2014) 160-17. The MAPK/ERK pathway is often overactive in certain cancers. The MEK-ERK is a pathway that regulates cell growth, proliferation, differentiation, and apoptosis in response to growth factors, cytokines, and hormones. This pathway transmits signals from multiple cell surface receptors to transcription factors in the nucleus which regulates gene expression. This pathway operates downstream of Ras which is upregulated or mutated in human tumors. MEK is a critical effector of Ras function. Many cancers involve activating Ras mutations. Inhibition of the ERK pathway and inhibition of MEK kinase activity can produce anti-metastatic and anti-angiogenic effects by reducing cell-cell contact and motility in addition to downregulation of vascular endothelial growth factor (VEGF) expression.

Proteasomes play a role in the degradation process of proteins. Proteins are tagged for degradation with a small protein called ubiquitin. The tagging reaction is catalyzed by enzymes called ubiquitin ligases. Once a protein is tagged with a single ubiquitin molecule, this is a signal to other ligases to attach additional ubiquitin molecules. The result is a polyubiquitin chain that is bound by the proteasome, allowing it to degrade the tagged protein. This degradation process is important for many cellular processes, including the cell cycle, the regulation of gene expression, and responses to oxidative stress. Proteasomes play certain roles in the apoptotic process. The involvement of the proteasome in this process is indicated by both the increase in protein ubiquitination, and of E1, E2, and E3 enzymes that is observed in advance of apoptosis. Proteasome inhibition has different effects on apoptosis induction in different cell types. Apoptosis is mediated through disrupting the regulated degradation of pro-growth cell cycle proteins. The ability of proteasome inhibitors to induce apoptosis in rapidly dividing cells indicates that they can be used in cancer therapy. Proteasomes are protein complexes that degrade unneeded or damaged proteins by proteolysis, a chemical reaction that breaks peptide bonds. Richardson et al., Cell Cycle 4:2, 290-296; February 2005.

There is a need for an effective and safe combination therapy involving a PI3K inhibitor, and a MEK, AKT, mTOR, or proteasome inhibitor for treating cancers.

In certain embodiments, provided herein are pharmaceutical compositions comprising a PI3K inhibitor, or a pharmaceutically acceptable form thereof, in combination with a second agent or a pharmaceutically acceptable form thereof, wherein the second agent is selected from one or more of 1) a MEK inhibitor, 2) a mTOR inhibitor, 3) an AKT inhibitor, 4) a proteasome inhibitor, 5) an immune modulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor. In certain embodiments, the combination is therapeutically effective. In certain embodiments, the combination is synergistic, e.g., has one or more synergistic effects, e.g., synergistic therapeutic effects.

Also provided herein are methods of treating (e.g., inhibiting, managing, or preventing) a cancer in a subject comprising administering to the subject a PI3K inhibitor, or a pharmaceutically acceptable form thereof, in combination with a second agent (e.g., one or more second agents), or a pharmaceutically acceptable form thereof, wherein the second agent is selected from one or more of 1) a MEK inhibitor, 2) a mTOR inhibitor, 3) an AKT inhibitor, 4) a proteasome inhibitor, 5) an immunomodulator, 6) a glucocorticosteroid, 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor. In certain embodiments, the combination is therapeutically effective. In certain embodiments, the combination is synergistic.

In certain embodiments, the compositions and methods provided herein are utilized where a monotherapy of one of the therapeutic agents is becoming less effective due to drug resistance or where the relatively high dosage of monotherapy lead to undesirable side effects.

2.1 PI3K Inhibitors

PI3K inhibitors that can be used in the compositions and methods provided herein include, but are not limited to, those described in, e.g., WO 09/088990, WO 09/088086, WO 2011/008302, WO 2010/036380, WO 2010/006086, WO 09/114870, WO 05/113556, WO2014072937, WO2014071125, US 2009/0312310, and US 2011/0046165, the entirety of each incorporated herein by reference. Additional PI3K inhibitors that can be used in the compositions and methods provided herein include, but are not limited to, AMG-319, GSK 2126458 (2,4-Difluoro-N-{2-(methyloxy)-5-[4-(4-pyridazinyl)-6-quinolinyl]-3-pyridinyl}benzenesulfonamide), GSK 1059615 (5Z-[[4-(4-pyridinyl)-6-quinolinyl]methylene]-2,4-thiazolidinedione), GDC-0032 (4-[5,6-dihydro-2-[3-methyl-1-(1-methylethyl)-1H-1,2,4-triazol-5-yl]imidazo[1,2-d][1,4]benzoxazepin-9-yl]-α,α-dimethyl-1H-Pyrazole-1-acetamide), GDC-0980 ((S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one), GDC-0941 (2-(1H-indazol-4-yl)-6-((4-(methylsulfonyl)piperazin-1-yl)methyl)-4-morpholinothieno[3,2-d]pyrimidine), XL147 (N-(3-(benzo[c][1,2,5]thiadiazol-5-ylamino)quinoxalin-2-yl)-4-methylbenzenesulfonamide), XL499, XL765 (SAR245409, N-[4-[[[3-[(3,5-dimethoxyphenyl)amino]-2-quinoxalinyl]amino]sulfonyl]phenyl]-3-methoxy-4-methyl-benzamide), PF-4691502 (2-amino-6-(6-methoxypyridin-3-yl)-4-methyl-8-[(1R,4R)-4-(2-hydroxyethoxyl)cyclohexyl]-7H,8H-pyrido[2,3-d]pyrimidin-7-one), BKM 120 (buparlisib, 5-(2,6-dimorpholinopyrimidin-4-yl)-4-(trifluoromethyl)pyridin-2-amine), Idelalisib (CAL-101, GS1101, (S)-2-(1-(9H-purin-6-ylamino)propyl)-5-fluoro-3-phenylquinazolin-4(3H)-one), CAL 263, SF1126 (3-[[2-[[5-[[amino(azaniumyl)methylidene]amino]-2-[[4-oxo-4-[4-(4-oxo-8-phenylchromen-2-yl)morpholin-4-ium-4-yl]oxybutanoyl]amino]pentanoyl]amino]acetyl]amino]-4-(1-carboxylatopropylamino)-4-oxobutanoate), PX-866 (sonolisib, [(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5-h]isochromen-10-yl]acetate), BEZ235 (2-methyl-2-(4-(3-methyl-2-oxo-8-(quinolin-3-yl)-2,3-dihydroimidazo[4,5-c]quinolin-1-yl)phenyl)propanenitrile), GS9820 (CAL-120, (S)-2-(1-((9H-purin-6-yl)amino)ethyl)-6-fluoro-3-phenylquinazolin-4(3H)-one), BYL719 ((2S)-1,2-Pyrrolidinedicarboxamide, N1-[4-methyl-5-[2-(2,2,2-trifluoro-1,1-dimethylethyl)-4-pyridinyl]-2-thiazolyl]), RP6503, RP6530, TGR1202 (((S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one)), INK1117 (MLN-1117), PX-866, BAY 80-6946 (2-amino-N-(7-methoxy-8-(3-morpholinopropoxy)-2,3-dihydroimidazo[1,2-c]quinazolin-5-yl)pyrimidine-5-carboxamide), IC87114 (2-((6-amino-9H-purin-9-yl)methyl)-5-methyl-3-o-tolylquinazolin-4(3H)-one), Palomid 529 (3-(4-methoxybenzyloxy)-8-(1-hydroxyethyl)-2-methoxy-6H-benzo[c]chromen-6-one), ZSTK474 (2-(difluoromethyl)-1-(4,6-dimorpholino-1,3,5-triazin-2-yl)-1H-benzo[d]imidazole), PWT33597, TG100-115 (6,7-Bis(3-hydroxyphenyl)pteridine-2,4-diamine), GNE-477 (5-[7-methyl-4-(morpholin-4-yl)-6-[(4-methylsulfonylpiperazin-1-yl)methyl]thieno[3,2-d]pyrimidin-2-yl]pyrimidin-2-amine), CUDC-907 (N-hydroxy-2-(((2-(6-methoxypyridin-3-yl)-4-morpholinothieno[3,2-d]pyrimidin-6-yl)methyl)(methyl)amino)pyrimidine-5-carboxamide), AEZS-136, BGT-226 (8-(6-methoxypyridin-3-yl)-3-methyl-1-(4-(piperazin-1-yl)-3-(trifluoromethyl)phenyl)-1H-imidazo[4,5-c]quinolin-2(3H)-one maleic acid), PF-05212384 (1-(4-(4-(dimethylamino)piperidine-1-carbonyl)phenyl)-3-(4-(4,6-dimorpholino-1,3,5-triazin-2-yl)phenyl)urea), LY3023414, PI-103 (3-[4-(4-morpholinyl)pyrido[3′,2′:4,5]furo[3,2-d]pyrimidin-2-yl]-phenol), INCB040093, CAL-130 ((S)-2-(1-((2-amino-9H-purin-6-yl)amino)ethyl)-5-methyl-3-(o-tolyl)quinazolin-4(3H)-one), LY294002 (2-Morpholin-4-yl-8-phenylchromen-4-one) and wortmannin.

In one embodiment, the PI3K inhibitor is Idelalisib (GS1101), CAL-130, BKM 120, GDC-0941, PX-866, GDC-0032, BAY 80-6946, BEZ235, BYL719, BGT-226, PF-4691502, GDC-0980, GSK 2126458, PF-05212384, XL765, or XL147.

In one embodiment, the PI3K inhibitor is Idelalisib (also known as GS1101 or CAL-101) and has the chemical name (S)-2-(1-(9H-purin-6-ylamino)propyl)-5-fluoro-3-phenylquinazolin-4(3H)-one and the following structure:

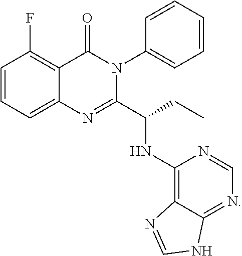 [SMILES: CC[C@H](Nc1ncnc2[nH]cnc12)c1nc2cccc(F)c2c(=O)n1-c1ccccc1]

In certain embodiments, a PI3K inhibitor is a compound that inhibits one or more PI3K isoforms, e.g., alpha, beta, delta, or gamma isoform. In one embodiment, a PI3K inhibitor is a compound that inhibits one or more PI3K isoforms with an IC₅₀ of less than about 1000 nM, less than about 900 nM, less than about 800 nM, less than about 700 nM, less than about 600 nM, less than about 500 nM, less than about 400 nM, less than about 300 nM, less than about 200 nM, less than about 100 nM, less than about 75 nM, less than about 50 nM, less than about 25 nM, less than about 20 nM, less than about 15 nM, less than about 10 nM, less than about 10 nM, less than about 5 nM, or less than about 1 nM.

In one embodiment, the PI3K inhibitor is a compound that inhibits alpha, beta, delta and gamma isoforms. In another embodiment, the PI3K inhibitor is a compound that inhibits beta, delta, and gamma isoforms. In another embodiment, the PI3K inhibitor is a compound that inhibits the delta and gamma isoforms.

In certain embodiments, the PI3K inhibitor is a PI3K isoform selective inhibitor. In one embodiment, the PI3K inhibitor is a PI3K alpha selective inhibitor. In another embodiment, the PI3K inhibitor is a PI3K beta selective inhibitor.

In certain embodiments, the PI3K inhibitor is a PI3K delta selective inhibitor. In one embodiment, the PI3K delta selective inhibitor selectively inhibits PI3K delta isoform over PI3K gamma isoform. In one embodiment, the PI3K delta selective inhibitor has a gamma/delta selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In one embodiment, the PI3K delta selective inhibitor has a gamma/delta selectivity ratio in the range of from greater than 1 to about 5, from about 5 to about 10, from about 10 to about 50, from about 50 to about 850, or greater than about 850. In one embodiment, the gamma/delta selectivity ratio is determined by dividing the inhibitor's IC₅₀ against PI3K gamma isoform by the inhibitor's IC₅₀ against PI3K delta isoform.

In certain embodiments, the PI3K inhibitor is a PI3K delta selective inhibitor. In one embodiment, the PI3K delta selective inhibitor selectively inhibits PI3K delta isoform over PI3K alpha isoform. In one embodiment, the PI3K delta selective inhibitor has an alpha/delta selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In one embodiment, the PI3K delta selective inhibitor has an alpha/delta selectivity ratio in the range of from greater than 1 to about 5, from about 5 to about 10, from about 10 to about 50, from about 50 to about 850, or greater than about 850. In one embodiment, the alpha/delta selectivity ratio is determined by dividing the inhibitor's IC₅₀ against PI3K alpha isoform by the inhibitor's IC₅₀ against PI3K delta isoform.

In certain embodiments, the PI3K inhibitor is a PI3K delta selective inhibitor. In one embodiment, the PI3K delta selective inhibitor selectively inhibits PI3K delta isoform over PI3K beta isoform. In one embodiment, the PI3K delta selective inhibitor has a beta/delta selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In one embodiment, the PI3K delta selective inhibitor has a beta/delta selectivity ratio in the range of from greater than 1 to about 5, from about 5 to about 10, from about 10 to about 50, from about 50 to about 850, or greater than about 850. In one embodiment, the beta/delta selectivity ratio is determined by dividing the inhibitor's IC₅₀ against PI3K beta isoform by the inhibitor's IC₅₀ against PI3K delta isoform.

In certain embodiments, the PI3K inhibitor is selective for both gamma and delta. In one embodiment, the PI3K gamma and delta selective inhibitor selectively inhibits PI3K gamma and delta isoforms over PI3K beta isoform. In one embodiment, the PI3K gamma and delta selective inhibitor has a beta/delta selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000 and a beta/gamma selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In one embodiment, the PI3K delta selective inhibitor has a beta/delta selectivity ratio in the range of from greater than 1 to about 5, from about 5 to about 10, from about 10 to about 50, from about 50 to about 850, or greater than about 850 and a beta/gamma selectivity ratio in the range of from greater than 1 to about 5, from about 5 to about 10, from about 10 to about 50, from about 50 to about 850, or greater than about 850. In one embodiment, the beta/delta selectivity ratio is determined by dividing the inhibitor's IC50 against PI3K beta isoform by the inhibitor's IC50 against PI3K delta isoform and the beta/gamma selectivity ratio is determined by dividing the inhibitor's IC50 against PI3K beta isoform by the inhibitor's IC50 against PI3K gamma isoform.

PI3K delta inhibitors that can be used in the compositions and methods provided herein include, but are not limited to, GSK-2269557 (2-(6-(1H-indol-4-yl)-1H-indazol-4-yl)-5-((4-isopropylpiperazin-1-yl)methyl)oxazole), GS-9820, GS-1101 (5-fluoro-3-phenyl-2-([S)]-1-[9H-purin-6-ylamino]-propyl)-3H-quinazolin-4-one), AMG319, or TGR-1202 (((S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one)), or a mixture thereof. In one embodiment, the PI3K delta inhibitor is GS1101.

In one embodiment, the PI3K inhibitor is a PI3K inhibitor as described in WO 2005/113556, the entirety of which is incorporated herein by reference. In one embodiment, the PI3K inhibitor is Compound Nos. 113 or 107 as described in WO2005/113556.

In one embodiment, the PI3K inhibitor is a PI3K inhibitor as described in WO2014/006572, the entirety of which is incorporated herein by reference. In one embodiment, the PI3K inhibitor is Compound Nos. A1, A2, B, B1, or B2 as described in WO2014/006572.

In certain embodiments, the PI3K inhibitor is a PI3K delta/gamma dual inhibitor. In one embodiment, the PI3K delta/gamma dual inhibitor has an IC₅₀ value against PI3K alpha that is at least 5×, 10×, 20×, 50×, 100×, 200×, 500×, or 1000× higher than its IC₅₀ values against delta and gamma.

In certain embodiments, the PI3K inhibitor is Compound 1 of the structure:

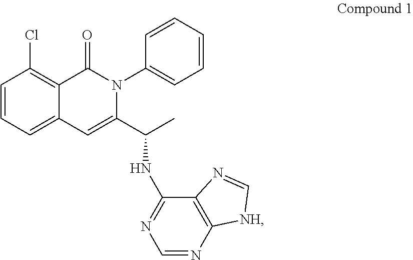 [SMILES: C[C@H](Nc1ncnc2[nH]cnc12)c1cc2cccc(Cl)c2c(=O)n1-c1ccccc1]

or a pharmaceutically acceptable form thereof.

Compound 1 has a chemical name of (S)-3-(1-((9H-purin-6-yl)amino)ethyl)-8-chloro-2-phenylisoquinolin-1(2H)-one. An exemplary method for synthesizing Compound 1 has been previously described in U.S. Pat. No. 8,193,182, which is incorporated by reference in its entirety. Compound 1 is a PI3K-δ,-γ inhibitor and can be used to treat cancers. See U.S. Pat. No. 8,193,182.

Compound 1 provided herein contains one chiral center, and can exist as a mixture of enantiomers, e.g., a racemic mixture. This application encompasses the use of stereomerically pure forms of such a compound, as well as the use of mixtures of those forms. For example, mixtures comprising equal or unequal amounts of the enantiomers of Compound 1 provided herein may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

In one embodiment, the PI3K inhibitor provided herein is a mixture of Compound 1 and its (R)-enantiomer. In one embodiment, the PI3K inhibitor provided herein is a racemic mixture of Compound 1 and its (R)-enantiomer. In other embodiments, the compound mixture has an (S)-enantiomeric purity of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric purity of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (R)-enantiomeric purity of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

As used herein, Compound 1 also refers to any crystal form or polymorph of (S)-3-(1-((9H-purin-6-yl)amino)ethyl)-8-chloro-2-phenylisoquinolin-1(2H)-one. In some embodiments, a polymorph of Compound 1, or a pharmaceutically form thereof, disclosed herein is used. Exemplary polymorphs are disclosed in U.S. Patent Publication No. 2012/0184568, which is hereby incorporated by reference in its entirety. In one embodiment, the compound is Form A of Compound 1. In one embodiment, the compound is Form B of Compound 1. In one embodiment, the compound is Form C of Compound 1. In one embodiment, the compound is Form D of Compound 1. In one embodiment, the compound is Form E of Compound 1. In one embodiment, the compound is Form F of Compound 1. In one embodiment, the compound is Form G of Compound 1. In one embodiment, the compound is Form H of Compound 1. In one embodiment, the compound is Form I of Compound 1. In one embodiment, the compound is Form J of Compound 1. In one embodiment, the compound is a mixture of solid forms (e.g., polymorphs and/or amorphous forms) of Compound 1 disclosed herein.

Any of the compounds disclosed herein can be in the form of pharmaceutically acceptable salts, hydrates, solvates, chelates, non-covalent complexes, isomers, prodrugs, isotopically labeled derivatives, or mixtures thereof.

2.2 Combinations of PI3K Inhibitors and MEK Inhibitors

Provided herein are pharmaceutical compositions comprising a therapeutically effective amount of a PI3K inhibitor, or a pharmaceutically acceptable form thereof, and a MEK inhibitor, or a pharmaceutically acceptable form thereof. In one embodiment, the MEK inhibitor is not pimasertib. In one embodiment, when the PI3K inhibitor is GS1101, the MEK inhibitor is not pimasertib.

Also provided herein are methods of treating, managing, or preventing a cancer in a subject comprising administering to the subject a therapeutically effective amount of a PI3K inhibitor, or a pharmaceutically acceptable form thereof, in combination with a MEK inhibitor, or a pharmaceutically acceptable form thereof.

MEK inhibitors that can be used in the compositions and methods provided herein include, but are not limited to, AZD8330, MEK162 (ARRY438162), PD-0325901, pimasertib (AS703026, MSC1935369), refametinib (BAY869766, RDEA119), R05126766, selumetinib, TAK733, trametinib (GSK1120212), WX-554, R04987655 (CH4987655), XL-518 (GDC-0973), PD184352 (CI-1040), AZD2644, and GDC0623.

In one embodiment, the MEK inhibitor is AZD8330 (2-(2-fluoro-4-iodophenylamino)-N-(2-hydroxyethoxy)-1,5-dimethyl-6-oxo-1,6-dihydropyridine-3-carboxamide), MEK162 (ARRY438162, 5-[(4-bromo-2-fluorophenyl)amino]-4-fluoro-N-(2-hydroxyethoxy)-1-methyl-1H-benzimidazole-6-carboxamide), PD-0325901 ((R)—N-(2,3-dihydroxypropoxy)-3,4-difluoro-2-(2-fluoro-4-iodophenylamino)benzamide), pimasertib (AS703026, MSC1935369, (S)—N-(2,3-dihydroxypropyl)-3-(2-fluoro-4-iodophenylamino)isonicotinamide), refametinib (BAY869766, RDEA119, N-(3,4-difluoro-2-(2-fluoro-4-iodophenylamino)-6-methoxyphenyl)-1-(2,3-dihydroxypropyl)cyclopropane-1-sulfonamide), RO5126766, selumetinib (6-(4-bromo-2-chlorophenylamino)-7-fluoro-N-(2-hydroxyethoxy)-3-methyl-3H-benzo[d]imidazole-5-carboxamide), TAK733 ((R)-3-(2,3-dihydroxypropyl)-6-fluoro-5-(2-fluoro-4-iodophenylamino)-8-methylpyrido[2,3-d]pyrimidine-4,7(3H,8H)-dione), trametinib (GSK1120212, N-[3-[3-Cyclopropyl-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide), WX-554, RO4987655 (CH4987655, 3,4-Difluoro-2-(2-fluoro-4-iodoanilino)-N-(2-hydroxyethoxy)-5-[(3-oxooxazinan-2-yl)methyl]benzamide), XL-518 (GDC-0973, [3,4-difluoro-2-[(2-fluoro-4-iodophenyl)amino]phenyl][3-hydroxy-3-[(2S)-2-piperidinyl]-1-azetidinyl]methanone), PD184352 (CI-1040, 2-(2-Chloro-4-iodophenylamino)-N-cyclopropylmethoxy-3,4-difluorobenzamide), AZD2644, GDC0623 (5-((2-fluoro-4-iodophenyl)amino)-N-(2-hydroxyethoxyl)imidazo[1,5-a]pyridine-6-carboxamide), or a mixture thereof.

In one embodiment, the MEK inhibitor is trametinib. Trametinib has a chemical name of N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide, and is of the structure:

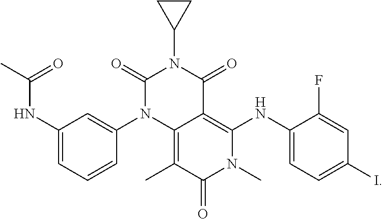 [SMILES: CC(=O)Nc1cccc(-n2c(=O)n(C3CC3)c(=O)c3c(Nc4ccc(I)cc4F)n(C)c(=O)c(C)c32)c1]

In one embodiment, the MEK inhibitor is PD-0325901. PD-0325901 has a chemical name of N-[(2R)-2,3-dihydroxypropoxy]-3,4-difluoro-2-(2-fluoro-4-iodoanilino)benzamide, and is of the structure:

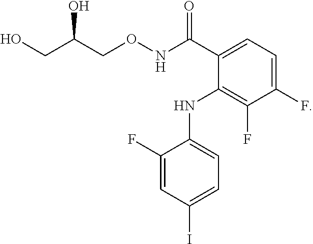 [SMILES: O=C(NOC[C@H](O)CO)c1ccc(F)c(F)c1Nc1ccc(I)cc1F]

In certain embodiments, the PI3K inhibitor is a compound that inhibits one or more PI3K isoforms, e.g., alpha, beta, delta, or gamma isoform. In one embodiment, a PI3K inhibitor is a compound that inhibits one or more PI3K isoforms with an IC₅₀ of less than about 1000 nM, less than about 900 nM, less than about 800 nM, less than about 700 nM, less than about 600 nM, less than about 500 nM, less than about 400 nM, less than about 300 nM, less than about 200 nM, less than about 100 nM, less than about 75 nM, less than about 50 nM, less than about 25 nM, less than about 20 nM, less than about 15 nM, less than about 10 nM, less than about 10 nM, less than about 5 nM, or less than about 1 nM.

In one embodiment, the PI3K inhibitor is a compound that inhibits alpha, beta, delta and gamma isoforms. In another embodiment, the PI3K inhibitor is a compound that inhibits beta, delta, and gamma isoforms. In another embodiment, the PI3K inhibitor is a compound that inhibits the delta and gamma isoforms.

In certain embodiments, the PI3K inhibitor is a PI3K isoform selective inhibitor. In one embodiment, the PI3K inhibitor is a PI3K alpha selective inhibitor. In another embodiment, the PI3K inhibitor is a PI3K beta selective inhibitor. In another embodiment, the PI3K inhibitor is a PI3K gamma selective inhibitor. In another embodiment, the PI3K inhibitor is a PI3K delta selective inhibitor.

In certain embodiments, provided herein is a pharmaceutical composition comprising a therapeutically effective amount of a PI3K delta inhibitor, or a pharmaceutically acceptable form thereof, and a MEK inhibitor, or a pharmaceutically acceptable form thereof. In one embodiment, the PI3K delta inhibitor is GS1101 (CAL-101). In one embodiment, the MEK inhibitor is AZD8330, MEK162 (ARRY438162), PD-0325901, pimasertib (AS703026, MSC1935369), refametinib (BAY869766, RDEA119), R05126766, selumetinib, TAK733, trametinib (GSK1120212), WX-554, R04987655 (CH4987655), XL-518 (GDC-0973), PD184352 (CI-1040), AZD2644, or GDC0623, or a mixture thereof. In one embodiment, the MEK inhibitor is trametinib. In another embodiment, the MEK inhibitor is PD-0325901. In one embodiment, provided herein is a pharmaceutical composition comprising a therapeutically effective amount of GS1101, or a pharmaceutically acceptable form thereof, and trametinib, or a pharmaceutically acceptable form thereof. In another embodiment, provided herein is a pharmaceutical composition comprising a therapeutically effective amount of GS1101, or a pharmaceutically acceptable form thereof, and PD-0325901, or a pharmaceutically acceptable form thereof.

In one embodiment, the MEK inhibitor is not pimasertib. In one embodiment, when the PI3K inhibitor is GS1101, the MEK inhibitor is not pimasertib.

In one embodiment of the compositions and methods described herein, the molar ratio of the PI3K delta inhibitor (e.g., GS1101), or a pharmaceutically acceptable form thereof, to the MEK inhibitor (e.g., trametinib or PD-0325901), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 300:1 to about 100:1, from about 300:1 to about 200:1, or about 250:1. In an embodiment, the MEK inhibitor is trametinib, and the molar ratio of the PI3K delta inhibitor to the MEK inhibitor is from about 1000:1 to about 1:1, from about 750:1 to about 10:1, from about 500:1 to about 10:1, from about 500:1 to about 100:1, from about 500:1 to about 200:1, from about 400:1 to about 200:1, from about 300:1 to about 200:1, or about 250:1. In an embodiment, the MEK inhibitor is PD-0325901, and the molar ratio of the PI3K delta inhibitor to the MEK inhibitor is from about 1000:1 to about 1:1, from about 500:1 to about 1:1, from about 100:1 to about 1:1, from about 20:1 to about 1:1, or about 17:1.

In one embodiment, the PI3K delta inhibitor (e.g., GS1101) is administered at an amount to reach an area under the plasma concentration-time curve at steady-state (AUCs) at about 5000 ng/mL*hr to about 10000 ng/mL*hr, about 5000 ng/mL*hr to about 9000 ng/mL*hr, about 6000 ng/mL*hr to about 9000 ng/mL*hr, about 6000 ng/mL*hr to about 8000 ng/mL*hr, about 6500 ng/mL*hr to about 7500 ng/mL*hr, or about 7000 ng/mL*hr; and

the MEK inhibitor (e.g., trametinib or PD-0325901) is administered at an amount to reach an AUCss at about 0.1 ng/mL*hr to about 2000 ng/mL*hr, about 1 ng/mL*hr to about 2000 ng/mL*hr, about 100 ng/mL*hr to about 1800 ng/mL*hr, about 200 ng/mL*hr to about 1800 ng/mL*hr, about 300 ng/mL*hr to about 1800 ng/mL*hr, about 370 ng/mL*hr, or about 1784 ng/mL*hr.

In one embodiment, the PI3K delta inhibitor (e.g., GS1101) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the MEK inhibitor (e.g., trametinib or PD-0325901) is administered at an amount to reach an AUCss at less than about 2000 ng/mL*hr, less than about 1800 ng/mL*hr, less than about 1500 ng/mL*hr, less than about 1000 ng/mL*hr, less than about 750 ng/mL*hr, less than about 500 ng/mL*hr, less than about 400 ng/mL*hr, less than about 300 ng/mL*hr, less than about 250 ng/mL*hr, less than about 100 ng/mL*hr, less than about 50 ng/mL*hr, less than about 25 ng/mL*hr, less than about 10 ng/mL*hr, less than about 1 ng/mL*hr, less than about 370 ng/mL*hr, or less than 1784 ng/mL*hr.

In one embodiment, the PI3K delta inhibitor (e.g., GS1101) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2300 ng/mL, about 2000 ng/mL to about 2300 ng/mL, or about 2200 ng/mL; and

the MEK inhibitor (e.g., trametinib or PD-0325901) is administered at an amount to reach Cmaxss at about 0.1 ng/mL to about 1000 ng/mL, about 0.1 ng/mL to about 500 ng/mL, about 0.1 ng/mL to about 250 ng/mL, about 1 ng/mL to about 100 ng/mL, about 1 ng/mL to about 50 ng/mL, about 1 ng/mL to about 25 ng/mL, about 10 ng/mL to about 25 ng/mL, about 22 ng/mL, or about 462 ng/mL.

In one embodiment, the PI3K delta inhibitor (e.g., GS1101) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the MEK inhibitor (e.g., trametinib or PD-0325901) is administered at an amount to reach Cmaxss at less than about 1000 ng/mL, less than about 750 ng/mL, less than about 500 ng/mL, less than about 400 ng/mL, less than about 250 ng/mL, less than about 100 ng/mL, less than about 50 ng/mL, less than about 25 ng/mL, less than about 1 ng/mL, less than about 22 ng/mL, or less than about 462 ng/mL.

In one embodiment, the composition comprises the PI3K delta inhibitor (e.g., GS1101), or a pharmaceutically acceptable form thereof, at an amount in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta inhibitor (e.g., GS1101), or a pharmaceutically acceptable form thereof, at an amount of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with a MEK inhibitor (e.g., trametinib or PD-0325901), or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma.

In some embodiments of the methods described herein, the PI3K delta inhibitor (e.g., GS1101), or a pharmaceutically acceptable form thereof, and the MEK inhibitor (e.g., trametinib or PD-0325901), or a pharmaceutically acceptable form thereof, are administered at certain dosages. In one embodiment, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with a MEK inhibitor, or a pharmaceutically acceptable form thereof, wherein the PI3K delta inhibitor (e.g., GS1101), or a pharmaceutically acceptable form thereof, is administered at a dosage of in the range of from about 0.01 mg to about 75 mg daily and the MEK inhibitor (e.g., trametinib or PD-0325901), or a pharmaceutically acceptable form thereof, is administered at a dosage of in the range of from about 0.01 mg to about 1100 mg daily.

In one embodiment, the PI3K delta inhibitor (e.g., GS1101), or a pharmaceutically acceptable form thereof, is administered at a dosage of in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta inhibitor (e.g., GS1101), or a pharmaceutically acceptable form thereof, is administered at a dosage of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a pharmaceutical composition comprising a therapeutically effective amount of a PI3K delta/gamma dual inhibitor, or a pharmaceutically acceptable form thereof, and a MEK inhibitor, or a pharmaceutically acceptable form thereof. In one embodiment, the MEK inhibitor is AZD8330, MEK162 (ARRY438162), PD-0325901, pimasertib (AS703026, MSC1935369), refametinib (BAY869766, RDEA119), R05126766, selumetinib, TAK733, trametinib (GSK1120212), WX-554, R04987655 (CH4987655), XL-518 (GDC-0973), PD184352 (CI-1040), AZD2644, or GDC0623, or a mixture thereof. In one embodiment, the MEK inhibitor is trametinib. In another embodiment, the MEK inhibitor is PD-0325901.

In one embodiment of the compositions and methods described herein, the molar ratio of the PI3K delta/gamma dual inhibitor, or a pharmaceutically acceptable form thereof, to the MEK inhibitor (e.g., trametinib or PD-0325901), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 5:1 to about 1:1, from about 3:1 to about 1:1, from about 500:1 to about 1:1, from about 200:1 to about 5:1, from about 100:1 to about 10:1, from about 50:1 to about 30:1, about 40:1, or about 3:1.

In one embodiment, the composition comprises the PI3K delta/gamma dual inhibitor, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta/gamma dual inhibitor, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta/gamma dual inhibitor, or a pharmaceutically acceptable form thereof, in combination with a MEK inhibitor (e.g., trametinib or PD-0325901), or a pharmaceutically acceptable form thereof, wherein the cancer is diffuse large B-cell lymphoma (activated B-cell-like), diffuse large B-cell lymphoma (germinal center B-cell-like), follicular lymphoma, T-cell lymphoma, mantle cell lymphoma, or multiple myeloma.

In some embodiments of the methods described herein, the PI3K delta/gamma dual inhibitor, or a pharmaceutically acceptable form thereof, and the MEK inhibitor (e.g., trametinib or PD-0325901), or a pharmaceutically acceptable form thereof, are administered at certain dosages. In one embodiment, provided herein is a method of treating, managing, or preventing a cancer in a subject comprising administering to the subject a therapeutically effective amount of a PI3K delta/gamma dual inhibitor, or a pharmaceutically acceptable form thereof, in combination with a MEK inhibitor, or a pharmaceutically acceptable form thereof, wherein the PI3K delta/gamma dual inhibitor, or a pharmaceutically acceptable form thereof, is administered at a dosage of in the range of from about 0.01 mg to about 75 mg daily and the MEK inhibitor (e.g., trametinib or PD-0325901), or a pharmaceutically acceptable form thereof, is administered at a dosage of in the range of from about 0.01 mg to about 1100 mg daily.

In one embodiment, the PI3K delta/gamma dual inhibitor, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta/gamma dual inhibitor, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In one embodiment, the PI3K delta/gamma dual inhibitor (e.g., Compound 1) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and the MEK inhibitor (e.g., trametinib or PD-0325901) is administered at an amount to reach an AUCss at about 0.1 ng/mL*hr to about 2000 ng/mL*hr, about 1 ng/mL*hr to about 2000 ng/mL*hr, about 100 ng/mL*hr to about 1800 ng/mL*hr, about 200 ng/mL*hr to about 1800 ng/mL*hr, about 300 ng/mL*hr to about 1800 ng/mL*hr, about 370 ng/mL*hr, about 1784 ng/mL*hr.

In one embodiment, the PI3K delta/gamma dual inhibitor (e.g., Compound 1) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the MEK inhibitor (e.g., trametinib or PD-0325901) is administered at an amount to reach an AUCss at less than about 2000 ng/mL*hr, less than about 1800 ng/mL*hr, less than about 1500 ng/mL*hr, less than about 1000 ng/mL*hr, less than about 750 ng/mL*hr, less than about 500 ng/mL*hr, less than about 400 ng/mL*hr, less than about 300 ng/mL*hr, less than about 250 ng/mL*hr, less than about 100 ng/mL*hr, less than about 50 ng/mL*hr, less than about 25 ng/mL*hr, less than about 10 ng/mL*hr, less than about 1 ng/mL*hr, less than about 370 ng/mL*hr, or less than 1784 ng/mL*hr.

In one embodiment, the PI3K delta/gamma dual inhibitor (e.g., Compound 1) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

the MEK inhibitor (e.g., trametinib or PD-0325901) is administered at an amount to reach Cmaxss at about 0.1 ng/mL to about 1000 ng/mL, about 0.1 ng/mL to about 500 ng/mL, about 0.1 ng/mL to about 250 ng/mL, about 1 ng/mL to about 100 ng/mL, about 1 ng/mL to about 50 ng/mL, about 1 ng/mL to about 25 ng/mL, about 10 ng/mL to about 25 ng/mL, about 22 ng/mL, or about 462 ng/mL.

In one embodiment, the PI3K delta/gamma dual inhibitor (e.g., Compound 1) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the MEK inhibitor (e.g., trametinib or PD-0325901) is administered at an amount to reach Cmaxss at less than about 1000 ng/mL, less than about 750 ng/mL, less than about 500 ng/mL, less than about 400 ng/mL, less than about 250 ng/mL, less than about 100 ng/mL, less than about 50 ng/mL, less than about 25 ng/mL, less than about 1 ng/mL, less than about 22 ng/mL, or less than about 462 ng/mL.

In one embodiment, the PI3K delta/gamma dual inhibitor (e.g., Compound 1) is administered at an amount that is decreased by about 1.5 fold to about 50 fold of the amount when administered individually and the MEK inhibitor (e.g., trametinib or PD-0325901) is administered at an amount that is decreased by about 1.1 fold to about 50 fold of the amount when administered individually.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and

the MEK inhibitor (e.g., trametinib or PD-0325901) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In certain embodiments, provided herein is a pharmaceutical composition comprising a therapeutically effective amount of Compound 1:

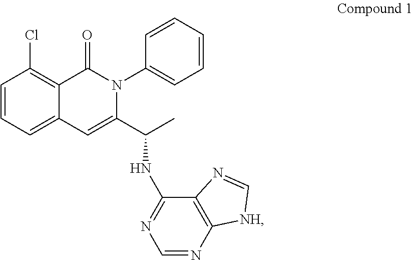 [SMILES: C[C@H](Nc1ncnc2[nH]cnc12)c1cc2cccc(Cl)c2c(=O)n1-c1ccccc1]

or a pharmaceutically acceptable form thereof, and a MEK inhibitor, or a pharmaceutically acceptable form thereof. In one embodiment, the MEK inhibitor is AZD8330, MEK162 (ARRY438162), PD-0325901, pimasertib (AS703026, MSC1935369), refametinib (BAY869766, RDEA119), R05126766, selumetinib, TAK733, trametinib (GSK1120212), WX-554, R04987655 (CH4987655), XL-518 (GDC-0973), PD184352 (CI-1040), AZD2644, or GDC0623, or a mixture thereof. In one embodiment, the MEK inhibitor is trametinib. In another embodiment, the MEK inhibitor is PD-0325901.

In certain embodiments, provided herein is a method of treating, managing, or preventing a cancer in a subject comprising administering to the subject a therapeutically effective amount of Compound 1:

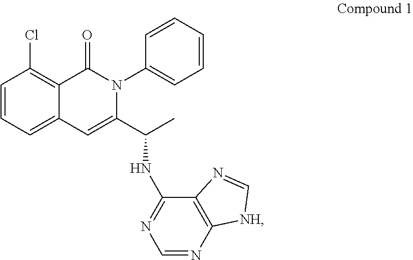 [SMILES: C[C@H](Nc1ncnc2[nH]cnc12)c1cc2cccc(Cl)c2c(=O)n1-c1ccccc1]

or a pharmaceutically acceptable form thereof, in combination with a MEK inhibitor, or a pharmaceutically acceptable form thereof. In one embodiment, the MEK inhibitor is AZD8330, MEK162 (ARRY438162), PD-0325901, pimasertib (AS703026, MSC1935369), refametinib (BAY869766, RDEA119), R05126766, selumetinib, TAK733, trametinib (GSK1120212), WX-554, R04987655 (CH4987655), XL-518 (GDC-0973), PD184352 (CI-1040), AZD2644, or GDC062, or a mixture thereof. In one embodiment, the MEK inhibitor is trametinib. In another embodiment, the MEK inhibitor is PD-0325901.

In some embodiments of the compositions and methods described herein, Compound 1, or a pharmaceutically acceptable form thereof, is used in combination with a MEK inhibitor (e.g., trametinib or PD-0325901), or a pharmaceutically acceptable form thereof, at certain molar ratios. In one embodiment, provided herein is a pharmaceutical composition comprising a therapeutically effective amount of Compound 1:

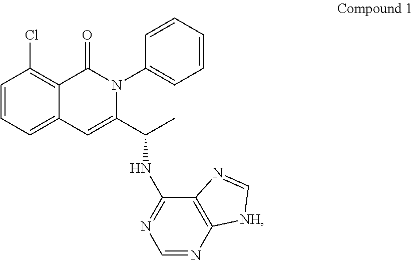 [SMILES: C[C@H](Nc1ncnc2[nH]cnc12)c1cc2cccc(Cl)c2c(=O)n1-c1ccccc1]

or a pharmaceutically acceptable form thereof, and a MEK inhibitor, or a pharmaceutically acceptable form thereof, wherein the molar ratio of Compound 1, or a pharmaceutically acceptable form thereof, to the MEK inhibitor (e.g., trametinib or PD-0325901), or a pharmaceutically acceptable form thereof, is in the range of from about 1000:1 to about 1:1000.

In one embodiment of the compositions and methods described herein, the molar ratio of Compound 1, or a pharmaceutically acceptable form thereof, to the MEK inhibitor (e.g., trametinib or PD-0325901), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, or from about 5:1 to about 1:5. In an embodiment, the PI3K inhibitor is Compound 1 and the MEK inhibitor is trametinib, and the molar ratio of the PI3K inhibitor to the MEK inhibitor is from about 500:1 to about 1:1, from about 200:1 to about 5:1, from about 100:1 to about 10:1, from about 50:1 to about 30:1, or about 40:1.

In one embodiment, the composition comprises the PI3K delta inhibitor (e.g. GS1101), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta inhibitor (e.g., GS1101), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta inhibitor which is GS1101, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the MEK inhibitor, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the MEK inhibitor, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the MEK inhibitor which is trametinib, or a pharmaceutically acceptable form thereof, at an amount sufficient to deliver a blood plasma concentration profile with an AUC (area under curve) of from about 0.1 μg/mL*h to about 1 μg/mL*h, from about 0.2 μg/mL*h to about 0.5 μg/mL*h, or from about 0.3 μg/mL*h to about 0.4 μg/mL*h.

In one embodiment, Compound 1 is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and

trametinib or PD-0325901 is administered at an amount to reach an AUCss at about 0.1 ng/mL*hr to about 2000 ng/mL*hr, about 1 ng/mL*hr to about 2000 ng/mL*hr, about 100 ng/mL*hr to about 1800 ng/mL*hr, about 200 ng/mL*hr to about 1800 ng/mL*hr, about 300 ng/mL*hr to about 1800 ng/mL*hr, about 370 ng/mL*hr, or about 1784 ng/mL*hr.

In one embodiment, Compound 1 is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

the MEK inhibitor (e.g., trametinib or PD-0325901) is administered at an amount to reach Cmaxss at about 0.1 ng/mL to about 1000 ng/mL, about 0.1 ng/mL to about 500 ng/mL, about 0.1 ng/mL to about 250 ng/mL, about 1 ng/mL to about 100 ng/mL, about 1 ng/mL to about 50 ng/mL, about 1 ng/mL to about 25 ng/mL, about 10 ng/mL to about 25 ng/mL, about 22 ng/mL, or about 462 ng/mL.

In one embodiment, Compound 1 is administered at an amount that is decreased by about 1.5 fold to about 50 fold of the amount when administered individually and trametinib or PD-0325901 is administered at an amount that is decreased by about 1.1 fold to about 50 fold of the amount when administered individually.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and

trametinib or PD-0325901 is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually. In one embodiment of the compositions and methods described herein, the weight ratio of Compound 1, or a pharmaceutically acceptable form thereof, to trametinib, or a pharmaceutically acceptable form thereof, is in the range of from about 7.5-37.5 of Compound 1 to from 0.2-1 of trametinib. In one embodiment, the weight ratio is in the range of from about 180:1 to about 7.5:1. In one embodiment, the weight ratio is in the range of from about 90:1 to about 15:1. In one embodiment, the weight ratio is in the range of from about 60:1 to about 22.5:1. In one embodiment, the weight ratio is in the range of from about 30:1 to about 20:1. In one embodiment, the weight ratio is about 25:1.

In one embodiment of the compositions and methods described herein, the weight ratio of Compound 1, or a pharmaceutically acceptable form thereof, to PD-0325901, or a pharmaceutically acceptable form thereof, is in the range of from about 7.5-37.5 of Compound 1 to from 0.4-2 of PD-0325901. In one embodiment, the weight ratio is in the range of from about 90:1 to about 4:1. In one embodiment, the weight ratio is in the range of from about 45:1 to about 8:1. In one embodiment, the weight ratio is in the range of from about 30:1 to about 12:1. In one embodiment, the weight ratio is in the range of from about 30:1 to about 20:1. In one embodiment, the weight ratio is about 25:1.

In some embodiments of the compositions and methods described herein, the composition comprises Compound 1, or a pharmaceutically acceptable form thereof, and the MEK inhibitor (e.g., trametinib or PD-0325901), or a pharmaceutically acceptable form thereof, at certain amounts. In one embodiment, provided herein is a pharmaceutical composition comprising a therapeutically effective amount of Compound 1:

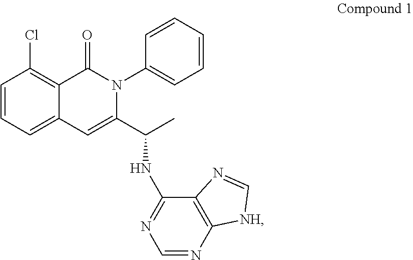 [SMILES: C[C@H](Nc1ncnc2[nH]cnc12)c1cc2cccc(Cl)c2c(=O)n1-c1ccccc1]

or a pharmaceutically acceptable form thereof, and a MEK inhibitor, or a pharmaceutically acceptable form thereof, wherein the composition comprises Compound 1, or a pharmaceutically acceptable form thereof, at an amount in the range of from about 0.01 mg to about 75 mg and the MEK inhibitor (e.g., trametinib or PD-0325901), or a pharmaceutically acceptable form thereof, at an amount of in the range of from about 0.01 mg to about 1100 mg.

In one embodiment, the composition comprises Compound 1, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg. In one embodiment, the composition comprises Compound 1, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg. In one embodiment, the composition comprises Compound 1, or a pharmaceutically acceptable form thereof, at an amount of about 50 mg, about 37.5 mg, about 25 mg, about 20 mg, about 15 mg, about 10 mg, about 5 mg, or about 1 mg.

In one embodiment, the composition comprises the MEK inhibitor (e.g., trametinib or PD-0325901), or a pharmaceutically acceptable form thereof, at an amount in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg. In one embodiment, the composition comprises the MEK inhibitor (e.g., trametinib or PD-0325901), or a pharmaceutically acceptable form thereof, at an amount of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, or less than about 25 mg.

In one embodiment, the composition comprises trametinib, or a pharmaceutically acceptable form thereof, at an amount in the range of from about 0.005 mg to about 2 mg, from about 0.005 mg to about 1 mg, from about 0.025 mg to about 0.75 mg, from about 0.05 mg to about 0.5 mg, from about 0.1 mg to about 0.4 mg, or from about 0.2 mg to about 0.3 mg. In one embodiment, the composition comprises trametinib, or a pharmaceutically acceptable form thereof, at an amount of less than about 2 mg, less than about 1.5 mg, less than about 1.25 mg, less than about 1 mg, less than about 0.75 mg, less than about 0.5 mg, less than about 0.375 mg, less than about 0.25 mg, or less than about 0.125 mg. In one embodiment, the composition comprises trametinib, or a pharmaceutically acceptable form thereof, at an amount of about 2 mg, about 1.5 mg, about 1.25 mg, about 1 mg, about 0.75 mg, about 0.5 mg, about 0.375 mg, about 0.25 mg, or about 0.125 mg.

In one embodiment, the composition comprises PD-0325901, or a pharmaceutically acceptable form thereof, at an amount in the range of from about 0.01 mg to about 4 mg, from about 0.01 mg to about 2 mg, from about 0.05 mg to about 1.5 mg, from about 0.1 mg to about 1 mg, from about 0.2 mg to about 0.8 mg, or from about 0.4 mg to about 0.6 mg. In one embodiment, the composition comprises PD-0325901, or a pharmaceutically acceptable form thereof, at an amount of less than about 4 mg, less than about 3 mg, less than about 2.5 mg, less than about 2 mg, less than about 1.5 mg, less than about 1 mg, less than about 0.75 mg, less than about 0.5 mg, or less than about 0.25 mg. In one embodiment, the composition comprises PD-0325901, or a pharmaceutically acceptable form thereof, at an amount of about 4 mg, about 3 mg, about 2.5 mg, about 2 mg, about 1.5 mg, about 1 mg, about 0.75 mg, about 0.5 mg, or about 0.25 mg.

In certain embodiments, provided herein is a method of treating, managing, or preventing a cancer in a subject comprising administering to the subject a therapeutically effective amount of Compound 1, or a pharmaceutically acceptable form thereof, in combination with a MEK inhibitor, or a pharmaceutically acceptable form thereof, wherein the cancer is diffuse large B-cell lymphoma (activated B-cell-like), diffuse large B-cell lymphoma (germinal center B-cell-like), follicular lymphoma, T-cell lymphoma, mantle cell lymphoma, or multiple myeloma. In one embodiment, the MEK inhibitor is trametinib. In another embodiment, the MEK inhibitor is PD-0325901.

In some embodiments of the methods described herein, Compound 1, or a pharmaceutically acceptable form thereof, and the MEK inhibitor (e.g., trametinib or PD-0325901), or a pharmaceutically acceptable form thereof, are administered at certain dosages. In one embodiment, provided herein is a method of treating, managing, or preventing a cancer in a subject comprising administering to the subject a therapeutically effective amount of Compound 1:

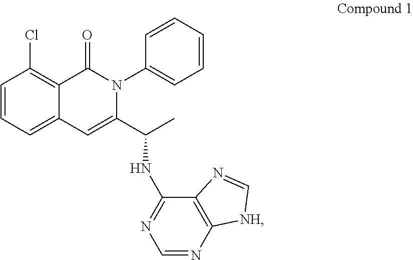 [SMILES: C[C@H](Nc1ncnc2[nH]cnc12)c1cc2cccc(Cl)c2c(=O)n1-c1ccccc1]

or a pharmaceutically acceptable form thereof, in combination with a MEK inhibitor, or a pharmaceutically acceptable form thereof, wherein Compound 1, or a pharmaceutically acceptable form thereof, is administered at a dosage of in the range of from about 0.01 mg to about 75 mg daily and the MEK inhibitor (e.g., trametinib or PD-0325901), or a pharmaceutically acceptable form thereof, is administered at a dosage of in the range of from about 0.01 mg to about 1100 mg daily.

In one embodiment, Compound 1, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily. In one embodiment, Compound 1, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily. In one embodiment, Compound 1, or a pharmaceutically acceptable form thereof, is administered at a dosage of about 50 mg, about 37.5 mg, about 25 mg, about 20 mg, about 15 mg, about 10 mg, about 5 mg, or about 1 mg daily.

In one embodiment, the MEK inhibitor (e.g., trametinib or PD-0325901), or a pharmaceutically acceptable form thereof, is administered at a dosage of in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg daily. In one embodiment, the MEK inhibitor (e.g., trametinib or PD-0325901), or a pharmaceutically acceptable form thereof, is administered at a dosage of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, or less than about 25 mg daily.

In one embodiment, trametinib, or a pharmaceutically acceptable form thereof, is administered at a dosage of in the range of from about 0.005 mg to about 2 mg, from about 0.005 mg to about 1 mg, from about 0.025 mg to about 0.75 mg, from about 0.05 mg to about 0.5 mg, from about 0.1 mg to about 0.4 mg, or from about 0.2 mg to about 0.3 mg daily. In one embodiment, trametinib, or a pharmaceutically acceptable form thereof, is administered at a dosage of less than about 2 mg, less than about 1.5 mg, less than about 1.25 mg, less than about 1 mg, less than about 0.75 mg, less than about 0.5 mg, less than about 0.375 mg, less than about 0.25 mg, or less than about 0.125 mg daily. In one embodiment, trametinib, or a pharmaceutically acceptable form thereof, is administered at a dosage of about 2 mg, about 1.5 mg, about 1.25 mg, about 1 mg, about 0.75 mg, about 0.5 mg, about 0.375 mg, about 0.25 mg, or about 0.125 mg daily.

In one embodiment, PD-0325901, or a pharmaceutically acceptable form thereof, is administered at a dosage of in the range of from about 0.01 mg to about 4 mg, from about 0.01 mg to about 2 mg, from about 0.05 mg to about 1.5 mg, from about 0.1 mg to about 1 mg, from about 0.2 mg to about 0.8 mg, or from about 0.4 mg to about 0.6 mg daily. In one embodiment, PD-0325901, or a pharmaceutically acceptable form thereof, is administered at a dosage of less than about 4 mg, less than about 3 mg, less than about 2.5 mg, less than about 2 mg, less than about 1.5 mg, less than about 1 mg, less than about 0.75 mg, less than about 0.5 mg, or less than about 0.25 mg daily. In one embodiment, PD-0325901, or a pharmaceutically acceptable form thereof, is administered at a dosage of about 4 mg, about 3 mg, about 2.5 mg, about 2 mg, about 1.5 mg, about 1 mg, about 0.75 mg, about 0.5 mg, or about 0.25 mg daily.

In one embodiment, PD-0325901, or a pharmaceutically acceptable form thereof, is administered at a dosage of in the range of from about 0.005 mg to about 2 mg, from about 0.005 mg to about 1 mg, from about 0.025 mg to about 0.75 mg, from about 0.05 mg to about 0.5 mg, from about 0.1 mg to about 0.4 mg, or from about 0.2 mg to about 0.3 mg twice daily. In one embodiment, PD-0325901, or a pharmaceutically acceptable form thereof, is administered at a dosage of less than about 2 mg, less than about 1.5 mg, less than about 1.25 mg, less than about 1 mg, less than about 0.75 mg, less than about 0.5 mg, less than about 0.375 mg, less than about 0.25 mg, or less than about 0.125 mg twice daily. In one embodiment, PD-0325901, or a pharmaceutically acceptable form thereof, is administered at a dosage of about 2 mg, about 1.5 mg, about 1.25 mg, about 1 mg, about 0.75 mg, about 0.5 mg, about 0.375 mg, about 0.25 mg, or about 0.125 mg twice daily.

In one embodiment, the MEK inhibitor (e.g., trametinib or PD-0325901), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the MEK inhibitor (e.g., trametinib or PD-0325901), or a pharmaceutically acceptable form thereof, is administered concurrently with the PI3K inhibitor (e.g., Compound 1), or a pharmaceutically acceptable form thereof, in a single dosage form or separate dosage forms. In yet another embodiment, the MEK inhibitor (e.g., trametinib or PD-0325901), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered. In one embodiment, the MEK inhibitor is trametinib. In another embodiment, the MEK inhibitor is PD-0325901.

In certain embodiments, the PI3K inhibitor (e.g., Compound 1), or a pharmaceutically acceptable form thereof, and the MEK inhibitor (e.g., trametinib or PD-0325901), or a pharmaceutically acceptable form thereof, are in a single dosage form. In other embodiments, the PI3K inhibitor (e.g., Compound 1), or a pharmaceutically acceptable form thereof, and the MEK inhibitor (e.g., trametinib or PD-0325901), or a pharmaceutically acceptable form thereof, are in separate dosage forms.

In certain embodiments, the PI3K inhibitor (e.g., Compound 1), or a pharmaceutically acceptable form thereof, and the MEK inhibitor (e.g., trametinib or PD-0325901), are administered via a same route, e.g., both are administered orally. In other embodiments, the PI3K inhibitor (e.g., Compound 1), or a pharmaceutically acceptable form thereof, and the MEK inhibitor (e.g., trametinib or PD-0325901), are administered via different routes, e.g., one is administered orally and the other is administered intravenously. In one embodiment, Compound 1 is administered orally once per day and trametinib is administered orally once per day. In one embodiment, Compound 1 is administered orally once per day and PD-0325901 is administered orally once per day. In one embodiment, Compound 1 is administered orally once per day and PD-0325901 is administered orally twice per day.

In certain embodiments, the PI3K inhibitor (e.g., Compound 1), or a pharmaceutically acceptable form thereof, and the MEK inhibitor (e.g., trametinib or PD-0325901), or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., GS1101), a PI3K delta/gamma dual inhibitor, and a MEK inhibitor (e.g., trametinib or PD-0325901).

2.3 Combinations of PI3K Inhibitors and mTOR Inhibitors

Provided herein are pharmaceutical compositions comprising a therapeutically effective amount of a PI3K inhibitor, or a pharmaceutically acceptable form thereof, and a mTOR inhibitor, or a pharmaceutically acceptable form thereof. In one embodiment, the mTOR inhibitor is not rapamycin.

Also provided herein are methods of treating, managing, or preventing a cancer in a subject comprising administering to the subject a therapeutically effective amount of a PI3K inhibitor, or a pharmaceutically acceptable form thereof, and a mTOR inhibitor, or a pharmaceutically acceptable form thereof. In one embodiment, the mTOR inhibitor is not rapamycin.

mTOR inhibitors that can be used in the compositions and methods provided herein include, but are not limited to, AP23841, AZD8055, BEZ235, BGT226, deferolimus (AP23573/MK-8669), EM101/LY303511, everolimus (RAD001), EX2044, EX3855, EX7518, GDC0980, INK-128, KU-0063794, NV-128, OSI-027, PF-4691502, rapalogs, rapamycin, ridaforolimus, SAR543, SF1126, temsirolimus (CCI-779), WYE-125132, XL765, zotarolimus (ABT578), torin 1, GSK2126458, AZD2014, GDC-0349, and XL388.

In one embodiment, the mTOR inhibitor is AP23841, AZD8055 ((5-(2,4-bis((S)-3-methylmorpholino)pyrido[2,3-d]pyrimidin-7-yl)-2-methoxyphenyl)methanol), BEZ235 (2-methyl-2-(4-(3-methyl-2-oxo-8-(quinolin-3-yl)-2,3-dihydroimidazo[4,5-c]quinolin-1-yl)phenyl)propanenitrile), BGT226 (8-(6-methoxypyridin-3-yl)-3-methyl-1-(4-(piperazin-1-yl)-3-(trifluoromethyl)phenyl)-1H-imidazo[4,5-c]quinolin-2(3H)-one maleic acid), deforolimus (AP23573/MK-8669, (1R,2R,4S)-4-R2R)-2-[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate), EM101/LY303511 (2-(1-Piperazinyl)-8-phenyl-4H-1-benzopyran-4-one), everolimus (RAD001, dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0]hexatriaconta-16,24,26,28-tetraene-2,3,10,14,20-pentone), EX2044, EX3855, EX7518, GDC0980 ((S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one), INK-128 (3-(2-aminobenzo[d]oxazol-5-yl)-1-isopropyl-1H-pyrazolo[3,4-d]pyrimidin-4-amine), KU-0063794 ((5-(2-((2R,6S)-2,6-dimethylmorpholino)-4-morpholinopyrido[2,3-d]pyrimidin-7-yl)-2-methoxyphenyl)methanol), NV-128, OSI-027 ((1r,4r)-4-(4-amino-5-(7-methoxy-1H-indol-2-yl)imidazo[1,5-f][1,2,4]triazin-7-yl)cyclohexanecarboxylic acid), PF-4691502 (2-amino-6-(6-methoxypyridin-3-yl)-4-methyl-8-[(1r,4r)-4-(2-hydroxyethoxyl)cyclohexyl]-7h,8h-pyrido[2,3-d]pyrimidin-7-one), rapalogs, rapamycin ((3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29 (4H,6H,31H)-pentone), ridaforolimus ((1R,2R,4S)-4-[(2R)-2-[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0⁴′⁹]hexatriaconta-16,24,26,28-tetraen-12-yl]propyl]-2-methoxycyclohexyl dimethylphosphinate), SAR543, SF1126 (3-[[2-[[5-[[amino(azaniumyl)methylidene]amino]-2-[[4-oxo-4-[4-(4-oxo-8-phenylchromen-2-yl)morpholin-4-ium-4-yl]oxybutanoyl]amino]pentanoyl]amino]acetyl]amino]-4-(1-carboxylatopropylamino)-4-oxobutanoate), temsirolimus (CCI-779, (1R,2R,4S)-4-{(2R)-2-[(3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate), WYE-125132 (N-[4-[1-(1,4-dioxaspiro[4.5]dec-8-yl)-4-(8-oxa-3-azabicyclo[3.2.1]oct-3-yl)-1H-pyrazolo[3,4-d]pyrimidin-6-yl]phenyl]-N′-methyl-urea), XL765 (N-[4-[[[3-[(3,5-dimethoxyphenyl)amino]-2-quinoxalinyl]amino]sulfonyl]phenyl]-3-methoxy-4-methyl-benzamide), zotarolimus (ABT578, (3S,6R,7E,9R,10R,12R,14S,15E,17E,19E,21S,23S,26R,27R,34aS)-9,27-dihydroxy-10,21-dimethoxy-3-{(1R)-2-[(1S,3R,4S)-3-methoxy-4-(1H-tetrazol-1-yl)cyclohexyl]-1-methylethyl)-6,8,12,14,20,26-hexamethyl-4,9,10,12,13,14,21,22,23,24,25,26,27,32,33,34,34a-heptadecahydro-3H-23,27-epoxypyrido[2,1-c][1,4]oxazacyclohentriacontine-1,5,11,28,29(6H,31H)-pentone), torin 1 (1-[4-[4-(1-oxopropyl)-1-piperazinyl]-3-(trifluoromethyl)phenyl]-9-(3-quinolinyl)-benzo[h]-1,6-naphthyridin-2(1H)-one), GSK2126458 (2,4-Difluoro-N-{2-(methyloxy)-5-[4-(4-pyridazinyl)-6-quinolinyl]-3-pyridinyl}benzenesulfonamide), AZD2014 (3-[2,4-Bis((3S)-3-methylmorpholin-4-yl)pyrido[5,6-e]pyrimidin-7-yl]-N-methylbenzamide), GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), or XL388 ((7-(6-aminopyridin-3-yl)-2,3-dihydrobenzo[f][1,4]oxazepin-4(5H)-yl)(3-fluoro-2-methyl-4-(methylsulfonyl)phenyl)methanone), or a mixture thereof.

In one embodiment, the mTOR inhibitor is everolimus. Everolimus has a chemical name of dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0]hexatriaconta-16,24,26,28-tetraene-2,3,10,14,20-pentone, and is of the structure:

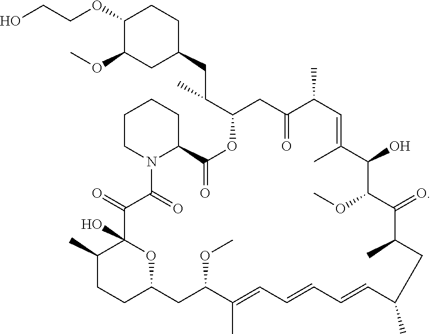 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In one embodiment, the mTOR inhibitor is AZD8055. AZD8055 has a chemical name of (5-{2,4-bis[(3S)-3-methylmorpholin-4-yl]pyrido[2,3-d]pyrimidin-7-yl}-2-methoxyphenyl)methanol, and is of the structure:

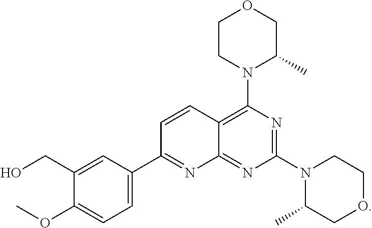 [SMILES: COc1ccc(-c2ccc3c(N4CCOC[C@@H]4C)nc(N4CCOC[C@@H]4C)nc3n2)cc1CO]

In certain embodiments, provided herein is a composition, e.g., a pharmaceutical composition, comprising a therapeutically effective amount of a PI3K delta inhibitor, or a pharmaceutically acceptable form thereof, and a mTOR inhibitor, or a pharmaceutically acceptable form thereof. In one embodiment, the PI3K delta inhibitor is GS1101 (CAL-101). In one embodiment, the mTOR inhibitor is AP23841, AZD8055, BEZ235, BGT226, deferolimus (AP23573/MK-8669), EM101/LY303511, everolimus (RAD001), EX2044, EX3855, EX7518, GDC0980, INK-128, KU-0063794, NV-128, OSI-027, PF-4691502, rapalogs, rapamycin, ridaforolimus, SAR543, SF1126, temsirolimus (CCI-779), WYE-125132, XL765, zotarolimus (ABT578), torin 1, GSK2126458, AZD2014, GDC-0349, or XL388, or a mixture thereof. In one embodiment, the mTOR inhibitor is everolimus. In another embodiment, the mTOR inhibitor is AZD8055. In one embodiment, provided herein is a pharmaceutical composition comprising a therapeutically effective amount of GS1101, or a pharmaceutically acceptable form thereof, and everolimus, or a pharmaceutically acceptable form thereof. In another embodiment, provided herein is a pharmaceutical composition comprising a therapeutically effective amount of GS1101, or a pharmaceutically acceptable form thereof, and AZD8055, or a pharmaceutically acceptable form thereof.

In one embodiment of the compositions and methods described herein, the molar ratio of the PI3K delta inhibitor (e.g., GS1101), or a pharmaceutically acceptable form thereof, to the mTOR inhibitor (e.g., everolimus or AZD8055), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, or from about 5:1 to about 1:5. In an embodiment, the mTOR inhibitor is everolimus, and the molar ratio of the PI3K delta inhibitor to the mTOR inhibitor is from about 1000:1 to about 1:1, from about 750:1 to about 10:1, from about 500:1 to about 10:1, from about 500:1 to about 100:1, from about 500:1 to about 200:1, from about 500:1 to about 300:1, from about 500:1 to about 400:1, or about 460:1. In an embodiment, the mTOR inhibitor is AZD8055, and the molar ratio of the PI3K delta inhibitor to the mTOR inhibitor is from about 100:1 to about 1:100, from about 50:1 to about 1:10, from about 50:1 to about 1:1, from about 40:1 to about 1:1, from about 35:1 to about 5:1, about 33:1, or about 3:1.

In one embodiment, the composition comprises the PI3K delta selective inhibitor (e.g. GS1101), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta selective inhibitor (e.g. GS1101), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta selective inhibitor which is GS1101, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the mTOR inhibitor, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the mTOR inhibitor, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the mTOR inhibitor which is everolimus or AZD8055, or a pharmaceutically acceptable form thereof, at an amount sufficient to deliver a blood plasma concentration profile with an AUC (area under curve) of from about 10 ng/mL*h to about 1 μg/mL*h, from about 50 ng/mL*h to about 0.2 μg/mL*h, or from about 70 ng/mL*h to about 150 ng/mL*h.

In one embodiment, the PI3K delta inhibitor (e.g., GS1101) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 6000 ng/mL*hr to about 8000 ng/mL*hr, about 6500 ng/mL*hr to about 7500 ng/mL*hr, or about 7000 ng/mL*hr; and

the mTOR inhibitor (e.g., everolimus or AZD8055) is administered at an amount to reach an AUCss at about 0.1 ng/mL*hr to about 1000 ng/mL*hr, about 1 ng/mL*hr to about 500 ng/mL*hr, about 50 ng/mL*hr to about 200 ng/mL*hr, about 80 ng/mL*hr to about 120 ng/mL*hr, about 90 ng/mL*hr, or about 111 ng/mL*hr. In one embodiment, the mTOR inhibitor is everolimus and is administered at an amount to reach an AUCss at about 90 ng/mL*h. In one embodiment, the mTOR inhibitor is AZD 8055 and is administered at an amount to reach an AUCss at about 111 ng/mL*h.

In one embodiment, the PI3K delta inhibitor (e.g., GS1101) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the mTOR inhibitor (e.g., everolimus or AZD8055) is administered at an amount to reach an AUCss at less than about 1000 ng/mL*hr, less than about 750 ng/mL*hr, less than about 500 ng/mL*hr, less than about 250 ng/mL*hr, less than about 200 ng/mL*hr, less than about 100 ng/mL*hr, less than about 50 ng/mL*hr, less than about 25 ng/mL*hr, less than about 10 ng/mL*hr, less than about 1 ng/mL*hr, less than about 111 ng/mL*hr, or less than about 90 ng/mL*hr.

In one embodiment, the PI3K delta inhibitor (e.g., GS1101) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2300 ng/mL, about 2000 ng/mL to about 2300 ng/mL, or about 2200 ng/mL; and

the mTOR inhibitor (e.g., everolimus or AZD8055) is administered at an amount to reach Cmaxss at about 0.1 ng/mL to about 1000 ng/mL, about 0.1 ng/mL to about 500 ng/mL, about 0.1 ng/mL to about 250 ng/mL, about 1 ng/mL to about 100 ng/mL, about 10 ng/mL to about 80 ng/mL, about 10 ng/mL to about 70 ng/mL, about 12 ng/mL, or about 62 ng/mL. In one embodiment, the mTOR inhibitor is everolimus and is administered at an amount to reach Cmaxss at about 12 ng/mL. In one embodiment, the mTOR inhibitor is AZD 8055 and is administered at an amount to reach Cmaxss at about 62 ng/mL.

In one embodiment, the PI3K delta inhibitor (e.g., GS1101) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the mTOR inhibitor (e.g., everolimus or AZD8055) is administered at an amount to reach Cmaxss at less than about 1000 ng/mL, less than about 750 ng/mL, less than about 500 ng/mL, less than about 250 ng/mL, less than about 200 ng/mL, less than about 100 ng/mL, less than about 50 ng/mL, less than about 25 ng/mL, less than about 10 ng/mL, less than about 1 ng/mL, less than about 62 ng/mL, or less than about 12 ng/mL.

In one embodiment, the composition comprises the PI3K delta inhibitor (e.g., GS1101), or a pharmaceutically acceptable form thereof, at an amount in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta inhibitor (e.g., GS1101), or a pharmaceutically acceptable form thereof, at an amount of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with a mTOR inhibitor (e.g., everolimus or AZD8055), or a pharmaceutically acceptable form thereof, wherein the cancer is diffuse large B-cell lymphoma (activated B-cell-like), diffuse large B-cell lymphoma (germinal center B-cell-like), follicular lymphoma, T-cell lymphoma, mantle cell lymphoma, or multiple myeloma.

In some embodiments of the methods described herein, the PI3K delta inhibitor (e.g., GS1101), or a pharmaceutically acceptable form thereof, and the mTOR inhibitor (e.g., everolimus or AZD8055), or a pharmaceutically acceptable form thereof, are administered at certain dosages. In one embodiment, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with a mTOR inhibitor, or a pharmaceutically acceptable form thereof, wherein the PI3K delta inhibitor (e.g., GS1101), or a pharmaceutically acceptable form thereof, is administered at a dosage of in the range of from about 0.01 mg to about 75 mg daily and the mTOR inhibitor (e.g., everolimus or AZD8055), or a pharmaceutically acceptable form thereof, is administered at a dosage of in the range of from about 0.01 mg to about 1100 mg daily.

In one embodiment, the PI3K delta inhibitor (e.g., GS1101), or a pharmaceutically acceptable form thereof, is administered at a dosage of in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the dosage is in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta inhibitor (e.g., GS1101), or a pharmaceutically acceptable form thereof, is administered at a dosage of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a pharmaceutical composition comprising a therapeutically effective amount of a PI3K delta/gamma dual inhibitor, or a pharmaceutically acceptable form thereof, and a mTOR inhibitor, or a pharmaceutically acceptable form thereof. In one embodiment, the mTOR inhibitor is AP23841, AZD8055, BEZ235, BGT226, deferolimus (AP23573/MK-8669), EM101/LY303511, everolimus (RAD001), EX2044, EX3855, EX7518, GDC0980, INK-128, KU-0063794, NV-128, OSI-027, PF-4691502, rapalogs, rapamycin, ridaforolimus, SAR543, SF1126, temsirolimus (CCI-779), WYE-125132, XL765, zotarolimus (ABT578), torin 1, GSK2126458, AZD2014, GDC-0349, or XL388, or a mixture thereof. In one embodiment, the mTOR inhibitor is everolimus. In another embodiment, the mTOR inhibitor is AZD8055.

In one embodiment of the compositions and methods described herein, the molar ratio of the PI3K delta/gamma dual inhibitor, or a pharmaceutically acceptable form thereof, to the mTOR inhibitor (e.g., everolimus or AZD8055), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 100:1 to about 1:5, from about 80:1 to about 1:5, or from about 75:1 to about 1:5.

In one embodiment, the composition comprises the PI3K delta/gamma inhibitor (e.g., Compound 1), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta/gamma inhibitor (e.g., Compound 1), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta/gamma inhibitor which is Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the mTOR inhibitor, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the mTOR inhibitor, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the mTOR inhibitor which is everolimus or AZD8055, or a pharmaceutically acceptable form thereof, at an amount sufficient to deliver a blood plasma concentration profile with an AUC (area under curve) of from about 10 ng/mL*h to about 1 μg/mL*h, from about 50 ng/mL*h to about 0.2 μg/mL*h, or from about 70 ng/mL*h to about 150 ng/mL*h. In one embodiment, the PI3K delta/gamma dual inhibitor (e.g., Compound 1) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and

the mTOR inhibitor (e.g., everolimus or AZD8055) is administered at an amount to reach an AUCss at about 0.1 ng/mL*hr to about 1000 ng/mL*hr, about 1 ng/mL*hr to about 500 ng/mL*hr, about 50 ng/mL*hr to about 200 ng/mL*hr, about 80 ng/mL*hr to about 120 ng/mL*hr, about 90 ng/mL*hr, or about 111 ng/mL*hr. In one embodiment, the mTOR inhibitor is everolimus and is administered at an amount to reach an AUCss at about 90 ng/mL*h. In one embodiment, the mTOR inhibitor is AZD 8055 and is administered at an amount to reach an AUCss at about 111 ng/mL*h.

In one embodiment, the PI3K delta/gamma dual inhibitor (e.g., Compound 1) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the mTOR inhibitor (e.g., everolimus or AZD8055) is administered at an amount to reach an AUCss at less than about 1000 ng/mL*hr, less than about 750 ng/mL*hr, less than about 500 ng/mL*hr, less than about 250 ng/mL*hr, less than about 200 ng/mL*hr, less than about 100 ng/mL*hr, less than about 50 ng/mL*hr, less than about 25 ng/mL*hr, less than about 10 ng/mL*hr, less than about 1 ng/mL*hr, less than about 111 ng/mL*hr, or less than about 90 ng/mL*hr.

In one embodiment, the PI3K delta/gamma dual inhibitor (e.g., Compound 1) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

the mTOR inhibitor (e.g., everolimus or AZD8055) is administered at an amount to reach Cmaxss at about 0.1 ng/mL to about 1000 ng/mL, about 0.1 ng/mL to about 500 ng/mL, about 0.1 ng/mL to about 250 ng/mL, about 1 ng/mL to about 100 ng/mL, about 10 ng/mL to about 80 ng/mL, about 10 ng/mL to about 70 ng/mL, about 12 ng/mL, or about 62 ng/mL. In one embodiment, the mTOR inhibitor is everolimus and is administered at an amount to reach Cmaxss at about 12 ng/mL. In one embodiment, the mTOR inhibitor is AZD 8055 and is administered at an amount to reach Cmaxss at about 62 ng/mL.

In one embodiment, the PI3K delta/gamma dual inhibitor (e.g., Compound 1) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the mTOR inhibitor (e.g., everolimus or AZD8055) is administered at an amount to reach Cmaxss at less than about 1000 ng/mL, less than about 750 ng/mL, less than about 500 ng/mL, less than about 250 ng/mL, less than about 200 ng/mL, less than about 100 ng/mL, less than about 50 ng/mL, less than about 25 ng/mL, less than about 10 ng/mL, less than about 1 ng/mL, less than about 62 ng/mL, or less than about 12 ng/mL.

In one embodiment, the PI3K delta/gamma dual inhibitor (e.g., Compound 1) is administered at an amount that is decreased by about 1.5 fold to about 50 fold of the amount when administered individually and the mTOR inhibitor (e.g., everolimus or AZD8055) is administered at an amount that is decreased by about 1.1 fold to about 50 fold of the amount when administered individually.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and

the mTOR inhibitor (e.g., everolimus or AZD8055) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, the composition comprises the PI3K delta/gamma dual inhibitor, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta/gamma dual inhibitor, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta/gamma dual inhibitor, or a pharmaceutically acceptable form thereof, in combination with a mTOR inhibitor (e.g., everolimus or AZD8055), or a pharmaceutically acceptable form thereof, wherein the cancer is diffuse large B-cell lymphoma (activated B-cell-like), diffuse large B-cell lymphoma (germinal center B-cell-like), follicular lymphoma, T-cell lymphoma, mantle cell lymphoma, or multiple myeloma.

In some embodiments of the methods described herein, the PI3K delta/gamma dual inhibitor, or a pharmaceutically acceptable form thereof, and the mTOR inhibitor (e.g., everolimus or AZD8055), or a pharmaceutically acceptable form thereof, are administered at certain dosages. In one embodiment, provided herein is a method of treating, managing, or preventing a cancer in a subject comprising administering to the subject a therapeutically effective amount of a PI3K delta/gamma dual inhibitor, or a pharmaceutically acceptable form thereof, in combination with a mTOR inhibitor, or a pharmaceutically acceptable form thereof, wherein the PI3K delta/gamma dual inhibitor, or a pharmaceutically acceptable form thereof, is administered at a dosage of in the range of from about 0.01 mg to about 75 mg daily and the mTOR inhibitor (e.g., everolimus or AZD8055), or a pharmaceutically acceptable form thereof, is administered at a dosage of in the range of from about 0.01 mg to about 1100 mg daily.

In one embodiment, the PI3K delta/gamma dual inhibitor, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta/gamma dual inhibitor, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a composition, e.g., a pharmaceutical composition, comprising a therapeutically effective amount of Compound 1:

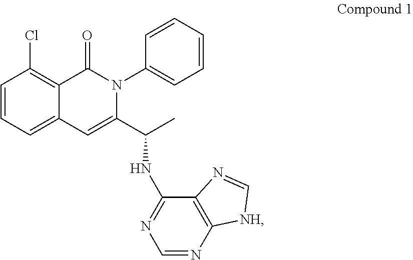 [SMILES: C[C@H](Nc1ncnc2[nH]cnc12)c1cc2cccc(Cl)c2c(=O)n1-c1ccccc1]

or a pharmaceutically acceptable form thereof, in combination with a mTOR inhibitor, or a pharmaceutically acceptable form thereof. In one embodiment, the mTOR inhibitor is AP23841, AZD8055, BEZ235, BGT226, deferolimus (AP23573/MK-8669), EM101/LY303511, everolimus (RAD001), EX2044, EX3855, EX7518, GDC0980, INK-128, KU-0063794, NV-128, OSI-027, PF-4691502, rapalogs, rapamycin, ridaforolimus, SAR543, SF1126, temsirolimus (CCI-779), WYE-125132, XL765, zotarolimus (ABT578), torin 1, GSK2126458, AZD2014, GDC-0349, or XL388, or a mixture thereof. In one embodiment, the mTOR inhibitor is everolimus. In another embodiment, the mTOR inhibitor is AZD8055.

In certain embodiments, provided herein is a method of treating, managing, or preventing a cancer in a subject comprising administering to the subject a therapeutically effective amount of Compound 1:

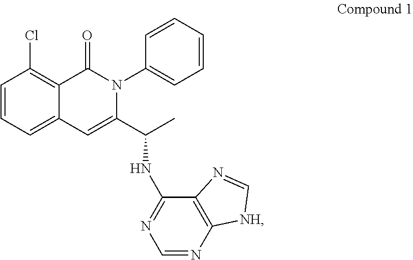 [SMILES: C[C@H](Nc1ncnc2[nH]cnc12)c1cc2cccc(Cl)c2c(=O)n1-c1ccccc1]

or a pharmaceutically acceptable form thereof, and a mTOR inhibitor, or a pharmaceutically acceptable form thereof. In one embodiment, the mTOR inhibitor is AP23841, AZD8055, BEZ235, BGT226, deferolimus (AP23573/MK-8669), EM101/LY303511, everolimus (RAD001), EX2044, EX3855, EX7518, GDC0980, INK-128, KU-0063794, NV-128, OSI-027, PF-4691502, rapalogs, rapamycin, ridaforolimus, SAR543, SF1126, temsirolimus (CCI-779), WYE-125132, XL765, zotarolimus (ABT578), torin 1, GSK2126458, AZD2014, GDC-0349, or XL388, or a mixture thereof. In one embodiment, the mTOR inhibitor is everolimus. In another embodiment, the mTOR inhibitor is AZD8055.

In some embodiments of the compositions and methods described herein, Compound 1, or a pharmaceutically acceptable form thereof, is used in combination with a mTOR inhibitor (e.g., everolimus or AZD8055), or a pharmaceutically acceptable form thereof, at certain molar ratios. In one embodiment, provided herein is a pharmaceutical composition comprising a therapeutically effective amount of Compound 1:

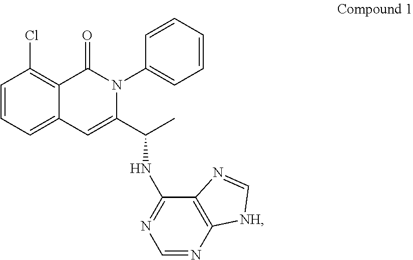 [SMILES: C[C@H](Nc1ncnc2[nH]cnc12)c1cc2cccc(Cl)c2c(=O)n1-c1ccccc1]

or a pharmaceutically acceptable form thereof, and a mTOR inhibitor, or a pharmaceutically acceptable form thereof, wherein the molar ratio of Compound 1, or a pharmaceutically acceptable form thereof, to the mTOR inhibitor (e.g., everolimus or AZD8055), or a pharmaceutically acceptable form thereof, is in the range of from about 1000:1 to about 1:1000.

In one embodiment of the compositions and methods described herein, the molar ratio of Compound 1, or a pharmaceutically acceptable form thereof, to the mTOR inhibitor (e.g., everolimus or AZD8055), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, or from about 5:1 to about 1:5. In one embodiment, the PI3K delta/gamma dual inhibitor is Compound 1, the mTOR inhibitor is everolimus, and the molar ratio of Compound 1 to everolimus is from about 100:1 to about 1:5, from about 80:1 to about 1:5, from about 75:1 to about 1:5, or about 75:1. In one embodiment, the PI3K delta/gamma dual inhibitor is Compound 1, the mTOR inhibitor is AZD 8055, and the molar ratio of Compound 1 to AZD 8055 is from about 100:1 to about 1:5, from about 80:1 to about 1:5, from about 75:1 to about 1:5, from about 10:1 to about 1:5, from about 5:1 to about 1:2, about 5:1, or about 1:1.7.

In one embodiment, the composition comprises the PI3K delta inhibitor (e.g., Compound 1 or GS1101), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta inhibitor (e.g., Compound 1 or GS1101), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta inhibitor which is Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the mTOR inhibitor, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the mTOR inhibitor, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the mTOR inhibitor which is everolimus or AZD8055, or a pharmaceutically acceptable form thereof, at an amount sufficient to deliver a blood plasma concentration profile with an AUC (area under curve) of from about 10 ng/mL*h to about 1 μg/mL*h, from about 50 ng/mL*h to about 0.1 μg/mL*h, or from about 70 ng/mL*h to about 150 ng/mL*h.

In one embodiment, Compound 1 is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and

the mTOR inhibitor (e.g., everolimus or AZD8055) is administered at an amount to reach an AUCss at about 0.1 ng/mL*hr to about 1000 ng/mL*hr, about 1 ng/mL*hr to about 500 ng/mL*hr, about 50 ng/mL*hr to about 200 ng/mL*hr, about 80 ng/mL*hr to about 120 ng/mL*hr, about 90 ng/mL*hr, or about 111 ng/mL*hr. In one embodiment, the mTOR inhibitor is everolimus and is administered at an amount to reach an AUCss at about 90 ng/mL*h. In one embodiment, the mTOR inhibitor is AZD 8055 and is administered at an amount to reach an AUCss at about 111 ng/mL*h.

In one embodiment, Compound 1 is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

the mTOR inhibitor (e.g., everolimus or AZD8055) is administered at an amount to reach Cmaxss at about 0.1 ng/mL to about 1000 ng/mL, about 0.1 ng/mL to about 500 ng/mL, about 0.1 ng/mL to about 250 ng/mL, about 1 ng/mL to about 100 ng/mL, about 10 ng/mL to about 80 ng/mL, about 10 ng/mL to about 70 ng/mL, about 12 ng/mL, or about 62 ng/mL. In one embodiment, the mTOR inhibitor is everolimus and is administered at an amount to reach Cmaxss at about 12 ng/mL. In one embodiment, the mTOR inhibitor is AZD 8055 and is administered at an amount to reach Cmaxss at about 62 ng/mL.

In one embodiment, the PI3K delta/gamma dual inhibitor (e.g., Compound 1) is administered at an amount that is decreased by about 1.5 fold to about 50 fold of the amount when administered individually and the mTOR inhibitor (e.g., everolimus or AZD8055) is administered at an amount that is decreased by about 1.1 fold to about 50 fold of the amount when administered individually.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and the mTOR inhibitor (e.g., everolimus or AZD8055) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment of the compositions and methods described herein, the weight ratio of Compound 1, or a pharmaceutically acceptable form thereof, to everolimus, or a pharmaceutically acceptable form thereof, is in the range of from about 7.5-37.5 of Compound 1 to from 0.5-2.5 of everolimus. In one embodiment, the weight ratio is in the range of from about 75:1 to about 3:1. In one embodiment, the weight ratio is in the range of from about 37.5:1 to about 6:1. In one embodiment, the weight ratio is in the range of from about 25:1 to about 9:1. In one embodiment, the weight ratio is in the range of from about 35:1 to about 30:1. In another embodiment, the weight ratio is about 33:1.

In one embodiment of the compositions and methods described herein, the weight ratio of Compound 1, or a pharmaceutically acceptable form thereof, to AZD8055, or a pharmaceutically acceptable form thereof, is in the range of from about 7.5-37.5 of Compound 1 to from 12-60 of AZD8055. In one embodiment, the weight ratio is in the range of from about 3:1 to about 1:8. In one embodiment, the weight ratio is in the range of from about 1.5:1 to about 1:4. In one embodiment, the weight ratio is in the range of from about 1:1 to about 1:2.7. In one embodiment, the weight ratio is in the range from about 10:1 to about 1:5. In another embodiment, the weight ratio is in the range from about 5:1 to about 1:2. In another embodiment, the weight ratio is in the range from about 5:1 to about 1:1.8.

In some embodiments of the compositions and methods described herein, the composition comprises Compound 1, or a pharmaceutically acceptable form thereof, and the mTOR inhibitor (e.g., everolimus or AZD8055), or a pharmaceutically acceptable form thereof, at certain amounts. In one embodiment, provided herein is a pharmaceutical composition comprising a therapeutically effective amount of Compound 1:

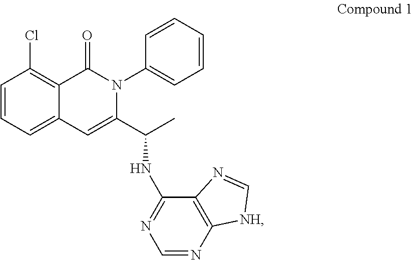 [SMILES: C[C@H](Nc1ncnc2[nH]cnc12)c1cc2cccc(Cl)c2c(=O)n1-c1ccccc1]

or a pharmaceutically acceptable form thereof, and a mTOR inhibitor, or a pharmaceutically acceptable form thereof, wherein the composition comprises Compound 1, or a pharmaceutically acceptable form thereof, at an amount in the range of from about 0.01 mg to about 75 mg and the mTOR inhibitor (e.g., everolimus or AZD8055), or a pharmaceutically acceptable form thereof, at an amount of in the range of from about 0.01 mg to about 1100 mg.

In one embodiment, the composition comprises Compound 1, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg. In one embodiment, the composition comprises Compound 1, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg. In one embodiment, the composition comprises Compound 1, or a pharmaceutically acceptable form thereof, at an amount of about 50 mg, about 37.5 mg, about 25 mg, about 20 mg, about 15 mg, about 10 mg, about 5 mg, or about 1 mg.

In one embodiment, the composition comprises the mTOR inhibitor (e.g., everolimus or AZD8055), or a pharmaceutically acceptable form thereof, at an amount in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg. In one embodiment, the composition comprises the mTOR inhibitor (e.g., everolimus or AZD8055), or a pharmaceutically acceptable form thereof, at an amount of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, or less than about 25 mg.

In one embodiment, the composition comprises everolimus, or a pharmaceutically acceptable form thereof, at an amount in the range of from about 0.01 mg to about 5 mg, from about 0.01 mg to about 2.5 mg, from about 0.05 mg to about 2 mg, from about 0.1 mg to about 1.5 mg, from about 0.2 mg to about 1 mg, or from about 0.4 mg to about 0.75 mg. In one embodiment, the composition comprises everolimus, or a pharmaceutically acceptable form thereof, at an amount of less than about 5 mg, less than about 3 mg, less than about 2.5 mg, less than about 2 mg, less than about 1.5 mg, less than about 1 mg, less than about 0.75 mg, less than about 0.5 mg, or less than about 0.25 mg. In one embodiment, the composition comprises everolimus, or a pharmaceutically acceptable form thereof, at an amount of about 5 mg, about 3 mg, about 2.5 mg, about 2 mg, about 1.5 mg, about 1 mg, about 0.75 mg, about 0.5 mg, or about 0.25 mg.

In one embodiment, the composition comprises AZD8055, or a pharmaceutically acceptable form thereof, at an amount in the range of from about 1 mg to about 120 mg, from about 2 mg to about 80 mg, from about 5 mg to about 60 mg, from about 10 mg to about 40 mg, from about 15 mg to about 30 mg, or from about 20 mg to about 25 mg. In one embodiment, the composition comprises AZD8055, or a pharmaceutically acceptable form thereof, at an amount of less than about 120 mg, less than about 80 mg, less than about 60 mg, less than about 40 mg, less than about 30 mg, less than about 25 mg, less than about 20 mg, less than about 15 mg, or less than about 10 mg. In one embodiment, the composition comprises AZD8055, or a pharmaceutically acceptable form thereof, at an amount of about 120 mg, about 80 mg, about 60 mg, about 40 mg, about 30 mg, about 25 mg, about 20 mg, about 15 mg, or about 10 mg.

In certain embodiments, provided herein is a method of treating, managing, or preventing a cancer in a subject comprising administering to the subject a therapeutically effective amount of Compound 1, or a pharmaceutically acceptable form thereof, in combination with a mTOR inhibitor, or a pharmaceutically acceptable form thereof, wherein the cancer is diffuse large B-cell lymphoma (activated B-cell-like), diffuse large B-cell lymphoma (germinal center B-cell-like), follicular lymphoma, T-cell lymphoma, mantle cell lymphoma, or multiple myeloma. In one embodiment, the mTOR inhibitor is everolimus. In another embodiment, the mTOR inhibitor is AZD8055.

In some embodiments of the methods described herein, Compound 1, or a pharmaceutically acceptable form thereof, and the mTOR inhibitor (e.g., everolimus or AZD8055), or a pharmaceutically acceptable form thereof, are administered at certain dosages. In one embodiment, provided herein is a method of treating, managing, or preventing a cancer in a subject comprising administering to the subject a therapeutically effective amount of Compound 1:

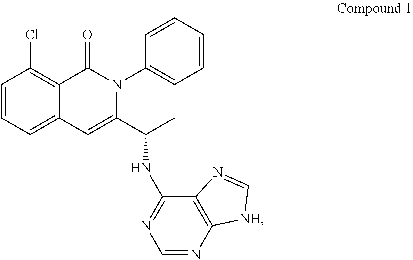 [SMILES: C[C@H](Nc1ncnc2[nH]cnc12)c1cc2cccc(Cl)c2c(=O)n1-c1ccccc1]

or a pharmaceutically acceptable form thereof, in combination with a mTOR inhibitor, or a pharmaceutically acceptable form thereof, wherein Compound 1, or a pharmaceutically acceptable form thereof, is administered at a dosage of in the range of from about 0.01 mg to about 75 mg daily and the mTOR inhibitor (e.g., everolimus or AZD8055), or a pharmaceutically acceptable form thereof, is administered at a dosage of in the range of from about 0.01 mg to about 1100 mg daily.

In one embodiment, Compound 1, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily. In one embodiment, Compound 1, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily. In one embodiment, Compound 1, or a pharmaceutically acceptable form thereof, is administered at a dosage of about 50 mg, about 37.5 mg, about 25 mg, about 20 mg, about 15 mg, about 10 mg, about 5 mg, or about 1 mg daily.

In one embodiment, the mTOR inhibitor (e.g., everolimus or AZD8055), or a pharmaceutically acceptable form thereof, is administered at a dosage of in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg daily. In one embodiment, the mTOR inhibitor (e.g., everolimus or AZD8055), or a pharmaceutically acceptable form thereof, is administered at a dosage of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, or less than about 25 mg daily.

In one embodiment, everolimus, or a pharmaceutically acceptable form thereof, is administered at a dosage of in the range of from about 0.01 mg to about 5 mg, from about 0.01 mg to about 2.5 mg, from about 0.05 mg to about 2 mg, from about 0.1 mg to about 1.5 mg, from about 0.2 mg to about 1 mg, or from about 0.4 mg to about 0.75 mg daily. In one embodiment, everolimus, or a pharmaceutically acceptable form thereof, is administered at a dosage of less than about 5 mg, less than about 3 mg, less than about 2.5 mg, less than about 2 mg, less than about 1.5 mg, less than about 1 mg, less than about 0.75 mg, less than about 0.5 mg, or less than about 0.25 mg daily. In one embodiment, everolimus, or a pharmaceutically acceptable form thereof, is administered at a dosage of about 5 mg, about 3 mg, about 2.5 mg, about 2 mg, about 1.5 mg, about 1 mg, about 0.75 mg, about 0.5 mg, or about 0.25 mg daily.

In one embodiment, AZD8055, or a pharmaceutically acceptable form thereof, is administered at a dosage of in the range of from about 1 mg to about 120 mg, from about 2 mg to about 80 mg, from about 5 mg to about 60 mg, from about 10 mg to about 40 mg, from about 15 mg to about 30 mg, or from about 20 mg to about 25 mg daily. In one embodiment, AZD8055, or a pharmaceutically acceptable form thereof, is administered at a dosage of less than about 120 mg, less than about 80 mg, less than about 60 mg, less than about 40 mg, less than about 30 mg, less than about 25 mg, less than about 20 mg, less than about 15 mg, or less than about 10 mg daily. In one embodiment, AZD8055, or a pharmaceutically acceptable form thereof, is administered at a dosage of about 120 mg, about 80 mg, about 60 mg, about 40 mg, about 30 mg, about 25 mg, about 20 mg, about 15 mg, or about 10 mg daily.

In one embodiment, the mTOR inhibitor (e.g., everolimus or AZD8055), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before Compound 1, or a pharmaceutically acceptable form thereof, is administered. In another embodiment, the mTOR inhibitor (e.g., everolimus or AZD8055), or a pharmaceutically acceptable form thereof, is administered concurrently with Compound 1, or a pharmaceutically acceptable form thereof, in a single dosage form or separate dosage forms. In yet another embodiment, the mTOR inhibitor (e.g., everolimus or AZD8055), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after Compound 1, or a pharmaceutically acceptable form thereof, is administered. In one embodiment, the mTOR inhibitor is everolimus. In another embodiment, the mTOR inhibitor is AZD8055.

In certain embodiments, the PI3K inhibitor (e.g., Compound 1), or a pharmaceutically acceptable form thereof, and the mTOR inhibitor (e.g., everolimus or AZD8055), or a pharmaceutically acceptable form thereof, are in a single dosage form. In other embodiments, the PI3K inhibitor (e.g., Compound 1), or a pharmaceutically acceptable form thereof, and the mTOR inhibitor (e.g., everolimus or AZD8055), or a pharmaceutically acceptable form thereof, are in separate dosage forms.

In certain embodiments, the PI3K inhibitor (e.g., Compound 1), or a pharmaceutically acceptable form thereof, and the mTOR inhibitor (e.g., everolimus or AZD8055), are administered via a same route, e.g., both are administered orally. In other embodiments, the PI3K inhibitor (e.g., Compound 1), or a pharmaceutically acceptable form thereof, and the mTOR inhibitor (e.g., everolimus or AZD8055), are administered via different routes, e.g., one is administered orally and the other is administered intravenously. In one embodiment, Compound 1 is administered orally once per day and everolimus is administered orally once per day. In one embodiment, Compound 1 is administered orally once per day and AZD8055 is administered orally once per day.

In certain embodiments, the PI3K inhibitor (e.g., Compound 1), or a pharmaceutically acceptable form thereof, and the mTOR inhibitor (e.g., everolimus or AZD8055), or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., GS1101), a PI3K delta/gamma dual inhibitor, and a mTOR inhibitor (e.g., everolimus or AZD8055).

2.4 Combinations of PI3K Inhibitors and AKT Inhibitors

Provided herein are compositions, e.g., pharmaceutical compositions, comprising a therapeutically effective amount of a PI3K inhibitor, or a pharmaceutically acceptable form thereof, and an AKT inhibitor, or a pharmaceutically acceptable form thereof.

Also provided herein are methods of treating, managing, or preventing a cancer in a subject comprising administering to the subject a therapeutically effective amount of a PI3K inhibitor, or a pharmaceutically acceptable form thereof, in combination with an AKT inhibitor, or a pharmaceutically acceptable form thereof.

AKT inhibitors that can be used in the compositions and methods provided herein include, but are not limited to, AZD5363, miltefosine, perifosine, VQD-002, MK-2206, GSK690693, GDC-0068, triciribine, CCT128930, PHT-427, and honokiol.

In one embodiment, the AKT inhibitor is AZD5363 (4-amino-N-[(1S)-1-(4-chlorophenyl)-3-hydroxypropyl]-1-(7H-pyrrolo[2,3-d]pyrimidin-4-yl)-4-piperidinecarboxamide,), miltefosine (2-(hexadecoxy-oxido-phosphoryl)oxyethyl-trimethyl-azanium), perifosine (1,1-dimethylpiperidinium-4-yl octadecyl phosphate), VQD-002 (triciribine phosphate monohydrate, 6-Amino-4-methyl-8-((β-D-ribofuranosyl)-4H,8H-pyrrolo[4,3,2-de]pyrimido[4,5-c]pyridazine phosphate monohydrate), MK-2206 (8-[4-(1-aminocyclobutyl)phenyl]-9-phenyl-2H-[1,2,4]triazolo[3,4-f][1,6]naphthyridin-3-one), GSK690693 (4-(2-(4-Amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol), GDC-0068 ((2S)-2(4-chlorophenyl)-1-[4-[(5R,7R)-7-hydroxy-5-methyl-6,7-dihydro-5H-cyclopenta[d]pyrimidin-4-yl]piperazin-1-yl]-3-(propan-2-ylamino)propan-1-one), triciribine (1,5-dihydro-5-methyl-1-β-D-ribofuranosyl-1,2,5,6,8-pentaazaacenaphthylen-3-amine), CCT128930 (4-(4-chlorobenzyl)-1-(7H-pyrrolo[2,3-d]pyrimidin-4-yl)piperidin-4-amine), PHT-427 (4-dodecyl-N-(1,3,4-thiadiazol-2-yl)benzenesulfonamide), or honokiol (2-(4-hydroxy-3-prop-2-enyl-phenyl)-4-prop-2-enyl-phenol), or a mixture thereof.

In one embodiment, the AKT inhibitor is perifosine. Perifosine has a chemical name of 1,1-dimethylpiperidinium-4-yl octadecyl phosphate, and is of the structure:

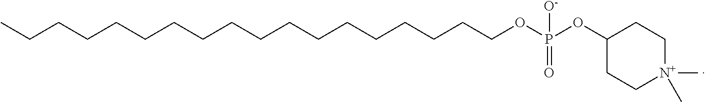 [SMILES: CCCCCCCCCCCCCCCCCCOP(=O)([O-])OC1CC[N+](C)(C)CC1 ; "."]

In one embodiment, the AKT inhibitor is MK-2206. MK-2206 has a chemical name of 8-[4-(1-aminocyclobutyl)phenyl]-9-phenyl-2H-[1,2,4]triazolo[3,4-f][1,6]naphthyridin-3-one, and is of the structure:

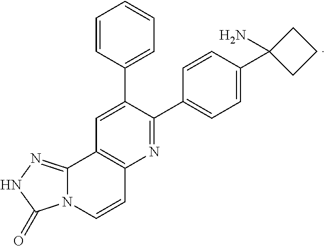 [SMILES: NC1(c2ccc(-c3nc4ccn5c(=O)[nH]nc5c4cc3-c3ccccc3)cc2)CCC1 ; "."]

In certain embodiments, provided herein is a composition, e.g., a pharmaceutical composition, comprising a therapeutically effective amount of a PI3K delta inhibitor, or a pharmaceutically acceptable form thereof, and an AKT inhibitor, or a pharmaceutically acceptable form thereof. In one embodiment, the PI3K delta inhibitor is GS1101 (CAL-101). In one embodiment, the AKT inhibitor is AZD5363, miltefosine, perifosine, VQD-002, MK-2206, GSK690693, GDC-0068, triciribine, CCT128930, PHT-427, or honokiol, or a mixture thereof. In one embodiment, the AKT inhibitor is perifosine. In another embodiment, the AKT inhibitor is MK-2206. In one embodiment, provided herein is a pharmaceutical composition comprising a therapeutically effective amount of GS1101, or a pharmaceutically acceptable form thereof, and perifosine, or a pharmaceutically acceptable form thereof. In another embodiment, provided herein is a composition comprising a therapeutically effective amount of GS1101, or a pharmaceutically acceptable form thereof, and MK-2206, or a pharmaceutically acceptable form thereof.

In one embodiment of the compositions and methods described herein, the molar ratio of the PI3K delta inhibitor (e.g., GS1101), or a pharmaceutically acceptable form thereof, to the AKT inhibitor (e.g., perifosine or MK-2206), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 10:1 to about 1:1, from about 6:1 to about 2:1, from about 5:1 to about 3:1, about 6:1, or about 3:1.

In one embodiment, the composition comprises the PI3K delta inhibitor (e.g., GS1101), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta inhibitor (e.g., GS1101), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta inhibitor which is GS1101, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the AKT inhibitor, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the AKT inhibitor, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h.

In one embodiment, the PI3K delta inhibitor (e.g., GS1101) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 6000 ng/mL*hr to about 8000 ng/mL*hr, about 6500 ng/mL*hr to about 7500 ng/mL*hr, or about 7000 ng/mL*hr; and the AKT inhibitor (e.g., perifosine or MK-2206) is administered at an amount to reach an AUCss at about 0.1 nmol/mL*hr to about 10000 nmol/mL*hr, about 1 nmol/mL*hr to about 8000 nmol/mL*hr, about 1000 nmol/mL*hr to about 7000 nmol/mL*hr, about 4000 nmol/mL*hr to about 7000 nmol/mL*hr, about 5000 nmol/mL*hr to about 6000 nmol/mL*hr, or about 5,860 nmol/mL*hr. In one embodiment, the AKT inhibitor and is administered at an amount to reach an AUCss at about 5,860 nmol/mL*hr.

In one embodiment, the PI3K delta inhibitor (e.g., GS1101) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the AKT inhibitor (e.g., perifosine or MK-2206) is administered at an amount to reach an AUCss at less than about 10000 nmol/mL*hr, less than about 9000 nmol/mL*hr, less than about 8000 nmol/mL*hr, less than about 7000 nmol/mL*hr, less than about 6000 nmol/mL*hr, less than about 5000 nmol/mL*hr, less than about 4000 nmol/mL*hr, less than about 3000 nmol/mL*hr, less than about 2000 nmol/mL*hr, less than about 1000 nmol/mL*hr, less than about 500 nmol/mL*hr, less than about 250 nmol/mL*hr, less than about 100 nmol/mL*hr, less than about 10 nmol/mL*hr, less than about 1 nmol/mL*hr, or less than about 1 nmol/mL*hr.

In one embodiment, the PI3K delta inhibitor (e.g., GS1101) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2300 ng/mL, about 2000 ng/mL to about 2300 ng/mL, or about 2200 ng/mL; and

the AKT inhibitor (e.g., perifosine or MK-2206) is administered at an amount to reach Cmaxss at about 0.1 ng/mL to about 10000 ng/mL, about 1 ng/mL to about 8000 ng/mL, about 10 ng/mL to about 7000 ng/mL, about 50 ng/mL to about 6000 ng/mL, about 6000 ng/mL, or about 78 ng/mL. In one embodiment, the AKT inhibitor (e.g., perifosine) is administered at an amount to reach Cmaxss at about 6000 ng/mL. In one embodiment, the AKT inhibitor (e.g., MK-2206) is administered at an amount to reach Cmaxss at about 78 ng/mL.

In one embodiment, the PI3K delta inhibitor (e.g., GS1101) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the AKT inhibitor (e.g., perifosine or MK-2206) is administered at an amount to reach Cmaxss at less than about 10000 ng/mL, less than about 8000 ng/mL, less than about 7000 ng/mL, less than about 6000 ng/mL, less than about 5000 ng/mL, less than about 1000 ng/mL, less than about 100 ng/mL, less than about 50 ng/mL, less than about 25 ng/mL, less than about 10 ng/mL, less than about 1 ng/mL, less than about 6000 ng/mL, or less than about 78 ng/mL. In one embodiment, the composition comprises the PI3K delta inhibitor (e.g., GS1101), or a pharmaceutically acceptable form thereof, at an amount in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta inhibitor (e.g., GS1101), or a pharmaceutically acceptable form thereof, at an amount of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with an AKT inhibitor (e.g., perifosine or MK-2206), or a pharmaceutically acceptable form thereof, wherein the cancer is diffuse large B-cell lymphoma (activated B-cell-like), diffuse large B-cell lymphoma (germinal center B-cell-like), follicular lymphoma, T-cell lymphoma, mantle cell lymphoma, or multiple myeloma.

In some embodiments of the methods described herein, the PI3K delta inhibitor (e.g., GS1101), or a pharmaceutically acceptable form thereof, and the AKT inhibitor (e.g., perifosine or MK-2206), or a pharmaceutically acceptable form thereof, are administered at certain dosages. In one embodiment, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with an AKT inhibitor, or a pharmaceutically acceptable form thereof, wherein the PI3K delta inhibitor (e.g., GS1101), or a pharmaceutically acceptable form thereof, is administered at a dosage of in the range of from about 0.01 mg to about 75 mg daily and the AKT inhibitor (e.g., perifosine or MK-2206), or a pharmaceutically acceptable form thereof, is administered at a dosage of in the range of from about 0.01 mg to about 1100 mg daily.

In one embodiment, the PI3K delta inhibitor (e.g., GS1101), or a pharmaceutically acceptable form thereof, is administered at a dosage of in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta inhibitor (e.g., GS1101), or a pharmaceutically acceptable form thereof, is administered at a dosage of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a pharmaceutical composition comprising a therapeutically effective amount of a PI3K delta/gamma dual inhibitor, or a pharmaceutically acceptable form thereof, and an AKT inhibitor, or a pharmaceutically acceptable form thereof. In one embodiment, the AKT inhibitor is AZD5363, miltefosine, perifosine, VQD-002, MK-2206, GSK690693, GDC-0068, triciribine, CCT128930, PHT-427, or honokiol, or a mixture thereof. In one embodiment, the AKT inhibitor is perifosine. In another embodiment, the AKT inhibitor is MK-2206.

In one embodiment of the compositions and methods described herein, the molar ratio of the PI3K delta/gamma dual inhibitor, or a pharmaceutically acceptable form thereof, to the AKT inhibitor (e.g., perifosine or MK-2206), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, or from about 1:1 to about 1:2.

In one embodiment, the composition comprises the PI3K delta/gamma dual inhibitor, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta/gamma dual inhibitor, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta/gamma dual inhibitor, or a pharmaceutically acceptable form thereof, in combination with an AKT inhibitor (e.g., perifosine or MK-2206), or a pharmaceutically acceptable form thereof, wherein the cancer is diffuse large B-cell lymphoma (activated B-cell-like), diffuse large B-cell lymphoma (germinal center B-cell-like), follicular lymphoma, T-cell lymphoma, mantle cell lymphoma, or multiple myeloma.

In some embodiments of the methods described herein, the PI3K delta/gamma dual inhibitor, or a pharmaceutically acceptable form thereof, and the AKT inhibitor (e.g., perifosine or MK-2206), or a pharmaceutically acceptable form thereof, are administered at certain dosages. In one embodiment, provided herein is a method of treating, managing, or preventing a cancer in a subject comprising administering to the subject a therapeutically effective amount of a PI3K delta/gamma dual inhibitor, or a pharmaceutically acceptable form thereof, in combination with an AKT inhibitor, or a pharmaceutically acceptable form thereof, wherein the PI3K delta/gamma dual inhibitor, or a pharmaceutically acceptable form thereof, is administered at a dosage of in the range of from about 0.01 mg to about 75 mg daily and the AKT inhibitor (e.g., perifosine or MK-2206), or a pharmaceutically acceptable form thereof, is administered at a dosage of in the range of from about 0.01 mg to about 1100 mg daily.

In one embodiment, the PI3K delta/gamma dual inhibitor, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta/gamma dual inhibitor, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a pharmaceutical composition comprising a therapeutically effective amount of Compound 1:

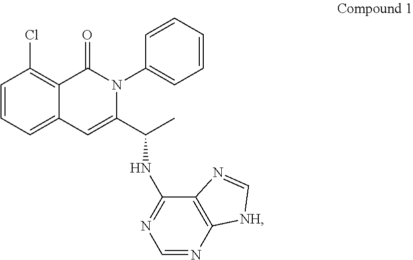 [SMILES: C[C@H](Nc1ncnc2[nH]cnc12)c1cc2cccc(Cl)c2c(=O)n1-c1ccccc1]

or a pharmaceutically acceptable form thereof, and an AKT inhibitor, or a pharmaceutically acceptable form thereof. In one embodiment, the AKT inhibitor is AZD5363, miltefosine, perifosine, VQD-002, MK-2206, GSK690693, GDC-0068, triciribine, CCT128930, PHT-427, or honokiol, or a mixture thereof. In one embodiment, the AKT inhibitor is perifosine. In another embodiment, the AKT inhibitor is MK-2206.

In certain embodiments, provided herein is a method of treating, managing, or preventing a cancer in a subject comprising administering to the subject a therapeutically effective amount of Compound 1:

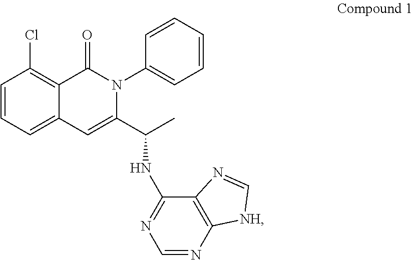 [SMILES: C[C@H](Nc1ncnc2[nH]cnc12)c1cc2cccc(Cl)c2c(=O)n1-c1ccccc1]

or a pharmaceutically acceptable form thereof, in combination with an AKT inhibitor, or a pharmaceutically acceptable form thereof. In one embodiment, the AKT inhibitor is AZD5363, miltefosine, perifosine, VQD-002, MK-2206, GSK690693, GDC-0068, triciribine, CCT128930, PHT-427, or honokiol, or a mixture thereof. In one embodiment, the AKT inhibitor is perifosine. In another embodiment, the AKT inhibitor is MK-2206.

In some embodiments of the compositions and methods described herein, Compound 1, or a pharmaceutically acceptable form thereof, is used in combination with an AKT inhibitor (e.g., perifosine or MK-2206), or a pharmaceutically acceptable form thereof, at certain molar ratios. In one embodiment, provided herein is a pharmaceutical composition comprising a therapeutically effective amount of Compound 1:

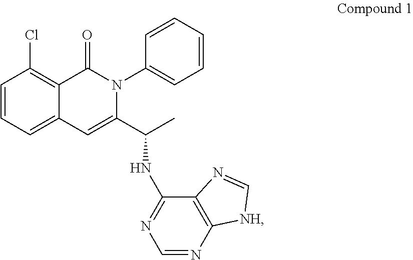 [SMILES: C[C@H](Nc1ncnc2[nH]cnc12)c1cc2cccc(Cl)c2c(=O)n1-c1ccccc1]

or a pharmaceutically acceptable form thereof, and an AKT inhibitor, or a pharmaceutically acceptable form thereof, wherein the molar ratio of Compound 1, or a pharmaceutically acceptable form thereof, to the AKT inhibitor (e.g., perifosine or MK-2206), or a pharmaceutically acceptable form thereof, is in the range of from about 1000:1 to about 1:1000.

In one embodiment of the compositions and methods described herein, the molar ratio of Compound 1, or a pharmaceutically acceptable form thereof, to the AKT inhibitor (e.g., perifosine or MK-2206), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, or from about 1:1 to about 1:2.

In one embodiment, the PI3K delta/gamma dual inhibitor (e.g., Compound 1) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and

the AKT inhibitor (e.g., perifosine or MK-2206) is administered at an amount to reach an AUCss at about 0.1 nmol/mL*hr to about 10000 nmol/mL*hr, about 1 nmol/mL*hr to about 8000 nmol/mL*hr, about 1000 nmol/mL*hr to about 7000 nmol/mL*hr, about 4000 nmol/mL*hr to about 7000 nmol/mL*hr, about 5000 nmol/mL*hr to about 6000 nmol/mL*hr, or about 5,860 nmol/mL*hr. In one embodiment, the AKT inhibitor and is administered at an amount to reach an AUCss at about 5,860 nmol/mL*hr.

In one embodiment, the PI3K delta/gamma dual inhibitor (e.g., Compound 1) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the AKT inhibitor (e.g., perifosine or MK-2206) is administered at an amount to reach an AUCss at less than about 10000 nmol/mL*hr, less than about 9000 nmol/mL*hr, less than about 8000 nmol/mL*hr, less than about 7000 nmol/mL*hr, less than about 6000 nmol/mL*hr, less than about 5000 nmol/mL*hr, less than about 4000 nmol/mL*hr, less than about 3000 nmol/mL*hr, less than about 2000 nmol/mL*hr, less than about 1000 nmol/mL*hr, less than about 500 nmol/mL*hr, less than about 250 nmol/mL*hr, less than about 100 nmol/mL*hr, less than about 10 nmol/mL*hr, less than about 1 nmol/mL*hr, or less than about 1 nmol/mL*hr.

In one embodiment, the PI3K delta/gamma dual inhibitor (e.g., Compound 1) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

the AKT inhibitor (e.g., perifosine or MK-2206) is administered at an amount to reach Cmaxss at about 0.1 ng/mL to about 10000 ng/mL, about 1 ng/mL to about 8000 ng/mL, about 10 ng/mL to about 7000 ng/mL, about 50 ng/mL to about 6000 ng/mL, about 6000 ng/mL, or about 78 ng/mL. In one embodiment, the AKT inhibitor (e.g., perifosine) is administered at an amount to reach Cmaxss at about 6000 ng/mL. In one embodiment, the AKT inhibitor (e.g., MK-2206) is administered at an amount to reach Cmaxss at about 78 ng/mL.

In one embodiment, the PI3K delta/gamma dual inhibitor (e.g., Compound 1) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the AKT inhibitor (e.g., perifosine or MK-2206) is administered at an amount to reach Cmaxss at less than about 10000 ng/mL, less than about 8000 ng/mL, less than about 7000 ng/mL, less than about 6000 ng/mL, less than about 5000 ng/mL, less than about 1000 ng/mL, less than about 100 ng/mL, less than about 50 ng/mL, less than about 25 ng/mL, less than about 10 ng/mL, less than about 1 ng/mL, less than about 6000 ng/mL, or less than about 78 ng/mL.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and

the AKT inhibitor (e.g., perifosine or MK-2206) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment of the compositions and methods described herein, the weight ratio of Compound 1, or a pharmaceutically acceptable form thereof, to perifosine, or a pharmaceutically acceptable form thereof, is in the range of from about 7.5-37.5 of Compound 1 to from 15-75 of perifosine. In one embodiment, the weight ratio is in the range of from about 2.5:1 to about 1:10. In one embodiment, the weight ratio is in the range of from about 1.25:1 to about 1:5. In one embodiment, the weight ratio is in the range of from about 1:1.2 to about 1:3.3.

In one embodiment of the compositions and methods described herein, the weight ratio of Compound 1, or a pharmaceutically acceptable form thereof, to MK-2206, or a pharmaceutically acceptable form thereof, is in the range of from about 7.5-37.5 of Compound 1 to from 3-15 of MK-2206. In one embodiment, the weight ratio is in the range of from about 12.5:1 to about 1:2. In one embodiment, the weight ratio is in the range of from about 6.25:1 to about 1:1. In one embodiment, the weight ratio is in the range of from about 4.2:1 to about 1.5:1. In one embodiment, the weight ratio is in the range of from about 2:1 to about 1.2:1.

In some embodiments of the compositions and methods described herein, the composition comprises Compound 1, or a pharmaceutically acceptable form thereof, and the AKT inhibitor (e.g., perifosine or MK-2206), or a pharmaceutically acceptable form thereof, at certain amounts. In one embodiment, provided herein is a pharmaceutical composition comprising a therapeutically effective amount of Compound 1:

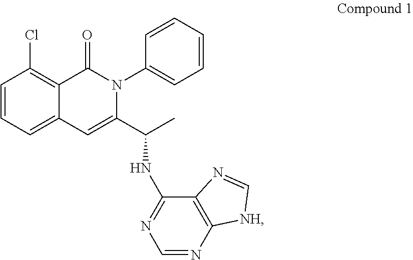 [SMILES: C[C@H](Nc1ncnc2[nH]cnc12)c1cc2cccc(Cl)c2c(=O)n1-c1ccccc1]

or a pharmaceutically acceptable form thereof, and an AKT inhibitor, or a pharmaceutically acceptable form thereof, wherein the composition comprises Compound 1, or a pharmaceutically acceptable form thereof, at an amount in the range of from about 0.01 mg to about 75 mg and the AKT inhibitor (e.g., perifosine or MK-2206), or a pharmaceutically acceptable form thereof, at an amount of in the range of from about 0.01 mg to about 1100 mg.

In one embodiment, the composition comprises Compound 1, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg. In one embodiment, the composition comprises Compound 1, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg. In one embodiment, the composition comprises Compound 1, or a pharmaceutically acceptable form thereof, at an amount of about 50 mg, about 37.5 mg, about 25 mg, about 20 mg, about 15 mg, about 10 mg, about 5 mg, or about 1 mg.

In one embodiment, the composition comprises the AKT inhibitor (e.g., perifosine or MK-2206), or a pharmaceutically acceptable form thereof, at an amount in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg. In one embodiment, the composition comprises the AKT inhibitor (e.g., perifosine or MK-2206), or a pharmaceutically acceptable form thereof, at an amount of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, or less than about 25 mg.

In one embodiment, the composition comprises perifosine, or a pharmaceutically acceptable form thereof, at an amount in the range of from about 1 mg to about 150 mg, from about 2 mg to about 100 mg, from about 5 mg to about 75 mg, from about 10 mg to about 50 mg, from about 15 mg to about 40 mg, or from about 20 mg to about 30 mg. In one embodiment, the composition comprises perifosine, or a pharmaceutically acceptable form thereof, at an amount of less than about 150 mg, less than about 100 mg, less than about 75 mg, less than about 50 mg, less than about 40 mg, less than about 30 mg, less than about 20 mg, less than about 10 mg, or less than about 5 mg. In one embodiment, the composition comprises perifosine, or a pharmaceutically acceptable form thereof, at an amount of about 150 mg, about 100 mg, about 75 mg, about 50 mg, about 40 mg, about 30 mg, about 20 mg, about 10 mg, or about 5 mg.

In one embodiment, the composition comprises MK-2206, or a pharmaceutically acceptable form thereof, at an amount in the range of from about 0.1 mg to about 100 mg, 1 mg to about 60 mg, 0.1 mg to about 30 mg, from about 0.2 mg to about 20 mg, from about 0.5 mg to about 15 mg, from about 1 mg to about 10 mg, from about 2 mg to about 8 mg, or from about 4 mg to about 6 mg. In one embodiment, the composition comprises MK-2206, or a pharmaceutically acceptable form thereof, at an amount of less than about 100 mg, less than about 60 mg, less than about 30 mg, less than about 20 mg, less than about 15 mg, less than about 10 mg, less than about 8 mg, less than about 6 mg, less than about 4 mg, less than about 2 mg, or less than about 1 mg. In one embodiment, the composition comprises MK-2206, or a pharmaceutically acceptable form thereof, at an amount of about 30 mg, about 20 mg, about 15 mg, about 10 mg, about 8 mg, about 6 mg, about 4 mg, about 2 mg, or about 1 mg.

In certain embodiments, provided herein is a method of treating, managing, or preventing a cancer in a subject comprising administering to the subject a therapeutically effective amount of Compound 1, or a pharmaceutically acceptable form thereof, in combination with an AKT inhibitor, or a pharmaceutically acceptable form thereof, wherein the cancer is diffuse large B-cell lymphoma (activated B-cell-like), diffuse large B-cell lymphoma (germinal center B-cell-like), follicular lymphoma, T-cell lymphoma, mantle cell lymphoma, or multiple myeloma. In one embodiment, the AKT inhibitor is perifosine. In another embodiment, the AKT inhibitor is MK-2206.

In some embodiments of the methods described herein, Compound 1, or a pharmaceutically acceptable form thereof, and the AKT inhibitor (e.g., perifosine or MK-2206), or a pharmaceutically acceptable form thereof, are administered at certain dosages. In one embodiment, provided herein is a method of treating, managing, or preventing a cancer in a subject comprising administering to the subject a therapeutically effective amount of Compound 1:

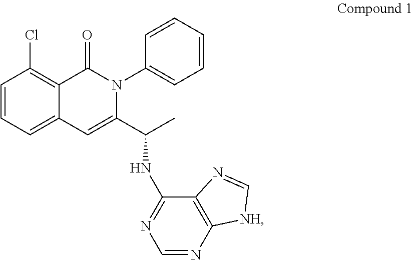 [SMILES: C[C@H](Nc1ncnc2[nH]cnc12)c1cc2cccc(Cl)c2c(=O)n1-c1ccccc1]

or a pharmaceutically acceptable form thereof, in combination with an AKT inhibitor, or a pharmaceutically acceptable form thereof, wherein Compound 1, or a pharmaceutically acceptable form thereof, is administered at a dosage of in the range of from about 0.01 mg to about 75 mg daily and the AKT inhibitor (e.g., perifosine or MK-2206), or a pharmaceutically acceptable form thereof, is administered at a dosage of in the range of from about 0.01 mg to about 1100 mg daily.

In one embodiment, Compound 1, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily. In one embodiment, Compound 1, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily. In one embodiment, Compound 1, or a pharmaceutically acceptable form thereof, is administered at a dosage of about 50 mg, about 37.5 mg, about 25 mg, about 20 mg, about 15 mg, about 10 mg, about 5 mg, or about 1 mg daily.

In one embodiment, the AKT inhibitor (e.g., perifosine or MK-2206), or a pharmaceutically acceptable form thereof, is administered at a dosage of in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg daily. In one embodiment, the AKT inhibitor (e.g., perifosine or MK-2206), or a pharmaceutically acceptable form thereof, is administered at a dosage of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, or less than about 25 mg daily.

In one embodiment, perifosine, or a pharmaceutically acceptable form thereof, is administered at a dosage of in the range of from about 1 mg to about 150 mg, from about 2 mg to about 100 mg, from about 5 mg to about 75 mg, from about 10 mg to about 50 mg, from about 15 mg to about 40 mg, or from about 20 mg to about 30 mg daily. In one embodiment, perifosine, or a pharmaceutically acceptable form thereof, is administered at a dosage of less than about 150 mg, less than about 100 mg, less than about 75 mg, less than about 50 mg, less than about 40 mg, less than about 30 mg, less than about 20 mg, less than about 10 mg, or less than about 5 mg daily. In one embodiment, perifosine, or a pharmaceutically acceptable form thereof, is administered at a dosage of about 150 mg, about 100 mg, about 75 mg, about 50 mg, about 40 mg, about 30 mg, about 20 mg, about 10 mg, or about 5 mg daily.

In one embodiment, MK-2206, or a pharmaceutically acceptable form thereof, is administered at a dosage of in the range of from about 0.1 mg to about 100 mg, from about 1 mg to about 60 mg, from about 0.1 mg to about 30 mg, from about 0.2 mg to about 20 mg, from about 0.5 mg to about 15 mg, from about 1 mg to about 10 mg, from about 2 mg to about 8 mg, or from about 4 mg to about 6 mg daily. In one embodiment, MK-2206, or a pharmaceutically acceptable form thereof, is administered at a dosage of less than about 30 mg, less than about 20 mg, less than about 15 mg, less than about 10 mg, less than about 8 mg, less than about 6 mg, less than about 4 mg, less than about 2 mg, or less than about 1 mg daily. In one embodiment, MK-2206, or a pharmaceutically acceptable form thereof, is administered at a dosage of about 30 mg, about 20 mg, about 15 mg, about 10 mg, about 8 mg, about 6 mg, about 4 mg, about 2 mg, or about 1 mg daily.

In one embodiment, MK-2206, or a pharmaceutically acceptable form thereof, is administered at a dosage of in the range of from about 0.2 mg to about 60 mg, from about 0.4 mg to about 40 mg, from about 1 mg to about 30 mg, from about 2 mg to about 20 mg, from about 4 mg to about 16 mg, or from about 8 mg to about 12 mg every other day. In one embodiment, MK-2206, or a pharmaceutically acceptable form thereof, is administered at a dosage of less than about 60 mg, less than about 40 mg, less than about 30 mg, less than about 20 mg, less than about 16 mg, less than about 12 mg, less than about 8 mg, less than about 4 mg, or less than about 2 mg every other day. In one embodiment, MK-2206, or a pharmaceutically acceptable form thereof, is administered at a dosage of about 60 mg, about 40 mg, about 35 mg, about 20 mg, about 16 mg, about 12 mg, about 8 mg, about 4 mg, or about 2 mg every other day.

In one embodiment, the AKT inhibitor (e.g., perifosine or MK-2206), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before Compound 1, or a pharmaceutically acceptable form thereof, is administered. In another embodiment, the AKT inhibitor (e.g., perifosine or MK-2206), or a pharmaceutically acceptable form thereof, is administered concurrently with Compound 1, or a pharmaceutically acceptable form thereof, in a single dosage form or separate dosage forms. In yet another embodiment, the AKT inhibitor (e.g., perifosine or MK-2206), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after Compound 1, or a pharmaceutically acceptable form thereof, is administered. In one embodiment, the AKT inhibitor is perifosine. In another embodiment, the AKT inhibitor is MK-2206.

In certain embodiments, the PI3K inhibitor (e.g., Compound 1), or a pharmaceutically acceptable form thereof, and the AKT inhibitor (e.g., perifosine or MK-2206), or a pharmaceutically acceptable form thereof, are in a single dosage form. In other embodiments, the PI3K inhibitor (e.g., Compound 1), or a pharmaceutically acceptable form thereof, and the AKT inhibitor (e.g., perifosine or MK-2206), or a pharmaceutically acceptable form thereof, are in separate dosage forms.

In certain embodiments, the PI3K inhibitor (e.g., Compound 1), or a pharmaceutically acceptable form thereof, and the AKT inhibitor (e.g., perifosine or MK-2206), are administered via a same route, e.g., both are administered orally. In other embodiments, the PI3K inhibitor (e.g., Compound 1), or a pharmaceutically acceptable form thereof, and the AKT inhibitor (e.g., perifosine or MK-2206), are administered via different routes, e.g., one is administered orally and the other is administered intravenously. In one embodiment, Compound 1 is administered orally once per day and perifosine is administered orally once per day. In one embodiment, Compound 1 is administered orally once per day and MK-2206 is administered orally once per day. In one embodiment, Compound 1 is administered orally once per day and MK-2206 is administered orally every other day.

In certain embodiments, the PI3K inhibitor (e.g., Compound 1), or a pharmaceutically acceptable form thereof, and the AKT inhibitor (e.g., perifosine or MK-2206), or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., GS1101), a PI3K delta/gamma dual inhibitor, and an AKT inhibitor (e.g., perifosine or MK-2206).

2.5 Combinations of PI3K Inhibitors and Proteasome Inhibitors

PI3K inhibitors can be effective for treatment of T-cell lymphoma. Flinn, I. W. et al. Clinical Safety and Activity in a Phase 1 Trial of IPI-145, a Potent Inhibitor of Phosphoinositide-3-Kinase-{delta}, {gamma}, in Patients with Advanced Hematologic Malignancies. ASH Annual Meeting Abstracts 120, 3663 (2012). Bortezomib can be used as a monotherapy for treatment of PTCL and CTCL. Zinzani, P L et al. Phase II trial of proteasome inhibitor bortezomib in patients with relapsed or refractory cutaneous T-cell lymphoma. Journal of clinical oncology: official journal of the American Society of Clinical Oncology 25, 4293-4297, doi:10.1200/JCO.2007.11.4207 (2007). In certain lymphoma cell lines, inhibition of the PI3K/mTOR/AKT pathway may overcome resistance to proteasome inhibitors. Kim, A. et al. The dual PI3K and mTOR inhibitor NVP-BEZ235 exhibits anti-proliferative activity and overcomes bortezomib resistance in mantle cell lymphoma cells. Leukemia research 36, 912-920, doi:10.1016/j.leukres.2012.02.010 (2012).

Provided herein are pharmaceutical compositions comprising a therapeutically effective amount of a PI3K inhibitor, or a pharmaceutically acceptable form thereof, and a proteasome inhibitor, or a pharmaceutically acceptable form thereof.

Also provided herein are methods of treating (e.g., inhibiting, managing, or preventing) a cancer in a subject comprising administering to the subject a therapeutically effective amount of a PI3K inhibitor, or a pharmaceutically acceptable form thereof, in combination with a proteasome inhibitor, or a pharmaceutically acceptable form thereof. In specific embodiments, the cancer is a T cell lymphoma, e.g., PTCL and/or CTCL.

Proteasome inhibitors that can be used in the compositions and methods provided herein include, but are not limited to, bortezomib, carfilzomib, CEP-18770, disulfiram, epigallocatechin-3-gallate, epoxomicin, lactacystin, MG132, MLN9708, ONX 0912, and salinosporamide A.

In one embodiment, the proteasome inhibitor is bortezomib ([(1R)-3-methyl-1-({(2S)-3-phenyl-2-[(pyrazin-2-ylcarbonyl)amino]propanoyl}amino)butyl]boronic acid), carfilzomib ((S)-4-methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide), CEP-18770 ((R)-1-((2S,3R)-3-hydroxy-2-(2-phenylpicolinamido)butanamido)-3-methylbutan-2-ylboronic acid), disulfiram (1,1′,1″,1′″-[disulfanediylbis(carbonothioylnitrilo)]tetraethane), epigallocatechin-3-gallate ((2R,3R)-5,7-dihydroxy-2-(3,4,5-trihydroxyphenyl)-3,4-dihydro-2H-1-benzopyran-3-yl 3,4,5-trihydroxybenzoate), epoxomicin (N-acetyl-N-methyl-L-isoleucyl-L-isoleucyl-N-[(1S)-3-methyl-1-[[(2R)-2-methyloxiranyl]carbonyl]butyl]-L-threoninamide), lactacystin (2-(acetylamino)-3-[({3-hydroxy-2-[1-hydroxy-2-methylpropyl]-4-methyl-5-oxopyrrolidin-2-yl}carbonyl)sulfanyl]propanoic acid), MG132 (benzyl(S)-4-methyl-1-((S)-4-methyl-1-((S)-4-methyl-1-oxopentan-2-ylamino)-1-oxopentan-2-ylamino)-1-oxopentan-2-ylcarbamate), MLN9708 (4-(carboxymethyl)-2-((R)-1-(2-(2,5-dichlorobenzamido)acetamido)-3-methylbutyl)-6-oxo-1,3,2-dioxaborinane-4-carboxylic acid), ONX 0912 (O-methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide), or salinosporamide A ((4R,5S)-4-(2-chloroethyl)-1-((1S)-cyclohex-2-enyl(hydroxy)methyl)-5-methyl-6-oxa-2-azabicyclo[3.2.0]heptane-3,7-dione), or a mixture thereof.

In one embodiment, the proteasome inhibitor is bortezomib. Bortezomib has a chemical name of [(1R)-3-methyl-1-({(2S)-3-phenyl-2-[(pyrazin-2-ylcarbonyl)amino]propanoyl}amino)butyl]boronic acid, and is of the structure:

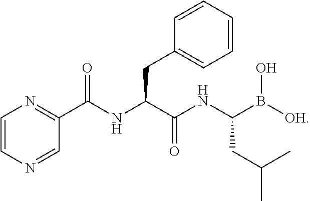 [SMILES: CC(C)C[C@H](NC(=O)[C@H](Cc1ccccc1)NC(=O)c1cnccn1)B(O)O]

In one embodiment, the proteasome inhibitor is carfilzomib. Carfilzomib has a chemical name of (S)-4-methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide, and is of the structure:

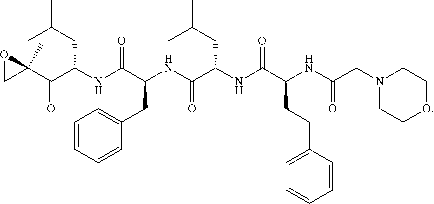 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

In certain embodiments, provided herein is a pharmaceutical composition comprising a therapeutically effective amount of a PI3K delta inhibitor, or a pharmaceutically acceptable form thereof, and a proteasome inhibitor, or a pharmaceutically acceptable form thereof. In one embodiment, the PI3K delta inhibitor is GS1101 (CAL-101). In one embodiment, the proteasome inhibitor is bortezomib, carfilzomib, CEP-18770, disulfiram, epigallocatechin-3-gallate, epoxomicin, lactacystin, MG132, MLN9708, ONX 0912, or salinosporamide A, or a mixture thereof. In one embodiment, the proteasome inhibitor is bortezomib. In another embodiment, the proteasome inhibitor is carfilzomib. In one embodiment, provided herein is a pharmaceutical composition comprising a therapeutically effective amount of GS1101, or a pharmaceutically acceptable form thereof, and bortezomib, or a pharmaceutically acceptable form thereof. In another embodiment, provided herein is a pharmaceutical composition comprising a therapeutically effective amount of GS1101, or a pharmaceutically acceptable form thereof, and carfilzomib, or a pharmaceutically acceptable form thereof.

In one embodiment of the compositions and methods described herein, the molar ratio of the PI3K delta inhibitor (e.g., GS1101), or a pharmaceutically acceptable form thereof, to the proteasome inhibitor (e.g., bortezomib or carfilzomib), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 200:1 to about 1:1, from about 175:1 to about 5:1, from about 165:1 to about 10:1, about 163:1, or about 12:1.

In one embodiment, the composition comprises the PI3K delta inhibitor (e.g., GS1101), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta inhibitor (e.g., GS1101), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta inhibitor which is Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the proteasome inhibitor, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the proteasome inhibitor, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the proteasome inhibitor which is bortezomib or carfilzomib, or a pharmaceutically acceptable form thereof, at an amount sufficient to deliver a blood plasma concentration profile with an AUC (area under curve) of from about 100 ng/mL*h to about 1 μg/mL*h, from about 200 ng/mL*h to about 500 ng/mL*h, or from about 300 ng/mL*h to about 400 ng/mL*h. In one embodiment, the PI3K delta inhibitor (e.g., GS1101) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 6000 ng/mL*hr to about 8000 ng/mL*hr, about 6500 ng/mL*hr to about 7500 ng/mL*hr, or about 7000 ng/mL*hr; and

the proteasome inhibitor (e.g., bortezomib or carfilzomib) is administered at an amount to reach an AUCss at about 0.1 ng/mL*hr to about 1000 ng/mL*hr, about 1 ng/mL*hr to about 500 ng/mL*hr, about 50 ng/mL*hr to about 500 ng/mL*hr, about 100 ng/mL*hr to about 400 ng/mL*hr, about 200 ng/mL*hr, about 400 ng/mL*hr, about 300 ng/mL*hr, or about 400 ng/mL*hr. In one embodiment, the proteasome inhibitor is bortezomib and is administered at an amount to reach an AUCss at about 359 ng/mL*h. In one embodiment, the proteasome inhibitor is carfilzomib and is administered at an amount to reach an AUCss at about 379 ng/mL*h.

In one embodiment, the PI3K delta inhibitor (e.g., GS1101) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the proteasome inhibitor (e.g., bortezomib or carfilzomib) is administered at an amount to reach an AUCss at less than about 1000 ng/mL*hr, less than about 750 ng/mL*hr, less than about 500 ng/mL*hr, less than about 250 ng/mL*hr, less than about 200 ng/mL*hr, less than about 100 ng/mL*hr, less than about 50 ng/mL*hr, less than about 25 ng/mL*hr, less than about 10 ng/mL*hr, less than about 1 ng/mL*hr, less than about 379 ng/mL*hr, or less than about 359 ng/mL*hr.

In one embodiment, the PI3K delta inhibitor (e.g., GS1101) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2300 ng/mL, about 2000 ng/mL to about 2300 ng/mL, or about 2200 ng/mL; and

the proteasome inhibitor (e.g., bortezomib or carfilzomib) is administered at an amount to reach Cmaxss at about 0.1 ng/mL to about 10000 ng/mL, about 0.1 ng/mL to about 5000 ng/mL, about 1 ng/mL to about 5000 ng/mL, about 10 ng/mL to about 5000 ng/mL, about 50 ng/mL to about 4500 ng/mL, about 84 ng/mL, or about 4323 ng/mL. In one embodiment, the proteasome inhibitor is bortezomib and is administered at an amount to reach Cmaxss at about 50 ng/mL to about 100 ng/mL, about 60 ng/mL to about 90 ng/mL, or about 84 ng/mL. In one embodiment, the proteasome inhibitor is carfilzomib and is administered at an amount to reach Cmaxss at about 2000 ng/mL to about 5000 ng/mL, about 3000 ng/mL to about 5000 ng/mL, about 4000 ng/mL to about 4500 ng/mL, or about 4232 ng/mL.

In one embodiment, the PI3K delta inhibitor (e.g., GS1101) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the proteasome inhibitor (e.g., bortezomib or carfilzomib) is administered at an amount to reach Cmaxss at less than about 1000 ng/mL, less than about 750 ng/mL, less than about 500 ng/mL, less than about 250 ng/mL, less than about 200 ng/mL, less than about 100 ng/mL, less than about 50 ng/mL, less than about 25 ng/mL, less than about 10 ng/mL, less than about 1 ng/mL, less than about 4232 ng/mL, or less than about 84 ng/mL.

In one embodiment, the composition comprises the PI3K delta inhibitor (e.g., GS1101), or a pharmaceutically acceptable form thereof, at an amount in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta inhibitor (e.g., GS1101), or a pharmaceutically acceptable form thereof, at an amount of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating (e.g., inhibiting, managing, or preventing) a cancer in a subject comprising administering to the subject a combination of a PI3K delta inhibitor (e.g., GS1101 or Compound 1), or a pharmaceutically acceptable form thereof, and a proteasome inhibitor (e.g., bortezomib or carfilzomib), or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma (e.g., CTCL or PTCL), mantle cell lymphoma, or multiple myeloma. In certain embodiments, the combination is therapeutically effective. In certain embodiments, the combination is synergistic. In a specific embodiment, the combination is effective for treatment of a T cell lymphoma, e.g., PTCL and/or CTCL. In other embodiments, the combination is effective for treatment of CLL.

In certain embodiments, provided herein is a method of treating (e.g., inhibiting, managing, or preventing) a cancer in a subject comprising administering to the subject a a PI3K delta inhibitor (e.g., GS1101 or Compound 1), or a pharmaceutically acceptable form thereof, in combination with a proteasome inhibitor (e.g., bortezomib or carfilzomib), or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma (e.g., PTCL and/or CTCL), mantle cell lymphoma, or multiple myeloma. In certain embodiments, the combination is therapeutically effective. In certain embodiments, the combination is synergistic. In a specific embodiment, the combination is effective for treatment of a T cell lymphoma, e.g., PTCL and/or CTCL.

In some embodiments of the methods described herein, the PI3K delta inhibitor (e.g., GS1101), or a pharmaceutically acceptable form thereof, and the proteasome inhibitor (e.g., bortezomib or carfilzomib), or a pharmaceutically acceptable form thereof, are administered at certain dosages. In one embodiment, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with a proteasome inhibitor, or a pharmaceutically acceptable form thereof, wherein the PI3K delta inhibitor (e.g., GS1101), or a pharmaceutically acceptable form thereof, is administered at a dosage of in the range of from about 0.01 mg to about 75 mg daily and the proteasome inhibitor (e.g., bortezomib or carfilzomib), or a pharmaceutically acceptable form thereof, is administered at a dosage of in the range of from about 0.01 mg to about 1100 mg daily.

In one embodiment, the PI3K delta inhibitor (e.g., GS1101), or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta inhibitor (e.g., GS1101), or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a pharmaceutical composition comprising a therapeutically effective amount of a PI3K delta/gamma dual inhibitor, or a pharmaceutically acceptable form thereof, and a proteasome inhibitor, or a pharmaceutically acceptable form thereof. In one embodiment, the proteasome inhibitor is bortezomib, carfilzomib, CEP-18770, disulfiram, epigallocatechin-3-gallate, epoxomicin, lactacystin, MG132, MLN9708, ONX 0912, or salinosporamide A, or a mixture thereof. In one embodiment, the proteasome inhibitor is bortezomib. In another embodiment, the proteasome inhibitor is carfilzomib.

In one embodiment of the compositions and methods described herein, the molar ratio of the PI3K delta/gamma dual inhibitor, or a pharmaceutically acceptable form thereof, to the proteasome inhibitor (e.g., bortezomib or carfilzomib), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 30:1 to about 1:1, about 27:1 to about 1:1, about 26:1 to about 2:1, about 26:1, or about 2:1. In one embodiment, the PI3K delta/gamma dual inhibitor is Compound 1, the proteasome inhibitor is bortezomib, and the molar ratio of Compound 1 to bortezomib is from about 100:1 to about 1:1, from about 50:1 to about 1:1, from about 30:1 to about 1:1, or about 26:1. In one embodiment, the PI3K delta/gamma dual inhibitor is Compound 1, the proteasome inhibitor is carfilzomib, and the molar ratio of Compound 1 to carfilzomib is from about 50:1 to about 1:1, from about 25:1 to about 1:1, from about 10:1 to about 1:1, from about 5:1 to about 1:1, or about 2:1.

In one embodiment, the composition comprises the PI3K delta/gamma dual inhibitor (e.g., Compound 1), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta/gamma dual inhibitor (e.g., Compound 1), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta/gamma dual inhibitor which is Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the proteasome inhibitor, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the proteasome inhibitor, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the proteasome inhibitor which is bortezomib or carfilzomib, or a pharmaceutically acceptable form thereof, at an amount sufficient to deliver a blood plasma concentration profile with an AUC (area under curve) of from about 100 ng/mL*h to about 1 μg/mL*h, from about 200 ng/mL*h to about 500 ng/mL*h, or from about 300 ng/mL*h to about 400 ng/mL*h. In one embodiment, the PI3K delta/gamma dual inhibitor (e.g., Compound 1) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and

the proteasome inhibitor (e.g., bortezomib or carfilzomib) is administered at an amount to reach an AUCss at about 0.1 ng/mL*hr to about 1000 ng/mL*hr, about 1 ng/mL*hr to about 500 ng/mL*hr, about 50 ng/mL*hr to about 500 ng/mL*hr, about 100 ng/mL*hr to about 400 ng/mL*hr, about 200 ng/mL*hr, about 400 ng/mL*hr, about 300 ng/mL*hr, about 400 ng/mL*hr. In one embodiment, the proteasome inhibitor is bortezomib and is administered at an amount to reach an AUCss at about 359 ng/mL*h. In one embodiment, the proteasome inhibitor is carfilzomib and is administered at an amount to reach an AUCss at about 379 ng/mL*h.

In one embodiment, the PI3K delta/gamma dual inhibitor (e.g., Compound 1) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the proteasome inhibitor (e.g., bortezomib or carfilzomib) is administered at an amount to reach an AUCss at less than about 1000 ng/mL*hr, less than about 750 ng/mL*hr, less than about 500 ng/mL*hr, less than about 250 ng/mL*hr, less than about 200 ng/mL*hr, less than about 100 ng/mL*hr, less than about 50 ng/mL*hr, less than about 25 ng/mL*hr, less than about 10 ng/mL*hr, less than about 1 ng/mL*hr, less than about 379 ng/mL*hr, or less than about 359 ng/mL*hr.

In one embodiment, the PI3K delta/gamma dual inhibitor (e.g., Compound 1) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

the proteasome inhibitor (e.g., bortezomib or carfilzomib) is administered at an amount to reach Cmaxss at about 0.1 ng/mL to about 10000 ng/mL, about 0.1 ng/mL to about 5000 ng/mL, about 1 ng/mL to about 5000 ng/mL, about 10 ng/mL to about 5000 ng/mL, about 50 ng/mL to about 4500 ng/mL, about 84 ng/mL, or about 4323 ng/mL. In one embodiment, the proteasome inhibitor is bortezomib and is administered at an amount to reach Cmaxss at about 50 ng/mL to about 100 ng/mL, about 60 ng/mL to about 90 ng/mL, or about 84 ng/mL. In one embodiment, the proteasome inhibitor is carfilzomib and is administered at an amount to reach Cmaxss at about 2000 ng/mL to about 5000 ng/mL, about 3000 ng/mL to about 5000 ng/mL, about 4000 ng/mL to about 4500 ng/mL, or about 4232 ng/mL.

In one embodiment, the PI3K delta/gamma dual inhibitor (e.g., Compound 1) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the proteasome inhibitor (e.g., bortezomib or carfilzomib) is administered at an amount to reach Cmaxss at less than about 1000 ng/mL, less than about 750 ng/mL, less than about 500 ng/mL, less than about 250 ng/mL, less than about 200 ng/mL, less than about 100 ng/mL, less than about 50 ng/mL, less than about 25 ng/mL, less than about 10 ng/mL, less than about 1 ng/mL, less than about 4232 ng/mL, or less than about 84 ng/mL.

In one embodiment, the PI3K delta/gamma dual inhibitor (e.g., Compound 1) is administered at an amount that is decreased by about 1.5 fold to about 50 fold of the amount when administered individually and the proteasome inhibitor (e.g., bortezomib or carfilzomib) is administered at an amount that is decreased by about 1.1 fold to about 50 fold of the amount when administered individually.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and

the proteasome inhibitor (e.g., bortezomib or carfilzomib) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, the composition comprises the PI3K delta/gamma dual inhibitor, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta/gamma dual inhibitor, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta/gamma dual inhibitor, or a pharmaceutically acceptable form thereof, in combination with a proteasome inhibitor (e.g., bortezomib or carfilzomib), or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma.

In some embodiments of the methods described herein, the PI3K delta/gamma dual inhibitor, or a pharmaceutically acceptable form thereof, and the proteasome inhibitor (e.g., bortezomib or carfilzomib), or a pharmaceutically acceptable form thereof, are administered at certain dosages. In one embodiment, provided herein is a method of treating, managing, or preventing a cancer in a subject comprising administering to the subject a therapeutically effective amount of a PI3K delta/gamma dual inhibitor, or a pharmaceutically acceptable form thereof, in combination with a proteasome inhibitor, or a pharmaceutically acceptable form thereof, wherein the PI3K delta/gamma dual inhibitor, or a pharmaceutically acceptable form thereof, is administered at a dosage of in the range of from about 0.01 mg to about 75 mg daily and the proteasome inhibitor (e.g., bortezomib or carfilzomib), or a pharmaceutically acceptable form thereof, is administered at a dosage of in the range of from about 0.01 mg to about 1100 mg daily.

In one embodiment, the PI3K delta/gamma dual inhibitor, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta/gamma dual inhibitor, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a pharmaceutical composition comprising a therapeutically effective amount of Compound 1:

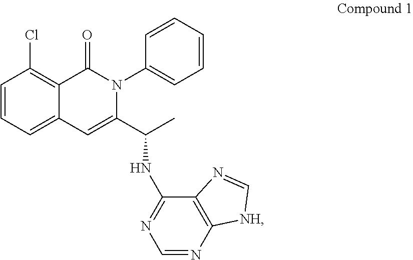 [SMILES: C[C@H](Nc1ncnc2[nH]cnc12)c1cc2cccc(Cl)c2c(=O)n1-c1ccccc1]

or a pharmaceutically acceptable form thereof, and a proteasome inhibitor, or a pharmaceutically acceptable form thereof. In one embodiment, the proteasome inhibitor is bortezomib, carfilzomib, CEP-18770, disulfiram, epigallocatechin-3-gallate, epoxomicin, lactacystin, MG132, MLN9708, ONX 0912, or salinosporamide A, or a mixture thereof. In one embodiment, the proteasome inhibitor is bortezomib. In another embodiment, the proteasome inhibitor is carfilzomib.

In certain embodiments, provided herein is a method of treating, managing, or preventing a cancer in a subject comprising administering to the subject a therapeutically effective amount of Compound 1:

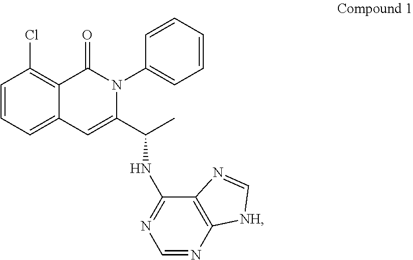 [SMILES: C[C@H](Nc1ncnc2[nH]cnc12)c1cc2cccc(Cl)c2c(=O)n1-c1ccccc1]

or a pharmaceutically acceptable form thereof, in combination with a proteasome inhibitor, or a pharmaceutically acceptable form thereof. In one embodiment, the proteasome inhibitor is bortezomib, carfilzomib, CEP-18770, disulfiram, epigallocatechin-3-gallate, epoxomicin, lactacystin, MG132, MLN9708, ONX 0912, or salinosporamide A, or a mixture thereof. In one embodiment, the proteasome inhibitor is bortezomib. In another embodiment, the proteasome inhibitor is carfilzomib.

In some embodiments of the compositions and methods described herein, Compound 1, or a pharmaceutically acceptable form thereof, is used in combination with a proteasome inhibitor (e.g., bortezomib or carfilzomib), or a pharmaceutically acceptable form thereof, at certain molar ratios. In one embodiment, provided herein is a pharmaceutical composition comprising a therapeutically effective amount of Compound 1:

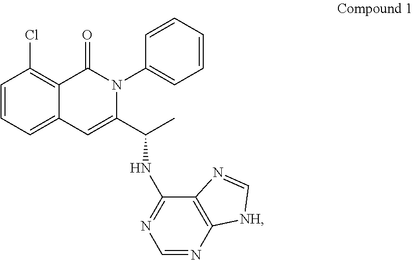 [SMILES: C[C@H](Nc1ncnc2[nH]cnc12)c1cc2cccc(Cl)c2c(=O)n1-c1ccccc1]

or a pharmaceutically acceptable form thereof, and a proteasome inhibitor, or a pharmaceutically acceptable form thereof, wherein the molar ratio of Compound 1, or a pharmaceutically acceptable form thereof, to the proteasome inhibitor (e.g., bortezomib or carfilzomib), or a pharmaceutically acceptable form thereof, is in the range of from about 1000:1 to about 1:1000.

In one embodiment of the compositions and methods described herein, the molar ratio of Compound 1, or a pharmaceutically acceptable form thereof, to the proteasome inhibitor (e.g., bortezomib or carfilzomib), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, or from about 5:1 to about 1:5.

In one embodiment of the compositions and methods described herein, the weight ratio of Compound 1, or a pharmaceutically acceptable form thereof, to bortezomib, or a pharmaceutically acceptable form thereof, is in the range of from about 7.5-37.5 of Compound 1 to from 0.4-2 of bortezomib. In one embodiment, the weight ratio is in the range of from about 90:1 to about 4:1. In one embodiment, the weight ratio is in the range of from about 45:1 to about 8:1. In one embodiment, the weight ratio is in the range of from about 30:1 to about 12:1. In one embodiment, the weight ratio is in the range of from about 30:1 to about 1:1. In one embodiment, the weight ratio is about 29:1. In another embodiment, the weight ratio is about 1.1:1.

In one embodiment of the compositions and methods described herein, the weight ratio of Compound 1, or a pharmaceutically acceptable form thereof, to bortezomib, or a pharmaceutically acceptable form thereof, is in the range of from about 7.5-37.5 of Compound 1 to from 0.25-1.25 of bortezomib. In one embodiment, the weight ratio is in the range of from about 150:1 to about 6:1. In one embodiment, the weight ratio is in the range of from about 75:1 to about 12:1. In one embodiment, the weight ratio is in the range of from about 50:1 to about 18:1.

In one embodiment of the compositions and methods described herein, the weight ratio of Compound 1, or a pharmaceutically acceptable form thereof, to bortezomib, or a pharmaceutically acceptable form thereof, is in the range of from about 7.5-37.5 of Compound 1 to from 3.8-19 of bortezomib. In one embodiment, the weight ratio is in the range of from about 10:1 to about 1:2.5. In one embodiment, the weight ratio is in the range of from about 5:1 to about 1:1.25. In one embodiment, the weight ratio is in the range of from about 3.3:1 to about 1.2:1.

In some embodiments of the compositions and methods described herein, the composition comprises Compound 1, or a pharmaceutically acceptable form thereof, and the proteasome inhibitor (e.g., bortezomib or carfilzomib), or a pharmaceutically acceptable form thereof, at certain amounts. In one embodiment, provided herein is a pharmaceutical composition comprising a therapeutically effective amount of Compound 1:

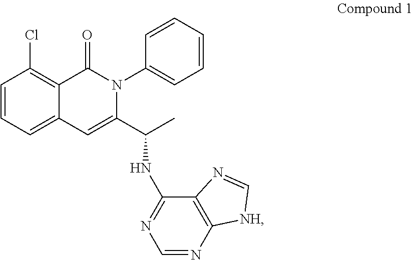 [SMILES: C[C@H](Nc1ncnc2[nH]cnc12)c1cc2cccc(Cl)c2c(=O)n1-c1ccccc1]

or a pharmaceutically acceptable form thereof, and a proteasome inhibitor, or a pharmaceutically acceptable form thereof, wherein the composition comprises Compound 1, or a pharmaceutically acceptable form thereof, at an amount in the range of from about 0.01 mg to about 75 mg and the proteasome inhibitor (e.g., bortezomib or carfilzomib), or a pharmaceutically acceptable form thereof, at an amount of in the range of from about 0.01 mg to about 1100 mg.

In one embodiment, the composition comprises Compound 1, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg. In one embodiment, the composition comprises Compound 1, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg. In one embodiment, the composition comprises Compound 1, or a pharmaceutically acceptable form thereof, at an amount of about 50 mg, about 37.5 mg, about 25 mg, about 20 mg, about 15 mg, about 10 mg, about 5 mg, or about 1 mg.

In one embodiment, the composition comprises the proteasome inhibitor (e.g., bortezomib or carfilzomib), or a pharmaceutically acceptable form thereof, at an amount in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg. In one embodiment, the composition comprises the proteasome inhibitor (e.g., bortezomib or carfilzomib), or a pharmaceutically acceptable form thereof, at an amount of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, or less than about 25 mg.

In one embodiment, the composition comprises bortezomib, or a pharmaceutically acceptable form thereof, at an amount in the range of from about 0.01 mg to about 2.5 mg, from about 0.01 mg to about 2 mg, from about 0.05 mg to about 1.5 mg, from about 0.1 mg to about 1 mg, from about 0.2 mg to about 0.8 mg, or from about 0.4 mg to about 0.6 mg. In one embodiment, the composition comprises bortezomib, or a pharmaceutically acceptable form thereof, at an amount of less than about 2.5 mg, less than about 2 mg, less than about 1.5 mg, less than about 1.2 mg, less than about 1 mg, less than about 0.8 mg, less than about 0.6 mg, less than about 0.4 mg, or less than about 0.2 mg. In one embodiment, the composition comprises bortezomib, or a pharmaceutically acceptable form thereof, at an amount of about 2.5 mg, about 2 mg, about 1.5 mg, about 1.2 mg, about 1 mg, about 0.8 mg, about 0.6 mg, about 0.4 mg, or about 0.2 mg.

In one embodiment, the composition comprises carfilzomib, or a pharmaceutically acceptable form thereof, at an amount in the range of from about 0.1 mg to about 38 mg, from about 0.2 mg to about 30 mg, from about 0.5 mg to about 19 mg, from about 1 mg to about 15 mg, from about 2 mg to about 10 mg, or from about 4 mg to about 8 mg. In one embodiment, the composition comprises carfilzomib, or a pharmaceutically acceptable form thereof, at an amount of less than about 38 mg, less than about 30 mg, less than about 19 mg, less than about 15 mg, less than about 10 mg, less than about 8 mg, less than about 6 mg, less than about 4 mg, or less than about 2 mg. In one embodiment, the composition comprises carfilzomib, or a pharmaceutically acceptable form thereof, at an amount of about 38 mg, about 30 mg, about 19 mg, about 15 mg, about 10 mg, about 8 mg, about 6 mg, about 4 mg, or about 2 mg.

In certain embodiments, provided herein is a method of treating, managing, or preventing a cancer in a subject comprising administering to the subject a therapeutically effective amount of Compound 1, or a pharmaceutically acceptable form thereof, in combination with a proteasome inhibitor, or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma. In one embodiment, the proteasome inhibitor is bortezomib. In another embodiment, the proteasome inhibitor is carfilzomib.

In some embodiments of the methods described herein, Compound 1, or a pharmaceutically acceptable form thereof, and the proteasome inhibitor (e.g., bortezomib or carfilzomib), or a pharmaceutically acceptable form thereof, are administered at certain dosages. In one embodiment, provided herein is a method of treating, managing, or preventing a cancer in a subject comprising administering to the subject a therapeutically effective amount of Compound 1:

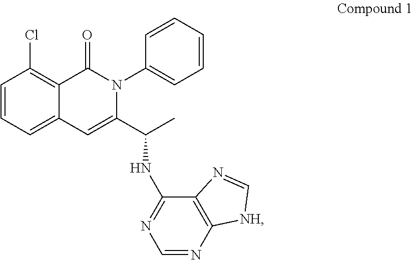 [SMILES: C[C@H](Nc1ncnc2[nH]cnc12)c1cc2cccc(Cl)c2c(=O)n1-c1ccccc1]

or a pharmaceutically acceptable form thereof, in combination with a proteasome inhibitor, or a pharmaceutically acceptable form thereof, wherein Compound 1, or a pharmaceutically acceptable form thereof, is administered at a dosage of in the range of from about 0.01 mg to about 75 mg daily and the proteasome inhibitor (e.g., bortezomib or carfilzomib), or a pharmaceutically acceptable form thereof, is administered at a dosage of in the range of from about 0.01 mg to about 1100 mg daily.

In one embodiment, Compound 1, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily. In one embodiment, Compound 1, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily. In one embodiment, Compound 1, or a pharmaceutically acceptable form thereof, is administered at a dosage of about 50 mg, about 37.5 mg, about 25 mg, about 20 mg, about 15 mg, about 10 mg, about 5 mg, or about 1 mg daily.

In one embodiment, the proteasome inhibitor (e.g., bortezomib or carfilzomib), or a pharmaceutically acceptable form thereof, is administered at a dosage of in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg daily. In one embodiment, the proteasome inhibitor (e.g., bortezomib or carfilzomib), or a pharmaceutically acceptable form thereof, is administered at a dosage of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, or less than about 25 mg daily.

In one embodiment, bortezomib, or a pharmaceutically acceptable form thereof, is administered at a dosage of in the range of from about 0.001 mg/m̂2 to about 1.3 mg/m̂2, from about 0.005 mg/m̂2 to about 1 mg/m̂2, from about 0.025 mg/m̂2 to about 0.75 mg/m̂2, from about 0.05 mg/m̂2 to about 0.5 mg/m̂2, from about 0.1 mg/m̂2 to about 0.4 mg/m̂2, or from about 0.2 mg/m̂2 to about 0.3 mg/m̂2 IV once about every three days (e.g., days 1, 4, 8 and 11 of each 21-day cycle). In one embodiment, bortezomib, or a pharmaceutically acceptable form thereof, is administered at a dosage of less than about 1.3 mg/m̂2, less than about 1 mg/m̂2, less than about 0.75 mg/m̂2, less than about 0.5 mg/m̂2, less than about 0.4 mg/m̂2, less than about 0.3 mg/m̂2, less than about 0.2 mg/m̂2, less than about 0.1 mg/m̂2, or less than about 0.05 mg/m̂2 IV once about every three days (e.g., days 1, 4, 8 and 11 of each 21-day cycle). In one embodiment, bortezomib, or a pharmaceutically acceptable form thereof, is administered at a dosage of about 1.3 mg/m̂2, about 1 mg/m̂2, about 0.75 mg/m̂2, about 0.5 mg/m̂2, about 0.4 mg/m̂2, about 0.3 mg/m̂2, about 0.2 mg/m̂2, about 0.1 mg/m̂2, or about 0.05 mg/m̂2 IV once about every three days (e.g., days 1, 4, 8 and 11 of each 21-day cycle).

In one embodiment, carfilzomib, or a pharmaceutically acceptable form thereof, is administered at a dosage of in the range of from about 0.1 mg/m̂2 to about 20 mg/m̂2, from about 0.2 mg/m̂2 to about 15 mg/m̂2, from about 0.5 mg/m̂2 to about 10 mg/m̂2, from about 1 mg/m̂2 to about 7.5 mg/m̂2, from about 2 mg/m̂2 to about 6 mg/m̂2, or from about 3 mg/m̂2 to about 4 mg/m̂2 IV once about every one to three days (e.g., days 1, 2, 8, 9, 15, and 16 of 28 day cycle). In one embodiment, carfilzomib, or a pharmaceutically acceptable form thereof, is administered at a dosage of less than about 20 mg/m̂2, less than about 15 mg/m̂2, less than about 10 mg/m̂2, less than about 7.5 mg/m̂2, less than about 6 mg/m̂2, less than about 4 mg/m̂2, less than about 3 mg/m̂2, less than about 2 mg/m̂2, or less than about 1 mg/m̂2 IV once about every one to three days (e.g., days 1, 2, 8, 9, 15, and 16 of 28 day cycle). In one embodiment, carfilzomib, or a pharmaceutically acceptable form thereof, is administered at a dosage of about 20 mg/m̂2, about 15 mg/m̂2, about 10 mg/m̂2, about 7.5 mg/m̂2, about 6 mg/m̂2, about 4 mg/m̂2, about 3 mg/m̂2, about 2 mg/m̂2, or about 1 mg/m̂2 IV once about every one to three days (e.g., days 1, 2, 8, 9, 15, and 16 of 28 day cycle).

In one embodiment, the proteasome inhibitor (e.g., bortezomib or carfilzomib), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before Compound 1, or a pharmaceutically acceptable form thereof, is administered. In another embodiment, the proteasome inhibitor (e.g., bortezomib or carfilzomib), or a pharmaceutically acceptable form thereof, is administered concurrently with Compound 1, or a pharmaceutically acceptable form thereof, in a single dosage form or separate dosage forms. In yet another embodiment, the proteasome inhibitor (e.g., bortezomib or carfilzomib), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after Compound 1, or a pharmaceutically acceptable form thereof, is administered. In one embodiment, the proteasome inhibitor is bortezomib. In another embodiment, the proteasome inhibitor is carfilzomib.

In certain embodiments, the PI3K inhibitor (e.g., Compound 1), or a pharmaceutically acceptable form thereof, and the proteasome inhibitor (e.g., bortezomib or carfilzomib), or a pharmaceutically acceptable form thereof, are in a single dosage form. In other embodiments, the PI3K inhibitor (e.g., Compound 1), or a pharmaceutically acceptable form thereof, and the proteasome inhibitor (e.g., bortezomib or carfilzomib), or a pharmaceutically acceptable form thereof, are in separate dosage forms.

In certain embodiments, the PI3K inhibitor (e.g., Compound 1), or a pharmaceutically acceptable form thereof, and the proteasome inhibitor (e.g., bortezomib or carfilzomib), are administered via a same route, e.g., both are administered orally. In other embodiments, the PI3K inhibitor (e.g., Compound 1), or a pharmaceutically acceptable form thereof, and the proteasome inhibitor (e.g., bortezomib or carfilzomib), are administered via different routes, e.g., one is administered orally and the other is administered intravenously. In one embodiment, Compound 1 is administered orally once per day and bortezomib is administered intravenously once about every three days (e.g., days 1, 4, 8 and 11 of each 21-day cycle). In one embodiment, Compound 1 is administered orally once per day and carfilzomib is administered intravenously once about every one to three days (e.g., days 1, 2, 8, 9, 15, and 16 of 28 day cycle).

In certain embodiments, the PI3K inhibitor (e.g., Compound 1), or a pharmaceutically acceptable form thereof, and the proteasome inhibitor (e.g., bortezomib or carfilzomib), or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., GS1101), a PI3K delta/gamma dual inhibitor, and a proteasome inhibitor (e.g., bortezomib or carfilzomib).

2.6 Combinations of PI3K Inhibitors and Immunomodulators

Provided herein are pharmaceutical compositions comprising a therapeutically effective amount of a PI3K inhibitor, or a pharmaceutically acceptable form thereof, and an immunomodulator, or a pharmaceutically acceptable form thereof.

Also provided herein are methods of treating, managing, or preventing a cancer in a subject comprising administering to the subject a therapeutically effective amount of a PI3K inhibitor, or a pharmaceutically acceptable form thereof, in combination with an immunomodulator, or a pharmaceutically acceptable form thereof.

Immunomodulators that can be used in the compositions and methods provided herein include, but are not limited to, lenalidomide, pomalidomide, and thalidomide.

In one embodiment, the immunomodulator is lenalidomide. Lenalidomide has a chemical name of 3-(4-Amino-1-oxo 1,3-dihydro-2H-isoindol-2-yl)piperidine-2,6-dione, and is of the structure:

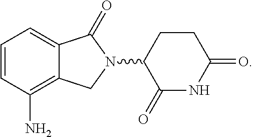 [SMILES: Nc1cccc2c1CN(C1CCC(=O)NC1=O)C2=O]

In one embodiment, the immunomodulator is pomalidomide. Pomalidomide has a chemical name of 4-Amino-2-(2,6-dioxopiperidin-3-yl)isoindole-1,3-dione, and is of the structure:

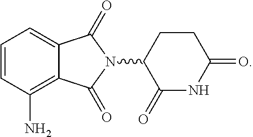 [SMILES: Nc1cccc2c1C(=O)N(C1CCC(=O)NC1=O)C2=O]

In one embodiment, the immunomodulator is thalidomide. Thalidomide has a chemical name of 2-(2,6-dioxopiperidin-3-yl)-1H-isoindole-1,3(2H)-dione, and is of the structure:

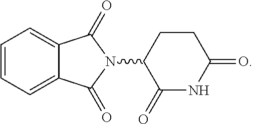 [SMILES: O=C1CCC(N2C(=O)c3ccccc3C2=O)C(=O)N1]

In certain embodiments, provided herein is a pharmaceutical composition comprising a therapeutically effective amount of a PI3K delta inhibitor, or a pharmaceutically acceptable form thereof, and an immunomodulator, or a pharmaceutically acceptable form thereof. In one embodiment, the PI3K delta inhibitor is GS1101 (CAL-101). In one embodiment, the immunomodulator is lenalidomide, pomalidomide or thalidomide. In one embodiment, the immunomodulator is lenalidomide. In another embodiment, the immunomodulator is pomalidomide. In one embodiment, the immunomodulator is thalidomide. In one embodiment, provided herein is a pharmaceutical composition comprising a therapeutically effective amount of GS1101, or a pharmaceutically acceptable form thereof, and thalidomide, or a pharmaceutically acceptable form thereof.

In one embodiment of the compositions and methods described herein, the molar ratio of the PI3K delta inhibitor (e.g., GS1101), or a pharmaceutically acceptable form thereof, to the immunomodulator (e.g., lenalidomide), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 50:1 to about 1:1, from about 25:1 to about 10:1, from about 20:1 to about 10:1, from about 20:1 to about 15:1, or about 19:1.

In one embodiment, the composition comprises the PI3K delta inhibitor (e.g., GS1101), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta inhibitor (e.g., GS1101), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta inhibitor which is Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the immunomodulator, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the immunomodulator, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the immunomodulator which is lenalidomide, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the PI3K delta inhibitor (e.g., GS1101) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 6000 ng/mL*hr to about 8000 ng/mL*hr, about 6500 ng/mL*hr to about 7500 ng/mL*hr, or about 7000 ng/mL*hr; and

the immunomodulator (e.g., lenalidomide) is administered at an amount to reach an AUCss at about 0.1 ng/mL*hr to about 10000 ng/mL*hr, about 1 ng/mL*hr to about 9000 ng/mL*hr, about 1000 ng/mL*hr to about 9000 ng/mL*hr, about 5000 ng/mL*hr to about 9000 ng/mL*hr, about 6000 ng/mL*hr to about 8000 ng/mL*h, about 7000 ng/mL*hr to about 8000 ng/mL*hr, or about 7311 ng/mL*hr. In one embodiment, the immunomodulator is lenalidomide and is administered at an amount to reach an AUCss at about 7311 ng/mL*h.

In one embodiment, the PI3K delta inhibitor (e.g., GS1101) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the immunomodulator (e.g., lenalidomide) is administered at an amount to reach an AUCss at less than about 1000 ng/mL*hr, less than about 750 ng/mL*hr, less than about 500 ng/mL*hr, less than about 250 ng/mL*hr, less than about 200 ng/mL*hr, less than about 100 ng/mL*hr, less than about 50 ng/mL*hr, less than about 25 ng/mL*hr, less than about 10 ng/mL*hr, less than about 1 ng/mL*hr, or less than about 7311 ng/mL*hr.

In one embodiment, the PI3K delta inhibitor (e.g., GS1101) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2300 ng/mL, about 2000 ng/mL to about 2300 ng/mL, or about 2200 ng/mL; and

the immunomodulator (e.g., lenalidomide) is administered at an amount to reach Cmaxss at about 0.1 ng/mL to about 1000 ng/mL, about 0.1 ng/mL to about 500 ng/mL, about 1 ng/mL to about 250 ng/mL, about 10 ng/mL to about 200 ng/mL, about 100 ng/mL to about 200 ng/mL, about 150 ng/mL to about 200 ng/mL, or about 176 ng/mL. In one embodiment, the immunomodulator is lenalidomide and is administered at an amount to reach Cmaxss at about 176 ng/mL.

In one embodiment, the PI3K delta inhibitor (e.g., GS1101) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the immunomodulator (e.g., lenalidomide) is administered at an amount to reach Cmaxss at less than about 1000 ng/mL, less than about 750 ng/mL, less than about 500 ng/mL, less than about 250 ng/mL, less than about 200 ng/mL, less than about 100 ng/mL, less than about 50 ng/mL, less than about 25 ng/mL, less than about 10 ng/mL, less than about 1 ng/mL, or less than about 176 ng/mL.

In one embodiment, the composition comprises the PI3K delta inhibitor (e.g., GS1101), or a pharmaceutically acceptable form thereof, at an amount in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta inhibitor (e.g., GS1101), or a pharmaceutically acceptable form thereof, at an amount of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with an immunomodulator (e.g. lenalidomide), or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma.

In some embodiments of the methods described herein, the PI3K delta inhibitor (e.g., GS1101), or a pharmaceutically acceptable form thereof, and the immunomodulator (e.g. lenalidomide), or a pharmaceutically acceptable form thereof, are administered at certain dosages. In one embodiment, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with an immunomodulator, or a pharmaceutically acceptable form thereof, wherein the PI3K delta inhibitor (e.g., GS1101), or a pharmaceutically acceptable form thereof, is administered at a dosage of in the range of from about 0.01 mg to about 75 mg daily and the immunomodulator (e.g. lenalidomide), or a pharmaceutically acceptable form thereof, is administered at a dosage of in the range of from about 0.01 mg to about 1100 mg daily.

In one embodiment, the PI3K delta inhibitor (e.g., GS1101), or a pharmaceutically acceptable form thereof, is administered at a dosage of in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta inhibitor (e.g., GS1101), or a pharmaceutically acceptable form thereof, is administered at a dosage of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a pharmaceutical composition comprising a therapeutically effective amount of a PI3K delta/gamma dual inhibitor, or a pharmaceutically acceptable form thereof, and an immunomodulator, or a pharmaceutically acceptable form thereof. In one embodiment, the immunomodulator is lenalidomide, pomolidomide or thalidomide. In one embodiment, the immunomodulator is lenalidomide.

In one embodiment of the compositions and methods described herein, the molar ratio of the PI3K delta/gamma dual inhibitor, or a pharmaceutically acceptable form thereof, to the immunomodulator (e.g., lenalidomide), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 5:1 to about 1:1, from about 4:1 to about 2:1, or about 3:1.

In one embodiment, the composition comprises the PI3K delta/gamma dual inhibitor (e.g., Compound 1), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta/gamma dual inhibitor (e.g., Compound 1), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta/gamma dual inhibitor which is Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the immunomodulator, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the immunomodulator, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the immunomodulator which is lenalidomide, or a pharmaceutically acceptable form thereof, at an amount sufficient to deliver a blood plasma concentration profile with an AUC (area under curve) of from about 100 ng/mL*h to about 1 μg/mL*h, from about 200 ng/mL*h to about 500 ng/mL*h, or from about 300 ng/mL*h to about 400 ng/mL*h.

In one embodiment, the PI3K delta/gamma dual inhibitor (e.g., Compound 1) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and

the immunomodulator (e.g., lenalidomide) is administered at an amount to reach an AUCss at about 0.1 ng/mL*hr to about 10000 ng/mL*hr, about 1 ng/mL*hr to about 9000 ng/mL*hr, about 1000 ng/mL*hr to about 9000 ng/mL*hr, about 5000 ng/mL*hr to about 9000 ng/mL*hr, about 6000 ng/mL*hr to about 8000 ng/mL*h, about 7000 ng/mL*hr to about 8000 ng/mL*hr, or about 7311 ng/mL*hr. In one embodiment, the immunomodulator is lenalidomide and is administered at an amount to reach an AUCss at about 7311 ng/mL*h.

In one embodiment, the PI3K delta/gamma dual inhibitor (e.g., Compound 1) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the immunomodulator (e.g., lenalidomide) is administered at an amount to reach an AUCss at less than about 1000 ng/mL*hr, less than about 750 ng/mL*hr, less than about 500 ng/mL*hr, less than about 250 ng/mL*hr, less than about 200 ng/mL*hr, less than about 100 ng/mL*hr, less than about 50 ng/mL*hr, less than about 25 ng/mL*hr, less than about 10 ng/mL*hr, less than about 1 ng/mL*hr, or less than about 7311 ng/mL*hr.

In one embodiment, the PI3K delta/gamma dual inhibitor (e.g., Compound 1) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

the immunomodulator (e.g., lenalidomide) is administered at an amount to reach Cmaxss at about 0.1 ng/mL to about 1000 ng/mL, about 0.1 ng/mL to about 500 ng/mL, about 1 ng/mL to about 250 ng/mL, about 10 ng/mL to about 200 ng/mL, about 100 ng/mL to about 200 ng/mL, about 150 ng/mL to about 200 ng/mL, or about 176 ng/mL. In one embodiment, the immunomodulator is lenalidomide and is administered at an amount to reach Cmaxss at about 176 ng/mL.

In one embodiment, the PI3K delta/gamma dual inhibitor (e.g., Compound 1) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the immunomodulator (e.g., lenalidomide) is administered at an amount to reach Cmaxss at less than about 1000 ng/mL, less than about 750 ng/mL, less than about 500 ng/mL, less than about 250 ng/mL, less than about 200 ng/mL, less than about 100 ng/mL, less than about 50 ng/mL, less than about 25 ng/mL, less than about 10 ng/mL, less than about 1 ng/mL, or less than about 176 ng/mL.

In one embodiment, the PI3K delta/gamma dual inhibitor (e.g., Compound 1) is administered at an amount that is decreased by about 1.5 fold to about 50 fold of the amount when administered alone and the immunomodulator (e.g., lenalidomide) is administered at an amount that is decreased by about 1.1 fold to about 50 fold of the amount when administered alone.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered alone; and

the immunomodulator (e.g., lenalidomide) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered alone.

In one embodiment, the composition comprises the PI3K delta/gamma dual inhibitor, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta/gamma dual inhibitor, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta/gamma dual inhibitor, or a pharmaceutically acceptable form thereof, in combination with an immunomodulator (e.g. lenalidomide), or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma.

In some embodiments of the methods described herein, the PI3K delta/gamma dual inhibitor, or a pharmaceutically acceptable form thereof, and the immunomodulator (e.g. lenalidomide), or a pharmaceutically acceptable form thereof, are administered at certain dosages. In one embodiment, provided herein is a method of treating, managing, or preventing a cancer in a subject comprising administering to the subject a therapeutically effective amount of a PI3K delta/gamma dual inhibitor, or a pharmaceutically acceptable form thereof, in combination with an immunomodulator, or a pharmaceutically acceptable form thereof, wherein the PI3K delta/gamma dual inhibitor, or a pharmaceutically acceptable form thereof, is administered at a dosage of in the range of from about 0.01 mg to about 75 mg daily and the immunomodulator (e.g. lenalidomide), or a pharmaceutically acceptable form thereof, is administered at a dosage of in the range of from about 0.01 mg to about 1100 mg daily.

In one embodiment, the PI3K delta/gamma dual inhibitor, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta/gamma dual inhibitor, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a composition, e.g., a pharmaceutical composition, comprising a therapeutically effective amount of Compound 1:

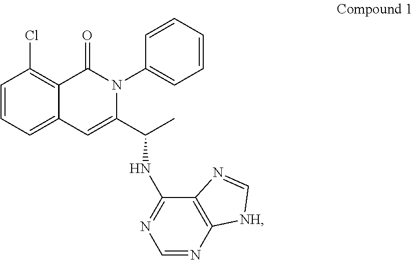 [SMILES: C[C@H](Nc1ncnc2[nH]cnc12)c1cc2cccc(Cl)c2c(=O)n1-c1ccccc1]

or a pharmaceutically acceptable form thereof, and an immunomodulator, or a pharmaceutically acceptable form thereof. In one embodiment, the immunomodulator is lenalidomide, pomalidomide, thalidomide, or a mixture thereof. In one embodiment, the immunomodulator is lenalidomide.

In certain embodiments, provided herein is a method of treating, managing, or preventing a cancer in a subject comprising administering to the subject a therapeutically effective amount of Compound 1:

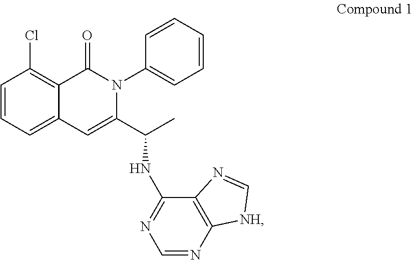 [SMILES: C[C@H](Nc1ncnc2[nH]cnc12)c1cc2cccc(Cl)c2c(=O)n1-c1ccccc1]

or a pharmaceutically acceptable form thereof, in combination with an immunomodulator, or a pharmaceutically acceptable form thereof. In one embodiment, the immunomodulator is lenalidomide, pomalidomide or thalidomide, or a mixture thereof. In one embodiment, the immunomodulator is lenalidomide.

In some embodiments of the compositions and methods described herein, Compound 1, or a pharmaceutically acceptable form thereof, is used in combination with an immunomodulator (e.g. lenalidomide), or a pharmaceutically acceptable form thereof, at certain molar ratios. In one embodiment, provided herein is a pharmaceutical composition comprising a therapeutically effective amount of Compound 1:

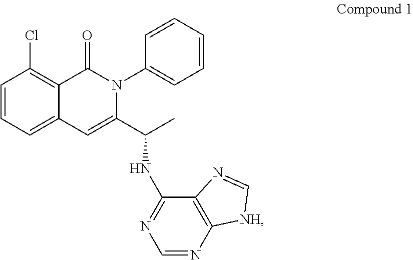 [SMILES: C[C@H](Nc1ncnc2[nH]cnc12)c1cc2cccc(Cl)c2c(=O)n1-c1ccccc1]

or a pharmaceutically acceptable form thereof, and a proteasome inhibitor, or a pharmaceutically acceptable form thereof, wherein the molar ratio of Compound 1, or a pharmaceutically acceptable form thereof, to the immunomodulator (e.g. lenalidomide), or a pharmaceutically acceptable form thereof, is in the range of from about 1000:1 to about 1:1000.

In one embodiment of the compositions and methods described herein, the molar ratio of Compound 1, or a pharmaceutically acceptable form thereof, to the immunomodulator (e.g. lenalidomide), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, or from about 5:1 to about 1:5. In one embodiment, the PI3K delta/gamma dual inhibitor is Compound 1, the immunomodulator is lenalidomide, and the molar ratio of Compound 1 to lenalidomide is from about 10:1 to about 1:10, from about 5:1 to about 1:5, from about 5:1 to about 1:1, from about 4:1 to about 2:1, or about 3:1.

In one embodiment of the compositions and methods described herein, the weight ratio of Compound 1, or a pharmaceutically acceptable form thereof, to lenalidomide, or a pharmaceutically acceptable form thereof, is in the range of from about 7.5-37.5 of Compound 1 to from 0.4-2 of bortezomib. In one embodiment, the weight ratio is in the range of from about 90:1 to about 4:1. In one embodiment, the weight ratio is in the range of from about 45:1 to about 8:1. In one embodiment, the weight ratio is in the range of from about 30:1 to about 12:1. In one embodiment, the weight ratio is in the range of from about 10:1 to about 1:1. In one embodiment, the weight ratio is in the range from about 7:1 to about 3:1. In one embodiment, the weight ratio is about 5:1.

In one embodiment of the compositions and methods described herein, the weight ratio of Compound 1, or a pharmaceutically acceptable form thereof, to lenalidomide, or a pharmaceutically acceptable form thereof, is in the range of from about 7.5-37.5 of Compound 1 to from 0.25-1.25 of bortezomib. In one embodiment, the weight ratio is in the range of from about 150:1 to about 6:1. In one embodiment, the weight ratio is in the range of from about 75:1 to about 12:1. In one embodiment, the weight ratio is in the range of from about 50:1 to about 18:1.

In one embodiment of the compositions and methods described herein, the weight ratio of Compound 1, or a pharmaceutically acceptable form thereof, to lenalidomide, or a pharmaceutically acceptable form thereof, is in the range of from about 7.5-37.5 of Compound 1 to from 3.8-19 of bortezomib. In one embodiment, the weight ratio is in the range of from about 10:1 to about 1:2.5. In one embodiment, the weight ratio is in the range of from about 5:1 to about 1:1.25. In one embodiment, the weight ratio is in the range of from about 3.3:1 to about 1.2:1.

In one embodiment, Compound 1 is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and

lenalidomide is administered at an amount to reach an AUCss at about 0.1 ng/mL*hr to about 10000 ng/mL*hr, about 1 ng/mL*hr to about 9000 ng/mL*hr, about 1000 ng/mL*hr to about 9000 ng/mL*hr, about 5000 ng/mL*hr to about 9000 ng/mL*hr, about 6000 ng/mL*hr to about 8000 ng/mL*h, about 7000 ng/mL*hr to about 8000 ng/mL*hr, or about 7311 ng/mL*hr. In one embodiment, lenalidomide is administered at an amount to reach an AUCss at about 7311 ng/mL*h.

In one embodiment, Compound 1 is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

lenalidomide is administered at an amount to reach Cmaxss at about 0.1 ng/mL to about 1000 ng/mL, about 0.1 ng/mL to about 500 ng/mL, about 1 ng/mL to about 250 ng/mL, about 10 ng/mL to about 200 ng/mL, about 100 ng/mL to about 200 ng/mL, about 150 ng/mL to about 200 ng/mL, or about 176 ng/mL. In one embodiment, lenalidomide is administered at an amount to reach Cmaxss at about 176 ng/mL.

In one embodiment, Compound 1 is administered at an amount that is decreased by about 1.5 fold to about 50 fold of the amount when administered alone and lenalidomide is administered at an amount that is decreased by about 1.1 fold to about 50 fold of the amount when administered alone.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered alone; and lenalidomide is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered alone.

In some embodiments of the compositions and methods described herein, the composition comprises Compound 1, or a pharmaceutically acceptable form thereof, and the immunomodulator (e.g. lenalidomide), or a pharmaceutically acceptable form thereof, at certain amounts. In one embodiment, provided herein is a pharmaceutical composition comprising a therapeutically effective amount of Compound 1:

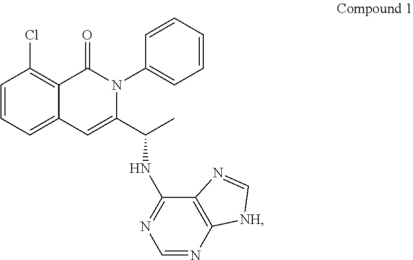 [SMILES: C[C@H](Nc1ncnc2[nH]cnc12)c1cc2cccc(Cl)c2c(=O)n1-c1ccccc1]

or a pharmaceutically acceptable form thereof, and an immunomodulator, or a pharmaceutically acceptable form thereof, wherein the composition comprises Compound 1, or a pharmaceutically acceptable form thereof, at an amount in the range of from about 0.01 mg to about 75 mg and the immunomodulator (e.g. lenalidomide), or a pharmaceutically acceptable form thereof, at an amount of in the range of from about 0.01 mg to about 1100 mg.

In one embodiment, the composition comprises Compound 1, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, from about 10 mg to about 20 mg, from about 1 mg to 10 mg, or from about 5 mg to about 10 mg. In one embodiment, the composition comprises Compound 1, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg. In one embodiment, the composition comprises Compound 1, or a pharmaceutically acceptable form thereof, at an amount of about 50 mg, about 37.5 mg, about 25 mg, about 20 mg, about 15 mg, about 10 mg, about 5 mg, or about 1 mg.

In one embodiment, the composition comprises the immunomodulator (e.g. lenalidomide), or a pharmaceutically acceptable form thereof, at an amount in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg. In one embodiment, the composition comprises the immunomodulator (e.g. lenalidomide), or a pharmaceutically acceptable form thereof, at an amount of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 25 mg, less than about 20 mg, less than 15 mg, less than about 10 mg, less than about 5 mg, or less than about 1 mg.

In one embodiment, the composition comprises lenalidomide, or a pharmaceutically acceptable form thereof, at an amount in the range of from about 0.1 mg to about 25 mg, from about 0.1 mg to about 20 mg, or from about 5 mg to about 15 mg.

In certain embodiments, provided herein is a method of treating, managing, or preventing a cancer in a subject comprising administering to the subject a therapeutically effective amount of Compound 1, or a pharmaceutically acceptable form thereof, in combination with a proteasome inhibitor, or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma. In one embodiment, the proteasome inhibitor is bortezomib. In another embodiment, the proteasome inhibitor is carfilzomib.

In some embodiments of the methods described herein, Compound 1, or a pharmaceutically acceptable form thereof, and the immunomodulator (e.g. lenalidomide), or a pharmaceutically acceptable form thereof, are administered at certain dosages. In one embodiment, provided herein is a method of treating, managing, or preventing a cancer in a subject comprising administering to the subject a therapeutically effective amount of Compound 1:

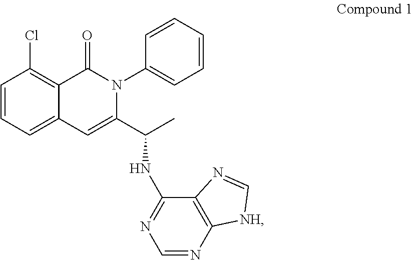 [SMILES: C[C@H](Nc1ncnc2[nH]cnc12)c1cc2cccc(Cl)c2c(=O)n1-c1ccccc1]

or a pharmaceutically acceptable form thereof, in combination with an immunomodulator, or a pharmaceutically acceptable form thereof, wherein Compound 1, or a pharmaceutically acceptable form thereof, is administered at a dosage of in the range of from about 0.01 mg to about 75 mg daily and the immunomodulator (e.g. lenaliomide), or a pharmaceutically acceptable form thereof, is administered at a dosage of in the range of from about 0.01 mg to about 1100 mg daily.

In one embodiment, Compound 1, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily. In one embodiment, Compound 1, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily. In one embodiment, Compound 1, or a pharmaceutically acceptable form thereof, is administered at a dosage of about 50 mg, about 37.5 mg, about 25 mg, about 20 mg, about 15 mg, about 10 mg, about 5 mg, or about 1 mg daily.

In one embodiment, the immunomodulator (e.g. lenalidomide), or a pharmaceutically acceptable form thereof, is administered at a dosage of in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, or from about 50 mg to about 250 mg daily. In one embodiment, the immunomodulator (e.g. lenalidomide), or a pharmaceutically acceptable form thereof, is administered at a dosage of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, or less than about 25 mg daily.

In one embodiment, the immunomodulator (e.g. lenalidomide), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before Compound 1, or a pharmaceutically acceptable form thereof, is administered. In another embodiment, the immunomodulator (e.g. lenalidomide), or a pharmaceutically acceptable form thereof, is administered concurrently with Compound 1, or a pharmaceutically acceptable form thereof, in a single dosage form or separate dosage forms. In yet another embodiment, the immunomodulator (e.g. lenalidomide), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after Compound 1, or a pharmaceutically acceptable form thereof, is administered. In one embodiment, the immunomodulator is lenalidomide.

In certain embodiments, the PI3K inhibitor (e.g., Compound 1), or a pharmaceutically acceptable form thereof, and the immunomodulator (e.g. lenalidomide), or a pharmaceutically acceptable form thereof, are in a single dosage form. In other embodiments, the PI3K inhibitor (e.g., Compound 1), or a pharmaceutically acceptable form thereof, and the immunomodulator (e.g. lenalidomide), or a pharmaceutically acceptable form thereof, are in separate dosage forms.

In certain embodiments, the PI3K inhibitor (e.g., Compound 1), or a pharmaceutically acceptable form thereof, and the immunomodulator (e.g. lenalidomide), are administered via a same route, e.g., both are administered orally. In other embodiments, the PI3K inhibitor (e.g., Compound 1), or a pharmaceutically acceptable form thereof, and the immunomodulator (e.g. lenalidomide), are administered via different routes, e.g., one is administered orally and the other is administered intravenously.

In certain embodiments, the PI3K inhibitor (e.g., Compound 1), or a pharmaceutically acceptable form thereof, and the immunomodulator (e.g. lenalidomide), or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., GS1101), a PI3K delta/gamma dual inhibitor (e.g. Compound 1), and an immunomodulator (e.g. lenalidomide).

### 2.7 Combinations of PI3K Inhibitors and Glucocortocosteroids

Glucocorticoids have anti-inflammatory and immunosuppressant effects. They bind to the glucocorticoid receptor, which is a transcription factor, and activate cell death machinery through both extrinsic and intrinsic apoptotic pathways. The balance of pro- and anti-apoptotic Bcl-2 family members is important in induction of glucocorticoid-dependent programmed cell death. See Berrou, I. et al. Molecular Mechanisms Conferring Resistance/Sensitivity to Glucocorticoid-Induced Apoptosis (Chapter 7 of Glucocorticoids—New Recognition of Our Familiar Friend, Book edited by Xiaoxiao Qian, ISBN 978-953-51-0872-6, Published: Nov. 28, 2012; available on the World Wide Web at dx.doi.org/10.5772/51467 (hereinafter Berrou et al.).

Interactions between glucocorticoids and the apoptosis pathway are reviewed in Berrou et al. Over-expression of Bcl-2 (B-cell lymphoma 2) or Bcl-xL (B-cell lymphoma extra large) in human ALL cells can prevent glucocorticoid induced apopotosis. Knockdown of Bim (BCL2 Like 11) confers resistance of ALL cells to glucocortocoid induced apoptosis, whereas upregulation of Bim sensitizes cells to glucocorticoid induced apoptosis. Knockdown of Mc1-1 (myeloid cell leukemia 1) sensitizies ALL cells to the apoptotic effect of glucocorticoids. Noxa (phorbol-12-myristate-13-acetate-induced protein 1) regulates Mc1-1 protein stability and Moxa/Mcl-1 balance determines cell survival or death. Puma (p53 upregulated modulator of apoptosis) facilitates glucocorticoid-induced apoptosis of lymphocytes. Bax (Bcl-2-associated X) protein regulates glucocorticoid induced apoptosis in thymocytes, and double knockouts of Bax and Bak (Bcl-2 homologous antagonist/killer) confer resistance to glucocorticoid-induced apoptosis in thymocytes.

Interactions between glucocorticoids and the PI3K pathway have been observed. Dexamathasone has a direct effect on PI3K pathway activity in proliferating chondrocytes. It was found that dexamethasone induced apoptosis in proliferative chondrocytes through activation of caspases and suppression of the Akt-phosphatidylinositol 3′-kinase signaling pathway. Chrysis, D. et al. Endocrinology 2005 146(3):1391-1397. Dexamethasome also prevents ischemia/reperfusion injury-induced cytokine expression and renal injury by suppressing PI3K/AKT signaling. Int J Clin Exp Pathol 2013:6(11):2366-2375. Treatment with dexamethasome and a dual PI3K/mTOR inhibitor increased pro-apoptotic Bim levels in T-ALL (T-cell ALL). Hall, C. & Kang, M. Cancer Research: Apr. 15, 2013; Vol. 73(8), Supplement 1; doi: 10.1158/1538-7445.AM2013-2752, Proceedings: AACR 104^{th} Annual Meeting 2013; Apr. 6-10, 2013, Washington D.C. Furthermore, synergistic activity between rapamycin and dexamethasone was observed in vitro in T-ALL cell lines and in vivo in acute lymphoblastic leukemia. Zhang, C. et al. Leukemia Research 36 (2012) 342-349.

In addition to their effects on apoptosis, glucocorticoids can also have a direct effect on the PI3K pathway, for example leading to suppression of pAKT. See Chrysis, D. et al. Endocrinology 2005 146(3):1391-1397; Connor Hall, Min Kang. Proceedings of the 104th Annual Meeting of the American Association for Cancer Research; 2013 Apr. 6-10; Washington, D.C. Philadelphia (Pa.): AACR; Cancer Res 2013; 73(8 Suppl):Abstract nr 2757. doi:10.1158/1538-7445.AM2013-2757. Without wishing to be bound by theory, it is expected that this effect, in addition to the induction of apoptosis, can synergize with PI3K suppression (e.g., PI3K delta and PI3K gamma suppression by Compound 1).

Provided herein are pharmaceutical compositions, e.g., synergistic pharmaceutical compositions, comprising a therapeutically effective amount of a PI3K inhibitor, or a pharmaceutically acceptable form thereof, and a glucocorticosteroid, or a pharmaceutically acceptable form thereof.

Also provided herein are methods of treating, managing, or preventing a cancer in a subject comprising administering to the subject a therapeutically effective amount of a PI3K inhibitor, or a pharmaceutically acceptable form thereof, in combination with a glucocorticosteroid, or a pharmaceutically acceptable form thereof. In certain embodiments, the combination is synergistic.

In some embodiments, an effect of the glucocorticosteroid, or the combination of the glucocorticosteroid and PI3K inhibitor, can be assessed, e.g., based on a reduction of pAKT, an increase in p-p85 regulatory subunit, or a change in one or more AKT targets. In certain embodiments, an effect of the glucocorticosteroid, or the combination of the glucocorticosteroid and PI3K inhibitor, is an effect, e.g., an increase, in apoptosis. In certain embodiments, the effect on apoptosis is assessed, e.g., based on one or more of caspase 3/7/8/9 activation, PARP cleavage, apoptosis by annexin/PI, CytC release, or MOMP. In certain embodiments, an effect of the pharmaceutical composition or method is an effect on BLC2 family proteins. In certain embodiments, the effect on BCL2 family proteins is an effect on a Bim level, pBAD target of AKT, or an anti-apoptotic protein (e.g., Bcl-2, Mc1-1, etc.) level. In certain embodiments, one or more such effects is enhanced, or shows synergy, due to the combination of the glucocorticosteroid with the PI3K inhibitor, e.g., compared with a monotherapy (e.g., a monotherapy with the glucocorticosteroid or the PI3K inhibitor).

Glucocorticosteroids that can be used in the compositions and methods provided herein include, but are not limited to, dexamethasone, aldosterone, beclomethasone, betamethasone, hydrocortisone, cortisone, deoxycorticosterone acetate (DOCA), fludrocortisone acetate, methylprednisolone, prednisolone, and prednisone, and mixtures thereof.

In one embodiment, the glucocorticosteroid is dexamethasone. Dexamethasone has a chemical name of (8S,9R,10S,11S,13S,14S,16R,17R)-9-Fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one, and is of the structure:

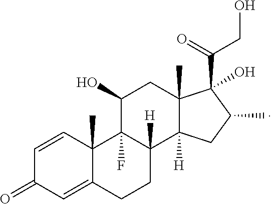

In certain embodiments, provided herein is a composition, e.g., a pharmaceutical composition, comprising a therapeutically effective amount of a PI3K delta inhibitor, or a pharmaceutically acceptable form thereof, and a glucocorticosteroid, or a pharmaceutically acceptable form thereof. In one embodiment, the PI3K delta inhibitor is GS1101 (CAL-101). In one embodiment, the glucocorticosteroid is selected from dexamethasone, aldosterone, beclomethasone, betamethasone, hydrocortisone, cortisone, deoxycorticosterone acetate (DOCA), fludrocortisone acetate, methylprednisolone, and prednisolone. In one embodiment, the glucocorticosteroid is dexamethasone. In one embodiment, provided herein is a pharmaceutical composition comprising a therapeutically effective amount of GS1101, or a pharmaceutically acceptable form thereof, and dexamethasone, or a pharmaceutically acceptable form thereof.

In some embodiments, the CAL-101 is administered at a dose of 60 mg daily (+/−0%, 10%, 20%, 30%, 40%, or 50%) and the dexamethasone is administered at a dose of 22 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the dexamethasone is administered at a dose of 18 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the dexamethasone is administered at a dose of 4 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the dexamethasone is administered at a dose of 10 mg or mg/m2 (+/−0%, 10%, 20%, 30%, 40%, or 50%) to treat a cancer such as ABC DLBCL. In some embodiments, the CAL-101 is administered at a dose of 60 mg daily (+/−0%, 10%, 20%, 30%, 40%, or 50%) and the dexamethasone is administered at a dose of 15 mg or mg/m2 (+/−0%, 10%, 20%, 30%, 40%, or 50%) to treat a cancer such as follicular lymphoma. In some embodiments, the CAL-101 is administered at a dose of 60 mg daily (+/−0%, 10%, 20%, 30%, 40%, or 50%) and the dexamethasone is administered at a dose of 13 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the dexamethasone is administered at a dose of 14 mg or mg/m2 (+/−0%, 10%, 20%, 30%, 40%, or 50%) to treat a cancer such as follicular lymphoma. In some embodiments, the CAL-101 is administered at a dose of 60 mg daily (+/−0%, 10%, 20%, 30%, 40%, or 50%) and the dexamethasone is administered at a dose of 4 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the dexamethasone is administered at a dose of 4 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the dexamethasone is administered at a dose of 7 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the dexamethasone is administered at a dose of 18 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the dexamethasone is administered at a dose of 11 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the dexamethasone is administered at a dose of 8 mg or mg/m2 (+/−0%, 10%, 20%, 30%, 40%, or 50%) to treat a cancer such as ABC DLBCL. In some embodiments, the CAL-101 is administered at a dose of 60 mg daily (+/−0%, 10%, 20%, 30%, 40%, or 50%) and the dexamethasone is administered at a dose of 4 mg or mg/m2 (+/−0%, 10%, 20%, 30%, 40%, or 50%) to treat a cancer such as ABC DLBCL. In some embodiments, the CAL-101 is administered at a dose of 60 mg daily (+/−0%, 10%, 20%, 30%, 40%, or 50%) and the dexamethasone is administered at a dose of 13 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the dexamethasone is administered at a dose of 11 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the dexamethasone is administered at a dose of 18 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the dexamethasone is administered at a dose of 4 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the dexamethasone is administered at a dose of 23 mg or mg/m2 (+/−0%, 10%, 20%, 30%, 40%, or 50%) to treat a cancer such as ABC DLBCL. In some embodiments, the Compound 1 is administered at a dose of 14 mg daily (+/−0%, 10%, 20%, 30%, 40%, or 50%) and the dexamethasone is administered at a dose of 13 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the dexamethasone is administered at a dose of 4 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the dexamethasone is administered at a dose of 16 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the dexamethasone is administered at a dose of 4 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the dexamethasone is administered at a dose of 4 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the dexamethasone is administered at a dose of 5 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the dexamethasone is administered at a dose of 14 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the dexamethasone is administered at a dose of 14 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the dexamethasone is administered at a dose of 11 mg or mg/m2 (+/−0%, 10%, 20%, 30%, 40%, or 50%) to treat a cancer such as ABC DLBCL. In some embodiments, the Compound 1 is administered at a dose of 14 mg daily (+/−0%, 10%, 20%, 30%, 40%, or 50%) and the dexamethasone is administered at a dose of 4 mg or mg/m2 (+/−0%, 10%, 20%, 30%, 40%, or 50%) to treat a cancer such as ABC DLBCL. In some embodiments, the Compound 1 is administered at a dose of 14 mg daily (+/−0%, 10%, 20%, 30%, 40%, or 50%) and the dexamethasone is administered at a dose of 9 mg or mg/m2 (+/−0%, 10%, 20%, 30%, 40%, or 50%) to treat a cancer such as follicular lymphoma.

In one embodiment of the compositions and methods described herein, the molar ratio of the PI3K delta inhibitor (e.g., GS1101), or a pharmaceutically acceptable form thereof, to the glucocorticosteroid (e.g. dexamethasone), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 500:1 to about 1:1, from about 250:1 to about 50:1, from about 200:1 to about 100:1, from about 200:1 to about 150:1, or about 190:1.

In one embodiment, the composition comprises the PI3K delta inhibitor (e.g., GS1101), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μs/mL*h, from about 100 ng/mL*h to about 10 μs/mL*h, from about 1 μs/mL*h to about 10 μs/mL*h. In one embodiment the composition comprises the PI3K delta inhibitor (e.g., GS1101), or a pharmaceutically acceptable form thereof, at an amount sufficient to deliver a blood plasma concentration profile with an AUC (area under curve) of from about 0.1 μs/mL*h to about 10 μs/mL*h, from about 0.2 μs/mL*h to about 9 μs/mL*h, from about 0.3 μs/mL*h to about 8 μs/mL*h, from about 0.4 μs/mL*h to about 7 μs/mL*h, from about 0.5 μs/mL*h to about 6 μs/mL*h, from about 0.6 μs/mL*h to about 5 μs/mL*h, from about 0.7 μs/mL*h to about 4 μs/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta inhibitor which is Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the glucocorticosteroid, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the glucocorticosteroid, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the glucocorticosteroid which is dexamethasone, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the PI3K delta inhibitor (e.g., GS1101) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 6000 ng/mL*hr to about 8000 ng/mL*hr, about 6500 ng/mL*hr to about 7500 ng/mL*hr, or about 7000 ng/mL*hr; and

the glucocorticosteroid (e.g. dexamethasone) is administered at an amount to reach an AUCss at about 0.1 ng/mL*hr to about 1000 ng/mL*hr, about 1 ng/mL*hr to about 900 ng/mL*hr, about 10 ng/mL*hr to about 500 ng/mL*hr, about 100 ng/mL*hr to about 250 ng/mL*hr, about 100 ng/mL*hr to about 200 ng/mL*h, about 100 ng/mL*hr to about 150 ng/mL*hr, or about 113 ng/mL*hr. In one embodiment, glucocorticosteroid is dexamethasone and is administered at an amount to reach an AUCss at about 113 ng/mL*h.

In one embodiment, the PI3K delta inhibitor (e.g., GS1101) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the glucocorticosteroid (e.g. dexamethasone) is administered at an amount to reach an AUCss at less than about 1000 ng/mL*hr, less than about 750 ng/mL*hr, less than about 500 ng/mL*hr, less than about 250 ng/mL*hr, less than about 200 ng/mL*hr, less than about 100 ng/mL*hr, less than about 50 ng/mL*hr, less than about 25 ng/mL*hr, less than about 10 ng/mL*hr, less than about 1 ng/mL*hr, or less than about 113 ng/mL*hr.

In one embodiment, the PI3K delta inhibitor (e.g., GS1101) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2300 ng/mL, about 2000 ng/mL to about 2300 ng/mL, or about 2200 ng/mL; and

the glucocorticosteroid (e.g. dexamethasone) is administered at an amount to reach Cmaxss at about 0.1 ng/mL to about 1000 ng/mL, about 0.1 ng/mL to about 500 ng/mL, about 1 ng/mL to about 250 ng/mL, about 1 ng/mL to about 100 ng/mL, about 1 ng/mL to about 50 ng/mL, about 10 ng/mL to about 25 ng/mL, or about 14 ng/mL. In one embodiment, the glucocorticosteroid is dexamethasone and is administered at an amount to reach Cmaxss at about 14 ng/mL.

In one embodiment, the PI3K delta inhibitor (e.g., GS1101) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the glucocorticosteroid (e.g. dexamethasone) is administered at an amount to reach Cmaxss at less than about 1000 ng/mL, less than about 750 ng/mL, less than about 500 ng/mL, less than about 250 ng/mL, less than about 200 ng/mL, less than about 100 ng/mL, less than about 50 ng/mL, less than about 25 ng/mL, less than about 10 ng/mL, less than about 1 ng/mL, or less than about 14 ng/mL.

In one embodiment, the composition comprises the PI3K delta inhibitor (e.g., GS1101), or a pharmaceutically acceptable form thereof, at an amount in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta inhibitor (e.g., GS1101), or a pharmaceutically acceptable form thereof, at an amount of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with a glucocorticosteroid (e.g. dexamethasone), or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma.

In some embodiments of the methods described herein, the PI3K delta inhibitor (e.g., GS1101), or a pharmaceutically acceptable form thereof, and the glucocorticosteroid (e.g. dexamethasone), or a pharmaceutically acceptable form thereof, are administered at certain dosages. In one embodiment, provided herein is a method of treating, managing, or preventing a cancer in a subject comprising administering to the subject a therapeutically effective amount of a PI3K delta inhibitor (e.g., GS1101), or a pharmaceutically acceptable form thereof, in combination with a glucocorticosteroid, or a pharmaceutically acceptable form thereof, wherein the PI3K delta inhibitor (e.g., GS1101), or a pharmaceutically acceptable form thereof, is administered at a dosage of in the range of from about 0.01 mg to about 75 mg daily and the glucocorticosteroid (e.g. dexamethasone), or a pharmaceutically acceptable form thereof, is administered at a dosage of in the range of from about 0.01 mg to about 1100 mg daily.

In one embodiment, the PI3K delta inhibitor (e.g., GS1101), or a pharmaceutically acceptable form thereof, is administered at a dosage of in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., GS1101), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta inhibitor (e.g., GS1101), or a pharmaceutically acceptable form thereof, is administered at a dosage of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a pharmaceutical composition comprising a therapeutically effective amount of a PI3K delta/gamma dual inhibitor, or a pharmaceutically acceptable form thereof, and a glucocorticosteroid (e.g. dexamethasone), or a pharmaceutically acceptable form thereof. In one embodiment, the glucocorticosteroid is dexamethasone.

In one embodiment of the compositions and methods described herein, the molar ratio of the PI3K delta/gamma dual inhibitor, or a pharmaceutically acceptable form thereof, to the glucocorticosteroid (e.g. dexamethasone), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5, from about 50:1 to about 1:1, from about 50:1 to about 10:1, from about 40:1 to about 20:1, or about 30:1.

In one embodiment, the composition comprises the PI3K delta/gamma dual inhibitor (e.g., Compound 1), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta/gamma dual inhibitor (e.g., Compound 1), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta/gamma dual inhibitor which is Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the composition comprises the glucocorticosteroid, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the glucocorticosteroid, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the glucocorticosteroid which is dexamethasone, or a pharmaceutically acceptable form thereof, at an amount sufficient to deliver a blood plasma concentration profile with an AUC (area under curve) of from about 1 ng/mL*h to about 1 μg/mL*h, from about 10 ng/mL*h to about 500 ng/mL*h, or from about 50 ng/mL*h to about 200 ng/mL*h.

In one embodiment, the PI3K delta/gamma dual inhibitor (e.g., Compound 1) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and the glucocorticosteroid (e.g. dexamethasone) is administered at an amount to reach an AUCss at about 0.1 ng/mL*hr to about 1000 ng/mL*hr, about 1 ng/mL*hr to about 900 ng/mL*hr, about 10 ng/mL*hr to about 500 ng/mL*hr, about 100 ng/mL*hr to about 250 ng/mL*hr, about 100 ng/mL*hr to about 200 ng/mL*h, about 100 ng/mL*hr to about 150 ng/mL*hr, or about 113 ng/mL*hr. In one embodiment, glucocorticosteroid is dexamethasone and is administered at an amount to reach an AUCss at about 113 ng/mL*h.

In one embodiment, the PI3K delta/gamma dual inhibitor (e.g., Compound 1) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the glucocorticosteroid (e.g. dexamethasone) is administered at an amount to reach an AUCss at less than about 1000 ng/mL*hr, less than about 750 ng/mL*hr, less than about 500 ng/mL*hr, less than about 250 ng/mL*hr, less than about 200 ng/mL*hr, less than about 100 ng/mL*hr, less than about 50 ng/mL*hr, less than about 25 ng/mL*hr, less than about 10 ng/mL*hr, less than about 1 ng/mL*hr, or less than about 113 ng/mL*hr.

In one embodiment, the PI3K delta/gamma dual inhibitor (e.g., Compound 1) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

the glucocorticosteroid (e.g. dexamethasone) is administered at an amount to reach Cmaxss at about 0.1 ng/mL to about 1000 ng/mL, about 0.1 ng/mL to about 500 ng/mL, about 1 ng/mL to about 250 ng/mL, about 1 ng/mL to about 100 ng/mL, about 1 ng/mL to about 50 ng/mL, about 10 ng/mL to about 25 ng/mL, or about 14 ng/mL. In one embodiment, the glucocorticosteroid is dexamethasone and is administered at an amount to reach Cmaxss at about 14 ng/mL.

In one embodiment, the PI3K delta/gamma dual inhibitor (e.g., Compound 1) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the glucocorticosteroid (e.g. dexamethasone) is administered at an amount to reach Cmaxss at less than about 1000 ng/mL, less than about 750 ng/mL, less than about 500 ng/mL, less than about 250 ng/mL, less than about 200 ng/mL, less than about 100 ng/mL, less than about 50 ng/mL, less than about 25 ng/mL, less than about 10 ng/mL, less than about 1 ng/mL, or less than about 14 ng/mL.

In one embodiment, the PI3K delta/gamma dual inhibitor (e.g., Compound 1) is administered at an amount that is decreased by about 1.5 fold to about 50 fold of the amount when administered alone and the glucocorticosteroid (e.g. dexamethasone) is administered at an amount that is decreased by about 1.1 fold to about 50 fold of the amount when administered alone.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered alone; and

the glucocorticosteroid (e.g. dexamethasone) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered alone.

In one embodiment, the composition comprises the PI3K delta/gamma dual inhibitor, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta/gamma dual inhibitor, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In certain embodiments, provided herein is a method of treating, managing, or preventing a cancer in a subject comprising administering to the subject a therapeutically effective amount of a PI3K delta/gamma dual inhibitor, or a pharmaceutically acceptable form thereof, in combination with a glucocorticosteroid (e.g. dexamethasone), or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma.

In some embodiments of the methods described herein, the PI3K delta/gamma dual inhibitor, or a pharmaceutically acceptable form thereof, and the glucocorticosteroid (e.g. dexamethasone), or a pharmaceutically acceptable form thereof, are administered at certain dosages. In one embodiment, provided herein is a method of treating, managing, or preventing a cancer in a subject comprising administering to the subject a therapeutically effective amount of a PI3K delta/gamma dual inhibitor, or a pharmaceutically acceptable form thereof, in combination with a glucocorticosteroid, or a pharmaceutically acceptable form thereof, wherein the PI3K delta/gamma dual inhibitor, or a pharmaceutically acceptable form thereof, is administered at a dosage of in the range of from about 0.01 mg to about 75 mg daily and the glucocorticosteroid (e.g. dexamethasone), or a pharmaceutically acceptable form thereof, is administered at a dosage of in the range of from about 0.01 mg to about 1100 mg daily.

In one embodiment, the PI3K delta/gamma dual inhibitor, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta/gamma dual inhibitor, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In certain embodiments, provided herein is a composition, e.g., a pharmaceutical composition, comprising a therapeutically effective amount of Compound 1:

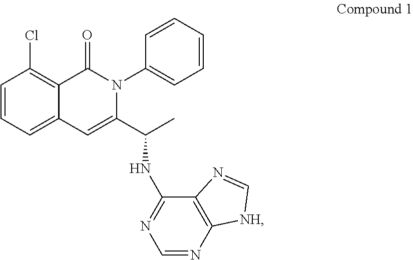 [SMILES: C[C@H](Nc1ncnc2[nH]cnc12)c1cc2cccc(Cl)c2c(=O)n1-c1ccccc1]

or a pharmaceutically acceptable form thereof, and a glucocorticosteroid, or a pharmaceutically acceptable form thereof. In one embodiment, the glucocorticosteriod is selected from dexamethasone, aldosterone, beclomethasone, betamethasone, hydrocortisone, cortisone, deoxycorticosterone acetate (DOCA), fludrocortisone acetate, methylprednisolone, prednisolone, and prednisone, and mixtures thereof, or a mixture thereof. In one embodiment, the glucocorticosteroid is dexamethasone.

In certain embodiments, provided herein is a method of treating, managing, or preventing a cancer in a subject comprising administering to the subject a therapeutically effective amount of Compound 1:

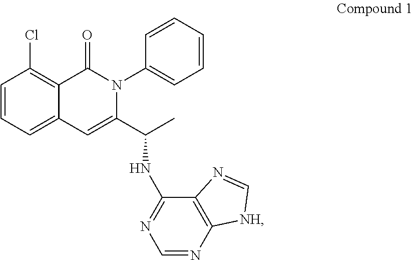 [SMILES: C[C@H](Nc1ncnc2[nH]cnc12)c1cc2cccc(Cl)c2c(=O)n1-c1ccccc1]

or a pharmaceutically acceptable form thereof, in combination with a glucocorticosteroid, or a pharmaceutically acceptable form thereof. In one embodiment, the glucocorticosteroid is dexamethasone, aldosterone, beclomethasone, betamethasone, hydrocortisone, cortisone, deoxycorticosterone acetate (DOCA), fludrocortisone acetate, methylprednisolone, prednisolone, and prednisone, and mixtures thereof, or a mixture thereof. In one embodiment, the glucocorticosteroid is dexamethasone.

In some embodiments of the compositions and methods described herein, Compound 1, or a pharmaceutically acceptable form thereof, is used in combination with a glucocorticosteroid (e.g. dexamethasone), or a pharmaceutically acceptable form thereof, at certain molar ratios. In one embodiment, provided herein is a pharmaceutical composition comprising a therapeutically effective amount of Compound 1:

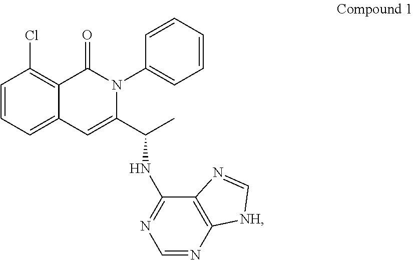 [SMILES: C[C@H](Nc1ncnc2[nH]cnc12)c1cc2cccc(Cl)c2c(=O)n1-c1ccccc1]

or a pharmaceutically acceptable form thereof, and glucocorticosteroid (e.g. dexamethasone), or a pharmaceutically acceptable form thereof, wherein the molar ratio of Compound 1, or a pharmaceutically acceptable form thereof, to the glucocorticosteroid (e.g. dexamethasone), or a pharmaceutically acceptable form thereof, is in the range of from about 1000:1 to about 1:1000.

In one embodiment of the compositions and methods described herein, the molar ratio of Compound 1, or a pharmaceutically acceptable form thereof, to the glucocorticosteroid (e.g. dexamethasone), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, or from about 5:1 to about 1:5.

In one embodiment of the compositions and methods described herein, the weight ratio of Compound 1, or a pharmaceutically acceptable form thereof, to dexamethasone, or a pharmaceutically acceptable form thereof, is in the range of from about 7.5-37.5 of Compound 1 to from 0.4-2 of dexamethasone. In one embodiment, the weight ratio is in the range of from about 90:1 to about 4:1. In one embodiment, the weight ratio is in the range of from about 45:1 to about 8:1. In one embodiment, the weight ratio is in the range of from about 40:1 to about 15:1. In one embodiment, the weight ratio is in the range of from about 10:1 to about 1:1. In one embodiment, the weight ratio is in the range from about 5:1 to about 1:1. In one embodiment, the weight ratio is in the range from about 4:1 to about 2:1. In one embodiment, the weight ratio is about 3.5:1.

In one embodiment, Compound 1 is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr; and

dexamethasone is administered at an amount to reach an AUCss at about 0.1 ng/mL*hr to about 1000 ng/mL*hr, about 1 ng/mL*hr to about 900 ng/mL*hr, about 10 ng/mL*hr to about 500 ng/mL*hr, about 100 ng/mL*hr to about 250 ng/mL*hr, about 100 ng/mL*hr to about 200 ng/mL*h, about 100 ng/mL*hr to about 150 ng/mL*hr, or about 113 ng/mL*hr. In one embodiment, g dexamethasone is administered at an amount to reach an AUCss at about 113 ng/mL*h.

In one embodiment, Compound 1 is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL; and

dexamethasone is administered at an amount to reach Cmaxss at about 0.1 ng/mL to about 1000 ng/mL, about 0.1 ng/mL to about 500 ng/mL, about 1 ng/mL to about 250 ng/mL, about 1 ng/mL to about 100 ng/mL, about 1 ng/mL to about 50 ng/mL, about 10 ng/mL to about 25 ng/mL, or about 14 ng/mL. In one embodiment, dexamethasone is administered at an amount to reach Cmaxss at about 14 ng/mL.

In one embodiment, Compound 1 is administered at an amount that is decreased by about 1.5 fold to about 50 fold of the amount when administered alone and dexamethasone is administered at an amount that is decreased by about 1.1 fold to about 50 fold of the amount when administered alone.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered alone; and dexamethasone is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered alone.

In some embodiments of the compositions and methods described herein, the composition comprises Compound 1, or a pharmaceutically acceptable form thereof, and the glucocorticosteroid (e.g. dexamethasone), or a pharmaceutically acceptable form thereof, at certain amounts. In one embodiment, provided herein is a pharmaceutical composition comprising a therapeutically effective amount of Compound 1:

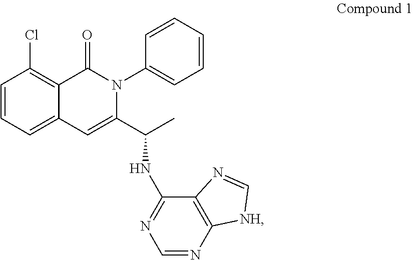 [SMILES: C[C@H](Nc1ncnc2[nH]cnc12)c1cc2cccc(Cl)c2c(=O)n1-c1ccccc1]

or a pharmaceutically acceptable form thereof, and a glucocorticosteriod, or a pharmaceutically acceptable form thereof, wherein the composition comprises Compound 1, or a pharmaceutically acceptable form thereof, at an amount in the range of from about 0.01 mg to about 75 mg and the glucocorticosteroid (e.g. dexamethasone), or a pharmaceutically acceptable form thereof, at an amount of in the range of from about 0.01 mg to about 1100 mg.

In one embodiment, the composition comprises Compound 1, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg. In one embodiment, the composition comprises Compound 1, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg. In one embodiment, the composition comprises Compound 1, or a pharmaceutically acceptable form thereof, at an amount of about 50 mg, about 37.5 mg, about 25 mg, about 20 mg, about 15 mg, about 10 mg, about 5 mg, or about 1 mg.

In one embodiment, the composition comprises the glucocorticosteroid (e.g. dexamethasone), or a pharmaceutically acceptable form thereof, at an amount in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, from about 50 mg to about 250 mg, from about 1 mg to about 50 mg, from about 1 mg to about 25 mg, from about 1 mg to about 20 mg, from about 1 mg to about 15 mg, or from about 10 mg to about 15 mg. In one embodiment, the composition comprises the glucocorticosteroid (e.g. dexamethasone), or a pharmaceutically acceptable form thereof, at an amount of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 25 mg, less than about 20 mg, less than about 15 mg, less than about 10 mg, less than about 5 mg, or less than about 1 mg.

In one embodiment, the composition comprises glucocorticosteriod (e.g. dexamethasone), or a pharmaceutically acceptable form thereof, at an amount in the range of from about 0.1 mg to about 25 mg, from about 0.1 mg to about 20 mg, or from about 5 mg to about 15 mg.

In certain embodiments, provided herein is a method of treating, managing, or preventing a cancer in a subject comprising administering to the subject a therapeutically effective amount of Compound 1, or a pharmaceutically acceptable form thereof, in combination with a glucocorticosteroid, or a pharmaceutically acceptable form thereof, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma. In one embodiment, the glucocorticosteroid is dexamethasone.

In some embodiments of the methods described herein, Compound 1, or a pharmaceutically acceptable form thereof, and the immunomodulator (e.g. lenalidomide), or a pharmaceutically acceptable form thereof, are administered at certain dosages. In one embodiment, provided herein is a method of treating, managing, or preventing a cancer in a subject comprising administering to the subject a therapeutically effective amount of Compound 1:

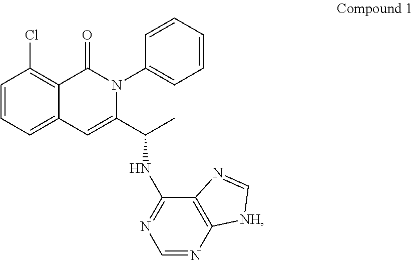 [SMILES: C[C@H](Nc1ncnc2[nH]cnc12)c1cc2cccc(Cl)c2c(=O)n1-c1ccccc1]

or a pharmaceutically acceptable form thereof, in combination with a glucocorticosteroid, or a pharmaceutically acceptable form thereof, wherein Compound 1, or a pharmaceutically acceptable form thereof, is administered at a dosage of in the range of from about 0.01 mg to about 75 mg daily and the glucocorticosteroid (e.g. dexamethasone), or a pharmaceutically acceptable form thereof, is administered at a dosage of in the range of from about 0.01 mg to about 1100 mg daily.

In one embodiment, Compound 1, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily. In one embodiment, Compound 1, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily. In one embodiment, Compound 1, or a pharmaceutically acceptable form thereof, is administered at a dosage of about 50 mg, about 37.5 mg, about 25 mg, about 20 mg, about 15 mg, about 10 mg, about 5 mg, or about 1 mg daily.

In one embodiment, the glucocorticosteroid (e.g. dexamethasone), or a pharmaceutically acceptable form thereof, is administered at a dosage of in the range of from about 0.1 mg to about 800 mg, from about 0.1 mg to about 750 mg, from about 0.1 mg to about 600 mg, from about 1 mg to about 500 mg, from about 1 mg to about 400 mg, from about 10 mg to about 300 mg, from about 50 mg to about 250 mg, from about 1 mg to about 50 mg, from about 1 mg to about 25 mg, from about 1 mg to about 20 mg, from about 1 mg to about 15 mg, or from about 10 mg to about 15 mg daily. In one embodiment, the glucocorticosteroid (e.g. dexamethasone), or a pharmaceutically acceptable form thereof, is administered at a dosage of less than about 1000 mg, less than about 800 mg, less than about 750 mg, less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 25 mg, less than about 20 mg, less than about 15 mg, less than about 10 mg, less than about 5 mg, or less than about 1 mg daily.

In one embodiment, the glucocorticosteroid (e.g. dexamethasone), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before Compound 1, or a pharmaceutically acceptable form thereof, is administered. In another embodiment, the glucocorticosteroid (e.g. dexamethasone), or a pharmaceutically acceptable form thereof, is administered concurrently with Compound 1, or a pharmaceutically acceptable form thereof, in a single dosage form or separate dosage forms. In yet another embodiment, the glucocorticosteroid (e.g. dexamethasone), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after Compound 1, or a pharmaceutically acceptable form thereof, is administered. In one embodiment, the glucocorticosteroid is dexamethasone.

In certain embodiments, the PI3K inhibitor (e.g., Compound 1), or a pharmaceutically acceptable form thereof, and the glucocorticosteroid (e.g. dexamethasone), or a pharmaceutically acceptable form thereof, are in a single dosage form. In other embodiments, the PI3K inhibitor (e.g., Compound 1), or a pharmaceutically acceptable form thereof, and the glucocorticoid (e.g. dexamethasone), or a pharmaceutically acceptable form thereof, are in separate dosage forms.

In certain embodiments, the PI3K inhibitor (e.g., Compound 1), or a pharmaceutically acceptable form thereof, and the glucocorticosteroid (e.g. dexamethasone), are administered via a same route, e.g., both are administered orally. In other embodiments, the PI3K inhibitor (e.g., Compound 1), or a pharmaceutically acceptable form thereof, and the glucocorticosteroid (e.g. dexamethasone), are administered via different routes, e.g., one is administered orally and the other is administered intravenously. In certain embodiments, the PI3K inhibitor (e.g., Compound 1), or a pharmaceutically acceptable form thereof, and the glucocorticosteroid (e.g. dexamethasone), or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., GS1101), a PI3K delta/gamma dual inhibitor, and a glucocorticosteroid (e.g. dexamethasone).

### 2.8 Combinations of PI3K Inhibitors and CDK4/6 Inhibitors

Activation of the phosphoinositide 3-kinase (PI3K) pathway occurs frequently in certain solid tumors such as breast cancer. In some instances, PI3K inhibitors, e.g., PI3K-alpha inhibitors show only modest activity, e.g., modest therapeutic effects. A combinatorial drug screen on PIK3CA mutant cancers with decreased sensitivity to PI3K inhibitors revealed that combined CDK4/6-PI3K inhibition synergistically reduces cell viability. Vora et al. Cancer Cell 2014 26, 136-149. Similar combination effects are likely to be seen in the setting of dysregulated PI3K signaling in other tumors, e.g., tumors that show decreased sensitivity or resistance, e.g., acquired resistance, to a PI3K inhibitor, e.g., IPI-145. See also Chiron, D. et al. Cancer Discovery (published online Jul. 31, 2014) doi: 10.1158/2159-8290.CD-14-0098.

In certain embodiments, provided herein is a pharmaceutical composition comprising a PI3K inhibitor, e.g., one or more PI3K inhibitors (e.g., Compound 1 or GS1101, or both) or a pharmaceutically acceptable form thereof, and a CDK4/6 inhibitor (e.g., one or more inhibitors of CDK4, CDK6 or both) or a pharmaceutically acceptable form thereof. The PI3K inhibitor and the CDK4/6 inhibitor can be present in a single composition or as two or more different compositions. In some embodiments, the composition (e.g., one or more compositions comprising the combination of PI3K inhibitor and the CDK4/6 inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the CDK4/6 inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, (e.g., inhibiting, reducing, ameliorating, managing, or preventing) a cancer in a subject. The method comprises administering to the subject a PI3K inhibitor, e.g., one or more PI3K inhibitors (e.g., Compound 1 or GS1101, or both) or a pharmaceutically acceptable form thereof, in combination with a CDK4/6 inhibitor (e.g., one or more inhibitors of CDK4, CDK6 or both), or a pharmaceutically acceptable form thereof. In certain embodiments, the combination of the PI3K inhibitor and the CDK4/6 inhibitor is synergistic, e.g., has a synergistic effect in treating the cancer (e.g., in reducing cancer cell growth or viability, or both). In some embodiments, the amount or dosage of the PI3K inhibitor, the CDK4/6 inhibitor, or both, used in combination does not exceed the level at which each agent is used individually, e.g., as a monotherapy. In certain embodiments, the amount or dosage of the PI3K inhibitor, the CDK4/6 inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the CDK4/6 inhibitor, or both, used in combination that results in treatment of cancer is lower (e.g., at least 20%, at least 30%, at least 40%, or at least 50% lower) than the amount or dosage of each agent used individually, e.g., as a monotherapy.

Exemplary CDK4/6 inhibitors include, but are not limited to, e.g., LEE011 (Novartis), LY-2835219 (Eli Lilly), and PD 0332991 (Pfizer). In some embodiments, the CD4/6 inhibitor is selected from one or more of LEE011, PD0332991 (palbociclib), and LY2835219 (abemaciclib). In certain embodiments, the CD4/6 inhibitor is LEE011. In certain embodiments, the CD4/6 inhibitor is PD0332991 (palbociclib). In certain embodiments, the CD4/6 inhibitor is LY2835219 (abemaciclib). In one embodiment, the CDK4/6 inhibitor is LEE011 or PD0332991 or a mixture thereof. In one embodiment, the CDK4/6 inhibitor is LEE011 or LY2835219 or a mixture thereof. In one embodiment, the CDK4/6 inhibitor is LEE011 or LY2835219 or a mixture thereof. In one embodiment, the CDK4/6 inhibitor is PD0332991 or LY2835219 or a mixture thereof.

In some embodiments, the CDK4/6 inhibitor inhibits one or both of CDK4 or CDK6. In certain embodiments, the CDK4/6 inhibitor inhibits CDK4 and CDK6. Exemplary CDK4/6 inhibitors include, e.g., LEE011 (Novartis), LY-2835219, and PD 0332991 (Pfizer).

Exemplary CDK4/6 inhibitors are described in, e.g., WO 2007/140222, WO 2010/020675, WO 2013/006368, WO 2013/006532, WO 2011/130232, US 2013/0150342, W2011/101409, US 2013/184285, WO2006024945, WO2006024945, and EP1256578B1, all of which are hereby incorporated by reference in their entirety.

In another embodiment, the CDK4/6 inhibitor is chosen from LEE011 (Novartis); LY-2835219 (Eli Lilly); or PD 0332991 (Pfizer).

In one embodiment, the CDK 4/6 inhibitor has the following structure:

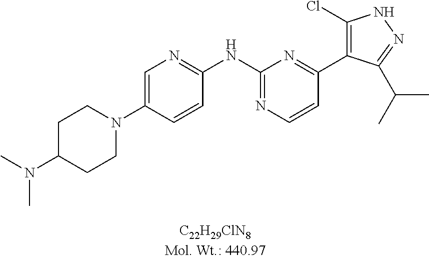 [SMILES: CC(C)c1n[nH]c(Cl)c1-c1ccnc(Nc2ccc(N3CCC(N(C)C)CC3)cn2)n1]

also referred to herein as LEE011. In one embodiment, the CDK 4/6 inhibitor has the following chemical name: 4-(5-chloro-3-isopropyl-1H-pyrazol-4-yl)-N-(5-(4-(dimethylamino)piperidin-1-yl)pyridin-2-yl)pyrimidin-2-amine.

In some embodiments, the CAL-101 is administered at a dose of 60 mg daily (+/−0%, 10%, 20%, 30%, 40%, or 50%) and the LEE011 is administered at a dose of 216 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the LEE011 is administered at a dose of 223 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the LEE011 is administered at a dose of 182 mg or mg/m2 (+/−0%, 10%, 20%, 30%, 40%, or 50%) to treat a cancer such as follicular lymphoma. In some embodiments, the CAL-101 is administered at a dose of 60 mg daily (+/−0%, 10%, 20%, 30%, 40%, or 50%) and the LEE011 is administered at a dose of 342 mg or mg/m2 (+/−0%, 10%, 20%, 30%, 40%, or 50%) to treat a cancer such as ABC DLBCL. In some embodiments, the CAL-101 is administered at a dose of 60 mg daily (+/−0%, 10%, 20%, 30%, 40%, or 50%) and the LEE011 is administered at a dose of 395 mg or mg/m2 (+/−0%, 10%, 20%, 30%, 40%, or 50%) to treat a cancer such as follicular lymphoma. In some embodiments, the CAL-101 is administered at a dose of 60 mg daily (+/−0%, 10%, 20%, 30%, 40%, or 50%) and the LEE011 is administered at a dose of 212 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the LEE011 is administered at a dose of 141 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the LEE011 is administered at a dose of 197 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the LEE011 is administered at a dose of 168 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the LEE011 is administered at a dose of 93 mg or mg/m2 (+/−0%, 10%, 20%, 30%, 40%, or 50%) to treat a cancer such as ABC DLBCL. In some embodiments, the CAL-101 is administered at a dose of 60 mg daily (+/−0%, 10%, 20%, 30%, 40%, or 50%) and the LEE011 is administered at a dose of 147 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the LEE011 is administered at a dose of 324 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the LEE011 is administered at a dose of 234 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the LEE011 is administered at a dose of 127 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the LEE011 is administered at a dose of 387 mg or mg/m2 (+/−0%, 10%, 20%, 30%, 40%, or 50%) to treat a cancer such as ABC DLBCL. In some embodiments, the Compound 1 is administered at a dose of 14 mg daily (+/−0%, 10%, 20%, 30%, 40%, or 50%) and the LEE011 is administered at a dose of 300 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the LEE011 is administered at a dose of 174 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the LEE011 is administered at a dose of 90 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the LEE011 is administered at a dose of 60 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the LEE011 is administered at a dose of 83 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the LEE011 is administered at a dose of 143 mg or mg/m2 (+/−0%, 10%, 20%, 30%, 40%, or 50%) to treat a cancer such as ABC DLBCL. In some embodiments, the Compound 1 is administered at a dose of 14 mg daily (+/−0%, 10%, 20%, 30%, 40%, or 50%) and the LEE011 is administered at a dose of 125 mg or mg/m2 (+/−0%, 10%, 20%, 30%, 40%, or 50%) to treat a cancer such as follicular lymphoma.

In another embodiment, the CDK 4/6 inhibitor has the following structure:

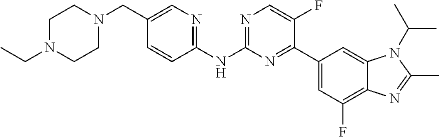 [SMILES: CCN1CCN(Cc2ccc(Nc3ncc(F)c(-c4cc(F)c5nc(C)n(C(C)C)c5c4)n3)nc2)CC1]

also referred to herein as LY-2835219. In one embodiment, the CDK 4/6 inhibitor has the following chemical name: (N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine).

In yet another embodiment, the CDK 4/6 inhibitor has the following structure:

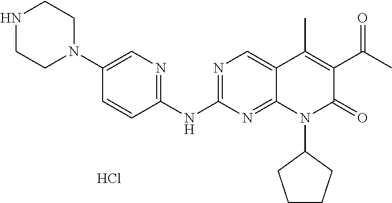 [SMILES: CC(=O)c1c(C)c2cnc(Nc3ccc(N4CCNCC4)cn3)nc2n(C2CCCC2)c1=O ; Cl]

also referred to herein as PD 0332991. In one embodiment, the CDK 4/6 inhibitor has the following chemical name: 6-acetyl-8-cyclopentyl-5-methyl-2-(5-(piperazin-1-yl)pyridin-2-ylamino)pyrido[2,3-d]pyrimidin-7(8H)-one hydrochloride.

Further examples of publications describing the aforesaid inhibitors and their activities include Finn, R S et al. (2009) Breast Cancer Res. 11(5):R77; Zhang, Y. in Proceedings of the AACR-NCI-EORTC International Conference: Molecular Targets and Cancer Therapeutics, 2011:10 (11 Suppl): Abstract nr A236; Clinical Trial Gov. Identifier NCT01237236; and Clinical Trial Gov. Identifier NCT01394016, incorporated herein by reference.

In another embodiment, the CDK inhibitor is BAY1000394. BAY1000394 is an orally bioavailable CDK inhibitor. It inhibits the activity of cell-cycle CDKs, including CDK1, CDK2, CDK3, CDK4, and of transcriptional CDKs CDK7 and CDK9 with IC50 values in the range between 5 and 25 nM. BAY1000394 has the chemical name: 2-Butanol, 3-[[2-[[4-[[S(R)]-S-cyclopropylsulfonimidoyl]pheny]amino]-5-(trifluoromethyl)-4-pyrimidinyl]oxy]-, (2R,3R)—; and has the following structure:

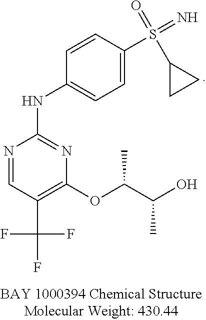 [SMILES: C[C@@H](O)[C@@H](C)Oc1nc(Nc2ccc(S(=N)(=O)C3CC3)cc2)ncc1C(F)(F)F]

In another embodiment, the CDK inhibitor is ZK-304709. ZK-304709 is a potent multi-target tumor growth inhibitor. ZK-304709 inhibits the activity of cell-cycle CDKs, including CDK1, CDK2, CDK4, and of transcriptional CDKs CDK7 and CDK9, with IC50 values in the nanomolar range. ZK-304709 also inhibits the activity of vascular endothelial growth factor receptor tyrosine kinases (VEGFRs), including VEGFR 1, VEGFR 2, and VEGFR3 and of platelet-derived growth factor receptor beta tyrosine kinase (PDGFR). ZK-304709 has the chemical name: (Z)-3,3-dimethyl-2′-oxo-[2,3′-biindolinylidene]-5′-sulfonamide; and has the following structure:

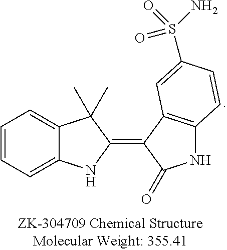 [SMILES: CC1(C)/C(=C2/C(=O)Nc3ccc(S(N)(=O)=O)cc32)Nc2ccccc21]

In another embodiment, the CDK inhibitor is SNS032. SNS032 inhibits the activity of cell-cycle CDKs, including CDK1, CDK2, and of transcriptional CDKs CDK4, CDK7 and CDK9. SNS-032 has low sensitivity to CDK1 and CDK4 with IC50 of 480 nM and 925 nM, respectively. SNS032 has the chemical name: N-(5-((5-tert-butyloxazol-2-yl)methylthio)thiazol-2-yl)piperidine-4-carboxamide; and has the following structure:

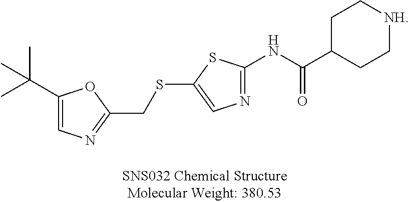 [SMILES: CC(C)(C)c1cnc(CSc2cnc(NC(=O)C3CCNCC3)s2)o1]

In another embodiment, the CDK inhibitor is NC381. NC381 inhibits the activity of cell-cycle CDKs, including CDK4. NC381 has the following structure:

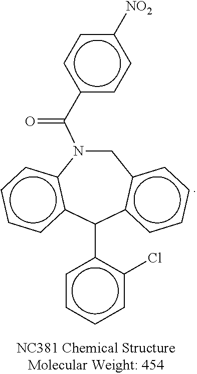

In another embodiment, the CDK inhibitor is Milciclib. Milciclib is an orally bioavailable inhibitor of cyclin-dependent kinases (CDKs) and thropomyosin receptor kinase A (TRKA). Milciclib inhibits the activity of cell-cycle CDKs, including CDK1, CDK2, and CDK4. Milciclib has the chemical name: N,1,4,4-tetramethyl-8-((4-(4-methylpiperazin-1-yl)phenyl)amino)-4,5-dihydro-1H-pyrazolo[4,3-h]quinazoline-3-carboxamide; and has the following structure:

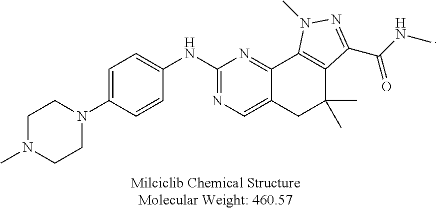

In another embodiment, the CDK inhibitor is ON123300. ON123300 inhibits the activity of cell-cycle CDKs, including CDK4. ON123300 has the chemical name: NH—(N—CH3piperazino)phenyl; and has the following structure:

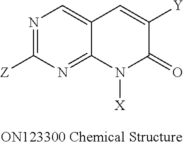

In another embodiment, the CDK inhibitor is PD0332991 (palbociclib). PD0332991 (palbociclib) inhibits the activity of CDKs, including CDK4 and CDK6, with IC50 of 11 nM and 16 nM, respectively. PD0332991 (palbociclib) has the chemical name: Ethanesulfonic acid, 2-hydroxy-, compd. with 6-acetyl-8-cyclopentyl-5-methyl-2-[[5-(1-piperazinyl)-2-pyridinyl]amino]pyrido[2,3-d]pyrimidin-7(8H)-one (1:1); and has the following structure:

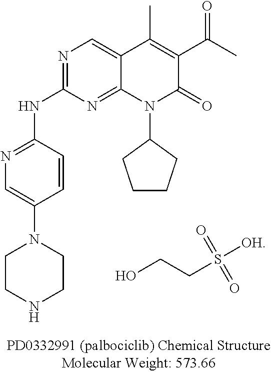 [SMILES: CC(=O)c1c(C)c2cnc(Nc3ccc(N4CCNCC4)cn3)nc2n(C2CCCC2)c1=O ; O=S(=O)(O)CCO]

In one embodiment, the CDK4/6 inhibitor (e.g., LEE011 or PD-0332991), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the CDK4/6 inhibitor (e.g., LEE011 or PD-0332991), or a pharmaceutically acceptable form thereof, is administered concurrently with the PI3K inhibitor (e.g., Compound 1), or a pharmaceutically acceptable form thereof, in a single dosage form or separate dosage forms. In yet another embodiment, the CDK4/6 inhibitor (e.g., LEE011 or PD-0332991), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered. In one embodiment, the CDK4/6 inhibitor is LEE011. In another embodiment, the CDK4/6 inhibitor is PD-0332991.

In some embodiments, the CAL-101 is administered at a dose of 60 mg daily (+/−0%, 10%, 20%, 30%, 40%, or 50%) and the PD-0332991 is administered at a dose of 58 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the PD-0332991 is administered at a dose of 42 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the PD-0332991 is administered at a dose of 41 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the PD-0332991 is administered at a dose of 13 mg or mg/m2 (+/−0%, 10%, 20%, 30%, 40%, or 50%) to treat a cancer such as follicular lymphoma. In some embodiments, the CAL-101 is administered at a dose of 60 mg daily (+/−0%, 10%, 20%, 30%, 40%, or 50%) and the PD-0332991 is administered at a dose of 13 mg or mg/m2 (+/−0%, 10%, 20%, 30%, 40%, or 50%) to treat a cancer such as ABC DLBCL. In some embodiments, the CAL-101 is administered at a dose of 60 mg daily (+/−0%, 10%, 20%, 30%, 40%, or 50%) and the PD-0332991 is administered at a dose of 49 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the PD-0332991 is administered at a dose of 19 mg or mg/m2 (+/−0%, 10%, 20%, 30%, 40%, or 50%) to treat a cancer such as follicular lymphoma. In some embodiments, the CAL-101 is administered at a dose of 60 mg daily (+/−0%, 10%, 20%, 30%, 40%, or 50%) and the PD-0332991 is administered at a dose of 34 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the PD-0332991 is administered at a dose of 13 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the PD-0332991 is administered at a dose of 16 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the PD-0332991 is administered at a dose of 41 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the PD-0332991 is administered at a dose of 54 mg or mg/m2 (+/−0%, 10%, 20%, 30%, 40%, or 50%) to treat a cancer such as GCB DLBCL. In some embodiments, the CAL-101 is administered at a dose of 60 mg daily (+/−0%, 10%, 20%, 30%, 40%, or 50%) and the PD-0332991 is administered at a dose of 28 mg or mg/m2 (+/−0%, 10%, 20%, 30%, 40%, or 50%) to treat a cancer such as ABC DLBCL. In some embodiments, the CAL-101 is administered at a dose of 60 mg daily (+/−0%, 10%, 20%, 30%, 40%, or 50%) and the PD-0332991 is administered at a dose of 13 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the PD-0332991 is administered at a dose of 98 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the PD-0332991 is administered at a dose of 53 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the PD-0332991 is administered at a dose of 35 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the PD-0332991 is administered at a dose of 13 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the PD-0332991 is administered at a dose of 71 mg or mg/m2 (+/−0%, 10%, 20%, 30%, 40%, or 50%) to treat a cancer such as ABC DLBCL. In some embodiments, the Compound 1 is administered at a dose of 14 mg daily (+/−0%, 10%, 20%, 30%, 40%, or 50%) and the PD-0332991 is administered at a dose of 19 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the PD-0332991 is administered at a dose of 13 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the PD-0332991 is administered at a dose of 27 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the PD-0332991 is administered at a dose of 22 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the PD-0332991 is administered at a dose of 16 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the PD-0332991 is administered at a dose of 17 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the PD-0332991 is administered at a dose of 25 mg or mg/m2 (+/−0%, 10%, 20%, 30%, 40%, or 50%) to treat a cancer such as GCB DLBCL. In some embodiments, the Compound 1 is administered at a dose of 14 mg daily (+/−0%, 10%, 20%, 30%, 40%, or 50%) and the PD-0332991 is administered at a dose of 30 mg or mg/m2 (+/−0%, 10%, 20%, 30%, 40%, or 50%) to treat a cancer such as ABC DLBCL. In some embodiments, the Compound 1 is administered at a dose of 14 mg daily (+/−0%, 10%, 20%, 30%, 40%, or 50%) and the PD-0332991 is administered at a dose of 13 mg or mg/m2 (+/−0%, 10%, 20%, 30%, 40%, or 50%) to treat a cancer such as follicular lymphoma. In some embodiments, the Compound 1 is administered at a dose of 14 mg daily (+/−0%, 10%, 20%, 30%, 40%, or 50%) and the PD-0332991 is administered at a dose of 13 mg or mg/m2 (+/−0%, 10%, 20%, 30%, 40%, or 50%) to treat a cancer such as follicular lymphoma.

2.9 Combinations of PI3K Inhibitors and HDAC Inhibitors

In certain embodiments, provided herein is a pharmaceutical composition comprising a PI3K inhibitor, e.g., one or more PI3K inhibitors (e.g., Compound 1 or GS1101, or both) or a pharmaceutically acceptable form thereof, and an HDAC inhibitor (e.g., one or more inhibitors of HDAC) or a pharmaceutically acceptable form thereof. The PI3K inhibitor and the HDAC inhibitor can be present in a single composition or as two or more different compositions. In some embodiments, the composition (e.g., one or more compositions comprising the combination of PI3K inhibitor and the HDAC inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the HDAC inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, (e.g., inhibiting, reducing, ameliorating, managing, or preventing) a cancer in a subject. The method comprises administering to the subject a PI3K inhibitor, e.g., one or more PI3K inhibitors (e.g., Compound 1 or GS1101, or both) or a pharmaceutically acceptable form thereof, in combination with an HDAC inhibitor (e.g., one or more inhibitors of HDAC), or a pharmaceutically acceptable form thereof. In certain embodiments, the combination of the PI3K inhibitor and the HDAC inhibitor is synergistic, e.g., has a synergistic effect in treating the cancer (e.g., in reducing cancer cell growth or viability, or both). In some embodiments, the amount or dosage of the PI3K inhibitor, the HDAC inhibitor, or both, used in combination does not exceed the level at which each agent is used individually, e.g., as a monotherapy. In certain embodiments, the amount or dosage of the PI3K inhibitor, the HDAC inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the HDAC inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiment, the HDAC inhibitor is chosen from one or more of a hydroxamate, m-carboxycinnamic acid bis-hydroxamide (CBHA), a cyclic peptide, an aliphatic acid, a benzamide, or a sulphonamide anilide.

Exemplary HDAC inhibitors include, but are not limited to vorinostat (SAHA), romidepsin (depsipeptide or FK-228), panobinostat, valproic acid, belinostat (PXD101), mocetinostat (MGCD0103), abrexinostat, SB939, resminostat, givinostat (ITF2357), CUDC-101, AR-42, CHR-2845, CHR-3996, 4SC-202, CG200745, LAQ824, ACY-1215, kevetrin, sodium butyrate, trichostatin A, MS-275 (Entinostat), trapoxin, apicidin, chlamydocin, phenylbutyrate, AN-93, pimelic diphenylamide, N-acetyldinaline, N-2-aminophenyl-3-[4-(4-methylbenzenesulfonylamino)-phenyl]-2-propenamide, LBH-589, SK7041, SK7068, tubacin, depudecin, CI994, Quisinostat (JNJ-26481585), ME-344, sulforaphane, BML-210, PCI-3405, PCI-24781, luteolin, VAHA, chidamide, PTACH, Oxamflatin, biphenyl-4-sulfonyl chloride, HC toxin, (S)-HDAC-42, 4-iodo-SAHA, cambinol, splitomycin, SBHA, scriptaid, resveratrol, or a combination thereof. In one embodiment, the HDAC inhibitor is belinostat. In another embodiment, the HDAC inhibitor is romidepsin. In one embodiment, the HDAC inhibitor is tubastatin A hydrochloride.

In another embodiment, the HDAC inhibitor is belinostat (PXD101). Belinostat has the chemical name: (2E)-N-Hydroxy-3-[3-(phenylsulfamoyl)phenyl]prop-2-enamide; and has the following structure:

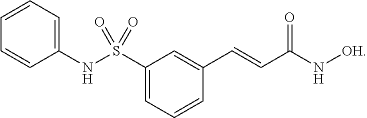 [SMILES: O=C(/C=C/c1cccc(S(=O)(=O)Nc2ccccc2)c1)NO]

In another embodiment, the HDAC inhibitor is romidepsin (depsipeptide or FK-228). Romidepsin has the chemical name: (1S,4S,7Z,10S,16E,21R)-7-ethylidene-4,21-diisopropyl-2-oxa-12,13-dithia-5,8,20,23-tetrazabicyclo[8.7.6]tricos-16-ene-3,6,9,19,22-pentone; and has the following structure:

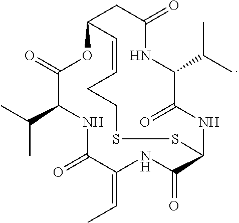 [SMILES: C/C=C1/NC(=O)[C@@H]2NC(=O)[C@@H](C(C)C)NC(=O)C[C@@H](/C=C\CCSS2)OC(=O)[C@H](C(C)C)NC1=O]

In another embodiment, the HDAC inhibitor is vorinostat (SAHA). Vorinostate has the chemical name: N-hydroxy-N′-phenyl-octanediamide; and has the following structure:

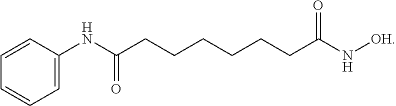 [SMILES: O=C(CCCCCCC(=O)Nc1ccccc1)NO]

In one embodiment, the HDAC inhibitor is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the HDAC inhibitor is administered concurrently with the PI3K inhibitor (e.g., Compound 1), or a pharmaceutically acceptable form thereof, in a single dosage form or separate dosage forms. In yet another embodiment, the HDAC inhibitor is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered.

While not wishing to be bound by theory, experiments described herein indicate that upstream MAPK mediators of AP1, such as ERK1/2, p38/MAPK, JUN, and FOS, are upregulated in cells resistant to Compound 1. This pathway promotes cell proliferation and survival; hence its activation can promote resistance to a PI3K inhibitor. Accordingly, by administering a combination of a PI3K inhibitor and a second agent that inhibits an upstream MAPK mediator of AP-1 activation, one can reduce resistance to the PI3K inhibitor. Thus, in certain aspects, provided herein are combinations of PI3K inhibitor, e.g., Compound 1, with an inhibitor of an upstream MAPK mediator of Activator Protein-1 (AP-1) activation. The MEK-ERK pathway regulates cell growth, proliferation, differentiation, and apoptosis. The AP-1 complex binds to promoter and enhancer regions of target genes and regulates gene expression. Exemplary upstream MAPK mediators of AP-1 activation include ERK1/2, p38/MAPK, JUN, and FOS.

In some embodiments, combinations of Compound 1 with an inhibitor of ERK1/2 are provided. ERK1 and ERK2 are phosphorylated upon activation of cell surface tyrosine kinases such as epidermal growth factor receptor (EGFR). Phosphorylation of ERK1/2 activates its kinase activity. ERK1/2 activates various protein kinases and transcription factors, including ETS domain-containing protein (ELK1). Dysregulation of the ERK pathway is commonly found in cancers. Exemplary inhibitors of ERK1/2 include SCH772984 (Merck; for example, described in Morris et al. Cancer Discov. 3.7(2013):742-50); BVD-523 (BioMed Valley Discoveries, Inc.; Clinical Trial Identifier No. NCT 02296242); and MEK162 (Novartis; Clinical trial identifier no. NCT01885195). In some embodiments, Compound 1 is administered in combination with an inhibitor of ERK1/2. In some embodiments, an inhibitor of ERK1/2 is administered to a subject that is resistant or that shows decreased responsiveness (e.g., is non-responsive) to Compound 1 treatment.

In some embodiments, the ERK inhibitor is SCH772984 (Moris et al., Cancer Discov. 2013 July; 3(7):742-50. doi: 10.1158/2159-8290), which has the chemical name (R)-1-(2-oxo-2-(4-(4-(pyrimidin-2-yl)phenyl)piperazin-1-yl)ethyl)-N-(3-(pyridin-4-yl)-1H-indazol-5-yl)pyrrolidine-3-carboxamide and has the following structure:

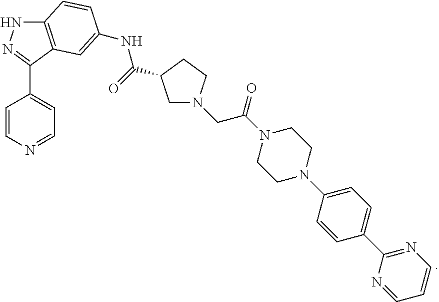 [SMILES: O=C(Nc1ccc2[nH]nc(-c3ccncc3)c2c1)[C@@H]1CCN(CC(=O)N2CCN(c3ccc(-c4ncccn4)cc3)CC2)C1]

In some embodiments, the ERK inhibitor is SCH772984 and the cancer is melanoma.

In some embodiments, the ERK inhibitor is hypothemycin, having the following structure:

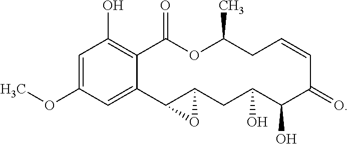 [SMILES: COc1cc(O)c2c(c1)[C@H]1O[C@@H]1C[C@H](O)[C@H](O)C(=O)/C=C\C[C@H](C)OC2=O]

In some embodiments, the ERK inhibitor is VX-11e, having the chemical name 4-[2-(2-chloro-4-fluoroanilino)-5-methylpyrimidin-4-yl]-N-[(1S)-1-(3-chlorophenyl)-2-hydroxyethyl]-1H-pyrrole-2-carboxamide and having the following structure:

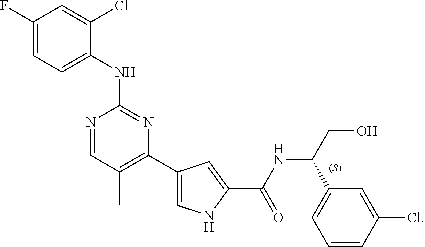

In some embodiments, the ERK inhibitor is BVD-523 (BioMed Valley Discoveries, Inc., Clinical Trial Identifier NCT02296242) and the cancer is Acute Myelogenous Leukemia or Myelodysplastic Syndrome).

In some embodiments, combinations of Compound 1 with an inhibitor of p38 are provided. P38 is a MAPK that responds to stress stimuli, e.g., cytokines, ultraviolet irradiation, heat shock, and osmotic shock. P38 is involved in cellular processes such as apoptosis, differentiation, and autophagy. Exemplary p38 inhibitors include SB-681323 (GSK; Clinical trial identifier No. NCT00390845); LY2228820 (Eli Lilly; clinical trial identifier no. NCT01663857); ARRY-371797 (Array BioPharma; clinical trial identifier no. NCT00663767); ARRY-797 (Array BioPharma); PH-797804 (Pfizer; Clinical Trial identifier No. NCT00620685); VX-702 (Vertex; Clinical trial identifier no. NCT00395577); Pamapimod (Roche Pharmaceuticals); Iosmapimod (GW856553; GlaxoSmithKline); Dilmapimod (SB681323; GlaxoSmithKline); Doramapimod (BIRB 796; Boehringer Ingelheim Pharmaceutical); BMS-582949 (Bristol-Myers Squibb); and SCIO-469 (Scios). See, e.g., Arthur et al. Nat. Reviews Immunol. 13(2013):679-92. In some embodiments, Compound 1 is administered in combination with an inhibitor of p38. In some embodiments, an inhibitor of p38 is administered to a subject that is resistant or that shows decreased responsiveness (e.g., is non-responsive) to Compound 1 treatment.

In some embodiments, combinations of Compound 1 with an inhibitor of c-Jun are provided. C-Jun is encoded by the JUN gene, which is a proto-oncogene. c-Jun binds with c-Fos to form the AP-1 early response transcription factor complex. c-Jun is phosphorylated by c-Jun N-terminal kinase (JNK), which is involved in responses to stress stimuli, such as cytokines, ultraviolet irradiation, heat shock, and osmotic shock. The JNK/c-Jun pathway is also involved in cell differentiation and apoptosis. C-Jun has been found to be overexpressed in several cancers. Exemplary inhibitors of the JNK/c-Jun pathway, e.g., inhibitors of JNK, include Doramapimod (BIRB 796; Boehringer Ingelheim Pharmaceutical) and Tanzisertib (CC-930; Celgene). In some embodiments, Compound 1 is administered in combination with an inhibitor of c-Jun or JNK. In some embodiments, an inhibitor of c-Jun or JNK is administered to a subject that is resistant or that shows decreased responsiveness (e.g., is non-responsive) to Compound 1 treatment.

Combinations of Compound 1 with an inhibitor of c-FOS (FBJ murine osteosarcoma viral oncogene homolog) are also provided. FOS is a proto-oncogene that is a member of the FOS gene family that includes four members: FOS, FOSB, FOSL1, and FOSL2. FOS is an early gene stimulated upon cellular stress stimuli or activated by posttranscriptional modifications. The FOS gene encodes a leucine zipper protein called c-Fos that can dimerize with proteins of the JUN family, thereby forming the transcription factor complex AP-1. FOS proteins have been implicated as regulators of cell proliferation, differentiation, and transformation. Increased levels of FOS/AP-1 have been shown to lead to accelerated cell cycle progression of B cells. Exemplary inhibitors of c-FOS include gefitinib, erlotinib (see, e.g., Jimeno et al. Cancer Res. 66.4(2006):2385-90); and T-5224 (Toyama Chemical/Kyushu University Beppu Hospital; Japan Clinical Trial No. JapicCTI-101359). In some embodiments, Compound 1 is administered in combination with an inhibitor of c-FOS, e.g., inhibitor of FOS, FOSB, FOSL1, and/or FOSL2. In some embodiments, an inhibitor of c-FOS is administered to a subject that is resistant that shows decreased responsiveness (e.g., is non-responsive) to Compound 1 treatment.

Any of the aforesaid combinations with Compound 1 (e.g., an inhibitor of ERK1/2, p38, c-Jun, or FOS) can further include an additional therapeutic agent, e.g., 1) a MEK inhibitor, 2) an mTOR inhibitor, 3) an AKT inhibitor, 4) a proteasome inhibitor, 5) immunomodulator, 6) a glucocorticosteroid, 7) a CDK4/6 inhibitor, 8) an histone deacetylase (HDAC), 9) a BET inhibitor, 10) an epigenetic inhibitor, 11) a PI3K alpha inhibitor, 12) a topoisomerase inhibitor, or 13) an ERK inhibitor.

### 2.10 Combinations of PI3K Inhibitors and BET Inhibitors

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with a BET inhibitor (e.g., one or more BET inhibitors), or a pharmaceutically acceptable form thereof. The PI3K inhibitor and the BET inhibitor can be present in a single composition or as two or more different compositions. In some embodiments, the composition (e.g., one or more compositions comprising the combination of PI3K inhibitor and the BET inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the BET inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with a BET inhibitor (e.g., one or more BET inhibitors), or a pharmaceutically acceptable form thereof. In certain embodiments, the combination of the PI3K inhibitor and the BET inhibitor is synergistic, e.g., has a synergistic effect in treating the cancer (e.g., in reducing cancer cell growth or viability, or both). In some embodiments, the amount or dosage of the PI3K inhibitor, the BET inhibitor, or both, used in combination does not exceed the level at which each agent is used individually, e.g., as a monotherapy. In certain embodiments, the amount or dosage of the PI3K inhibitor, the BET inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the BET inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiments, the BET inhibitor is chosen from one or more of (+)-JQ1, GSK525762, I-BET151, PF-6405761, I-BET-762, RVX-208, OF-1, MS436, I-BET726, PFI-3, or CPI-203, or a combination thereof. In another embodiment, the BET inhibitor is (+)-JQ1.

### 2.11 Combinations of PI3K Inhibitors and Epigenetic Inhibitors

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with an epigenetic inhibitor (e.g., one or more epigenetic inhibitors), or a pharmaceutically acceptable form thereof. The PI3K inhibitor and the epigenetic inhibitor can be present in a single composition or as two or more different compositions. In some embodiments, the composition (e.g., one or more compositions comprising the combination of PI3K inhibitor and the epigenetic inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the epigenetic inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with an epigenetic inhibitor (e.g., one or more epigenetic inhibitors), or a pharmaceutically acceptable form thereof. In certain embodiments, the combination of the PI3K inhibitor and the epigenetic inhibitor is synergistic, e.g., has a synergistic effect in treating the cancer (e.g., in reducing cancer cell growth or viability, or both). In some embodiments, the amount or dosage of the PI3K inhibitor, the epigenetic inhibitor, or both, used in combination does not exceed the level at which each agent is used individually, e.g., as a monotherapy. In certain embodiments, the amount or dosage of the PI3K inhibitor, the epigenetic inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the epigenetic inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiments, the epigenetic inhibitor is chosen from one or more of azacitidine, decitabine, RG108, thioguanine, zebularine, procainamide HCl, SGI-1027, or lomeguatrib or a combination thereof. In another embodiment, the epigenetic inhibitor is azacitidine.

### 2.12 Combinations of One or More PI3K Inhibitors

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with a PI3K alpha inhibitor (e.g., one or more PI3K alpha inhibitors), or a pharmaceutically acceptable form thereof. The PI3K inhibitor and the PI3K alpha inhibitor can be present in a single composition or as two or more different compositions. In some embodiments, the composition (e.g., one or more compositions comprising the combination of PI3K inhibitor and the PI3K alpha inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the PI3K alpha inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with a PI3K alpha inhibitor (e.g., one or more PI3K alpha inhibitors), or a pharmaceutically acceptable form thereof. In certain embodiments, the combination of the PI3K inhibitor and the PI3K alpha inhibitor is synergistic, e.g., has a synergistic effect in treating the cancer (e.g., in reducing cancer cell growth or viability, or both). In some embodiments, the amount or dosage of the PI3K inhibitor, the PI3K alpha inhibitor, or both, used in combination does not exceed the level at which each agent is used individually, e.g., as a monotherapy. In certain embodiments, the amount or dosage of the PI3K inhibitor, the PI3K alpha inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the PI3K alpha inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiments, the PI3K alpha inhibitor is chosen from one or more of GDC-0941, GDC-0032, HS-173, A66, PIK-75, Alpelisib, Gedatolisib, CH5132799, or Copanlisib, or a combination thereof. In some embodiments, the PI3K alpha inhibitor is GDC-0941.

2.13 Combinations of PI3K Inhibitors with Topoisomerase Inhibitors

In certain embodiments, provided herein is a composition (e.g., one or more pharmaceutical compositions or dosage forms), comprising a PI3K inhibitor, e.g., one or more PI3K inhibitors (e.g., Compound 1 or GS1101, or both) or a pharmaceutically acceptable form thereof, in combination with a topoisomerase inhibitor (e.g., one or more topoisomerase inhibitors), or a pharmaceutically acceptable form thereof. The PI3K inhibitor and the topoisomerase inhibitor can be present in a single composition or as two or more different compositions. In some embodiments, the composition (e.g., one or more compositions comprising the combination of PI3K inhibitor and the topoisomerase inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the topoisomerase inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating (e.g., inhibiting, reducing, ameliorating, managing, or preventing) a cancer in a subject comprising administering to the subject a PI3K inhibitor, e.g., one or more PI3K inhibitors (e.g., Compound 1 or GS1101, or both) or a pharmaceutically acceptable form thereof, in combination with a topoisomerase inhibitor (e.g., one or more topoisomerase inhibitors), or a pharmaceutically acceptable form thereof. In certain embodiments, the combination of the PI3K inhibitor and the topoisomerase inhibitor is synergistic, e.g., has a synergistic effect in treating the cancer (e.g., in reducing cancer cell growth or viability, or both). In some embodiments, the amount or dosage of the PI3K inhibitor, the topoisomerase inhibitor, or both, used in combination does not exceed the level at which each agent is used individually, e.g., as a monotherapy. In certain embodiments, the amount or dosage of the PI3K inhibitor, the topoisomerase inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the topoisomerase inhibitor, or both, used in combination that results in treatment of cancer is lower (e.g., at least 20%, at least 30%, at least 40%, or at least 50% lower) than the amount or dosage of each agent used individually, e.g., as a monotherapy.

In some embodiments, the topoisomerase inhibitor is chosen from one or more of doxorubicin HCl, Podophyllotoxin, Etoposide, Oxolinic Acid, Sedanolide, Mitoxantrone Dihydrochloride, 9-Hydroxyellipticine, or Amrubicin or a combination thereof. In some embodiments, the topoisomerase inhibitor is doxorubicin HCl.

### Cancers

Subjects that can be treated with a pharmaceutical composition as provided herein, or according to the methods as provided herein, include, but are not limited to, patients that have been diagnosed as having breast cancer such as a ductal carcinoma, lobular carcinoma, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, NK cell leukemia (e.g., blastic plasmacytoid dendritic cell neoplasm), acute myelogenous leukemia (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; cutaneous or intraocular (eye) melanoma; primary liver cancer; kidney cancer; thyroid cancer such as papillary, follicular, medullary and anaplastic; lymphoma such as diffuse large B-cell lymphoma, B-cell immunoblastic lymphoma, NK cell lymphoma (e.g., blastic plasmacytoid dendritic cell neoplasm), and Burkitt lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer; central nervous system cancers (CNS) such as primary brain tumor, which includes gliomas (astrocytoma, anaplastic astrocytoma, or glioblastoma multiforme), oligodendroglioma, ependymoma, meningioma, lymphoma, schwannoma, and medulloblastoma; peripheral nervous system (PNS) cancers such as acoustic neuromas and malignant peripheral nerve sheath tumor (MPNST) including neurofibromas and schwannomas, malignant fibrocytoma, malignant fibrous histiocytoma, malignant meningioma, malignant mesothelioma, and malignant mixed Müllerian tumor; oral cavity and oropharyngeal cancers such as, hypopharyngeal cancer, laryngeal cancer, nasopharyngeal cancer, and oropharyngeal cancer; stomach cancers such as lymphomas, gastric stromal tumors, and carcinoid tumors; testicular cancers such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin lymphoma, non-Hodgkin lymphomas carcinoids or carcinoid tumors; rectal cancer; and colon cancer.

In one embodiment, the cancer or disease that can be treated (e.g., inhibited or prevented) by methods, compositions, or kits provided herein includes a blood disorder or a hematologic malignancy.

In some embodiments, the cancer or disease that can be treated by by methods, compositions, or kits provided herein is selected from one or more of the following: acoustic neuroma, adenocarcinoma, adrenal gland cancer, anal cancer, angiosarcoma (e.g., lymphangiosarcoma, lymphangioendotheliosarcoma, hemangiosarcoma), benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer, esophageal cancer (e.g., adenocarcinoma of the esophagus, Barrett's adenocarinoma), Ewing sarcoma, familiar hypereosinophilia, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphocytic leukemia (ALL), which includes B-lineage ALL and T-lineage ALL, chronic lymphocytic leukemia (CLL), prolymphocytic leukemia (PLL), hairy cell leukemia (HCL) and Waldenstrom's macroglobulinemia (WM); peripheral T cell lymphomas (PTCL), adult T cell leukemia/lymphoma (ATL), cutaneous T-cell lymphoma (CTCL), large granular lymphocytic leukemia (LGF), Hodgkin's disease and Reed-Stemberg disease; acute myelocytic leukemia (AML), chronic myelocytic leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), leiomyosarcoma (LMS), mastocytosis (e.g., systemic mastocytosis), multiple myeloma (MM), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), Paget's disease of the vulva, Paget's disease of the penis, papillary adenocarcinoma, pancreatic cancer (e.g., pancreatic andenocarcinoma, intraductal papillary mucinous neoplasm (IPMN)), pinealoma, primitive neuroectodermal tumor (PNT), prostate cancer (e.g., prostate adenocarcinoma), rhabdomyosarcoma, retinoblastoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), and Waldenstrom's macroglobulinemia.

In one embodiment, the cancer or disease being treated or prevented, such as a blood disorder or hematologic malignancy, has a high expression level of one or more PI3K isoform(s) (e.g., PI3K-α, PI3K-β, PI3K-δ, or PI3K-γ, or a combination thereof).

In one embodiment, the cancer or disease that may be treated or prevented by methods, compositions, or kits provided herein includes a blood disorder or a hematologic malignancy, including, but not limited to, myeloid disorder, lymphoid disorder, leukemia, lymphoma, myelodysplastic syndrome (MDS), myeloproliferative disease (MPD), mast cell disorder, and myeloma (e.g., multiple myeloma), among others.

In one embodiment, the blood disorder or the hematologic malignancy includes, but is not limited to, acute lymphoblastic leukemia (ALL), T-cell ALL (T-ALL), B-cell ALL (B-ALL), acute myeloid leukemia (AML), chronic lymphocytic leukemia (CLL), chronic myelogenous leukemia (CML), blast phase CML, small lymphocytic lymphoma (SLL), CLL/SLL, blast phase CLL, Hodgkin lymphoma (HL), non-Hodgkin lymphoma (NHL), B-cell NHL, T-cell NHL, indolent NHL (iNHL), diffuse large B-cell lymphoma (DLBCL), mantle cell lymphoma (MCL), aggressive B-cell NHL, B-cell lymphoma (BCL), Richter's syndrome (RS), T-cell lymphoma (TCL), peripheral T-cell lymphoma (PTCL), cutaneous T-cell lymphoma (CTCL), transformed mycosis fungoides, Sézary syndrome, anaplastic large-cell lymphoma (ALCL), follicular lymphoma (FL), Waldenström macroglobulinemia (WM), lymphoplasmacytic lymphoma, Burkitt lymphoma, multiple myeloma (MM), amyloidosis, MPD, essential thrombocytosis (ET), myelofibrosis (MF), polycythemia vera (PV), chronic myelomonocytic leukemia (CMML), myelodysplastic syndrome (MDS), angioimmunoblastic lymphoma, high-risk MDS, and low-risk MDS. In one embodiment, the hematologic malignancy is relapsed. In one embodiment, the hematologic malignancy is refractory. In one embodiment, the cancer or disease is in a pediatric patient (including an infantile patient). In one embodiment, the cancer or disease is in an adult patient. Additional embodiments of a cancer or disease being treated or prevented by methods, compositions, or kits provided herein are described herein elsewhere.

In exemplary embodiments, the cancer or hematologic malignancy is CLL. In exemplary embodiments, the cancer or hematologic malignancy is CLL/SLL. In exemplary embodiments, the cancer or hematologic malignancy is blast phase CLL. In exemplary embodiments, the cancer or hematologic malignancy is SLL.

In exemplary embodiments, the cancer or hematologic malignancy is iNHL. In exemplary embodiments, the cancer or hematologic malignancy is DLBCL. In exemplary embodiments, the cancer or hematologic malignancy is B-cell NHL (e.g., aggressive B-cell NHL). In exemplary embodiments, the cancer or hematologic malignancy is MCL. In exemplary embodiments, the cancer or hematologic malignancy is RS. In exemplary embodiments, the cancer or hematologic malignancy is AML. In exemplary embodiments, the cancer or hematologic malignancy is MM. In exemplary embodiments, the cancer or hematologic malignancy is ALL. In exemplary embodiments, the cancer or hematologic malignancy is T-ALL. In exemplary embodiments, the cancer or hematologic malignancy is B-ALL. In exemplary embodiments, the cancer or hematologic malignancy is TCL. In exemplary embodiments, the cancer or hematologic malignancy is ALCL. In exemplary embodiments, the cancer or hematologic malignancy is leukemia. In exemplary embodiments, the cancer or hematologic malignancy is lymphoma. In exemplary embodiments, the cancer or hematologic malignancy is T-cell lymphoma. In exemplary embodiments, the cancer or hematologic malignancy is MDS (e.g., low grade MDS). In exemplary embodiments, the cancer or hematologic malignancy is MPD. In exemplary embodiments, the cancer or hematologic malignancy is a mast cell disorder. In exemplary embodiments, the cancer or hematologic malignancy is Hodgkin lymphoma (HL). In exemplary embodiments, the cancer or hematologic malignancy is non-Hodgkin lymphoma. In exemplary embodiments, the cancer or hematologic malignancy is PTCL. In exemplary embodiments, the cancer or hematologic malignancy is CTCL (e.g., mycosis fungoides or Sézary syndrome). In exemplary embodiments, the cancer or hematologic malignancy is WM. In exemplary embodiments, the cancer or hematologic malignancy is CML. In exemplary embodiments, the cancer or hematologic malignancy is FL. In exemplary embodiments, the cancer or hematologic malignancy is transformed mycosis fungoides. In exemplary embodiments, the cancer or hematologic malignancy is Sézary syndrome. In exemplary embodiments, the cancer or hematologic malignancy is acute T-cell leukemia. In exemplary embodiments, the cancer or hematologic malignancy is acute B-cell leukemia. In exemplary embodiments, the cancer or hematologic malignancy is Burkitt lymphoma. In exemplary embodiments, the cancer or hematologic malignancy is myeloproliferative neoplasms. In exemplary embodiments, the cancer or hematologic malignancy is splenic marginal zone. In exemplary embodiments, the cancer or hematologic malignancy is nodal marginal zone. In exemplary embodiments, the cancer or hematologic malignancy is extranodal marginal zone.

In one embodiment, the cancer or hematologic malignancy is a B cell lymphoma. In a specific embodiment, provided herein is a method of treating or managing a B cell lymphoma comprising administering to a patient a therapeutically effective amount of a compound provided herein, or a pharmaceutically acceptable derivative (e.g., salt or solvate) thereof. Also provided herein is a method of treating or lessening one or more of the symptoms associated with a B cell lymphoma comprising administering to a patient a therapeutically effective amount of a compound provided herein, or a pharmaceutically acceptable derivative (e.g., salt or solvate) thereof. In one embodiment, the B cell lymphoma is iNHL. In another embodiment, the B cell lymphoma is follicular lymphoma. In another embodiment, the B cell lymphoma is Waldenstrom macroglobulinemia (lymphoplasmacytic lymphoma). In another embodiment, the B cell lymphoma is marginal zone lymphoma (MZL) In another embodiment, the B cell lymphoma is MCL. In another embodiment, the B cell lymphoma is HL. In another embodiment, the B cell lymphoma is aNHL. In another embodiment, the B cell lymphoma is DLBCL. In another embodiment, the B cell lymphoma is Richters lymphoma.

In one embodiment, the cancer or hematologic malignancy is a T cell lymphoma. In a specific embodiment, provided herein is a method of treating or managing a T cell lymphoma comprising administering to a patient a therapeutically effective amount of a compound provided herein, or a pharmaceutically acceptable derivative (e.g., salt or solvate) thereof. Also provided herein is a method of treating or lessening one or more of the symptoms associated with a T cell lymphoma comprising administering to a patient a therapeutically effective amount of a compound provided herein, or a pharmaceutically acceptable derivative (e.g., salt or solvate) thereof. In one embodiment, the T cell lymphoma is peripheral T cell lymphoma (PTCL). In another embodiment, the T cell lymphoma is cutaneous T cell lymphoma (CTCL).

In one embodiment, the cancer or hematologic malignancy is Sézary syndrome. In a specific embodiment, provided herein is a method of treating or managing Sézary syndrome comprising administering to a patient a therapeutically effective amount of a compound provided herein, or a pharmaceutically acceptable derivative (e.g., salt or solvate) thereof Also provided herein is a method of treating or lessening one or more of the symptoms associated with Sézary syndrome comprising administering to a patient a therapeutically effective amount of a compound provided herein, or a pharmaceutically acceptable derivative (e.g., salt or solvate) thereof. The symptoms associated with Sézary syndrome include, but are not limited to, epidermotropism by neoplastic CD4+ lymphocytes, Pautrier's microabscesses, erythroderma, lymphadenopathy, atypical T cells in the peripheral blood, and hepatosplenomegalyln one embodiment, the therapeutically effective amount for treating or managing Sézary syndrome is from about 25 mg to 75 mg, administered twice daily. In other embodiments, the therapeutically effective amount is from about 50 mg to about 75 mg, from about 30 mg to about 65 mg, from about 45 mg to about 60 mg, from about 30 mg to about 50 mg, or from about 55 mg to about 65 mg, each of which is administered twice daily. In one embodiment, the effective amount is about 60 mg, administered twice daily.

It will be appreciated by one of skill in the medical arts that the exact manner of administering to said patient of a therapeutically effective amount of a PI3K inhibitor following a diagnosis of a patient's likely responsiveness to a PI3K inhibitor will be at the discretion of the attending physician. The mode of administration, including dosage, combination with other anti-cancer agents, timing and frequency of administration, and the like, may be affected by the diagnosis of a patient's likely responsiveness to a PI3K inhibitor, as well as the patient's condition and history. Thus, even patients diagnosed with tumors predicted to be relatively insensitive to PI3K inhibitors may still benefit from treatment with such inhibitors, particularly in combination with other anti-cancer agents, or agents that can alter a tumor's sensitivity to PI3K inhibitors.

The effectiveness of treatment in the preceding methods can for example be determined by measuring the decrease in size of tumors present in the patients with the neoplastic condition, or by assaying a molecular determinant of the degree of proliferation of the tumor cells.

Suitable test agents which can be tested in the preceding method include combinatorial libraries, defined chemical entities, peptide and peptide mimetics, oligonucleotides and natural product libraries, such as display (e.g. phage display libraries) and antibody products. Test agents may be used in an initial screen of, for example, 10 substances per reaction, and the substances of these batches which show inhibition or activation tested individually. Test agents may be used at a concentration of from 1 nM to 1000 μM, preferably from 1 μM to 100 μM, more preferably from 1 μM to 10 μM.

In certain embodiments, provided herein is a method of treating, managing, or preventing a cancer in a subject comprising administering to the subject a a PI3K inhibitor (e.g., one or more PI3K inhibitors, e.g., GS1101 and/or Compound 1), or a pharmaceutically acceptable form thereof, in combination with a second agent or a pharmaceutically acceptable form thereof, wherein the second agent is selected from one or more of 1) a MEK inhibitor (e.g., trametinib or PD-0325901), 2) a mTOR inhibitor (e.g., everolimus or AZD8055), 3) an AKT inhibitor (e.g., perifosine or MK-2206), 4) a proteasome inhibitor (e.g., bortezomib or carfilzomib), 5) an immunomodulator (e.g., lenalidomide), 6) a glucocorticosteroid (e.g. dexamethasone), 7) a CDK4/6 inhibitor, 8) an HDAC inhibitor, 9) a BET inhibitor, 10) an epigenetic inhibitor, 11) a PI3K alpha inhibitor, 12) a topoisomerase inhibitor, or 13) an ERK inhibitor, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma. In certain embodiments, the combination is therapeutically effective. In certain embodiments, the combination is synergistic.

In one embodiment of the methods provided herein, the subject shows decreased responsiveness to a PI3K inhibitor (e.g., is resistant or refractive to treatment with a PI3K inhibitor, e.g., Compound 1). In one embodiment, the subject is identified as having a decreased susceptibility (e.g., resistance or acquired resistance) to a monotherapy treatment of a PI3K inhibitor (e.g., Compound 1), or a pharmaceutically acceptable form thereof. In one embodiment, the subject is identified as having an increased susceptibility to a combination therapy treatment provided herein.

Also provided herein are methods of delaying resistance of a subject, or prolonging remission (e.g., complete remission or partial remission) of a subject, to a PI3K inhibitor, e.g., Compound 1 or CAL-101 or to a second agent such as a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein. In some embodiments, the method of delaying resistance of the subject, or prolonging remission (e.g., complete remission or partial remission) of the subject, comprises administering a combination of a PI3K inhibitor (e.g., Compound 1 or CAL-101) and a second agent (e.g., a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein to the subject before the subject develops resistance to the PI3K inhibitor (e.g., Compound 1 or CAL-101). In some embodiments, the method of delaying resistance of the subject, or prolonging remission (e.g., complete remission or partial remission) of the subject, comprises administering a combination of a PI3K inhibitor (e.g., Compound 1 or CAL-101) and a second agent (e.g., a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein) to the subject before the subject develops resistance to the second agent.

In some embodiments, the subject is not resistant to a PI3K inhibitor (e.g., Compound 1 or CAL-101). In some embodiments, the subject is not resistant to a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein. In some embodiments, the subject has previously been administered a PI3K inhibitor (e.g., Compound 1 or CAL-101) as a monotherapy or in combination with an agent other than a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein. In some embodiments, the subject has previously been administered a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein as a monotherapy or in combination with an agent other than a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein. In some embodiments, the subject has a cancer, e.g., a cancer described herein. In some embodiments, in accordance with the method, resistance is delayed compared to the time in which resistance generally develops when the subject is treated with any of the agents or inhibitors alone as monotherapy. In some embodiments, the resistance is delayed by at least 2 weeks, e.g., at least 2 weeks, 4 weeks, 1 month, 2 months, 3 months, 4 months, 5 months, 6 months, 8 months, 10 months, 12 months, 1 year, 2 years, 4 years, 6 years, 8 years, or more. In some embodiments, in accordance with the method, remission (e.g., complete remission or partial remission) is prolonged compared to the time in which remission generally lasts when the subject is treated with any of the agents or inhibitors alone as monotherapy. In some embodiments, remission (e.g., complete remission or partial remission) is prolonged by at least 2 weeks, e.g., at least 2 weeks, 4 weeks, 1 month, 2 months, 3 months, 4 months, 5 months, 6 months, 8 months, 10 months, 12 months, 1 year, 2 years, 4 years, 6 years, 8 years, or more.

In some embodiments, once the subject becomes resistant to the PI3K inhibitor (e.g., Compound 1 or CAL-101) or the second agent (e.g., a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein), the agent to which the subject is resistant is withdrawn. In other embodiments, once the subject becomes resistant to the PI3K inhibitor (e.g., Compound 1 or CAL-101) or the second agent (e.g., a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein), the agent to which the subject is resistant continued. In some embodiments, addition of the PI3K inhibitor or the second agent to the therapeutic regimen increases or restores sensitivity to the agent to which the cancer is resistant. For instance, in some embodiments, addition of the second agent to the therapeutic regimen increases or restores sensitivity to the PI3K inhibitor to which the cancer is resistant.

Provided herein is also a method of reducing, e.g., overcoming, resistance of a subject to a PI3K inhibitor (e.g., Compound 1 or CAL-101), comprising administering the PI3K inhibitor as a monotherapy to the subject until development of resistance in the subject to the PI3K inhibitor, and subsequently administering a second agent (e.g., a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein) to the subject. In some cases, the method comprises continuing administration of the PI3K inhibitor (e.g., at the same dosage, lower dosage, or higher dosage) to the subject in combination with the second agent. In other cases, the method comprises discontinuing administration of the PI3K inhibitor upon commencing administration of the second agent. For example the administration of the PI3K inhibitor is stopped before administration of the second agent commences. In other examples, the dosage of the PI3K inhibitor is decreased, e.g., gradually, upon commencing administration of the second agent. In some embodiments, provided herein is a method of reducing, e.g., overcoming, resistance of a subject to a PI3K inhibitor (e.g., Compound 1 or CAL-101), comprising administering the PI3K inhibitor and the second agent (e.g., a MEK inhibitor, mTOR inhibitor, AKT inhibitor, protease inhibitor, immunomodulator, glucocorticosteroid, CDK4/6 inhibitor, HDAC inhibitor, CD20 inhibitor, a BET inhibitor, an epigenetic inhibitor, a PI3K alpha inhibitor, a topoisomerase inhibitor, or an ERK inhibitor described herein) to the subject before the subject develops resistance to the PI3K inhibitor, in order to prevent resistance arising, reduce the likelihood of resistance developing, or increase the length of time before resistance develops.

In one embodiment, a method described herein further comprises administration of a CD20 inhibitor, e.g., an anti-CD20 antibody. In one embodiment, a pharmaceutical composition described herein further comprises a CD20 inhibitor, e.g., an anti-CD20 antibody. In some such embodiments, the CD20 inhibitor, e.g., the anti-CD20 antibody, is included in the same dosage form as the PI3K inhibitor and/or second agent. In some such embodiments, the CD20 inhibitor, e.g., the anti-CD20 antibody, is in a separate dosage form as the PI3K inhibitor and/or second agent. The CD20 inhibitor, e.g., the anti-CD20 antibody, can be administered before, after, or concurrent with the PI3K inhibitor and/or second agent. Exemplary CD20 inhibitors include, but are not limited to, anti-CD20 antibody and other inhibitors, such as rituximab, obinutuzumab (GA-101), tositumomab, ¹³¹I tositumomab, ⁹⁰Y ibritumomab, ¹¹¹I ibritumomab, ofatumumab, veltuzumab, and ocrelizumab), AME-133v, PRO131921 and TRU-015.

The combination of the PI3K inhibitor and the second agent can be administered together in a single dosage form or administered separately in two or more different dosage forms as described herein. In certain embodiments, the anti-CD20 antibody is selected from rituximab, ofatumumab and obinutuzumab.

In an embodiment, a composition described herein includes a combination of a PI3K inhibitor (e.g., a PI3K inhibitor described herein, e.g., Compound 1 or CAL-101) and an anti-CD20 antibody or fragment thereof, e.g., an anti-CD20 monoclonal antibody (mAb), such as obinutuzumab. In some embodiments, provided herein is a method of treating, managing, or preventing a cancer in a subject comprising administering to the subject a combination of a PI3K inhibitor (e.g., Compound 1 or CAL-101) with an anti-CD20 antibody or fragment thereof, e.g., an anti-CD20 monoclonal antibody (mAb), such as obinutuzumab. In some embodiments, the subject has a cancer, e.g., a cancer described herein, e.g., a hematological cancer, such as a lymphoma. In some embodiments, the effect of combining the Compound 1 or CAL-101 with obinutuzumab includes an additive effect on cell killing, e.g., cancer cell killing In some embodiments, the PI3K inhibitor (e.g., Compound 1 or CAL-101) is administered concurrently with, prior to, or subsequent to, the obinutuzumab. In some embodiments, combinations of the PI3K inhibitor (e.g., Compound 1 or CAL-101) and obinutuzumab allows the PI3K inhibitor and/or the obinutuzumab to be administered at a lower dosage or a lower frequency than would be required to achieve the same therapeutic effect compared to a monotherapy dose. Such a combination provides advantageous effects, e.g., in reducing, preventing, delaying, and/or decreasing the occurrence of one or more of: a side effect, toxicity, or resistance that would otherwise be associated with administration of a higher dose of one or both of the agents.

As a monotherapy, obinutuzumab can be administered according to the following regimen of 28-day cycles: 100 mg on C1D1 (cycle 1, day one), 900 mg on C1D2, 1000 mg on C1D8, 1000 mg on C1D15, and 1000 mg on day 1 of each subsequent cycle, e.g., cycles 2-6. In some embodiments, when administered in combination with a PI3K inhibitor, the dosage of obinutuzumab can be reduced compared to its monotherapy dose, e.g., 300-400, 400-500, 500-600, 600-700, 700-800, 800-900, or 900-1000 mg/cycle (e.g., for a 28-day cycle). In some embodiments, when administered in combination with a PI3K inhibitor, the frequency of administration of obinutuzumab can be reduced compared to its frequency as a monotherapy, e.g., to one administration every 28-30, 30-35, 35-40, 40-45, 45-50, 50-55, or 55-60 days.

Methods for monitoring minimal residual disease negativity (MRD) are known in the art. See, e.g., Zhou, J. et al., Blood, 2007, 110: 1607-1611 (Prepublished online May 7, 2007. doi: 10.1182/blood-2006-09-045369). Such methods include DNA based tests or RNA based tests. In certain embodiments, MRD is monitored using flow cytometry, sequencing, or PCR.

In some embodiments, the compositions and methods described herein are effective to reduce MRD.

In some embodiments, the methods described herein include selecting a subject for treatment with the combination of a PI3K inhibitor and the second agent. In certain embodiments, the subject (e.g., a patient with a cancer, e.g., a cancer described herein) is selected for treatment with the combination based on the MRD in the subject. In certain embodiments, the selection is based on the presence of an MRD above a preselected level (e.g., 1 malignant cell in 100 normal cells, 1 malignant cell in 1000 normal cells, or 1 malignant cell in 10,000 normal cells).

In some embodiments, the methods described herein further comprise monitoring the MRD in a subject, e.g., evaluating MRD at at least one, two, three, four, five, six, nine months after initiating, continuing or ceasing treatment (e.g., PI3K inhibitor monotherapy or a second agent monotherapy, or a combination therapy disclosed herein).

In some embodiments, the combination of a PI3K inhibitor (e.g. a PI3K inhibitor described herein) and a second agent (e.g., a second agent described herein) is effective to reduce the MRD in the subject, e.g., below a level previously measured in the subject (e.g., the level measured before the combination treatment). In certain embodiments, the combination of a PI3K inhibitor and a second agent is effective to reduce the MRD in the subject below the level observed during or after treatment with a monotherapy, e.g., a monotherapy comprising either the PI3K inhibitor or the second agent inhibitor. In certain embodiments, the MRD is decreased below the level observed during treatment with a monotherapy comprising the PI3K inhibitor. In certain embodiments, the MRD is decreased below the level observed during treatment with a monotherapy comprising the PI3K inhibitor.

In certain embodiments, the combination is effective to reduce the MRD below a preselected cutoff value (e.g., 1 malignant cell in 100 normal cells, 1 malignant cell in 1000 normal cells, or 1 malignant cell in 10,000 normal cells). In certain embodiments, the preselected cutoff value is 1 malignant cell in 1000 normal cells. In those embodiments where the MRD is below a preselected cutoff value (e.g., preselected cutoff value as described herein), the treatment (e.g., PI3K inhibitor monotherapy or a second agent monotherapy, or a combination therapy disclosed herein) can be altered or discontinued. If upon monitoring the MRD (at at least one, two, three, four, five, six, nine months after altering or discontinuing the therapy), the MRD levels are increased above a preselected cutoff (e.g., a preselected cutoff as described herein), a second treatment can be initiated (e.g., PI3K inhibitor monotherapy or the second agent monotherapy, a combination therapy disclosed herein, or a combination with a third agent, e.g., an anti-CD20 inhibitor or a BTK inhibitor such as ibrutinib).

In some embodiments provided herein is a method of treating cancer in a subject, the method comprising (i) administering to the subject a monotherapy (e.g., a monotherapy comprising a PI3K inhibitor or a second therapeutic agent as described herein) and monitoring the MRD in the subject, and (ii) if the MRD increases above a preselected cutoff value (e.g., 1 malignant cell in 100 normal cells, 1 malignant cell in 1000 normal cells, or 1 malignant cell in 10,000 normal cells), administering to the subject a PI3K inhibitor in combination with a second agent. In certain embodiments, the combination is effective to reduce the MRD, e.g. to reduce the MRD below the cutoff value. In certain embodiments, the preselected cutoff value is 1 malignant cell in 1000 or 10,000 normal cells.

In certain embodiments, provided herein is a method of treating a cancer in a subject, or a method of decreasing minimal residual disease (MRD) in a subject diagnosed with a cancer, the method comprising: (a) administering to the subject a PI3K inhibitor (e.g., Compound 1), or a pharmaceutically acceptable form thereof, in combination with a second agent (e.g., at least one second agent); (b) monitoring the MRD in the subject by one or more methods described herein or known in the art (e.g., flow cytometry, sequencing, or PCR), and administering a monotherapy comprising the PI3K inhibitor, or a pharmaceutically acceptable form thereof, to the subject if the MRD in the subject increases above a preselected cutoff value (e.g., 1 malignant cell in 100 normal cells, 1 malignant cell in 1000 normal cells, or 1 malignant cell in 10,000 normal cells); and (c) monitoring the amount of MRD negativity (by one or more methods described herein or known in the art (e.g., flow cytometry, sequencing, or PCR) in the subject receiving the monotherapy, and administering a further combination comprising the PI3K inhibitor, or a pharmaceutically acceptable form thereof, and a third agent (e.g., at least one third agent) to the subject if the MRD is greater than the preselected cutoff value. In one embodiment, the third agent is selected from one or more of an anti-CD20 antibody, a MEK inhibitor, dexamethasone, lenolidomide, an mTOR inhibitor, nitrogen mustard, and a nucleoside metabolic inhibitor.

In certain embodiments, provided herein is a method of increasing the depth of response resulting in MRD negativity, the method comprising: (a) administering to a patient with a cancer (e.g., a cancer disclosed herein) a PI3K inhibitor (e.g., Compound 1), or a pharmaceutically acceptable form thereof, and a second agent (e.g., at least one second agent); (b) monitoring for the presence of MRD negativity in the patient by one or more methods described herein or known in the art (e.g., flow cytometry, sequencing, or PCR). In one embodiment, the second agent is selected from anti-CD20 antibody, a MEK inhibitor, dexamethasone, lenolidomide, an mTOR inhibitor, nitrogen mustard, and nucleoside metabolic inhibitor.

In some embodiments, the second agent is a chemotherapeutic. In some embodiments, the chemotherapeutic is selected from mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (imatinib mesylate), Velcade® (bortezomib), Casodex™ (bicalutamide), Iressa® (gefitinib), Tarceva® (erlotinib), and Adriamycin® (doxorubicin) as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; BTK inhibitors such as ibrutinib (PCI-32765), AVL-292, Dasatinib, LFM-AI3, ONO-WG-307, and GDC-0834; HDAC inhibitors such as vorinostat, romidepsin, panobinostat, valproic acid, belinostat, mocetinostat, abrexinostat, entinostat, SB939, resminostat, givinostat, CUDC-101, AR-42, CHR-2845, CHR-3996, 4SC-202, CG200745, ACY-1215 and kevetrin; EZH2 inhibitors such as, but not limited to, EPZ-6438 (N-((4,6-dimethyl-2-oxo-1,2-dihydropyridin-3-yl)methyl)-5-(ethyl(tetrahydro-2H-pyran-4-yl)amino)-4-methyl-4′-(morpholinomethyl)-[1,1′-biphenyl]-3-carboxamide), GSK-126 ((S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide), GSK-343 (1-Isopropyl-N-((6-methyl-2-oxo-4-propyl-1,2-dihydropyridin-3-yl)methyl)-6-(2-(4-methylpiperazin-1-yl)pyridine-4-yl)-1H-indazole-4-carboxamide), Ell, 3-deazaneplanocin A (DNNep, 5R-(4-amino-1H-imidazo[4,5-c]pyridin-1-yl)-3-(hydroxymethyl)-3-cyclopentene-1S,2R-diol), small interfering RNA (siRNA) duplexes targeted against EZH2 (S. M. Elbashir et al., Nature 411:494-498 (2001)), isoliquiritigenin, and those provided in, for example, U.S. Publication Nos. 2009/0012031, 2009/0203010, 2010/0222420, 2011/0251216, 2011/0286990, 2012/0014962, 2012/0071418, 2013/0040906, and 2013/0195843, all of which are incorporated herein by reference; JAK/STAT inhibitors such as lestaurtinib, tofacitinib, ruxolitinib, pacritinib, CYT387, baricitinib, GLPG0636, TG101348, INCB16562, CP-690550, and AZD1480; PKC-β inhibitor such as Enzastaurin; SYK inhibitors such as, but not limited to, GS-9973, R788 (fostamatinib), PRT 062607, R406, (S)-2-(2-((3,5-dimethylphenyl)amino)pyrimidin-4-yl)-N-(1-hydroxypropan-2-yl)-4-methylthiazole-5-carboxamide, R112, GSK143, BAY61-3606, PP2, PRT 060318, R348, and those provided in, for example, U.S. Publication Nos. 2003/0113828, 2003/0158195, 2003/0229090, 2005/0075306, 2005/0232969, 2005/0267059, 2006/0205731, 2006/0247262, 2007/0219152, 2007/0219195, 2008/0114024, 2009/0171089, 2009/0306214, 2010/0048567, 2010/0152159, 2010/0152182, 2010/0316649, 2011/0053897, 2011/0112098, 2011/0245205, 2011/0275655, 2012/0027834, 2012/0093913, 2012/0101275, 2012/0130073, 2012/0142671, 2012/0184526, 2012/0220582, 2012/0277192, 2012/0309735, 2013/0040984, 2013/0090309, 2013/0116260, and 2013/0165431, all of which are incorporated herein by reference; SYK/JAK dual inhibitor such as PRT2070; nitrogen mustards such as bendamustine, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomycins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pralatrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatrexate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethyla-mine; urethan; vindesine; dacarbazine; mannomustine; mitobronitol; mitolactol; pipobroman; gacytosine; arabinoside (Ara-C); cyclophosphamide; thiotepa; taxanes, e.g., paclitaxel (e.g., TAXOL™) and docetaxel (e.g., TAXOTERE™) and ABRAXANE® (paclitaxel protein-bound particles); retinoic acid; esperamicins; capecitabine; and pharmaceutically acceptable forms (e.g., pharmaceutically acceptable salts, hydrates, solvates, isomers, prodrugs, and isotopically labeled derivatives) of any of the above. Also included as suitable chemotherapeutic cell conditioners are anti-hormonal agents that act to regulate or inhibit hormone action on tumors such as anti-estrogens including for example tamoxifen (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition as provided herein can be used in combination with commonly prescribed anti-cancer drugs such as Herceptin®, Avastin®, Erbitux®, Rituxan®, Taxol®, Arimidex®, Taxotere®, ABVD, AVICINE, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastic, antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, crizotinib, cell-cycle nonspecific antineoplastic agents, dichloroacetic acid, discodermolide, elsamitrucin, enocitabine, epothilone, eribulin, everolimus, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitor, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

In some embodiments, the chemotherapeutic is selected from hedgehog inhibitors including, but not limited to IPI-926 (See U.S. Pat. No. 7,812,164). Other suitable hedgehog inhibitors include, for example, those described and disclosed in U.S. Pat. No. 7,230,004, U.S. Patent Application Publication No. 2008/0293754, U.S. Patent Application Publication No. 2008/0287420, and U.S. Patent Application Publication No. 2008/0293755, the entire disclosures of which are incorporated by reference herein. Examples of other suitable hedgehog inhibitors include those described in U.S. Patent Application Publication Nos. US 2002/0006931, US 2007/0021493 and US 2007/0060546, and International Application Publication Nos. WO 2001/19800, WO 2001/26644, WO 2001/27135, WO 2001/49279, WO 2001/74344, WO 2003/011219, WO 2003/088970, WO 2004/020599, WO 2005/013800, WO 2005/033288, WO 2005/032343, WO 2005/042700, WO 2006/028958, WO 2006/050351, WO 2006/078283, WO 2007/054623, WO 2007/059157, WO 2007/120827, WO 2007/131201, WO 2008/070357, WO 2008/110611, WO 2008/112913, and WO 2008/131354, each incorporated herein by reference. Additional examples of hedgehog inhibitors include, but are not limited to, GDC-0449 (also known as RG3616 or vismodegib) described in, e.g., Von Hoff D. et al., N. Engl. J. Med. 2009; 361(12):1164-72; Robarge K. D. et al., Bioorg Med Chem Lett. 2009; 19(19):5576-81; Yauch, R. L. et al. (2009) Science 326: 572-574; Sciencexpress: 1-3 (10.1126/science.1179386); Rudin, C. et al. (2009) New England J of Medicine 361-366 (10.1056/nejma0902903); BMS-833923 (also known as XL139) described in, e.g., in Siu L. et al., J. Clin. Oncol. 2010; 28:15s (suppl; abstr 2501); and National Institute of Health Clinical Trial Identifier No. NCT006701891; LDE-225 described, e.g., in Pan S. et al., ACS Med. Chem. Lett., 2010; 1(3): 130-134; LEQ-506 described, e.g., in National Institute of Health Clinical Trial Identifier No. NCT01106508; PF-04449913 described, e.g., in National Institute of Health Clinical Trial Identifier No. NCT00953758; Hedgehog pathway antagonists disclosed in U.S. Patent Application Publication No. 2010/0286114; SMOi2-17 described, e.g., U.S. Patent Application Publication No. 2010/0093625; SANT-1 and SANT-2 described, e.g., in Rominger C. M. et al., J. Pharmacol. Exp. Ther. 2009; 329(3):995-1005; 1-piperazinyl-4-arylphthalazines or analogues thereof, described in Lucas B. S. et al., Bioorg. Med. Chem. Lett. 2010; 20(12):3618-22.

Other hormonal therapy and chemotherapeutic agents include, but are not limited to, anti-estrogens (e.g. tamoxifen, raloxifene, and megestrol acetate), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids or taxanes (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, raltitrexed, tegafur-uracil, capecitabine), cytosine analogs (e.g. cytarabine (ara C, cytosine arabinoside), and fludarabine), purine analogs (e.g. mercaptopurine and thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracyclines (e.g. daunorubicin, doxorubicin, pegylated liposomal doxorubicin, idarubicin, epirubicin, pirarubicin, zorubicin, mitoxantrone), MDR inhibitors (e.g. verapamil), Ca2+ ATPase inhibitors (e.g. thapsigargin), thalidomide, lenalidomide (REVLIMID®), tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbazine, prednisolone, dexamethasone, camptothecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In some embodiments, a PI3K inhibitor disclosed herein (e.g., Compound 1 or CAL-101), is administered in combination with an inhibitor of one or more members of TAM family, a receptor tyrosine kinase (RTK) subfamily comprising Tyro-3 (also called Sky), Axl and Mer. In one embodiment, the TAM inhibitor is BGB324 (R428), 549076, TP0903, CEP-40783, ONO-9330547, bosutinib (SKI606, PF5208763), cabozantinib (XL184), sunitinib (SU11248), foretinib (XL880, GSK1363089), MGCD265, BMS777607 (ASLAN002), LY2801653, SGI7079, amuvatinib (SGI-0470-02, MP470), SNS314, PF-02341066, diaminopyrimidine, spiroindoline, UNC569, UNC1062, UNC1666, UNC2025, or LDC1267. Additional TAM inhibitors include those described in Mollard et al., Med. Chem. Lett. 2011, 2, 907-912 and Feneyrolles et al., Mol. Cancer Ther. 13(9), Published OnlineFirst Aug. 19, 2014, the entireties of which are incorporated by reference herein.

### Methods of Evaluating a Cancer

In the methods described herein the tumor cell will typically be from a patient diagnosed with cancer, a precancerous condition, or another form of abnormal cell growth, and in need of treatment.

Accordingly, the present invention provides a method of predicting the sensitivity of tumor cell growth to inhibition by a PI3K inhibitor, comprising: assessing the level of a prognosis-positive biomarker expressed by a tumor cell; and predicting the sensitivity of tumor cell growth to inhibition by a PI3K inhibitor, wherein high expression levels of tumor cell prognosis-positive biomarkers correlate with high sensitivity to inhibition by a PI3K inhibitor, or wherein low expression levels of said tumor cell prognosis-positive biomarker correlate with low sensitivity to inhibition by PI3K inhibitors. In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In a more preferred embodiment the PI3K inhibitor is selected from Compound 1 and GS1101. In a particularly preferred embodiment the PI3K inhibitor is Compound 1. In one embodiment the tumor or tumor cell is selected from chronic lymphocytic leukemia, non-Hodgkin lymphoma, diffuse large B-cell lymphoma, mantle cell lymphoma, and adult T-cell lymphoma. In a particularly preferred embodiment the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma.

The present invention also provides a method of predicting the sensitivity of tumor cell growth to inhibition by a PI3K inhibitor, comprising: assessing the level of a prognosis-negative biomarker expressed by a tumor cell; and predicting the sensitivity of tumor cell growth to inhibition by a PI3K inhibitor, wherein high expression levels of tumor cell prognosis-negative biomarkers correlate with low sensitivity to inhibition by PI3K inhibitors, or wherein low expression levels of said tumor cell prognosis-negative biomarker correlates with high sensitivity to inhibition by a PI3K inhibitor. In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In a more preferred embodiment the PI3K inhibitor is selected from Compound 1 and GS1101. In a particularly preferred embodiment the PI3K inhibitor is Compound 1. In one embodiment the tumor or tumor cell is selected from chronic lymphocytic leukemia, non-Hodgkin lymphoma, diffuse large B-cell lymphoma, mantle cell lymphoma, and adult T-cell lymphoma. In a particularly preferred embodiment the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma. In one embodiment the prognosis-negative biomarker is selected from BRAF copy number gain, CTNNB1 copy number gain, FHIT copy number gain, IRF4 copy number gain, MITF copy number gain, MN1 copy number gain, NF2 copy number gain, NF2 copy number loss, RET copy number loss, STK11 copy number loss, TSC2 copy number loss, and RB1 loss of heterozygocity. In a more preferred embodiment, the prognosis-negative biomarker is selected from IRF4 copy number gain, STK11 copy number loss and TSC2 copy number loss.

The present invention further provides a method for treating a tumor in a patient, comprising the step of administering to the patient a PI3K inhibitor, wherein the patient possesses a tumor that has been determined as having high sensitivity to tumor cell growth inhibition by a PI3K inhibitor by assessing the level of at least one prognosis-positive biomarker expressed by a tumor cell from said tumor; and predicting the sensitivity of tumor cell growth to inhibition by a PI3K inhibitor, wherein high expression levels of said tumor cell prognosis-positive biomarker correlate with high sensitivity to inhibition by a PI3K inhibitor; or

assessing the level of at least one prognosis-negative biomarker expressed by a tumor cell from said tumor; and predicting the sensitivity of tumor cell growth to inhibition by a PI3K inhibitor, wherein low expression levels of said tumor cell prognosis-negative biomarker correlate with high sensitivity to inhibition by a PI3K inhibitor.

In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In a more preferred embodiment the PI3K inhibitor is selected from Compound 1 and GS1101. In a particularly preferred embodiment the PI3K inhibitor is Compound 1. In one embodiment the tumor or tumor cell is selected from chronic lymphocytic leukemia, non-Hodgkin lymphoma, diffuse large B-cell lymphoma, mantle cell lymphoma, and adult T-cell lymphoma. In a particularly preferred embodiment the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma.

A further embodiment of the invention is a method of treating a tumor in a patient, comprising the step of administering to the patient a PI3K inhibitor as a first-line therapy, wherein the patient possesses a tumor that has been determined as having high sensitivity to tumor cell growth inhibition

assessing the level of at least one prognosis-positive biomarker expressed by a tumor cell from said tumor; and predicting the sensitivity of tumor cell growth to inhibition by a PI3K inhibitor, wherein high expression levels of said tumor cell prognosis-positive biomarker correlate with high sensitivity to inhibition by a PI3K inhibitor; or

assessing the level of at least one prognosis-negative biomarker expressed by a tumor cell from said tumor; and predicting the sensitivity of tumor cell growth to inhibition by a PI3K inhibitor, wherein low expression levels of said tumor cell prognosis-negative biomarker correlate with high sensitivity to inhibition by a PI3K inhibitor.

In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In a more preferred embodiment the PI3K inhibitor is selected from Compound 1 and GS1101. In a particularly preferred embodiment the PI3K inhibitor is Compound 1. In one embodiment the tumor or tumor cell is selected from chronic lymphocytic leukemia, non-Hodgkin lymphoma, diffuse large B-cell lymphoma, mantle cell lymphoma, and adult T-cell lymphoma. In a particularly preferred embodiment the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma.

Also provided by the present invention are PI3K inhibitors for use in the herein-described methods. Further provided are compositions comprising a PI3K inhibitor for use in the herein-described methods.

Additionally, methods are provided for the identification of new prognosis-positive or prognosis-negative biomarkers that are predictive of responsiveness of tumors to PI3K inhibitors.

Thus, for example, the present invention further provides a method of identifying a prognosis-positive biomarker that is predictive for more effective treatment of a neoplastic condition with a PI3K inhibitor, comprising: measuring the level of a candidate prognosis-positive biomarker in neoplastic cell-containing samples from patients with a neoplastic condition, and identifying a correlation between the level of said candidate prognosis-positive biomarker in the sample from the patient with the effectiveness of treatment of the neoplastic condition with a PI3K inhibitor, wherein a correlation of high levels of the prognosis-positive biomarker with more effective treatment of the neoplastic condition with a PI3K inhibitor indicates that said prognosis-positive biomarker is diagnostic for more effective treatment of the neoplastic condition with a PI3K inhibitor. In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In a more preferred embodiment the PI3K inhibitor is selected from Compound 1 and GS1101. In a particularly preferred embodiment the PI3K inhibitor is Compound 1. In one embodiment neoplastic condition is selected from chronic lymphocytic leukemia, non-Hodgkin lymphoma, diffuse large B-cell lymphoma, mantle cell lymphoma, and adult T-cell lymphoma. In a particularly preferred embodiment the neoplastic condition is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma.

The present invention further provides a method of identifying a prognosis-negative biomarker that is diagnostic for less effective treatment of a neoplastic condition with a PI3K inhibitor, comprising: (a) measuring the level of a candidate prognosis-negative biomarker in neoplastic cell-containing samples from patients with a neoplastic condition, and (b) identifying a correlation between the level of said candidate prognosis-negative biomarker in the sample from the patient with the effectiveness of treatment of the neoplastic condition with a PI3K inhibitor, wherein a correlation of high levels of the prognosis-negative biomarker with less effective treatment of the neoplastic condition with a PI3K inhibitor indicates that said prognosis-negative biomarker is diagnostic for less effective treatment of the neoplastic condition with a PI3K inhibitor. In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In a more preferred embodiment the PI3K inhibitor is selected from Compound 1 and GS1101. In a particularly preferred embodiment the PI3K inhibitor is Compound 1. In one embodiment the neoplastic condition is selected from chronic lymphocytic leukemia, non-Hodgkin lymphoma, diffuse large B-cell lymphoma, mantle cell lymphoma, and adult T-cell lymphoma. In a particularly preferred embodiment the neoplastic condition is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma. In one embodiment the prognosis-negative biomarker is selected from BRAF copy number gain, CTNNB1 copy number gain, FHIT copy number gain, IRF4 copy number gain, MITF copy number gain, MN1 copy number gain, NF2 copy number gain, NF2 copy number loss, RET copy number loss, STK11 copy number loss, TSC2 copy number loss, and RB1 loss of heterozygocity. In a more preferred embodiment, the prognosis-negative biomarker is selected from IRF4 copy number gain, STK11 copy number loss and TSC2 copy number loss.

For any given prognosis-positive or prognosis-negative biomarker, the range of expression level between tumor cells that are relatively insensitive to PI3K inhibitors and those that are sensitive, can readily be assessed by one of skill in the art, for example by testing on a panel of tumor cells as described herein, or by testing in tumor biopsies from patients whose tumors display a range of sensitivities to a PI3K inhibitor.

One of skill in the medical arts, particularly pertaining to the application of prognostic tests and treatment with therapeutics, will recognize that biological systems are somewhat variable and not always entirely predictable, and thus many good diagnostic tests or therapeutics are occasionally ineffective. Thus, it is ultimately up to the judgment of the attending physician to determine the most appropriate course of treatment for an individual patient, based upon test results, patient condition and history, and his own experience. There may even be occasions, for example, when a physician will choose to treat a patient with a PI3K inhibitor even when a tumor is not predicted to be particularly sensitive to PI3K inhibitors, based on data from diagnostic tests or from other criteria, particularly if all or most of the other obvious treatment options have failed, or if some synergy is anticipated when given with another treatment. The fact that the PI3K inhibitors as a class of compounds are relatively well tolerated compared to many other anti-cancer compounds, such as more traditional chemotherapy or cytotoxic agents used in the treatment of cancer, makes this a more viable option.

Furthermore, this invention also provides additional methods wherein simultaneous assessment of the expression level in tumor cells of more than one biomarker level is utilized.

Accordingly, the present invention provides a method of predicting the sensitivity of tumor cell growth to inhibition by a PI3K inhibitor, comprising: assessing the level of at least one (or a panel of) prognosis-positive biomarkers expressed by a tumor cell; and predicting the sensitivity of tumor cell growth to inhibition by a PI3K inhibitor, wherein simultaneous high expression levels of all of the assessed tumor cell prognosis-positive biomarkers correlates with high sensitivity to inhibition by a PI3K inhibitor. In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In one embodiment the PI3K inhibitor is selected from Compound 1 and GS1101. In one embodiment the PI3K inhibitor is Compound 1. In an embodiment the tumor or tumor cell is selected from chronic lymphocytic leukemia, non-Hodgkin lymphoma, diffuse large B-cell lymphoma, mantle cell lymphoma, and adult T-cell lymphoma. In one embodiment the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma.

The present invention also provides a method of predicting the sensitivity of tumor cell growth to inhibition by a PI3K inhibitor, comprising: assessing the level of one or more (or a panel of) prognosis-negative biomarkers expressed by a tumor cell; and predicting the sensitivity of tumor cell growth to inhibition by a PI3K inhibitor, wherein simultaneous low or undetectable expression levels of all of the assessed tumor cell prognosis-negative biomarkers correlates with high sensitivity to inhibition by a PI3K inhibitor. In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In some embodiments the PI3K inhibitor is selected from Compound 1 and GS1101. In certain embodiments the PI3K inhibitor is Compound 1. In one embodiment the tumor or tumor cell is selected from chronic lymphocytic leukemia, non-Hodgkin lymphoma, diffuse large B-cell lymphoma, mantle cell lymphoma, and adult T-cell lymphoma. In some embodiments the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma. In one embodiment the prognosis-negative biomarker is selected from BRAF copy number gain, CTNNB1 copy number gain, FHIT copy number gain, IRF4 copy number gain, MITF copy number gain, MN1 copy number gain, NF2 copy number gain, NF2 copy number loss, RET copy number loss, STK11 copy number loss, TSC2 copy number loss, and RB1 loss of heterozygocity. In certain embodiments, the prognosis-negative biomarker is selected from IRF4 copy number gain, STK11 copy number loss and TSC2 copy number loss.

The present invention also provides a method of predicting the sensitivity of tumor cell growth to inhibition by a PI3K inhibitor, comprising: assessing the level of one or more prognosis-positive biomarker expressed by a tumor cell; assessing the level of one or more prognosis-negative biomarker expressed by a tumor cell; and predicting the sensitivity of tumor cell growth to inhibition by a PI3K inhibitor, wherein a high ratio of prognosis-positive to prognosis-negative biomarker expression levels correlates with high sensitivity to inhibition by a PI3K inhibitor. As used herein, a high ratio of prognosis-positive to prognosis-negative biomarker expression levels means greater than 1:1, preferably greater than 1.1:1, preferably greater than 1.5:1, more preferably greater than 2:1, more preferably greater than 5:1, more preferably greater than 10:1, even more preferably greater than 100:1, or greater than 1,000:1. In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In some embodiments, the PI3K inhibitor is selected from Compound 1 and GS1101. In certain embodiments, the PI3K inhibitor is Compound 1. In one embodiment the tumor or tumor cell is selected from chronic lymphocytic leukemia, non-Hodgkin lymphoma, diffuse large B-cell lymphoma, mantle cell lymphoma, and adult T-cell lymphoma. In some embodiments, the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma. In one embodiment the prognosis-negative biomarker is selected from BRAF copy number gain, CTNNB1 copy number gain, FHIT copy number gain, IRF4 copy number gain, MITF copy number gain, MN1 copy number gain, NF2 copy number gain, NF2 copy number loss, RET copy number loss, STK11 copy number loss, TSC2 copy number loss, and RB1 loss of heterozygocity. In some embodiments, the prognosis-negative biomarker is selected from IRF4 copy number gain, STK11 copy number loss and TSC2 copy number loss.

In methods of this invention, biomarker expression level can be assessed relative to the biomarker level in non-tumor cells of the same tissue, or another cell or tissue source used as an assay reference. The expression level of a biomarker is considered high if expression level relative to a suitable reference is greater than 1:1, preferably greater than 1.1:1, preferably greater than 1.5:1, more preferably greater than 2:1, more preferably greater than 5:1, more preferably greater than 10:1, even more preferably greater than 100:1, even more preferably greater than 1,000:1, even more preferably greater than 10,000:1, even more preferably greater than 1,000,000:1. The expression level of a biomarker is considered low if expression level relative to a suitable reference is less than 1:1, preferably less than 1:1.1, preferably less than 1:1.5, more preferably less than 1:2, more preferably less than 1:5, more preferably less than 1:10, even more preferably less than 1:100, even more preferably less than 1:1,000, even more preferably less than 1:10,000, even more preferably less than 1:1,000,000.

The present invention further provides a method of predicting the likelihood that a tumor will progress to a more aggressive tumor wherein the tumor is treatable with a PI3K inhibitor, comprising: assessing the level of at least one progression-positive biomarker expressed by a tumor cell from said tumor; and predicting the likelihood that the tumor cell will progress to a more aggressive tumor, wherein high expression levels of said tumor cell progression-positive biomarker correlate with high likelihood that the tumor cell will progress to a more aggressive tumor or wherein low expression levels of said tumor cell progression-positive biomarker correlate with low likelihood that the tumor cell will progress to a more aggressive tumor. In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In some embodiments, the PI3K inhibitor is selected from Compound 1 and GS1101. In some embodiments, the PI3K inhibitor is Compound 1. In one embodiment the tumor or tumor cell is selected from chronic lymphocytic leukemia, non-Hodgkin lymphoma, diffuse large B-cell lymphoma, mantle cell lymphoma, and adult T-cell lymphoma. In certain embodiments, the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma. In one embodiment the progression-positive biomarker is a genomic alteration in one or more gene in the 6q deletion region. In one embodiment, the progression-positive biomarker is a genomic alteration in an NF—B pathway gene. In one embodiment, the progression-positive biomarker is a del(6q13-16) or a del(6q23-24). In one embodiment the progression-positive biomarker is a TNFAIP3 mutation or copy number loss. In one embodiment the progression-positive biomarker is an EPHA7 mutation or copy number loss.

The present invention also provides a method of predicting the likelihood that a tumor cell from a tumor will progress to a more aggressive tumor wherein the tumor is treatable with a PI3K inhibitor, comprising: assessing the level of at least one progression-negative biomarker expressed by a tumor cell; and predicting the likelihood that the tumor cell will progress to a more aggressive tumor, wherein high expression levels of said tumor cell progression-negative biomarker correlate with low likelihood that the tumor cell will progress to a more aggressive tumor, or wherein low expression levels of said tumor cell progression-negative biomarker correlates with high sensitivity to inhibition by a PI3K inhibitor. In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In some embodiments, the PI3K inhibitor is selected from Compound 1 and GS1101. In certain embodiments, the PI3K inhibitor is Compound 1. In one embodiment the tumor or tumor cell is selected from chronic lymphocytic leukemia, non-Hodgkin lymphoma, diffuse large B-cell lymphoma, mantle cell lymphoma, and adult T-cell lymphoma. In some embodiments, the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma. In one embodiment the progression-positive biomarker is a genomic alteration in one or more gene in the 6q deletion region. In one embodiment, the progression-positive biomarker is a genomic alteration in an NF-B pathway gene. In one embodiment, the progression-positive biomarker is a del(6q13-16) or a del(6q23-24). In one embodiment the progression-positive biomarker is a TNFAIP3 mutation or copy number loss. In one embodiment the progression-positive biomarker is an EPHA7 mutation or copy number loss.

In a further aspect, the present invention provides a method for treating a tumor in a patient, comprising the step of administering to the patient a PI3K inhibitor, wherein there is a high likelihood that the patient will develop a more aggressive tumor and wherein said likelihood has been determined by:

assessing the level of at least one progression-positive biomarker expressed by a tumor cell from said tumor; and predicting the likelihood that the tumor cell will progress to a more aggressive tumor, wherein high expression levels of said tumor cell progression-positive biomarker correlate with high likelihood that the tumor cell will progress to a more aggressive tumor; or

assessing the level of at least one progression-negative biomarker expressed by a tumor cell from said tumor; and predicting the likelihood that the tumor cell will progress to a more aggressive tumor, wherein low expression levels of said tumor cell progression-negative biomarker correlate with high likelihood that the tumor cell will progress to a more aggressive tumor.

In one embodiment, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147. In some embodiments, the PI3K inhibitor is selected from Compound 1 and GS1101. In certain embodiments, the PI3K inhibitor is Compound 1. In one embodiment the tumor or tumor cell is selected from chronic lymphocytic leukemia, non-Hodgkin lymphoma, diffuse large B-cell lymphoma, mantle cell lymphoma, and adult T-cell lymphoma. In some embodiments, the tumor is selected from chronic lymphocytic leukemia, non-Hodgkin lymphoma and diffuse large B-cell lymphoma. In one embodiment, the PI3K inhibitor is Compound 1 and the tumor or tumor cell is indolent non-Hodgkin lymphoma. In one embodiment the progression-positive biomarker is a genomic alteration in one or more gene in the 6q deletion region. In one embodiment, the progression-positive biomarker is a genomic alteration in an NF-B pathway gene. In one embodiment, the progression-positive biomarker is a del(6q13-16) or a del(6q23-24). In one embodiment the progression-positive biomarker is a TNFAIP3 mutation or copy number loss. In one embodiment the progression-positive biomarker is an EPHA7 mutation or copy number loss.

In the methods of this invention, the level of a prognosis-positive or prognosis-negative biomarker expressed by a tumor cell can be assessed by using any of the standard bioassay procedures known in the art for determination of the level of expression of a gene, including for example ELISA, RIA, immunopreciptation, immunoblotting, immunofluorescence microscopy, RT-PCR, in situ hybridization, cDNA microarray, or the like, as described in more detail below.

In the methods of this invention, the expression level of a tumor cell prognosis-positive biomarker or prognosis-negative biomarker is preferably assessed by assaying a tumor biopsy. However, in an alternative embodiment, expression level of the tumor cell biomarker can be assessed in bodily fluids or excretions containing detectable levels of biomarkers originating from the tumor or tumor cells. Bodily fluids or excretions useful in the present invention include blood, urine, saliva, stool, pleural fluid, lymphatic fluid, sputum, ascites, prostatic fluid, cerebrospinal fluid (CSF), or any other bodily secretion or derivative thereof. By blood it is meant to include whole blood, plasma, serum or any derivative of blood. Assessment of tumor prognosis-positive or prognosis-negative biomarkers in such bodily fluids or excretions can sometimes be preferred in circumstances where an invasive sampling method is inappropriate or inconvenient.

In any of the above methods referring to a patient sample, an example of such a sample can be a tumor biopsy.

In one embodiment, the biomarkers provided herein include, but are not limited to, a target biomarker, a signaling pathway biomarker, a protein mutation biomarker, a protein expression biomarker, a gene mutation biomarker, a copy number alteration (CNA) biomarker, a gene expression biomarker, a cytokine biomarker, a chemokine biomarker, a matrix metalloproteinase biomarker, or a biomarker for particular cancer cells. In one embodiment, the biomarker can be used to evaluate the prognosis, and/or sensitivity to a treatment agent, of a particular type of cancer or disease, or of a particular patient or group of patients.

In one embodiment, the prognosis-positive or prognosis-negative biomarker is a genomic alteration. In one embodiment, the genomic alteration is a gene mutation or a copy number alteration. In one embodiment, the gene mutation is a non-dbSNP mutation. In another embodiment, the gene mutation is a single nucleotide polymorphism (SNP) mutation. In one embodiment, the prognosis-negative biomarker is associated with a mutation in one or more of the following genes: ALK, SF3B1, TP53, NOTCH1, MYD88, ATM, XPO1, POT1, NRAS, BCOR, KRAS, MED12, DDX3X, FBXW7, BTK and PLCG2. In one embodiment, the prognosis-negative biomarker is associated with a mutation in one or more of the following genes: SF3B1, TP53, NOTCH1, MYD88, ATM, XPO1, MED12, and FBXW7. In one embodiment, the prognosis-negative biomarker is associated with a chromosome deletion.

In one embodiment, the prognosis-negative biomarker is associated with one or more genomic alterations selected from the group consisting of del(11q21), del(13q14), trisomy 12, del(11q22-23), del(17p13), del(8p), TP53 mutation, TP53 pathway mutation, MAPK pathway mutation, TP53 copy number loss, STK11 copy number loss, TSC1 copy number loss, and TSC2 copy number loss. In one embodiment, the prognosis-negative biomarker is copy number loss in one or more of STK11, TSC1, and TSC2. In one embodiment, the prognosis-negative biomarker is copy number loss in STK11. In one embodiment, the prognosis-negative biomarker is copy number loss in TSC1. In one embodiment, the prognosis-negative biomarker is copy number loss in TSC2. In one embodiment, the prognosis-negative biomarker is copy number loss in STK11 and TSC1. In one embodiment, the prognosis-negative biomarker is copy number loss in STK11 and TSC2. In one embodiment, the prognosis-negative biomarker is TP53 pathway mutation or MAPK pathway mutation or both. In one embodiment, the prognosis-negative biomarker is TP53 pathway and MAPK pathway dual mutation. In one embodiment, the prognosis-negative biomarker is TP53 C141Y mutation. In another embodiment, the prognosis-negative biomarker is ALK E1028D mutation.

In one embodiment, the prognosis-negative biomarker is associated with one or more (e.g., 2, 3, 4, 5, or all) genomic alterations selected from the group consisting of del(11q21), del(13q14), trisomy 12, del(11q22-23), del(17p13), and del(8p).

In an embodiment, the prognosis-negative biomarker is one or more genomic alterations selected from the group consisting of BRAF copy number gain, CTNNB1 copy number gain, FHIT copy number gain, IRF4 copy number gain, MITF copy number gain, MN1 copy number gain, NF2 copy number gain, NF2 copy number loss, RET copy number loss, STK11 copy number loss, TSC2 copy number loss, RB1 loss of heterozygosity.

In an embodiment, the prognosis-positive biomarker is one or more of RANBP17 copy number gain, FGFR3 loss of heterozygosity, GMPS loss of heterozygosity, and WHSC1 loss of heterozygosity.

In one embodiment, the progression-positive or progression-negative biomarker is a genomic alteration. In one embodiment, the genomic alteration is a gene mutation or a copy number alteration. In one embodiment, the gene mutation is a non-dbSNP mutation. In another embodiment, the gene mutation is a single nucleotide polymorphism (SNP) mutation. In one embodiment, the progression-positive biomarker is a genomic alteration in one or more gene in the 6q deletion region. In an embodiment of the invention the progression-positive biomarker is a genomic alteration in an NF-B pathway gene. In an embodiment, the progression-positive biomarker is a del(6q13-16) or a del(6q23-24). In one embodiment the progression-positive biomarker is a TNFAIP3 mutation or copy number loss. In one embodiment the progression-positive biomarker is an EPHA7 mutation or copy number loss.

In certain aspects provided herein is a method of predicting the sensitivity of tumor cell growth to inhibition by a PI3K inhibitor, comprising: assessing the level of at least one prognosis-positive biomarker expressed by a tumor cell; and predicting the sensitivity of tumor cell growth to inhibition by a PI3K inhibitor, wherein high levels of a prognosis-positive biomarker expression by the tumor cells correlates with high sensitivity to inhibition by a PI3K inhibitor, or wherein low expression levels of said tumor cell prognosis-positive biomarker correlate with low sensitivity to inhibition by PI3K inhibitors.

In certain aspects, provided herein is a method of predicting the sensitivity of tumor cell growth to inhibition by a PI3K inhibitor, comprising: assessing the level of at least one prognosis-negative biomarker expressed by a tumor cell; and predicting the sensitivity of tumor cell growth to inhibition by a PI3K inhibitor, wherein high levels of prognosis-negative biomarker expression by the tumor cell correlates with low sensitivity to inhibition by a PI3K inhibitor, or wherein low expression levels of said tumor cell prognosis-negative biomarker correlates with high sensitivity to inhibition by a PI3K inhibitor.

In certain aspects provided herein is a method for treating a tumor in a patient comprising the step of administering to the patient a PI3K inhibitor, wherein the patient possesses a tumor that has been determined as having high sensitivity to tumor cell growth inhibition by a PI3K inhibitor by (a) assessing the level of at least one prognosis-positive biomarker expressed by a tumor cell from said tumor; and predicting the sensitivity of tumor cell growth to inhibition by a PI3K inhibitor, wherein high expression levels of said tumor cell prognosis-positive biomarker correlate with high sensitivity to inhibition by a PI3K inhibitor; or (b) assessing the level of at least one prognosis-negative biomarker expressed by a tumor cell from said tumor; and predicting the sensitivity of tumor cell growth to inhibition by a PI3K inhibitor, wherein low expression levels of said tumor cell prognosis-negative biomarker correlate with high sensitivity to inhibition by a PI3K inhibitor.

In certain aspects, provided herein is a method for treating a tumor in a patient comprising the step of administering to the patient a PI3K inhibitor as a first-line therapy, wherein the patient possesses a tumor that has been determined as having high sensitivity to tumor cell growth inhibition by a PI3K inhibitor by (a) assessing the level of at least one prognosis-positive biomarker expressed by a tumor cell from said tumor; and predicting the sensitivity of tumor cell growth to inhibition by a PI3K inhibitor, wherein high expression levels of said tumor cell prognosis-positive biomarker correlate with high sensitivity to inhibition by a PI3K inhibitor; or (b) assessing the level of at least one prognosis-negative biomarker expressed by a tumor cell from said tumor; and predicting the sensitivity of tumor cell growth to inhibition by a PI3K inhibitor, wherein low expression levels of said tumor cell prognosis-negative biomarker correlate with high sensitivity to inhibition by a PI3K inhibitor.

In some embodiments, the PI3K inhibitor can be selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147.

In some embodiments, the PI3K inhibitor is selected from Compound 1 and GS1101.

In some embodiments, the tumor is an acoustic neuroma, adenocarcinoma, adrenal gland cancer, anal cancer, angiosarcoma, benign monoclonal gammopathy, biliary cancer bladder cancer, breast cancer, brain cancer, bronchus cancer, cervical cancer, choriocarcinoma, chordoma, craniopharyngioma, colorectal cancer, epithelial carcinoma, ependymoma, endotheliosarcoma, endometrial cancer, esophageal cancer, Ewing sarcoma, familiar hypereosinophilia, gastric cancer, gastrointestinal stromal tumor (GIST), head and neck cancer, oral cancer, heavy chain disease, hemangioblastoma, inflammatory myofibroblastic tumors, immunocytic amyloidosis, kidney cancer, liver cancer, malignant hepatoma, lung cancer, leiomyosarcoma (LMS), mastocytosis, multiple myeloma (MM), myelodysplastic syndrome (MDS), mesothelioma, neuroblastoma, neurofibroma neuroendocrine cancer, osteosarcoma, ovarian cancer, Paget's disease of the vulva, Paget's disease of the penis, papillary adenocarcinoma, pancreatic cancer, pinealoma, primitive neuroectodermal tumor (PNT), prostate cancer, rhabdomyosarcoma, retinoblastoma, salivary gland cancer, skin cancer, small bowel cancer, soft tissue sarcoma, sebaceous gland carcinoma, sweat gland carcinoma, synovioma, testicular cancer, thyroid cancer, and Waldenstrom's macroglobulinemia.

In some embodiments, the tumor is a myeloid disorder, lymphoid disorder, leukemia, lymphoma, myelodysplastic syndrome (MDS), myeloproliferative disease (MPD), mast cell disorder, or a myeloma.

In some embodiments, the tumor is selected from acute lymphoblastic leukemia, T-cell acute lymphoblastic leukemia, B-cell acute lymphoblastic leukemia, acute myeloid leukemia, chronic lymphocytic leukemia (CLL), chronic myelogenous leukemia, blast phase chronic myelogenous leukemia, small lymphocytic lymphoma (SLL), CLL/SLL, blast phase CLL, Hodgkin lymphoma, non-Hodgkin lymphoma (NHL), B-cell NHL, T-cell NHL, indolent NHL, diffuse large B-cell lymphoma, mantle cell lymphoma, aggressive B-cell NHL, B-cell lymphoma, Richter's syndrome, T-cell lymphoma, peripheral T-cell lymphoma, cutaneous T-cell lymphoma, transformed mycosis fungoides, Sézary syndrome, anaplastic large-cell lymphoma, follicular lymphoma, Waldenström macroglobulinemia, lymphoplasmacytic lymphoma, Burkitt lymphoma, multiple myeloma, amyloidosis, MPD, essential thrombocytosis, myelofibrosis, polycythemia vera, chronic myelomonocytic leukemia, myelodysplastic syndrome, angioimmunoblastic lymphoma, high-risk MDS, and low-risk MDS.

In some embodiments, the tumor is selected from chronic lymphocytic leukemia, non-Hodgkin lymphoma (e.g., indolent Non-Hodgkin lymphoma), diffuse large B-cell lymphoma, mantle cell lymphoma, and adult T-cell lymphoma.

In some embodiments, the prognosis-positive or prognosis-negative biomarker is a genomic alteration.

In some embodiments, the prognosis-positive or prognosis-negative biomarker is selected from a gene mutation, a copy number alteration, a non-dbSNP mutation or an single nucleotide polymorphism (SNP) mutation.

In some embodiments, the prognosis-positive biomarker is associated with a mutation in a gene selected from RANBP17 copy number gain, FGFR3 loss of heterozygosity, GMPS loss of heterozygosity and WHSC1 loss of heterozygosity.

In some embodiments, the prognosis-negative biomarker is associated with a genomic alteration selected from the group consisting of del(11q21), del(13q14), del(8p), trisomy 12, del(11q22-23), del(17p13), TP53 mutation, TP53 pathway mutation, MAPK pathway mutation, TP53 copy number loss, STK11 copy number loss, TSC1 copy number loss, and TSC2 copy number loss.

In some embodiments, the prognosis-negative biomarker is associated with a mutation in a gene selected from SF3B1, TP53, NOTCH1, MYD88, ATM, XPO1, POT1, NRAS, BCOR, KRAS, MED12, DDX3X, FBXW7, BTK and PLCG2.

In some embodiments, the prognosis-negative biomarker is associated with an STK11 copy number loss, a TSC1 or a TSC2 copy number loss.

In certain aspects, provided herein is a PI3K inhibitor for use in the treatment of cancer, wherein said treatment comprises a method as described herein.

In certain aspects, provided herein is a PI3K inhibitor for use as a first line therapy for the treatment of cancer, wherein said treatment comprises a method as described herein.

In certain aspects, provided herein is a method of identifying a prognosis-positive biomarker that is predictive for more effective treatment of a neoplastic condition with a PI3K inhibitor, said method comprising: measuring the level of a candidate prognosis-positive biomarker in neoplastic cell-containing samples from patients with a neoplastic condition, and identifying a correlation between the level of said candidate prognosis-positive biomarker in the sample from the patient with the effectiveness of treatment of the neoplastic condition with a PI3K inhibitor, wherein a correlation of high levels of the prognosis-positive biomarker with more effective treatment of the neoplastic condition with a PI3K inhibitor indicates that said prognosis-positive biomarker is diagnostic for more effective treatment of the neoplastic condition with a PI3K inhibitor.

In certain aspects, provided herein is a method of identifying a prognosis-negative biomarker that is diagnostic for less effective treatment of a neoplastic condition with a PI3K inhibitor, comprising: measuring the level of a candidate prognosis-negative biomarker in neoplastic cell-containing samples from patients with a neoplastic condition, and identifying a correlation between the level of said candidate prognosis-negative biomarker in the sample from the patient with the effectiveness of treatment of the neoplastic condition with a PI3K inhibitor, wherein a correlation of high levels of the prognosis-negative biomarker with less effective treatment of the neoplastic condition with a PI3K inhibitor indicates that said prognosis-negative biomarker is diagnostic for less effective treatment of the neoplastic condition with a PI3K inhibitor.

In certain aspects, provided herein is a method of predicting the likelihood that a tumor will progress to a more aggressive tumor wherein the tumor is treatable with a PI3K inhibitor, said method comprising the steps of: assessing the level of at least one progression-positive biomarker expressed by a tumor cell from said tumor; and predicting the likelihood that the tumor cell will progress to a more aggressive tumor, wherein high expression levels of said tumor cell progression-positive biomarker correlate with high likelihood that the tumor cell will progress to a more aggressive tumor or wherein low expression levels of said tumor cell progression-positive biomarker correlate with low likelihood that the tumor cell will progress to a more aggressive tumor.

In certain aspects, provided herein is a method of predicting the likelihood that a tumor will progress to a more aggressive tumor wherein the tumor is treatable with a PI3K inhibitor, said method comprising the steps of: assessing the level of at least one progression-negative biomarker expressed by a tumor cell from said tumor; and predicting the likelihood that the tumor cell will progress to a more aggressive tumor, wherein high expression levels of said tumor cell progression-negative biomarker correlate with low likelihood that the tumor cell will progress to a more aggressive tumor or wherein low expression levels of said tumor cell progression-positive biomarker correlate with low likelihood that the tumor cell will progress to a more aggressive tumor.

In certain aspects, provided herein is a method of treating a tumor in a patient, comprising the step of administering to the patient a PI3K inhibitor, wherein there is a high likelihood that the patient will develop a more aggressive tumor and wherein said likelihood has been determined by: (a) assessing the level of at least one progression-positive biomarker expressed by a tumor cell from said tumor; and predicting the likelihood that the tumor cell will progress to a more aggressive tumor, wherein high expression levels of said tumor cell progression-positive biomarker correlate with high likelihood that the tumor cell will progress to a more aggressive tumor; or (b) assessing the level of at least one progression-negative biomarker expressed by a tumor cell from said tumor; and predicting the likelihood that the tumor cell will progress to a more aggressive tumor, wherein low expression levels of said tumor cell progression-negative biomarker correlate with high likelihood that the tumor cell will progress to a more aggressive tumor.

In certain aspects, provided herein is a method of treating a tumor in a patient, comprising the step of administering to the patient a PI3K inhibitor as a first-line therapy, wherein there is a high likelihood that the patient will develop a more aggressive tumor and wherein said likelihood has been determined by: (a) assessing the level of at least one progression-positive biomarker expressed by a tumor cell from said tumor; and predicting the likelihood that the tumor cell will progress to a more aggressive tumor, wherein high expression levels of said tumor cell progression-positive biomarker correlate with high likelihood that the tumor cell will progress to a more aggressive tumor; or (b) assessing the level of at least one progression-negative biomarker expressed by a tumor cell from said tumor; and predicting the likelihood that the tumor cell will progress to a more aggressive tumor, wherein low expression levels of said tumor cell progression-negative biomarker correlate with high likelihood that the tumor cell will progress to a more aggressive tumor.

In some embodiments, the PI3K inhibitor is selected from Compound 1, GS1101, BKM 120, GDC-0941, PX-866, GDC-0032, BAY 80-6946, BEZ235, BYL719, BGT-226, PF-4691502, GDC-0980, GSK 2126458, PF-05212384, XL765, or XL147.

In some embodiments, the tumor is an acoustic neuroma, adenocarcinoma, adrenal gland cancer, anal cancer, angiosarcoma, benign monoclonal gammopathy, biliary cancer bladder cancer, breast cancer, brain cancer, bronchus cancer, cervical cancer, choriocarcinoma, chordoma, craniopharyngioma, colorectal cancer, epithelial carcinoma, ependymoma, endotheliosarcoma, endometrial cancer, esophageal cancer, Ewing sarcoma, familiar hypereosinophilia, gastric cancer, gastrointestinal stromal tumor (GIST), head and neck cancer, oral cancer, heavy chain disease, hemangioblastoma, inflammatory myofibroblastic tumors, immunocytic amyloidosis, kidney cancer, liver cancer, malignant hepatoma, lung cancer, leiomyosarcoma (LMS), mastocytosis, multiple myeloma (MM), myelodysplastic syndrome (MDS), mesothelioma, neuroblastoma, neurofibroma neuroendocrine cancer, osteosarcoma, ovarian cancer, Paget's disease of the vulva, Paget's disease of the penis, papillary adenocarcinoma, pancreatic cancer, pinealoma, primitive neuroectodermal tumor (PNT), prostate cancer, rhabdomyosarcoma, retinoblastoma, salivary gland cancer, skin cancer, small bowel cancer, soft tissue sarcoma, sebaceous gland carcinoma, sweat gland carcinoma, synovioma, testicular cancer, thyroid cancer, and Waldenstrom's macroglobulinemia. In some embodiments, the tumor is a myeloid disorder, lymphoid disorder, leukemia, lymphoma, myelodysplastic syndrome (MDS), myeloproliferative disease (MPD), mast cell disorder, or a myeloma. In some embodiments, the tumor is indolent. In some embodiments, the tumor is selected from acute lymphoblastic leukemia, T-cell acute lymphoblastic leukemia, B-cell acute lymphoblastic leukemia, acute myeloid leukemia, chronic lymphocytic leukemia (CLL), chronic myelogenous leukemia, blast phase chronic myelogenous leukemia, small lymphocytic lymphoma (SLL), CLL/SLL, blast phase CLL, Hodgkin lymphoma, non-Hodgkin lymphoma (NHL), B-cell NHL, T-cell NHL, indolent NHL, diffuse large B-cell lymphoma, mantle cell lymphoma, aggressive B-cell NHL, B-cell lymphoma, Richter's syndrome, T-cell lymphoma, peripheral T-cell lymphoma, cutaneous T-cell lymphoma, transformed mycosis fungoides, Sézary syndrome, anaplastic large-cell lymphoma, follicular lymphoma, Waldenström macroglobulinemia, lymphoplasmacytic lymphoma, Burkitt lymphoma, multiple myeloma, amyloidosis, MPD, essential thrombocytosis, myelofibrosis, polycythemia vera, chronic myelomonocytic leukemia, myelodysplastic syndrome, angioimmunoblastic lymphoma, high-risk MDS, and low-risk MDS.

In some embodiments, the tumor is selected from chronic lymphocytic leukemia, non-Hodgkin lymphoma (e.g., indolent non-Hodgkin lymphoma), diffuse large B-cell lymphoma, mantle cell lymphoma, and adult T-cell lymphoma.

In some embodiments, the progression-positive or progression-negative biomarker is a genomic alteration.

In some embodiments, the progression-positive or progression-negative biomarker is selected from a gene mutation, a copy number alteration, a non-dbSNP mutation or an single nucleotide polymorphism (SNP) mutation.

In some embodiments, the progression-positive biomarker is associated with a mutation in a gene in the 6q deletion region.

In some embodiments, the progression-positive biomarker is a genomic alteration in an NF-B pathway gene.

In some embodiments, the progression-positive biomarker is a del(6q13-16) or a del(6q23-24).

In some embodiments, the progression-positive biomarker is a TNFAIP3 mutation or copy number loss.

In some embodiments, the progression-positive biomarker is an EPHA7 mutation or copy number loss.

In some aspects, the disclosure provides a method of treating a patient, comprising (i) administering a first treatment comprising a first PI3K inhibitor to the subject (ii) acquiring information regarding an alteration in a biomarker by comparing an assessment of the biomarker in a first sample taken from the subject before the first treatment is administered with an assessment of the biomarker in a second sample taken from the subject after the first treatment is administered, wherein the biomarker is selected from STK11, TSC1, TSC2, TP53, PTEN, CBFA2T3, YWHAE, PER1, GAS7, FSTL3, USP6, MAP2K4, or EGFR, and (iii) continuing administration of the first treatment if the alteration is absent, or administering a second treatment if the alteration is present.

In some aspects, the present disclosure provides a method of determining the further course of treatment for a subject who has undergone a first treatment with a first PI3K inhibitor, the method comprising: (i) acquiring information regarding the presence or absence of an alteration in one or more of STK11, TSC1, TSC2, TP53, PTEN, CBFA2T3, YWHAE, PER1, GAS7, FSTL3, USP6, MAP2K4, or EGFR in one or more samples from the subject; and (ii) selecting the subject for continuation of the first treatment with the first PI3K inhibitor if the alteration is absent and selecting the subject for a second treatment if the alteration is present.

In some aspects, the disclosure provides a method of determining decreased responsiveness, or resistance, of a subject to a first treatment comprising a first PI3K inhibitor, the method comprising (i) acquiring information regarding the presence or absence of an alteration in one or more of STK11, TSC1, TSC2, TP53, PTEN, CBFA2T3, YWHAE, PER1, GAS7, FSTL3, USP6, MAP2K4, or EGFR in one or more samples from the subject; and (ii) determining that the subject shows decreased responsiveness or resistance to the first treatment if the alteration is present.

In any of the above aspects or embodiments, the PI3K inhibitor can be selected from: Compound 1, AMG-319, GSK 2126458, GSK 1059615, GDC-0032, GDC-0980, GDC-0941, XL147, XL499, XL765, BKM 120 GS1101, CAL 263, SF1126, PX-866, BEZ235, CAL-120, BYL719, RP6503, RP6530, TGR1202, INK1117, PX-886, BAY 80-6946, IC87114, Palomid 529, ZSTK474, PWT33597, TG100-115, GNE-477, CUDC-907, AEZS-136, BGT-226, PF-05212384, LY3023414, PI-103, LY294002, INCB-040093, CAL-130 and wortmannin.

In certain aspects, the present disclosure also provides methods (e.g., diagnostic and prognostic methods) for evaluating, e.g., predicting, the responsiveness to a treatment of a cancer with a PI3K inhibitor such as Compound 1. The method includes:

acquiring a value (e.g., determining one or more of: the presence, absence, amount or level) of an alteration or biomarker chosen from one, two, three, four, five, six, seven, eight, nine, ten, 11, 12, 13, 15, 20, 25, 30, or all of: VNN1, PARVG, CLEC7A, EPB41L5, NOS3, FPR1, ITGA5, MTMR2, ZFYVE9, PACSIN1, SPP1, CTSH, ATN1, CLCF1, SIRPB1, VAV3, ENO2, AICDA, CARD6, DNAH, NCKAP1, BACH2, OSBCN, TCL1A, KLLN, LRP5, CLCN5, PTEN, GABARAPL1, FOS, ATM, GADD45A, CCNG2, and CDKN1B, thereby evaluating the responsiveness of the cancer or tumor, or the subject to the treatment.

In an embodiment, the alteration or biomarker is chosen from one or more of VNN1, PARVG, CLEC7A, EPB41L5, NOS3, FPR1, ITGA5, MTMR2, ZFYVE9, PACSIN1, SPP1, CTSH, ATN1, CLCF1, and SIRPB1. In an embodiment, elevated levels (compared to a control or reference value) of one or more of VNN1, PARVG, CLEC7A, EPB41L5, NOS3, FPR1, ITGA5, MTMR2, ZFYVE9, PACSIN1, SPP1, CTSH, ATN1, CLCF1, and SIRPB1 is indicative of resistance to a PI3K inhibitor such as Compound 1. In an embodiment, the alteration or biomarker is chosen from one or more of VAV3, ENO2, AICDA, CARD6, DNAH, NCKAP1, BACH2, OSBCN, TCL1A, KLLN, LRP5, CLCN5, PTEN, and GABARAPL1. In addition, decreased levels (compared to a control or reference value) of one or more of VAV3, ENO2, AICDA, CARD6, DNAH, NCKAP1, BACH2, OSBCN, TCL1A, KLLN, LRP5, CLCN5, PTEN, and GABARAPL1 is indicative of resistance to a PI3K inhibitor such as Compound 1. In an embodiment, increased levels of FOS are indicative of resistance to the PI3K inhibitor. In an embodiment, downregulation of ATM, GADD45A, and CCNG2 are indicative of resistance to a PI3K inhibitor.

In an embodiment, the alteration is an increase or decrease in mRNA or protein levels.

The aspects and embodiments above can further include one or more of the following embodiments:

In one embodiment, detection of one, two, three or all of the following is indicative of decreased responsiveness of the subject to the treatment over a time interval:

(i) a copy number loss of STK11 (e.g., a single copy loss);

(ii) a copy number loss of TSC1 or TSC2, or both;

(iii) a p53 pathway mutation, e.g., a mutation listed in Table 25 (e.g., TP53 C141Y); or

(iv) a MAPK pathway mutation, e.g., a mutation listed in Table 23.

The present invention also provides, at least in part, methods (e.g., diagnostic and prognostic methods) for evaluating, e.g., predicting, the responsiveness to a treatment of a cancer with a B-cell receptor (BCR) pathway inhibitor (e.g., a PI3K inhibitor). In one embodiment, it is shown herein that STK11 copy number loss (with or without copy number loss of TSC1, TSC2, or both) is associated with, or is predictive of, decreased responsiveness (e.g., acquired resistance) of a cancer (e.g., chronic lymphocytic leukemia (CLL)) to a PI3K inhibitor (e.g., Compound 1). In other embodiments, it has been discovered that an alteration in the MAP kinase and p53 (MAPK/p53) pathway is associated with, or is predictive of, decreased responsiveness (e.g., acquired resistance) of a cancer (e.g., CLL) to a PI3K inhibitor (e.g., Compound 1). Thus, compositions, methods, and kits for the identification, assessment and/or treatment of a cancer or tumor responsive to a PI3K inhibitor treatment (e.g., a treatment that includes a PI3K inhibitor as a single agent or in combination) are disclosed herein.

Accordingly, in one aspect, the invention features a method of evaluating the responsiveness of a cancer or tumor, or a subject having a cancer or tumor, to a treatment with a BCR pathway inhibitor (e.g., a treatment with an inhibitor of PI3K, BTK or SYK, alone or in combination). In one embodiment, responsiveness to a PI3K inhibitor is evaluated. The method includes: acquiring a value (e.g., determining one or more of: the presence, absence, amount or level) of an alteration or biomarker chosen from one, two, three, four or all of: an STK11 copy number, TSC1 copy number, TSC2 copy number, a p53 pathway mutation (e.g., a mutation disclosed in Table 25), or MAPK pathway mutation (e.g., a mutation disclosed in Table 23), or any combination thereof (e.g., a dual MAPK/p53 pathway mutation, e.g., a mutation disclosed in Table 23 and a mutation disclosed in Table 25).

In another aspect, the invention features a method of monitoring a treatment of a subject with a BCR pathway inhibitor (e.g., a treatment with an inhibitor of PI3K, BTK or SYK, alone or in combination). In one embodiment, treatment with a PI3K inhibitor is monitored. The method includes: acquiring, at two or more time intervals, a value (e.g., determining one or more of: the presence, absence, amount or level) of an alteration or biomarker chosen from one, two, three, four or all of: an STK11 copy number, TSC1 copy number, TSC2 copy number, a p53 pathway mutation (e.g., a mutation disclosed in Table 25), or MAPK pathway mutation (e.g., a mutation disclosed in Table 23), or any combination thereof (e.g., a dual MAPK/p53 mutation, e.g., a mutation disclosed in Table 23 and a mutation disclosed in Table 25).

In another aspect, the invention features a method of treating (e.g., inhibiting, reducing, ameliorating, managing, or preventing) a cancer or tumor in a subject. The method includes: acquiring a value (e.g., determining one or more of: the presence, absence, amount or level) of an alteration or biomarker chosen from one, two, three, four or all of: an STK11 copy number, TSC1 copy number, TSC2 copy number, a p53 pathway mutation (e.g., a mutation disclosed in Table 25), or MAPK pathway mutation (e.g., a mutation disclosed in Table 23), or any combination thereof (e.g., a dual MAPK/p53 mutation, e.g., a mutation disclosed in Table 23 and a mutation disclosed in Table 25), and responsive to said value, administering to the subject a BCR pathway inhibitor, e.g., a PI3K inhibitor (e.g., one or more PI3K inhibitors).

In some embodiments of the above aspects, the method further comprises administering the PI3K inhibitor to the subject. In an embodiment, the PI3K inhibitor is administered alone or in combination with a second therapeutic agent. In an embodiment, the second therapeutic agent is 1) a CDK 4/6 inhibitor, 2) an HDAC inhibitor, 3) a MEK inhibitor, 4) a mTOR inhibitor, 5) an AKT inhibitor, 6) a proteasome inhibitor, 7) an immunomodulator, 8) a glucocorticosteroid, 9) a BET inhibitor, 10) an epigenetic inhibitor, 11) a PI3K alpha inhibitor, 12) a topoisomerase inhibitor, or 13) an ERK inhibitor. In an embodiment, the BCR pathway mutation is a mutation disclosed in Table 24. In an embodiment, the p53 pathway mutation is a mutation disclosed in Table 25. In an embodiment, the MAPK pathway mutation is a mutation disclosed in Table 23. In an embodiment, the combination thereof is a dual MAPK/p53 mutation of which a mutation is disclosed in Table 23 and a mutation is disclosed in Table 25.

In another aspect, the present disclosure provides a method of evaluating the responsiveness of a cancer or tumor, of a subject having a cancer or tumor, to a treatment with a BCR pathway inhibitor (e.g., a treatment with an inhibitor of PI3K, BTK or SYK, alone or in combination). In one embodiment, responsiveness to a PI3K inhibitor is evaluated. The method includes: acquiring a value (e.g., determining one or more of: the presence, absence, amount or level) of one or more of (e.g., 2, 3, 4, or all of): FOS, ATM, GADD45A, CCNG2, and CDKN1B.

In another aspect, the invention features a method of monitoring a treatment of a subject with a BCR pathway inhibitor (e.g., a treatment with an inhibitor of PI3K, BTK or SYK, alone or in combination). In one embodiment, treatment with a PI3K inhibitor is monitored. The method includes: acquiring, at two or more time intervals, a value (e.g., determining one or more of: the presence, absence, amount or level) of one or more of (e.g., 2, 3, 4, or all of): FOS, ATM, GADD45A, CCNG2, and CDKN1B.

In another aspect, the invention features a method of treating (e.g., inhibiting, reducing, ameliorating, managing, or preventing) a cancer or tumor in a subject. The method includes: acquiring a value (e.g., determining one or more of: the presence, absence, amount or level) of one or more of (e.g., 2, 3, 4, or all of): FOS, ATM, GADD45A, CCNG2, and CDKN1B.

In some embodiments, the methods that include acquiring a value of one or more of: FOS, ATM, GADD45A, CCNG2, CDKN1B include acquiring a value (e.g., determining one or more of: the presence, absence, amount or level) of an additional factor relevant to chemosensitization. In some embodiments, one or more of (e.g., 2, 3, 4, or all of) an elevated level of FOS, a reduced level of ATM, a reduced level of GADD45A, a reduced level of CCNG2, and a reduced level of CDKN1B indicate increased sensitization. In some embodiments, one or more of (e.g., 2, 3, 4, or all of) an elevated level of FOS, a reduced level of ATM, a reduced level of GADD45A, a reduced level of CCNG2, and a reduced level of CDKN1B indicate resistance to a PI3K inhibitor. In some embodiments, one or more of (e.g., 2, 3, 4, or all of) a normal or reduced level of FOS, a normal or elevated level of ATM, a normal or level of GADD45A, a normal or of CCNG2, and a normal or of CDKN1B indicate responsiveness to a PI3K inhibitor. In some embodiments, the methods involve administering a chemotherapeutic agent (e.g., a chemotherapeutic agent described herein), optionally in combination with a PI3K inhibitor, to a subject having one or more of (e.g., 2, 3, 4, or all of) an elevated level of FOS, a reduced level of ATM, a reduced level of GADD45A, a reduced level of CCNG2, and a reduced level of CDKN1B. In some embodiments, the methods involve administering a PI3K inhibitor as a monotherapy to a subject having a normal or reduced level of FOS, a normal or elevated level of ATM, a normal or level of GADD45A, a normal or of CCNG2, and a normal or of CDKN1B. In some embodiments, the elevated, normal, or reduced levels of a biomarker are determined with reference to a non-cancerous control value.

In some embodiments of the above aspects, one, two, three or all of the following is indicative of decreased responsiveness of the cancer, or the subject, to the treatment:

(i) a copy number loss (e.g., a single copy loss) of STK11;

(ii) a copy number loss of TSC1 or TSC2, or both;

(iii) a copy number loss of TP53;

(iv) a copy number loss of PTEN;

(v) a copy number loss of CBFAT2T3;

(vi) a copy number loss of YWHAE;

(vii) a copy number loss of PER1;

(viii) a copy number loss of GAS7;

(ix) a copy number loss of FSTL3;

(x) a copy number loss of USP6;

(xi) a copy number loss of MAP2K4;

(xii) a BCR pathway mutation, e,g., a mutation listed in Table 24;

(xiii) a p53 pathway mutation, e.g., a mutation listed in Table 25 (e.g., TP53 C141Y); or

(xiv) a MAPK pathway mutation, e.g., a mutation listed in Table 23.

In some embodiments of the above aspects, the alteration or biomarker is a copy number loss (e.g., a single copy loss) of STK11. In one embodiment, detection of copy number loss of STK11 is indicative of decreased responsiveness of the cancer or tumor, or the subject, to the treatment. In some embodiments of the above aspects, the alteration or biomarker is a BCR pathway mutation. In one embodiment, detection of a BCR pathway mutation is indicative of decreased responsiveness of the cancer or tumor, or the subject, to the treatment. In some embodiments of the above aspects, detection of copy number loss of TP53 is indicative of decreased responsiveness of the cancer or tumor, or the subject, to the treatment. In some embodiments of the above aspects, detection of copy number loss of PTEN is indicative of decreased responsiveness of the cancer or tumor, or the subject, to the treatment. In some embodiments of the above aspects, detection of copy number loss of CBFAT2T3 is indicative of decreased responsiveness of the cancer or tumor, or the subject, to the treatment. In some embodiments of the above aspects, detection of copy number loss of YWHAE is indicative of decreased responsiveness of the cancer or tumor, or the subject, to the treatment. In some embodiments of the above aspects, detection of copy number loss of PER1 is indicative of decreased responsiveness of the cancer or tumor, or the subject, to the treatment. In some embodiments of the above aspects, detection of copy number loss of GAS7 is indicative of decreased responsiveness of the cancer or tumor, or the subject, to the treatment. In some embodiments of the above aspects, detection of copy number loss of FSTL3 is indicative of decreased responsiveness of the cancer or tumor, or the subject, to the treatment. In some embodiments of the above aspects, detection of copy number loss of USP6 is indicative of decreased responsiveness of the cancer or tumor, or the subject, to the treatment. In some embodiments of the above aspects, detection of copy number loss of MAP2K4 is indicative of decreased responsiveness of the cancer or tumor, or the subject, to the treatment. In some embodiments of the above aspects, detection of copy number loss of EGFR is indicative of increased responsiveness of the cancer or tumor, or the subject, to the treatment; or wherein detection of copy number gain of EGFR is indicative of decreased responsiveness of the cancer or tumor, or the subject, to the treatment, or both. In some embodiments of the above aspects, detection of copy number loss of EGFR is indicative of increased responsiveness of the cancer or tumor, or the subject, to the treatment, and wherein increased responsiveness is determined using nodal criteria.

In some embodiments of the above aspects, the alteration or biomarker is a dual MAPK/p53 pathway mutation. In one embodiment the dual mutation includes a mutation listed in Table 23 and/or Table 25. In one embodiment, detection of the dual MAPK/p53 pathway mutation is indicative of decreased responsiveness of the cancer or tumor, or the subject, to the treatment.

In some embodiments of the above aspects, no detectable copy number loss of STK11, TSC1, TSC2, TP53, PTEN, CBFA2T3, YWHAE, PER1, GAS7, FSTL3, USP6, or MAP2K4, or no detectable dual MAPK/p53 pathway mutation, or no detectable BCR pathway mutation, is indicative of continued responsiveness to the treatment.

In some embodiments of the above aspects, the alteration or biomarker is a copy number loss of STK11 in combination with a copy number loss of TSC1, TSC2, or both. In one embodiment, detection of copy number loss of STK11 in combination with a copy number loss of TSC1 is indicative of decreased responsiveness of the cancer or tumor, or the subject, to the treatment. In another embodiment, detection of copy number loss of STK11 in combination with a copy number loss of TSC2 is indicative of decreased responsiveness of the cancer or tumor, or the subject, to the treatment. In yet another embodiment, detection of copy number loss of STK11 in combination with a copy number loss of TSC1 and TSC2 is indicative of decreased responsiveness of the cancer or tumor, or the subject, to the treatment.

In some embodiments of the above aspects, the alteration is a prognosis-negative biomarker or a progression-positive biomarker, or both. In one embodiment, detection of a prognosis-negative biomarker or a progression-positive biomarker, or both, is indicative of decreased responsiveness of the cancer or tumor, or the subject, to the treatment.

In some embodiments of the above aspects, no detectable copy number loss of STK11, or no detectable dual MAPK/p53 pathway mutation, is indicative of continued responsiveness to the treatment. In one embodiment, if the subject is identified as being responsive to the treatment, the treatment is continued. In another embodiment, if the subject is identified as not being responsive to the treatment, the treatment is altered or discontinued, thereby having a first and second treatment.

In some embodiments of the above aspects, the subject is evaluated prior to undergoing, while undergoing, or after undergoing, treatment with the BCR pathway inhibitor, e.g., the PI3K inhibitor. In one embodiment, the subject is evaluated, at two or more time points, prior to undergoing, while undergoing, or after undergoing, treatment with the BCR pathway inhibitor, e.g., the PI3K inhibitor. In another embodiment, the subject is evaluated at at least two time intervals, e.g., prior to undergoing and while undergoing the treatment. In yet another embodiment, the subject is evaluated at at least three time points, e.g., prior to undergoing, while undergoing the treatment, and after undergoing the treatment.

In some embodiments of the above aspects, decreased responsiveness of the cancer or tumor, or the subject to the treatment, e.g., over a timecourse of the treatment, is indicative of increased resistance (e.g., acquired resistance) to the treatment, e.g., the PI3K inhibitor. In an embodiment, if the subject is identified as being responsive to the treatment, the treatment is continued. In an embodiment, if the subject is identified as not being responsive to the treatment, the treatment is altered or discontinued, thereby having a first and second treatment.

Alternatively, or in combination to the aforesaid methods, the method includes administration to the subject, e.g., a subject at risk, or having a cancer or tumor (e.g., a hematologic cancer as described herein), a treatment with the BCR pathway inhibitor, e.g., the PI3K inhibitor. In some embodiments of the above aspects, the treatment is a monotherapy with the PI3K inhibitor, e.g., Compound 1. In some embodiments, the subject is identified as developing resistance to a monotherapy with a PI3K inhibitor.

In some embodiments of the aforesaid methods, responsive to a determination of the value of the alteration or biomarker, the method further includes one, two, three, four, five, six, seven, eight, nine or all of the following:

(i) identifying the subject as being in need of a treatment, e.g., treatment with a PI3K inhibitor (e.g., a first treatment or a second (alternative) treatment);

(ii) identifying the subject as having an increased or a decreased responsiveness to the treatment, e.g., the treatment with the PI3K inhibitor (e.g., a monotherapy with Compound 1);

(iii) identifying the subject as being a responder to the treatment, e.g., identifying the subjects as being in complete remission (CR) or partial cancer remission (PR) (e.g., CR or PR subjects as described herein);

(iv) identifying the subject as being a non-responder to the treatment, e.g., identifying the subjects as having a progressive disease (PD) or stable disease (SD) (e.g., PD or SD subjects as described herein);

(v) identifying the subject as having developed resistance (e.g., partial or complete, acquired resistance) to the treatment, e.g., the PI3K inhibitor (e.g., Compound 1);

(vi) diagnosing and/or prognosing the subject;

(vii) determining a time course of disease progression in the subject;

(viii) determining the time course of acquisition of resistance to the treatment;

(ix) determining a treatment, e.g., selecting or altering the course of, a treatment (e.g., a first treatment), a dose, a treatment schedule or time course, and/or the use of an alternative, second treatment); and/or

(x) administering the treatment (e.g., the first treatment or a second (alternative) treatment) to the subject.

In one embodiment of the aforesaid methods, the subject is identified as having decreased responsiveness to the treatment by having at least one progression-positive biomarker. In one embodiment, the progression-positive biomarker is a genomic alteration in an NF-κB pathway gene. In an embodiment, the progression-positive biomarker is a 6q deletion region, e.g., a del(6q13-16) or a del(6q23-24). In one embodiment, the progression-positive biomarker is a TNFAIP3 mutation or copy number loss. In one embodiment, the progression-positive biomarker is an EPHA7 mutation or copy number loss.

In one embodiment, the subject is identified as having an increased responsiveness to a second treatment, e.g., a treatment comprising a reduced dose of the PI3K inhibitor, or a treatment comprising a combination of the PI3K inhibitor and a second agent, e.g., a second therapeutic agent. In one embodiment, the dose of the PI3K inhibitor, the second agent, or both, is reduced, e.g., at least 20%, at least 30%, at least 40%, or at least 50%, than the amount or dosage of each agent used individually, e.g., as a monotherapy.

In some embodiments of the methods described herein, the method further includes altering a treatment (e.g., a first treatment), a dose, a treatment schedule or time course, and/or the use of an alternative, second treatment.

In other embodiments of the methods described herein, the method further includes administering the treatment (e.g., the first treatment or a second (alternative) treatment) to the subject.

In other embodiments of the methods described herein, the method further includes administering a combination of the PI3K inhibitor and a second agent in an amount sufficient to treat the cancer, in the subject, e.g., for treatment of a cancer described herein. In some embodiments, the second agent is chosen from one or more of: a MEK inhibitor, an mTOR inhibitor, an AKT inhibitor, a proteasome inhibitor, immunomodulator, a glucocorticosteroid, a CDK4/6 inhibitor, and an MDM2 inhibitor. In one embodiment, the second agent is a MEK inhibitor. In one embodiment, the second agent is an mTOR inhibitor. In one embodiment, the second agent is a CDK4/6 inhibitor. In one embodiment, the second agent is an MDM2 inhibitor.

Exemplary MDM2 inhibitors are described in Hoe, K. K. et al. (2014) Nature Reviews Drug Discovery, 13: 217-236. In an embodiment, the MDM2 inhibitor is selected from one or more of RG7112 (Roche, also known as R05045337); MI-773 (Sanofi, also known as SAR405838); DS-3032b (Daiichi Sankyo); Nutlin; R05503781; PRIMA-1MET (also known as APR 246); nutlin 3a (Roche); RG7388 (Roche); Ro-2443 (Roche); MI-219 (Ascenta Therapeutics, Sanofi); MI-713 (Ascenta Therapeutics, Sanofi); MI-888 (Ascenta Therapeutics, Sanofi); TDP521252 (Johnson & Johnson); NSC279287 (Virginia Commonwealth University); AM-8553 (Amgen); PXN822 (Priaxon); naturally derived prenylated xanthones (Universidade do Porto); SAH-8 (stapled peptides); sMTide-02, sMTide-02a (stapled peptides) (LAB P53, A*STAR); ATSP-7041 (stapled peptide) (Aileron Therapeutics); spiroligomer (a helix mimic) (Temple University); PK083, PK5174, PK5196, PK7088, benzothiazoles (Centre for Protein Engineering, MRC Laboratory of Molecular Biology); stictic acid (University of California, Irvine); NSC319726 (The Cancer Institute of New Jersey); RO 5963.

In some embodiments, acquiring a value comprises acquiring information regarding the presence or absence of an alteration described herein.

In some embodiments, the methods herein comprise comparing an assessment of a biomarker in a first sample taken from the subject before the first treatment is administered with an assessment of the biomarker in a second sample taken from the subject after the first treatment is administered. In an embodiment, the method comprises determining the further course of treatment for the subject. In an embodiment, the method comprises a method of determining decreased responsiveness, or resistance, of the subject to the first treatment.

In some embodiments, the methods herein comprise administering a first treatment comprising a first PI3K inhibitor to the subject and continuing administration of the first treatment if an alteration is absent, or administering a second treatment if the alteration is present. In some embodiments, the methods herein comprise determining the further course of treatment for a subject, e.g., selecting the subject for continuation of the first treatment with the first PI3K inhibitor if the alteration is absent and selecting the subject for a second treatment if the alteration is present, wherein the second treatment includes administration of a BCL-2 inhibitor.

In some embodiments, the first treatment with the first PI3K inhibitor (e.g., Compound 1) is a monotherapy in which the first PI3K inhibitor is the only component of the first treatment known to have a substantial therapeutic activity. In some embodiments, the second treatment comprises an agent chosen from one or more of: a MEK inhibitor (e.g., a MEK inhibitor described herein), an mTOR inhibitor (e.g., an mTOR inhibitor described herein), a CDK4/6 inhibitor (e.g., a CDK4/6 inhibitor described herein), and an MDM2 inhibitor (e.g., a MDM2 inhibitor described herein).

In certain embodiments, the alteration is an STK11, TSC1, TSC2, TP53, PTEN, CBFA2T3, YWHAE, PER1, GAS7, FSTL3, USP6, or MAP2K4 copy number loss (e.g., single copy loss). In some embodiments, the STK11, TSC1, TSC2, TP53, PTEN, CBFA2T3, YWHAE, PER1, GAS7, FSTL3, USP6, or MAP2K4 copy number in a sample taken from the subject after the first treatment is lower than a corresponding STK11, TSC1, TSC2, TP53, PTEN, CBFA2T3, YWHAE, PER1, GAS7, FSTL3, USP6, MAP2K4 copy number in a sample taken from the subject before the first treatment (e.g., there is an STK11 single copy loss).

This disclosure also provides, in some aspects, a method of identifying a cell, e.g., a cancer cell, or a subject, as being less responsive, e.g., resistant, to a PI3K inhibitor such as Compound 1. The method can comprise evaluating the level, e.g., in a subject or a biological sample, of one or more of (e.g., 2, 5, 10, 25, 50, 75, 100, 150, 200, 250, 300, 350, or all of) the following biomarkers: ISG15, PRKCZ, ZBTB17, PINK1, LDLRAP1, FGR, PTAFR, PLK3, PIK3R3, ZFYVE9, JUN, CTH, VAV3, SORT1, NOTCH2, TXNIP, HIST2H4A, MLLT11, S100A13, IFI16, AIM2, SLAMF7, FCGR2B, LAMC1, PIK3C2B, PFKFB2, CD55, CD46, PROX1, ENAH, OBSCN, EGLN1, CAMK1D, COMMD3-BMI1, MAPK8, SRGN, SGPL1, DDIT4, KLLN, PTEN, LIPA, HHEX, HELLS, TCTN3, ENTPD1, BLNK, FRAT1, FRAT2, AVPI1, CHUK, BTRC, LDB1, NT5C2, SMNDC1, DUSP5, SMC3, PDCD4, SHOC2, CASP7, BAGS, BNIP3, IFITM2, SMPD1, APBB1, HIPK3, CD59, RAG1, LRP4, NR1H3, PTPRJ, UBE2L6, DTX4, DAK, FERMT3, PPP2R5B, CD248, CLCF1, LRP5, PAK1, GAB2, MTMR2, TRPC6, IL10RA, AMICA1, CD3E, THY1, CCND2, GNB3, ENO2, ATN1, AICDA, CLEC7A, GABARAPL1, CDKN1B, PRICKLE1, RAPGEF3, WNT10B, GPD1, ACVR1B, NR4A1, EIF4B, MAP3K12, LRP1, DDIT3, FRS2, E2F7, SELPLG, CORO1C, OAST, OAS2, HRK, PXN, HNF1A, TSC22D1, FGF14, CCNB1IP1, ZNF219, ARHGAP5, PRKCH, ESR2, DPF3, MLH3, FOS, RPS6KA5, TCL6, TCL1A, TRAF3, TNFAIP2, JAG2, BRF1, PACS2, SLC12A6, SPRED1, PLCB2, TYRO3, SHF, MYO5A, RAB27A, NEDD4, BBS4, PML, CTSH, IL16, ADAMTSL3, NMB, IGF1R, ALDH1A3, PIGQ, MAPK8IP3, LITAF, MYH11, DCUN1D3, LAT, MAPK3, BCL7C, MYLK3, MT1X, NLRC5, CSNK2A2, CKLF, NQO1, CBFA2T3, MYO1C, P2RX1, NLRP1, TNFSF13, EPN2, VTN, SARM1, ALDOC, CDK5R1, CCL5, RARA, DUSP3, TBKBP1, HOXB3, GNGT2, TMEM100, PECAM1, PRKCA, UNC13D, ASPSCR1, FASN, SLC16A3, SETBP1, SMAD7, ABCA7, TRIP10, INSR, FCER2, KANK2, DNASE2, NOTCH3, IFI30, HOMER3, MEF2B, LPAR2, PLEKHF1, NFKBID, SPRED3, MAP3K10, LTBP4, NUMBL, ERCC1, GIPR, DMPK, SPHK2, RPL13A, FPR1, TP53I3, SLC8A1, SPRED2, MEIS1, RTKN, EIF2AK3, DUSP2, INPP4A, EPB41L5, CCNT2, ITGA6, ZAK, TTN, NCKAP1, STAT1, IKZF2, STK36, DNER, RBCK1, SIRPB1, JAG1, ADA, ELMO2, PTPN1, BMP7, PMEPA1, MYT1, JAM2, TIAM1, ETS2, ITGB2, ADARB1, CLTCL1, PRAME, BCR, CBY1, ATF4, BIK, TSPO, PARVG, GRAMD4, MAPK12, MAPK11, MAPK8IP2, OXTR, SATB1, PRKAR2A, MST1R, HYAL2, MAPKAPK3, TLR9, ITIH4, WNT5A, ARHGEF3, FLNB, MITF, NFKBIZ, IFT57, MYLK, MGLL, PLXND1, CHST2, MME, HEST, TNK2, DGKQ, FGFRL1, SH3BP2, MFSD10, RHOH, TEC, ARHGAP24, SPP1, PKD2, PLA2G12A, IRF2, C1QTNF3, CARD6, IL6ST, PDE4D, ERBB2IP, OCLN, NAIP, FCHO2, SEMA6A, CAMLG, MZB1, TMEM173, HBEGF, CCNG1, TFAP2A, CD83, PRL, HIST1H1C, BTN3A2, PACSIN1, PPARD, CDKN1A, PIM1, TREM1, CRIP3, SUPT3H, TNFRSF21, MYO6, BACH2, FOXO3, TRAF3IP2, FYN, KPNA5, VNN1, MYB, CITED2, TAB2, ULBP2, ULBP3, TIAM2, FNDC1, PLG, THBS2, GNAl2, HOXA5, HOXA13, CREB5, PDE1C, SAMD9, SRPK2, BCAP29, ZC3HAV1, NOS3, PRKAG2, CLDN23, TNFRSF10B, TNFRSF10D, GPR124, LY96, E2F5, RRM2B, SCRIB, PLEC, PLGRKT, IL11RA, SHB, PIP5K1B, TJP2, FGD3, TNFSF15, TRIM32, C5, GSN, HSPA5, PBX3, CACFD1, CYBB, CLCN5, OCRL, BCORL1, ELF4, AIFM1, GPC4, PHF6, ARHGEF6, MTM1, MTMR1, IRAK1, FLNA, RPL10, F8, MTCP1, and CD24. Alternatively or in combination, the method can comprise evaluating the level, e.g., in a subject or biological sample, of one or more biomarkers in one or more (e.g., 1, 2, 3, 4, 5, 6, 7, 8, or all) of the following pathways: apoptotic signaling pathway, cellular response to cytokine stimulus pathway, cytokine mediated signaling pathway, endocytosis pathway, innate immune response signaling pathway, MAPK pathway, neurotrophin TRK receptor signaling pathway, PI3K pathway, and TLR pathway. The biomarkers evaluated in these pathways can be, e.g., genes described herein, e.g., in the Examples. In certain embodiments, the method comprises evaluating nucleic acid levels, e.g., RNA or DNA levels. In some embodiments, if levels of one or more of the aforementioned biomarkers, or biomarkers in the aforementioned pathways, are different from (e.g., higher or lower than) a reference, e.g., a control sample, the biological sample is classified as being less resistant, e.g., resistant, to a PI3K inhibitor such as Compound 1.

This disclosure also provides, in some aspects, a method of identifying a cell, e.g., a cancer cell, or a subject, as being less responsive, e.g., resistant, to a BTK inhibitor such as ibrutinib. The method can comprise evaluating the levels, e.g., in a biological sample or subject, of one or more biomarkers in one or more (e.g., 1, 2, 3, 4, 5, 6, 7, 8, or all) of the following pathways: apoptotic signaling pathway, cellular response to cytokine stimulus pathway, FOXO pathway, innate immune response pathway, MAPK pathway, neurotrophin TRK receptor signaling pathway, PI3K pathway, positive regulation of apoptosis pathway, and T cell activation pathway. The biomarkers evaluated in these pathways may be, e.g., genes described herein, e.g., in the Examples. In certain embodiments, the method comprises evaluating nucleic acid levels, e.g., RNA or DNA levels. In some embodiments, if levels of biomarkers in the aforementioned pathways, are different from (e.g., higher or lower than) a reference e.g., control sample, the subject or biological sample is classified as being less responsive, e.g., resistant, to a BTK inhibitor such as ibrutinib.

In some embodiments, the methods of treatment described herein comprise administering a combination of a PI3K inhibitor, one of more of 1) a CDK 4/6 inhibitor, 2) an HDAC inhibitor, 3) a MEK inhibitor, 4) a mTOR inhibitor, 5) an AKT inhibitor, 6) a proteasome inhibitor, 7) an immunomodulator, 8) a glucocorticosteroid, 9) a BET inhibitor, 10) an epigenetic inhibitor, 11) a PI3K alpha inhibitor, 12) a topoisomerase inhibitor, or 13) an ERK inhibitor, and a third agent. The third agent can be, e.g., a modulator of, e.g., inhibitor of, the apoptotic signaling pathway, cellular response to cytokine stimulus pathway, cytokine mediated signaling pathway, endocytosis pathway, innate immune response signaling pathway, MAPK pathway, neurotrophin TRK receptor signaling pathway, PI3K pathway, or TLR pathway. The modulator of one of these pathways may act on one of the pathway genes described herein, e.g., in the Examples. While not wishing to be bound by theory, the third agent can be an agent that normalizes signaling in a pathway that is differentially regulated in cells resistant to a PI3K inhibitor, e.g., Compound 1.

In some embodiments, the methods of treatment described herein comprise administering a combination of a PI3K inhibitor and a second agent. The second agent can be, e.g., a modulator of, e.g., inhibitor of, the apoptotic signaling pathway, cellular response to cytokine stimulus pathway, cytokine mediated signaling pathway, endocytosis pathway, innate immune response signaling pathway, MAPK pathway, neurotrophin TRK receptor signaling pathway, PI3K pathway, or TLR pathway. The modulator of one of these pathways may act on one of the pathway genes described herein, e.g., in the Examples. While not wishing to be bound by theory, the second agent can be an agent that normalizes signaling in a pathway that is differentially regulated in cells resistant to a PI3K inhibitor, e.g., Compound 1.

In some embodiments, the inhibitor of the apoptotic signaling pathway is an inhibitor of VAV3 such as a Vav3 siRNA (e.g., as described in Nomura et al., Mol Cancer. 2013 Apr. 8; 12:27.).

In some embodiments, the inhibitor of the cellular response to cytokine stimulus pathway is an inhibitor of WNT5A such as a t-butyloxycarbonyl-modified Wnt5a-derived hexapeptide, e.g., the Met-Asp-Gly-Cys-Glu-Leu peptide described in Jenei et al., Nov. 17, 2009, vol. 106 no. 46, 19473-19478.

In some embodiments, the inhibitor of the cytokine mediated signaling pathway is an inhibitor of MAPK3 such as PD98059 (Di Paola et al., Int J Immunopathol Pharmacol. 2009 October-December; 22(4):937-50).

In some embodiments, the inhibitor of the endocytosis pathway is an inhibitor of TYRO3, e.g., Sunitinib or BMS-777607.

In some embodiments, the inhibitor of the innate immune response signaling pathway is an inhibitor of TLR9 such as AT791 {3-[4-(6-(3-(dimethylamino)propoxy)benzo[d]oxazol-2-yl)phenoxyl-N,N-dimethylpropan-1-amine} and E6446 {6-[3-(pyrrolidin-1-yl)propoxy)-2-(4-(3-(pyrrolidin-1-yl)propoxy)phenyl]benzo[d]oxazole}, described in Lamphier et al., Mol Pharmacol. 2014 Mar.; 85(3):429-40.

In some embodiments, the inhibitor of the MAPK pathway is a PAK1 inhibitor such as IPA3 (Molosh et al., Nature Neuroscience 17, 1583-1590 (2014)), staurosporin, CEP-1347, KT D606, WR-PAK18 (Kichina et al., Expert Opin Ther Targets. 2010 July; 14(7): 703-725).

In some embodiments, the inhibitor of the neurotrophin TRK receptor signaling pathway is an inhibitor of PRKCA such as MT477 (Jasinski et al., Investigational New Drugs, February 2011, Volume 29, Issue 1, pp 33-40) or PKC alpha (C2-4) inhibitor peptide from Santa Cruz Biotechnology, Inc.

In some embodiments, the inhibitor of the PI3K pathway is a PI3K inhibitor described herein.

In some embodiments, the inhibitor of the TLR pathway is an inhibitor of TLR9 such as AT791 {3-[4-(6-(3-(dimethylamino)propoxy)benzo[d]oxazol-2-yl)phenoxy]-N,N-dimethylpropan-1-amine} and E6446 {6-[3-(pyrrolidin-1-yl)propoxy)-2-(4-(3-(pyrrolidin-1-yl)propoxy)phenyl]benzo[d]oxazole}, described in Lamphier et al., Mol Pharmacol. 2014 March; 85(3):429-40.

### Detection of Alterations

The genomic alteration biomarkers provided herein can be detected by the methods known in the art to detect genomic alterations. In one embodiment, the gene mutations or copy number alterations are detected by methods such as CytoScan Microarray (pre- and post-treatment), targeted exome sequencing (pre- and post-treatment), and Sanger sequencing. In one embodiment, the mutation or copy number alteration of STK11 is detected by STK11 FISH Probe or qPCR.

In one embodiment, the biomarkers provided herein can be used to identify, diagnose, predict efficacy, predict long term clinical outcome, predict prognosis, and/or select patients for a treatment described herein. In one embodiment, the biomarkers provided herein can be used for subsets of patients with different prognostic factors.

In the methods of the invention, one can detect expression of biomarker proteins having at least one portion which is displayed on the surface of tumor cells which express it. It is a simple matter for the skilled artisan to determine whether a marker protein, or a portion thereof, is exposed on the cell surface. For example, immunological methods may be used to detect such proteins on whole cells, or well known computer-based sequence analysis methods may be used to predict the presence of at least one extracellular domain (i.e. including both secreted proteins and proteins having at least one cell-surface domain). Expression of a marker protein having at least one portion which is displayed on the surface of a cell which expresses it may be detected without necessarily lysing the tumor cell (e.g. using a labeled antibody which binds specifically with a cell-surface domain of the protein).

Expression of a biomarkers described in this invention may be assessed by any of a wide variety of well known methods for detecting expression of a transcribed nucleic acid or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In one embodiment, expression of a biomarker is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a biomarker protein or fragment thereof, including a biomarker protein which has undergone either all or a portion of post-translational modifications to which it is normally patiented in the tumor cell (e.g. glycosylation, phosphorylation, methylation etc.).

In another embodiment, expression of a biomarker is assessed by preparing mRNA/cDNA (i.e. a transcribed polynucleotide) from cells in a patient sample, and by hybridizing the mRNA/cDNA with a reference polynucleotide which is a complement of a biomarker nucleic acid, or a fragment thereof. cDNA can, optionally, be amplified using any of a variety of polymerase chain reaction methods prior to hybridization with the reference polynucleotide. Expression of one or more biomarkers can likewise be detected using quantitative PCR to assess the level of expression of the biomarker(s).

In all embodiments of the invention, the expression level of a biomarker can be determined with reference to the effect on biomarker expression caused by a mutation or variant in a gene associated with said biomarker. Accordingly, for example, the consequences of a genomic alteration on the expression level of biomarkers referred to in the methods of the invention may be inferred directly from identification of the genomic alteration in the genome of a patient.

As used herein, the mutation can be a point mutation, e.g. SNP, an insertion, a deletion, an amplification, a deletion, a chromosomal translocation, an interstitial deletion, a chromosomal inversion or a loss of heterozygosity.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a biomarker nucleic acid. If polynucleotides complementary to or homologous with are differentially detectable on the substrate (e.g. detectable using different chromophores or fluorophores, or fixed to different selected positions), then the levels of expression of a plurality of biomarkers can be assessed simultaneously using a single substrate (e.g. a “gene chip” microarray of polynucleotides fixed at selected positions). When a method of assessing biomarker expression is used which involves hybridization of one nucleic acid with another, it is preferred that the hybridization be performed under stringent hybridization conditions.

When a plurality of biomarkers of the invention are used in the methods of the invention, the level of expression of each biomarker in a patient sample can be compared with the normal level of expression of each of the plurality of biomarkers in non-cancerous samples of the same type, either in a single reaction mixture (i.e. using reagents, such as different fluorescent probes, for each biomarker) or in individual reaction mixtures corresponding to one or more of the biomarkers.

The level of expression of a biomarker in normal (i.e. non-cancerous) human tissue can be assessed in a variety of ways. In one embodiment, this normal level of expression is assessed by assessing the level of expression of the biomarker in a portion of cells which appears to be non-cancerous, and then comparing this normal level of expression with the level of expression in a portion of the tumor cells. Alternately, and particularly as further information becomes available as a result of routine performance of the methods described herein, population-average values for normal expression of the biomarkers of the invention may be used. In other embodiments, the ‘normal’ level of expression of a biomarker may be determined by assessing expression of the biomarker in a patient sample obtained from a non-cancer-afflicted patient, from a patient sample obtained from a patient before the suspected onset of cancer in the patient, from archived patient samples, and the like.

An exemplary method for detecting the presence or absence of a biomarker protein or nucleic acid in a biological sample involves obtaining a biological sample (e.g. a tumor-associated body fluid) from a test patient and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a biomarker protein include enzyme linked immunosorbent assays (ELISAs), Western blots, immunoprecipitations and immunofluorescence. In vitro techniques for detection of genomic DNA include Southern hybridizations. In vivo techniques for detection of mRNA include polymerase chain reaction (PCR), Northern hybridizations and in situ hybridizations. Furthermore, in vivo techniques for detection of a biomarker protein include introducing into a patient a labeled antibody directed against the protein or fragment thereof. For example, the antibody can be labeled with a radioactive marker whose presence and location in a patient can be detected by standard imaging techniques.

A general principle of such diagnostic and prognostic assays involves preparing a sample or reaction mixture that may contain a biomarker, and a probe, under appropriate conditions and for a time sufficient to allow the biomarker and probe to interact and bind, thus forming a complex that can be removed and/or detected in the reaction mixture. These assays can be conducted in a variety of ways.

For example, one method to conduct such an assay would involve anchoring the biomarker or probe onto a solid phase support, also referred to as a substrate, and detecting target biomarker/probe complexes anchored on the solid phase at the end of the reaction. In one embodiment of such a method, a sample from a patient, which is to be assayed for presence and/or concentration of biomarker, can be anchored onto a carrier or solid phase support. In another embodiment, the reverse situation is possible, in which the probe can be anchored to a solid phase and a sample from a patient can be allowed to react as an unanchored component of the assay.

There are many established methods for anchoring assay components to a solid phase. These include, without limitation, biomarker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the biomarker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of biomarker/probe complexes anchored to the solid phase can be accomplished in a number of methods outlined herein.

In one embodiment, the probe, when it is the unanchored assay component, can be labeled for the purpose of detection and readout of the assay, either directly or indirectly, with detectable labels discussed herein and which are well-known to one skilled in the art.

It is also possible to directly detect biomarker/probe complex formation without further manipulation or labeling of either component (biomarker or probe), for example by utilizing the technique of fluorescence energy transfer (i.e. FET, see for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a biomarker can be accomplished without labeling either assay component (probe or biomarker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with biomarker and probe as solutes in a liquid phase. In such an assay, the complexed biomarker and probe are separated from uncomplexed components by any of a number of standard techniques, including but not limited to: differential centrifugation, chromatography, electrophoresis and immunoprecipitation. In differential centrifugation, biomarker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the biomarker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J. Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of biomarker mRNA can be determined both by in situ and by in vitro formats in a biological sample using methods known in the art. The term “biological sample” is intended to include tissues, cells, biological fluids and isolates thereof, isolated from a patient, as well as tissues, cells and fluids present within a patient. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a biomarker of the present invention. Other suitable probes for use in the diagnostic assays of the invention are described herein. Hybridization of an mRNA with the probe indicates that the biomarker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the biomarkers of the present invention.

An alternative method for determining the level of mRNA biomarker in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample is prepared/processed using known histological methods. The sample is then immobilized on a support, typically a glass slide, and then contacted with a probe that can hybridize to mRNA that encodes the biomarker.

An alternative method for determining the level of mRNA biomarker in a sample involves deep sequencing of cDNA generated from RNA. In some embodiments, mRNA is isolated from tumor cells, fragmented, and converted into cDNA libraries, and quantified using next generation sequencing.

As an alternative to making determinations based on the absolute expression level of the biomarker, determinations may be based on the normalized expression level of the biomarker. Expression levels are normalized by correcting the absolute expression level of a biomarker by comparing its expression to the expression of a gene that is not a biomarker, e.g., a housekeeping gene that is constitutively expressed. Suitable genes for normalization include housekeeping genes such as the actin gene, or prognosis-positive cell-specific genes. This normalization allows the comparison of the expression level in one sample, e.g., a patient sample, to another sample, e.g., a non-tumor sample, or between samples from different sources.

Alternatively, the expression level can be provided as a relative expression level. To determine a relative expression level of a biomarker (e.g. a prognosis-negative biomarker), the level of expression of the biomarker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the biomarker. The expression level of the biomarker determined for the test sample (absolute level of expression) is then divided by the mean expression value obtained for that biomarker. This provides a relative expression level.

In another embodiment of the present invention, a biomarker protein is detected. One agent for detecting biomarker protein of the invention is an antibody capable of binding to such a protein or a fragment thereof, preferably an antibody with a detectable label. Antibodies can be polyclonal, or more preferably, monoclonal. An intact antibody, or a fragment or derivative thereof (e.g., Fab or F(ab′)2 can be used. The term “labeled”, with regard to the probe or antibody, is intended to encompass direct labeling of the probe or antibody by coupling (e.g., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from tumor cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether tumor cells express a biomarker of the present invention.

In one format, antibodies, or antibody fragments or derivatives, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from tumor cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies. Exemplary non-immune pairs are biotin-streptavidin, intrinsic factor-vitamin B12, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin can be covalently coupled to antibodies by utilizing commercially available active derivatives. Some of these are biotin-N-hydroxy-succinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein can be coupled to protein amine groups using fluorescein isothiocyanate. Dinitrophenyl groups can be coupled to protein amine groups using 2,4-dinitrobenzene sulfate or 2,4-dinitrofluorobenzene. Other standard methods of conjugation can be employed to couple monoclonal antibodies to a member of a specific binding pair including dialdehyde, carbodiimide coupling, homofunctional crosslinking, and heterobifunctional crosslinking Carbodiimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional crosslinkers, including the bifunctional imidoesters and bifunctional N-hydroxysuccinimide esters, are commercially available and are employed for coupling amine groups on one substance to amine groups on another. Heterobifunctional crosslinkers are reagents which possess different functional groups. The most common commercially available heterobifunctional crosslinkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups.

The detectably-labeled antibody or detectably-labeled member of the specific binding pair is prepared by coupling to a reporter, which can be a radioactive isotope, enzyme, fluorogenic, chemiluminescent or electrochemical materials. Two commonly used radioactive isotopes are 1251 and 3H. Standard radioactive isotopic labeling procedures include the chloramine T, lactoperoxidase and Bolton-Hunter methods for 1251 and reductive methylation for 3H. The term “detectably-labeled” refers to a molecule labeled in such a way that it can be readily detected by the intrinsic enzymic activity of the label or by the binding to the label of another component, which can itself be readily detected.

Enzymes suitable for use in this invention include, but are not limited to, horseradish peroxidase, alkaline phosphatase, -galactosidase, glucose oxidase, luciferases, including firefly and renilla, -lactamase, urease, green fluorescent protein (GFP) and lysozyme. Enzyme labeling is facilitated by using dialdehyde, carbodiimide coupling, homobifunctional crosslinkers and heterobifunctional crosslinkers as described above for coupling an antibody with a member of a specific binding pair.

The labeling method chosen depends on the functional groups available on the enzyme and the material to be labeled, and the tolerance of both to the conjugation conditions. The labeling method used in the present invention can be one of, but not limited to, any conventional methods currently employed including those described by Engvall and Pearlmann, Immunochemistry 8, 871 (1971), Avrameas and Ternynck, Immunochemistry 8, 1175 (1975), Ishikawa et al., J. Immunoassay 4(3):209-327 (1983) and Jablonski, Anal. Biochem. 148:199 (1985).

Labeling can be accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, according to the present invention, the antibody used to detect can be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above.

Moreover, the unlabeled detector antibody can be detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody can be labeled directly or indirectly using any of the approaches discussed above. For example, the anti-antibody can be coupled to biotin which is detected by reacting with the streptavidin-horseradish peroxidase system discussed above.

In one embodiment of this invention biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA can be used to effect chromogenic detection.

In one immunoassay format for practicing this invention, a forward sandwich assay is used in which the capture reagent has been immobilized, using conventional techniques, on the surface of a support. Suitable supports used in assays include synthetic polymer supports, such as polypropylene, polystyrene, substituted polystyrene, e.g. aminated or carboxylated polystyrene, polyacrylamides, polyamides, polyvinylchloride, glass beads, agarose, or nitrocellulose.

Kits

The invention also encompasses kits for detecting the presence of a biomarker protein or nucleic acid in a biological sample. Such kits can be used to determine if a patient is suffering from or is at increased risk of developing a tumor that is less susceptible to inhibition by PI3K inhibitors. For example, the kit can comprise a labeled compound or agent capable of detecting a biomarker protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit.

For antibody-based kits, the kit can comprise, for example: (1) a first antibody (e.g., attached to a solid support) which binds to a biomarker protein; and, optionally, (2) a second, different antibody which binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a biomarker protein or (2) a pair of primers useful for amplifying a biomarker nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

In another aspect, the invention features a method of predicting the sensitivity of cancer or tumor cell growth to inhibition by a PI3K inhibitor, comprising: assessing the level of at least one prognosis-positive biomarker in a cancer cell; and predicting the sensitivity of cancer or tumor cell growth to inhibition by a PI3K inhibitor, wherein detection, or an elevated level, of said prognosis-positive biomarker correlate with high sensitivity to inhibition by PI3K inhibitors, or wherein absence or reduced detection of said prognosis-positive biomarker correlates with low sensitivity to inhibition by PI3K inhibitors.

The present invention also provides a method of predicting the sensitivity of cancer or tumor cell growth to inhibition by a PI3K inhibitor, comprising: assessing the level of at least one prognosis-negative biomarker in a cancer or tumor cell; and predicting the sensitivity of cancer or tumor cell growth to inhibition by a PI3K inhibitor, wherein detection of an alteration, or elevated level of said prognosis-negative biomarker correlates with low sensitivity to inhibition by a PI3K inhibitor, or wherein absence of the alteration or low levels of said prognosis-negative biomarker correlates with high sensitivity to inhibition by a PI3K inhibitor.

In one embodiment, a prognosis-negative biomarker is chosen from one, two, three or all of the following:

(i) a copy number loss of STK11;

(ii) a copy number loss of TSC1 or TSC2, or both;

(iii) a p53 pathway mutation, e.g., TP53 C141Y; or

(iv) a MAPK pathway mutation.

In one embodiment, a prognosis-negative biomarker is a copy number loss of STK11.

Improved methods for treating a cancer patient with a PI3K inhibitor that incorporate the methods described herein are also provided, whereby patients with high sensitivity to cancer or tumor cell growth inhibition by a PI3K inhibitor are determined by the methods of the present invention. Thus, the present invention further provides a method for treating cancer in a subject, e.g., a patient, comprising the step of administering to the subject a PI3K inhibitor, wherein the subject possesses a cancer that has been determined as having high sensitivity to cancer or tumor cell growth inhibition by a PI3K inhibitor by assessing the level of at least one prognosis-positive biomarker in a cancer or tumor cell from said cancer or tumor; and predicting the sensitivity of cancer or tumor cell growth to inhibition by a PI3K inhibitor, wherein detection or an elevated level of said prognosis-positive biomarker correlate with high sensitivity to inhibition by a PI3K inhibitor; or

assessing the level of at least one prognosis-negative biomarker in a cancer or tumor cell from said cancer or tumor; and predicting the sensitivity of cancer or tumor cell growth to inhibition by a PI3K inhibitor, wherein the presence or level of the alteration said prognosis-negative biomarker correlate with high sensitivity to inhibition by a PI3K inhibitor.

In one embodiment, a prognosis-negative biomarker is chosen from one, two, three or all of the following:

(i) a copy number loss of STK11;

(ii) a copy number loss of TSC1 or TSC2, or both;

(iii) a p53 pathway mutation, e.g., TP53 C141Y; or

(iv) a MAPK pathway mutation.

In one embodiment, a prognosis-negative biomarker is a copy number loss of STK11. In one embodiment, detection of copy number loss of STK11 is indicative of decreased responsiveness of the cancer or tumor, or the subject, to the treatment.

In one embodiment, a prognosis-negative biomarker is a dual MAPK/p53 mutation. In one embodiment, detection of the dual MAPK/p53 mutation is indicative of decreased responsiveness of the cancer or tumor, or the subject, to the treatment.

In one embodiment, a prognosis-negative biomarker is a copy number loss of STK11 in combination with a copy number loss of TSC1, TSC2, or both. In one embodiment, detection of copy number loss of STK11 in combination with a copy number loss of TSC1 is indicative of decreased responsiveness of the cancer or tumor, or the subject, to the treatment. In another embodiment, detection of copy number loss of STK11 in combination with a copy number loss of TSC2 is indicative of decreased responsiveness of the cancer or tumor, or the subject, to the treatment. In yet another embodiment, detection of copy number loss of STK11 in combination with a copy number loss of TSC1 and TSC2 is indicative of decreased responsiveness of the cancer or tumor, or the subject, to the treatment.

In another embodiment, the alteration is a prognosis-negative biomarker or a progression-positive biomarker, or both. In one embodiment, detection of a prognosis-negative biomarker or a progression-positive biomarker, or both, is indicative of decreased responsiveness of the cancer or tumor, or the subject, to the treatment.

A further embodiment of the invention is a method of treating a cancer or tumor or a metastasis thereof in a subject, comprising the step of administering to the subject a PI3K inhibitor, e.g., as a first-line therapy, wherein the subject possesses a cancer or tumor that has been determined as having high sensitivity to cancer or tumor cell growth inhibition by a PI3K inhibitor by assessing the level of at least one prognosis-positive biomarker by one of the following:

assessing the level of at least one prognosis-positive biomarker expressed by a cancer cell from said cancer or tumor; and predicting the sensitivity of cancer or tumor cell growth to inhibition by a PI3K inhibitor, wherein detection or an elevated level of said prognosis-positive biomarker correlate with high sensitivity to inhibition by a PI3K inhibitor; or

assessing the presence or an alteration at least one prognosis-negative biomarker in a a cancer or tumor cell from said cancer or tumor; and predicting the sensitivity of cancer or tumor cell growth to inhibition by a PI3K inhibitor, wherein low levels of said prognosis-negative biomarker correlate with high sensitivity to inhibition by a PI3K inhibitor.

Also provided by the present invention are PI3K inhibitors for use in the herein-described methods. Further provided are compositions comprising a PI3K inhibitor for use in the herein-described methods.

Also provided herein are kits for evaluating the alterations or biomarkers described herein.

Additionally, methods are provided for the identification of new prognosis-positive or prognosis-negative biomarkers that are predictive of responsiveness of tumors to PI3K inhibitors.

Thus, for example, the present invention provides a method of identifying a prognosis-positive biomarker that is predictive for more effective treatment of a neoplastic condition with a PI3K inhibitor, comprising: measuring the level of a candidate prognosis-positive biomarker in neoplastic cell-containing samples from patients with a neoplastic condition, and identifying a correlation between the level of said candidate prognosis-positive biomarker in the sample from the patient with the effectiveness of treatment of the neoplastic condition with a PI3K inhibitor, wherein a correlation of high levels of the prognosis-positive biomarker with more effective treatment of the neoplastic condition with a PI3K inhibitor indicates that said prognosis-positive biomarker is diagnostic for more effective treatment of the neoplastic condition with a PI3K inhibitor.

The present invention further provides a method of identifying a prognosis-negative biomarker that is diagnostic for less effective treatment of a neoplastic condition with a PI3K inhibitor, comprising: measuring the level of a candidate prognosis-negative biomarker in neoplastic cell-containing samples from patients with a neoplastic condition, and identifying a correlation between the level of said candidate prognosis-negative biomarker in the sample from the patient with the effectiveness of treatment of the neoplastic condition with a PI3K inhibitor, wherein a correlation of high levels of the prognosis-negative biomarker with less effective treatment of the neoplastic condition with a PI3K inhibitor indicates that said prognosis-negative biomarker is diagnostic for less effective treatment of the neoplastic condition with a PI3K inhibitor.

In a further aspect of the present invention, methods for identifying and treating patients with a tumor which is at risk of progressing to a more aggressive tumor are provided. Certain tumors, such as indolent tumors, for example indolent lymphomas, can grow very slowly and are characterized by long survival time. Median survival is typically around 10-15 years, and variance from the median is broad. Some patients can survive well beyond 15 years. Patients with indolent tumors sometimes do not start treatment when first diagnosed, instead adopting a ‘watch and wait’ approach in which treatment only begins after further symptoms have developed. However, in certain indolent tumors, such as indolent follicular lymphoma, up to 40% of patients progress to develop more aggressive forms of tumors. Survival for such patients is typically far shorter. Therefore, there is a need to provide methods of treating patients with indolent tumors who are at risk of progressing to more aggressive tumors.

Thus, the present invention provides a method of predicting the likelihood that a tumor will progress to a more aggressive tumor wherein the tumor is treatable with a PI3K inhibitor, comprising: assessing the level of at least one progression-positive biomarker expressed by a tumor cell from said tumor; and predicting the likelihood that the tumor cell will progress to a more aggressive tumor, wherein high expression levels of said tumor cell progression-positive biomarker correlate with high likelihood that the tumor cell will progress to a more aggressive tumor or wherein low expression levels of said tumor cell progression-positive biomarker correlate with low likelihood that the tumor cell will progress to a more aggressive tumor.

The present invention also provides a method of predicting the likelihood that a tumor cell from a tumor will progress to a more aggressive tumor wherein the tumor is treatable with a PI3K inhibitor, comprising: assessing the level of at least one progression-negative biomarker expressed by a tumor cell; and predicting the likelihood that the tumor cell will progress to a more aggressive tumor, wherein high expression levels of said tumor cell progression-negative biomarker correlate with low likelihood that the tumor cell will progress to a more aggressive tumor, or wherein low expression levels of said tumor cell progression-negative biomarker correlates with high sensitivity to inhibition by a PI3K inhibitor.

In a further aspect, the present invention provides a method for treating a cancer or tumor in a subject, e.g., a patient, comprising administering to the subject a PI3K inhibitor, wherein there is a high likelihood that the patient will develop a more aggressive tumor and wherein said likelihood has been determined by

assessing the level of at least one progression-positive biomarker expressed by a tumor cell from said tumor; and predicting the likelihood that the tumor cell will progress to a more aggressive tumor, wherein high expression levels of said tumor cell progression-positive biomarker correlate with high likelihood that the tumor cell will progress to a more aggressive tumor; or

assessing the level of at least one progression-negative biomarker expressed by a tumor cell from said tumor; and predicting the likelihood that the tumor cell will progress to a more aggressive tumor, wherein low expression levels of said tumor cell progression-negative biomarker correlate with high likelihood that the tumor cell will progress to a more aggressive tumor.

### 4. FORMULATIONS

The formulations or compositions described herein can include a PI3K inhibitor (e.g., one or more PI3K inhibitors as described herein) and/or one or more additional agents (e.g., a second agent, e.g., one or more second agents) as described herein. In certain embodiments, the PI3K inhibitor (e.g., one or more PI3K inhibitors as described herein) and the second agent are included in the same dosage form. In certain embodiments, the PI3K inhibitor (e.g., one or more PI3K inhibitors as described herein) and the second agent are included in separate dosage forms.

Pharmaceutical compositions may be specially formulated for administration in solid or liquid form, including those adapted for the following: oral administration, for example, drenches (aqueous or non-aqueous solutions or suspensions), tablets (e.g., those targeted for buccal, sublingual, and systemic absorption), capsules, boluses, powders, granules, pastes for application to the tongue, and intraduodenal routes; parenteral administration, including intravenous, intraarterial, subcutaneous, intramuscular, intravascular, intraperitoneal or infusion as, for example, a sterile solution or suspension, or sustained-release formulation; topical application, for example, as a cream, ointment, or a controlled-release patch or spray applied to the skin; intravaginally or intrarectally, for example, as a pessary, cream, stent or foam; sublingually; ocularly; pulmonarily; local delivery by catheter or stent; intrathecally, or nasally.

The amount of PI3K inhibitor administered and the timing of PI3K inhibitor administration will depend on the type (species, gender, age, weight, etc.) and condition of the patient being treated, the severity of the disease or condition being treated, and on the route of administration. For example, small molecule PI3K inhibitors can be administered to a patient in doses ranging from 0.001 to 100 mg/kg of body weight per day or per week in single or divided doses, or by continuous infusion. In particular, compounds such as Compound 1, or similar compounds, can be administered to a patient in doses ranging from 5-200 mg per day, or 100-1600 mg per week, in single or divided doses, or by continuous infusion. In one embodiment, the dose is 150 mg/day. Antibody-based PI3K inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

Examples of suitable aqueous and nonaqueous carriers which may be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

These compositions can also contain adjuvants such as preservatives, wetting agents, emulsifying agents, dispersing agents, lubricants, and/or antioxidants. Prevention of the action of microorganisms upon the compounds described herein may be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Methods of preparing these formulations or compositions include the step of bringing into association a compound described herein and/or the chemotherapeutic with the carrier and, optionally, one or more accessory ingredients. In general, the formulations are prepared by uniformly and intimately bringing into association a compound as disclosed herein with liquid carriers, or finely divided solid carriers, or both, and then, if necessary, shaping the product.

Preparations for such pharmaceutical compositions are well-known in the art. See, e.g., Anderson, Philip 0.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Twelfth Edition, McGraw Hill, 2011; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety. Except insofar as any conventional excipient medium is incompatible with the compounds provided herein, such as by producing any undesirable biological effect or otherwise interacting in a deleterious manner with any other component(s) of the pharmaceutically acceptable composition, the excipient's use is contemplated to be within the scope of this disclosure.

In some embodiments, the concentration of the PI3K inhibitor (e.g., Compound 1) or another agent (e.g., the second agent, e.g., one or more second agents as described herein) provided a pharmaceutical composition disclosed herein or administered in a method disclosed herein is less than about 100%, about 90%, about 80%, about 70%, about 60%, about 50%, about 40%, about 30%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001%, w/w, w/v or v/v.

In some embodiments, the concentration of the PI3K inhibitor (e.g., Compound 1) or another agent, (e.g., the second agent, e.g., one or more second agents as described herein) provided a pharmaceutical composition disclosed herein or administered in a method disclosed herein is greater than about 90%, about 80%, about 70%, about 60%, about 50%, about 40%, about 30%, about 20%, about 19.75%, about 19.50%, about 19.25%, about 19%, about 18.75%, about 18.50%, about 18.25%, about 18%, about 17.75%, about 17.50%, about 17.25%, about 17%, about 16.75%, about 16.50%, about 16.25%, about 16%, about 15.75%, about 15.50%, about 15.25%, about 15%, about 14.75%, about 14.50%, about 14.25%, about 14%, about 13.75%, about 13.50%, about 13.25%, about 13%, about 12.75%, about 12.50%, about 12.25%, about 12%, about 11.75%, about 11.50%, about 11.25%, about 11%, about 10.75%, about 10.50%, about 10.25%, about 10%, about 9.75%, about 9.50%, about 9.25%, about 9%, about 8.75%, about 8.50%, about 8.25%, about 8%, about 7.75%, about 7.50%, about 7.25%, about 7%, about 6.75%, about 6.50%, about 6.25%, about 6%, about 5.75%, about 5.50%, about 5.25%, about 5%, about 4.75%, about 4.50%, about 4.25%, about 4%, about 3.75%, about 3.50%, about 3.25%, about 3%, about 2.75%, about 2.50%, about 2.25%, about 2%, about 1.75%, about 1.50%, about 1.25%, about 1%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001%, w/w, w/v, or v/v.

In some embodiments, the concentration of the PI3K inhibitor (e.g., Compound 1) or another agent, (e.g., the second agent, e.g., one or more second agents as described herein) provided a pharmaceutical composition disclosed herein or administered in a method disclosed herein is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, or approximately 1% to approximately 10%, w/w, w/v or v/v.

In some embodiments, the concentration of the PI3K inhibitor (e.g., Compound 1) or another agent (e.g., the second agent, e.g., one or more second agents as described herein) provided a pharmaceutical composition disclosed herein or administered in a method disclosed herein is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, or approximately 0.1% to approximately 0.9%, w/w, w/v or v/v.

In some embodiments, the concentration of the PI3K inhibitor (e.g., Compound 1) or another agent (e.g., the second agent, e.g., one or more second agents as described herein) provided a pharmaceutical composition disclosed herein or administered in a method disclosed herein is equal to or less than about 10 g, about 9.5 g, about 9.0 g, about 8.5 g, about 8.0 g, about 7.5 g, about 7.0 g, about 6.5 g, about 6.0 g, about 5.5 g, about 5.0 g, about 4.5 g, about 4.0 g, about 3.5 g, about 3.0 g, about 2.5 g, about 2.0 g, about 1.5 g, about 1.0 g, about 0.95 g, about 0.9 g, about 0.85 g, about 0.8 g, about 0.75 g, about 0.7 g, about 0.65 g, about 0.6 g, about 0.55 g, about 0.5 g, about 0.45 g, about 0.4 g, about 0.35 g, about 0.3 g, about 0.25 g, about 0.2 g, about 0.15 g, about 0.1 g, about 0.09 g, about 0.08 g, about 0.07 g, about 0.06 g, about 0.05 g, about 0.04 g, about 0.03 g, about 0.02 g, about 0.01 g, about 0.009 g, about 0.008 g, about 0.007 g, about 0.006 g, about 0.005 g, about 0.004 g, about 0.003 g, about 0.002 g, about 0.001 g, about 0.0009 g, about 0.0008 g, about 0.0007 g, about 0.0006 g, about 0.0005 g, about 0.0004 g, about 0.0003 g, about 0.0002 g, or about 0.0001 g.

In some embodiments, the concentration of the PI3K inhibitor (e.g., Compound 1) or another agent, (e.g., the second agent, e.g., one or more second agents as described herein) provided a pharmaceutical composition disclosed herein or administered in a method disclosed herein is more than about 0.0001 g, about 0.0002 g, about 0.0003 g, about 0.0004 g, about 0.0005 g, about 0.0006 g, about 0.0007 g, about 0.0008 g, about 0.0009 g, about 0.001 g, about 0.0015 g, about 0.002 g, about 0.0025 g, about 0.003 g, about 0.0035 g, about 0.004 g, about 0.0045 g, about 0.005 g, about 0.0055 g, about 0.006 g, about 0.0065 g, about 0.007 g, about 0.0075 g, about 0.008 g, about 0.0085 g, about 0.009 g, about 0.0095 g, about 0.01 g, about 0.015 g, about 0.02 g, about 0.025 g, about 0.03 g, about 0.035 g, about 0.04 g, about 0.045 g, about 0.05 g, about 0.055 g, about 0.06 g, about 0.065 g, about 0.07 g, about 0.075 g, about 0.08 g, about 0.085 g, about 0.09 g, about 0.095 g, about 0.1 g, about 0.15 g, about 0.2 g, about 0.25 g, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, or about 10 g.

In some embodiments, the amount of Compound 1 or one or more of the therapeutic agent disclosed herein is in the range of about 0.0001 to about 10 g, about 0.0005 to about 9 g, about 0.001 to about 8 g, about 0.005 to about 7 g, about 0.01 to about 6 g, about 0.05 to about 5 g, about 0.1 to about 4 g, about 0.5 to about 4 g, or about 1 to about 3 g.

4.1 Formulations for Oral Administration

In some embodiments of the methods described herein, PI3K inhibitor (e.g., one or more PI3K inhibitors) and/or another agent (e.g., the second agent, e.g., one or more second agents as described herein) is administered orally. In certain embodiments of the compositions described herein, PI3K inhibitor (e.g., Compound 1) and/or another agent (e.g., the second agent, e.g., one or more second agents as described herein) is formulated for oral administration. Some embodiments pertaining to such methods and compositions include the following.

In some embodiments, provided herein are pharmaceutical compositions for oral administration containing a compound as disclosed herein, and a pharmaceutical excipient suitable for oral administration. In some embodiments, provided herein are pharmaceutical compositions for oral administration containing: (i) an effective amount of a disclosed compound; optionally (ii) an effective amount of one or more second agents; and (iii) one or more pharmaceutical excipients suitable for oral administration. In some embodiments, the pharmaceutical composition further contains: (iv) an effective amount of a third agent.

In some embodiments, the pharmaceutical composition can be a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more ingredients. In general, the pharmaceutical compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The present disclosure further encompasses anhydrous pharmaceutical compositions and dosage forms comprising an active ingredient, since water can facilitate the degradation of some compounds. For example, water can be added (e.g., about 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. For example, pharmaceutical compositions and dosage forms which contain lactose can be made anhydrous if substantial contact with moisture and/or humidity during manufacturing, packaging, and/or storage is expected. An anhydrous pharmaceutical composition can be prepared and stored such that its anhydrous nature is maintained. Accordingly, anhydrous pharmaceutical compositions can be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the pharmaceutical compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. In some embodiments, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants can be used in the pharmaceutical compositions as provided herein to provide tablets that disintegrate when exposed to an aqueous environment. Too much of a disintegrant can produce tablets which can disintegrate in the bottle. Too little can be insufficient for disintegration to occur and can thus alter the rate and extent of release of the active ingredient(s) from the dosage form. Thus, a sufficient amount of disintegrant that is neither too little nor too much to detrimentally alter the release of the active ingredient(s) can be used to form the dosage forms of the compounds disclosed herein. The amount of disintegrant used can vary based upon the type of formulation and mode of administration, and can be readily discernible to those of ordinary skill in the art. About 0.5 to about 15 weight percent of disintegrant, or about 1 to about 5 weight percent of disintegrant, can be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein can be combined with various sweetening or flavoring agents, coloring matter or dyes and, for example, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms include, but are not limited to, hydrophilic surfactants, lipophilic surfactants, and mixtures thereof. That is, a mixture of hydrophilic surfactants can be employed, a mixture of lipophilic surfactants can be employed, or a mixture of at least one hydrophilic surfactant and at least one lipophilic surfactant can be employed.

A suitable hydrophilic surfactant can generally have an HLB value of at least about 10, while suitable lipophilic surfactants can generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants can be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants can be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants can include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of triglycerides, vegetable oils, and hydrogenated vegetable oils. The polyol can be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of glycerides, vegetable oils, hydrogenated vegetable oils, fatty acids and sterols; oil-soluble vitamins/vitamin derivatives; and mixtures thereof. Within this group, non-limiting examples of lipophilic surfactants include glycerol fatty acid esters, propylene glycol fatty acid esters, and mixtures thereof, or are hydrophobic transesterification products of a polyol with at least one member of vegetable oils, hydrogenated vegetable oils, and triglycerides.

In one embodiment, the pharmaceutical composition can include a solubilizer to ensure good solubilization and/or dissolution of a compound as provided herein and to minimize precipitation of the compound. This can be especially important for pharmaceutical compositions for non-oral use, e.g., pharmaceutical compositions for injection. A solubilizer can also be added to increase the solubility of the hydrophilic drug and/or other components, such as surfactants, or to maintain the pharmaceutical composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, E-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers can also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. In some embodiments, solubilizers include sorbitol, glycerol, triacetin, ethyl alcohol, PEG-400, glycofurol and propylene glycol.

The amount of solubilizer that can be included is not particularly limited. The amount of a given solubilizer can be limited to a bioacceptable amount, which can be readily determined by one of skill in the art. In some circumstances, it can be advantageous to include amounts of solubilizers far in excess of bioacceptable amounts, for example to maximize the concentration of the drug, with excess solubilizer removed prior to providing the pharmaceutical composition to a subject using conventional techniques, such as distillation or evaporation. Thus, if present, the solubilizer can be in a weight ratio of about 10%, 25%, 50%, 100%, or up to about 200% by weight, based on the combined weight of the drug, and other excipients. If desired, very small amounts of solubilizer can also be used, such as about 5%, 2%, 1% or even less. Typically, the solubilizer can be present in an amount of about 1% to about 100%, more typically about 5% to about 25% by weight.

The pharmaceutical composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, oils, odorants, opacifiers, suspending agents, binders, fillers, plasticizers, lubricants, and mixtures thereof.

Exemplary preservatives can include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In addition, an acid or a base can be incorporated into the pharmaceutical composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Examples can include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

4.2 Formulations for Parenteral Administration

In some embodiments of the methods described herein, PI3K inhibitor (e.g., one or more PI3K inhibitors) and/or another agent (e.g., the second agent, e.g., one or more second agents as described herein) is administered parenterally. In certain embodiments of the compositions described herein, PI3K inhibitor (e.g., Compound 1) and/or another agent (e.g., the second agent, e.g., one or more second agents as described herein) is formulated for parenteral administration. Some embodiments pertaining to such methods and compositions include the following.

In some embodiments, provided herein are pharmaceutical compositions for parenteral administration containing a compound as disclosed herein, and a pharmaceutical excipient suitable for parenteral administration. In some embodiments, provided herein are pharmaceutical compositions for parenteral administration containing: (i) an effective amount of a disclosed compound; optionally (ii) an effective amount of one or more second agents; and (iii) one or more pharmaceutical excipients suitable for parenteral administration. In some embodiments, the pharmaceutical composition further contains: (iv) an effective amount of a third agent.

The forms in which the disclosed pharmaceutical compositions can be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils can also be employed.

Aqueous solutions in saline are also conventionally used for injection. Ethanol, glycerol, propylene glycol, liquid polyethylene glycol, and the like (and suitable mixtures thereof), cyclodextrin derivatives, and vegetable oils can also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound as disclosed herein in the required amount in the appropriate solvent with various other ingredients as enumerated above, as appropriate, followed by filtered sterilization. Generally, dispersions are prepared by incorporating the various sterilized active ingredients into a sterile vehicle which contains the basic dispersion medium and the appropriate other ingredients from those enumerated above. In the case of sterile powders for the preparation of sterile injectable solutions, certain methods of preparation are vacuum-drying and freeze-drying techniques which yield a powder of the active ingredient plus any additional ingredient from a previously sterile-filtered solution thereof.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Injectable compositions can contain from about 0.1 to about 5% w/w of a compound as disclosed herein.

### 5. DOSAGE

The PI3K inhibitor (e.g., Compound 1 or GS1101) or another agent disclosed herein (e.g., one or more of the second agents disclosed herein) may be delivered in the form of pharmaceutically acceptable compositions. In certain embodiments, the pharmaceutical compositions comprise the PI3K inhibitor (e.g., Compound 1) described herein and/or one or more additional therapeutic agents, formulated together with one or more pharmaceutically acceptable excipients. In some instances, the PI3K inhibitor (e.g., Compound 1) or one or more of the other therapeutic agents disclosed herein are administered in separate pharmaceutical compositions and may (e.g., because of different physical and/or chemical characteristics) be administered by different routes (e.g., one therapeutic is administered orally, while the other is administered intravenously). In other instances, the PI3K inhibitor (e.g., Compound 1) or one or more of the other therapeutic agents disclosed herein may be administered separately, but via the same route (e.g., both orally or both intravenously). In still other instances, the PI3K inhibitor (e.g., Compound 1) or one or more of the other therapeutic agents disclosed herein may be administered in the same pharmaceutical composition.

The selected dosage level will depend upon a variety of factors including, for example, the activity of the particular compound employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of Compound 1 described herein and/or a therapeutic agent will be that amount of the compound which, in some embodiments, may be the lowest dose effective to produce a therapeutic effect. Such an effective dose will generally depend upon the factors described herein. Generally, doses of Compound 1 or the therapeutic agent described herein for a patient, when used for the indicated effects, will range from about 0.0001 mg to about 100 mg per day, or about 0.001 mg to about 100 mg per day, or about 0.01 mg to about 100 mg per day, or about 0.1 mg to about 100 mg per day, or about 0.0001 mg to about 500 mg per day, or about 0.001 mg to about 500 mg per day, or about 0.01 mg to 1000 mg, or about 0.01 mg to about 500 mg per day, or about 0.1 mg to about 500 mg per day, or about 1 mg to 50 mg per day, or about 5 mg to 40 mg per day. An exemplary dosage is about 10 to 30 mg per day. In some embodiments, for a 70 kg human, a suitable dose would be about 0.05 to about 7 g/day, such as about 0.05 to about 2.5 g/day. Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to the patient. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, the compounds may be administered daily, every other day, three times a week, twice a week, weekly, or bi-weekly. The dosing schedule can include a “drug holiday,” e.g., the drug may be administered for two weeks on, one week off, or three weeks on, one week off, or four weeks on, one week off, etc., or continuously, without a drug holiday. The compounds may be administered orally, intravenously, intraperitoneally, topically, transdermally, intramuscularly, subcutaneously, intranasally, sublingually, or by any other route.

In some embodiments, Compound 1 or the therapeutic agent described herein may be administered in multiple doses. Dosing may be about once, twice, three times, four times, five times, six times, or more than six times per day. Dosing may be about once a month, about once every two weeks, about once a week, or about once every other day. In another embodiment, Compound 1 as disclosed herein and another therapeutic agent are administered together from about once per day to about 6 times per day. In another embodiment, the administration of Compound 1 as provided herein and a therapeutic agent continues for less than about 7 days. In yet another embodiment, the administration continues for more than about 6 days, about 10 days, about 14 days, about 28 days, about two months, about six months, or about one year. In some cases, continuous dosing is achieved and maintained as long as necessary.

Administration of the pharmaceutical compositions as disclosed herein may continue as long as necessary. In some embodiments, an agent as disclosed herein is administered for more than about 1, about 2, about 3, about 4, about 5, about 6, about 7, about 14, or about 28 days. In some embodiments, an agent as disclosed herein is administered for less than about 28, about 14, about 7, about 6, about 5, about 4, about 3, about 2, or about 1 day. In some embodiments, a therapeutic agent as disclosed herein is administered chronically on an ongoing basis, e.g., for the treatment of chronic effects.

Since Compound 1 described herein may be administered in combination with one or more therapeutic agent, the doses of each agent or therapy may be lower than the corresponding dose for single-agent therapy. The dose for single-agent therapy can range from, for example, about 0.0001 to about 200 mg, or about 0.001 to about 100 mg, or about 0.01 to about 100 mg, or about 0.1 to about 100 mg, or about 1 to about 50 mg per kilogram of body weight per day.

When Compound 1 provided herein, is administered in a pharmaceutical composition that comprises one or more therapeutic agents, and the agent has a shorter half-life than Compound 1, unit dose forms of the agent and Compound 1 can be adjusted accordingly.

### 6. KITS

In some embodiments, provided herein are kits. The kits may include a pharmaceutical composition as described herein, in suitable packaging, and written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information may be based on the results of various studies, for example, studies using experimental animals involving in vivo models and studies based on human clinical trials.

In some embodiments, a memory aid is provided with the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” may be a single tablet or capsule or several tablets or capsules to be taken on a given day.

The kit may contain Compound 1 and one or more therapeutic agents. In some embodiments, Compound 1 and the agent are provided as separate pharmaceutical compositions in separate containers within the kit. In some embodiments, Compound 1 as disclosed herein and the agent are provided as a single pharmaceutical composition within a container in the kit. Suitable packaging and additional articles for use (e.g., measuring cup for liquid preparations, foil wrapping to minimize exposure to air, and the like) are known in the art and may be included in the kit. In other embodiments, kits may further comprise devices that are used to administer the active agents. Examples of such devices include, but are not limited to, syringes, drip bags, patches, and inhalers. Kits described herein may be provided, marketed and/or promoted to health providers, including physicians, nurses, pharmacists, formulary officials, and the like. Kits can also, in some embodiments, be marketed directly to the consumer.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. The strength of the sheet is such that the tablets or capsules may be removed from the blister pack by manually applying pressure on the recesses whereby an opening is formed in the sheet at the place of the recess. The tablet or capsule can then be removed via said opening.

Kits may further comprise pharmaceutically acceptable vehicles that may be used to administer one or more active agents. For example, if an active agent is provided in a solid form that must be reconstituted for parenteral administration, the kit can comprise a sealed container of a suitable vehicle in which the active agent may be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The present disclosure further encompasses anhydrous pharmaceutical compositions and dosage forms comprising an active ingredient, since water can facilitate the degradation of some compounds. For example, water may be added (e.g., about 5%) in the pharmaceutical arts as a means of simulating long-term storage in order to determine characteristics such as shelf-life or the stability of formulations over time. Anhydrous pharmaceutical compositions and dosage forms may be prepared using anhydrous or low moisture containing ingredients and low moisture or low humidity conditions. For example, pharmaceutical compositions and dosage forms which contain lactose may be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous pharmaceutical compositions may be packaged using materials known to prevent exposure to water such that they may be included in suitable formulary kits. Examples of suitable packaging include, but are not limited to, hermetically sealed foils, plastic or the like, unit dose containers, blister packs, and strip packs.

### EXAMPLES

### Example 1

### Combination Studies

The synergistic effects of compounds provided herein and another therapeutic agent were carried out. The method is described as follows. Cells are thawed from a liquid nitrogen preserved state. Once cells have been expanded and divide at their expected doubling times, screening begins. Cells are seeded in growth media in either black 1536-well or 384-well tissue culture treated plates. Cells are then equilibrated in assay plates via centrifugation and placed in incubators attached to the Dosing Modules at 37° C. for 24 hours before treatment. At the time of treatment, a set of assay plates (which do not receive treatment) are collected and ATP levels are measured by adding ATPLite (Perkin Elmer). These Tzero (T₀) plates are read using ultra-sensitive luminescence on Envision plate readers (Perkin Elmer). Treated assay plates are incubated with compound for 72 hours. After 72 hours, plates are developed for endpoint analysis using ATPLite. All data points are collected via automated processes, quality controlled and analyzed using Zalicus software. Assay plates are accepted if they pass the following quality control standards: relative luciferase values are consistent throughout the entire experiment, Z-factor scores are greater than 0.6, untreated/vehicle controls behave consistently on the plate.

Inhibition (I) is defined as

I=(1−T/V)*100%

where T is treated cell count and V is untreated (vehicle) cell count (at 72 hours). I ranges from 0% (when T=V) to 100% (when T=0). The IC₅₀ value is defined as the drug concentration needed to inhibit 50% of the cell growth compared to growth of the vehicle treated cells (the drug concentration which gives I=50%). The measure of effect in the experiment can be the inhibition of cellular response relative to the untreated level (vehicle alone). For untreated vehicle and treated levels V and T, a fractional inhibition I=1−T/V is calculated. The inhibition ranges from 0% at the untreated level to 100% when T=0. Inhibition levels are negative for agents that actually increase levels. Other effect measures, such as an activity ratio r=T/V may be more appropriate for some assays. When activity ratios (e.g, fold increase over stimulated control) are being used, the effect can be measured using an induction I=ln(T/V). With this definition, all effect expressions are the same as for inhibition.

Growth Inhibition (GI) is used as a measure of cell viability. The cell viability of vehicle is measured at the time of dosing (T0) and after 72 hours (T72). A GI reading of 0% represents no growth inhibition—T72 compound-treated and T72 vehicle signals are matched. A GI reading of 100% represents complete growth inhibition—T72 compound-treated and T0 vehicle signals are matched. Cell numbers have not increased during the treatment period in wells with GI 100% and may suggest a cytostatic effect for compounds reaching a plateau at this effect level. A GI reading of 200% represents complete death of all cells in the culture well. Compounds reaching an activity plateau of GI 200% are considered cytotoxic. GI is calculated by applying the following test and equation:

$$If T < {V}_{0} \text{:} 100 * ( 1 - \frac{T - {V}_{0}}{{V}_{0}} )$$

$$If T \geq {V}_{0} \text{:} 100 * ( 1 - \frac{T - {V}_{0}}{V - {V}_{0}} )$$

where T is the signal measure for a test article, V is the vehicle-treated control measure, and V₀ is the vehicle control measure at time zero. This formula is derived from the Growth Inhibition calculation used in the National Cancer Institute's NCI-60 high-throughput screen.

Combination analysis data were collected in a 6×6 dose matrix. Synergy is calculated by comparing a combination's response to those of its single compound, against the drug-with-itself dose-additive reference model. Deviations from dose additivity may be assessed visually on an isobologram or numerically with a Combination Index (CI). See the tables below for CI at 50% inhibition and CI at 50% growth inhibition. Additive effect is CI=1.0. Synergistic effect is CI<1. Antagonistic effect is CI>1.0.

Potency shifting was evaluated using an isobologram, which demonstrates how much less drug is required in combination to achieve a desired effect level, when compared to the single agent doses needed to reach that effect. The isobologram was drawn by identifying the locus of concentrations that correspond to crossing the indicated inhibition level. This is done by finding the crossing point for each single agent concentration in a dose matrix across the concentrations of the other single agent. Practically, each vertical concentration C_{Y} is held fixed while a bisection algorithm is used to identify the horizontal concentration C_{X} in combination with that vertical dose that gives the chosen effect level in the response surface Z(C_{X},C_{Y}). These concentrations are then connected by linear interpolation to generate the isobologram display. For synergistic interactions, the isobologram contour fall below the additivity threshold and approaches the origin, and an antagonistic interaction would lie above the additivity threshold. The error bars represent the uncertainty arising from the individual data points used to generate the isobologram. The uncertainty for each crossing point is estimated from the response errors using bisection to find the concentrations where Z−σ_{Z}(C_{X},C_{Y}) and Z+σ_{Z}(C_{X},C_{Y}) cross I_{cut}, where σ_{Z} is the standard deviation of the residual error on the effect scale.

To measure combination effects in excess of Loewe additivity, a scalar measure to characterize the strength of synergistic interaction termed the Synergy Score is devised. The Synergy Score is calculated as:

Synergy Score=log f_{X} log f_{Y}Σmax(O,I_{data})(I_{data}−I_{Loewe})

The fractional inhibition for each component agent and combination point in the matrix is calculated relative to the median of all vehicle-treated control wells. The Synergy Score equation integrates the experimentally-observed activity volume at each point in the matrix in excess of a model surface numerically derived from the activity of the component agents using the Loewe model for additivity. Additional terms in the Synergy Score equation (above) are used to normalize for various dilution factors used for individual agents and to allow for comparison of synergy scores across an entire experiment. The inclusion of positive inhibition gating or an I_{data} multiplier removes noise near the zero effect level, and biases results for synergistic interactions at that occur at high activity levels.

The Synergy Score measure was used for the self-cross analysis. Synergy Scores of self-crosses are expected to be additive by definition and, therefore, maintain a synergy score of zero. However, while some self-cross synergy scores are near zero, many are greater suggesting that experimental noise or non-optimal curve fitting of the single agent dose responses are contributing to the slight perturbations in the score. This strategy was cell line-centric, focusing on self-cross behavior in each cell line versus a global review of cell line panel activity. Combinations where the synergy score is greater than the mean self-cross plus two standard deviations or three standard deviations can be considered candidate synergies at 95% and 99% confidence levels, respectively. Additivity should maintain a synergy score of zero, and synergy score of two or three standard deviations indicate that the combination is synergistic at statistically significant levels of 95% and 99%.

Loewe Volume (Loewe Vol) is used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, as in the current dataset, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself. The calculation for Loewe additivity is:

I_{Loewe} that satisfies(X/X₁)+(Y/Y₁)=1

where XI and YI are the single agent effective concentrations for the observed combination effect I. For example, if 50% inhibition is achieved separately by 1 μM of drug A or 1 μM of drug B, a combination of 0.5 μM of A and 0.5 μM of B should also inhibit by 50%.

### Results

The CI₅₀ values for growth inhibition and inhibition in Tables 1-6 are categorized as follows: S=0.01 to <0.5, T=0.5 to <0.7, U=0.7 to <1, and W=≧1. The synergy score values for growth inhibition and inhibition are categorized as follows: A1=0.0001 to <1, A2=1 to <3, and A3=>3.

The types of cell lines tested are diffuse large B-cell lymphoma (DBCL) activated B-cell-like (ABC), DBCL germinal center B-cell-like (GCB), follicular lymphoma, mantle cell lymphoma, multiple myeloma, and T-cell lymphoma. These cell lines may have different genomic profiles and thus, a combination of Compound 1 and a therapeutic agent can have different synergistic effects on these cell lines. Data show that a combination of Compound 1 and a therapeutic agent provides a synergistic effect in various types of cell lines.

### Diffuse Large B-Cell Lymphoma (Activated B-Cell-Like)

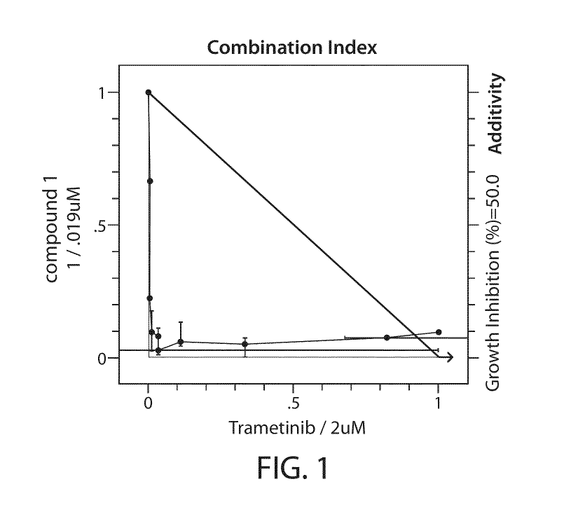

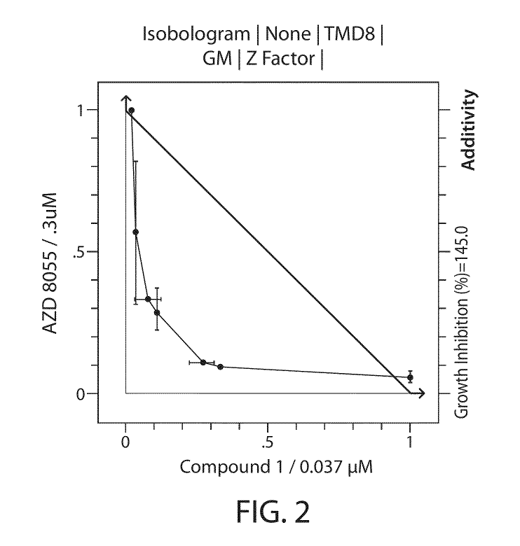

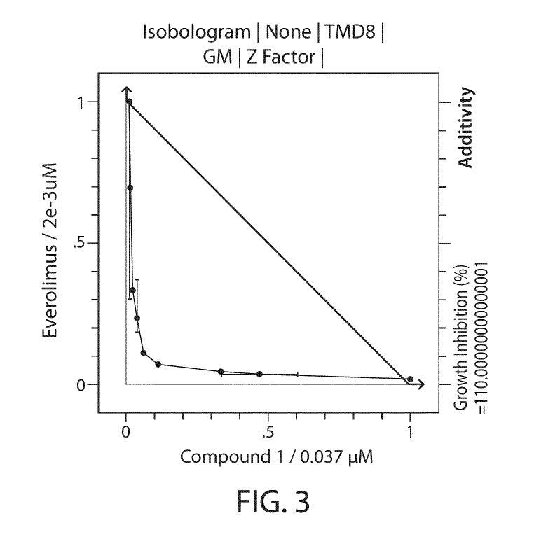

Cell lines related to diffuse large B-cell lymphoma (DBCL) activated B-cell-like (ABC) were exposed to a combination of Compound 1 and a therapeutic agent. These cell lines include HBL-1, OCI-Ly3, TMD8, and U2832. The results are shown in Table 1 below. An isobologram depicting the synergistic effect of the combination of Compound 1 and trametinib in TMD8 DLBCL cell line is provided in FIG. 1. An isobologram depicting the synergistic effect of the combination of Compound 1 and AZD8055 in TMD8 DLBCL cell line is provided in FIG. 2. An isobologram depicting the synergistic effect of the combination of Compound 1 and everolimus in TMD8 DLBCL cell line is provided in FIG. 3.

TABLE 1

| | | Synergy | | | |
|---|---|---|---|---|---|
| | | Score | CI₅₀ | Synergy | |
| | | growth | growth | Score | CI₅₀ |
| therapeutic | | inhibi- | inhibi- | inhibi- | inhibi- |
| agent | Cell Line | tion | tion | tion | tion |
| AZD 8055 | HBL-1 | A3 | S | A1 | T |
| AZD 8055 | OCI-Ly3 | A2 | S | A1 | T |
| AZD 8055 | U-2932 | A2 | S | A2 | U |
| AZD 8055 | TMD8 | A3 | S | A3 | S |
| Bortezomib | U-2932 | A1 | U | A1 | W |
| Bortezomib | OCI-Ly3 | A1 | U | A1 | W |
| Bortezomib | HBL-1 | A2 | U | A1 | U |
| Bortezomib | TMD8 | A3 | U | A1 | U |
| Carfilzomib | HBL-1 | A1 | T | A1 | U |
| Carfilzomib | OCI-Ly3 | A2 | U | A1 | W |
| Carfilzomib | U-2932 | A2 | U | A2 | U |
| Carfilzomib | TMD8 | A3 | U | A2 | U |
| Everolimus | OCI-Ly3 | A2 | U | A1 | |
| Everolimus | HBL-1 | A2 | T | A1 | U |
| Everolimus | U-2932 | A2 | S | A2 | |
| Everolimus | TMD8 | A3 | T | A3 | S |
| MK-2206 | U-2932 | A1 | U | A1 | U |
| MK-2206 | HBL-1 | A2 | T | A1 | W |
| MK-2206 | OCI-Ly3 | A2 | S | A1 | W |
| MK-2206 | TMD8 | A3 | S | A3 | S |
| PD0325901 | U-2932 | A2 | U | A1 | |
| PD0325901 | HBL-1 | A2 | T | A1 | |
| PD0325901 | OCI-Ly3 | A2 | S | A1 | |
| PD0325901 | TMD8 | A3 | U | A3 | U |
| Perifosine | OCI-Ly3 | A1 | W | A1 | W |
| Perifosine | U-2932 | A1 | W | A1 | W |
| Perifosine | HBL-1 | A3 | U | A1 | W |
| Perifosine | TMD8 | A2 | T | A2 | T |
| Trametinib | U-2932 | A1 | U | A1 | |
| Trametinib | HBL-1 | A2 | U | A1 | |
| Trametinib | OCI-Ly3 | A2 | W | A1 | |
| Trametinib | TMD8 | A3 | S | A3 | S |
| Lenalidomide | TMD8 | A3 | S | A3 | S |
| Lenalidomide | U-2932 | A1 | U | A1 | |
| Lenalidomide | OCI-Ly3 | A1 | W | A1 | |
| Lenalidomide | HBL-1 | A1 | W | A1 | |
| Dexamethasone | TMD8 | A3 | U | A3 | S |
| Dexamethasone | U-2932 | A3 | S | A2 | S |
| Dexamethasone | OCI-Ly3 | A1 | U | A1 | |
| Dexamethasone | HBL-1 | A3 | S | A2 | U |
| Romidepsin | HBL-1 | A2 | T | A1 | U |
| Romidepsin | OCI-Ly3 | A2 | T | A1 | U |
| Romidepsin | U-2932 | A3 | T | A1 | U |
| Romidepsin | TMD8 | A2 | U | A1 | U |
| Tubastatin A | HBL-1 | A2 | U | A1 | W |
| hydrochloride | | | | | |
| Tubastatin A | OCI-Ly3 | A2 | U | A1 | W |
| hydrochloride | | | | | |
| Tubastatin A | U-2932 | A1 | U | A1 | |
| hydrochloride | | | | | |
| Tubastatin A | TMD8 | A3 | U | A2 | U |
| hydrochloride | | | | | |
| (+)-JQ1 | HBL-1 | A2 | S | A1 | W |
| (+)-JQ1 | OCI-Ly3 | A3 | S | A2 | T |
| (+)-JQ1 | U-2932 | A2 | U | A2 | U |
| (+)-JQ1 | TMD8 | A3 | T | A2 | T |
| Azacitidine | HBL-1 | A2 | S | A1 | W |
| Azacitidine | OCI-Ly3 | A3 | S | A2 | |
| Azacitidine | U-2932 | A2 | S | A1 | W |
| Azacitidine | TMD8 | A2 | U | A2 | U |
| Doxorubicin | HBL-1 | A3 | S | A1 | U |
| HCl | | | | | |
| Doxorubicin | OCI-Ly3 | A2 | U | A1 | W |
| HCl | | | | | |
| Doxorubicin | U-2932 | A2 | U | A1 | W |
| HCl | | | | | |
| Doxorubicin | TMD8 | A3 | T | A2 | T |
| HCl | | | | | |
| GDC-0941 | HBL-1 | A2 | U | A1 | W |
| GDC-0941 | OCI-Ly3 | A1 | U | A1 | W |
| GDC-0941 | U-2932 | A1 | T | A1 | W |
| GDC-0941 | TMD8 | A3 | U | A2 | W |
| SCH772984 | HBL-1 | A1 | T | A1 | |
| SCH772984 | OCI-Ly3 | A1 | W | A1 | |
| SCH772984 | U-2932 | A1 | W | A1 | |
| SCH772984 | TMD8 | A1 | T | A1 | U |

### Diffuse Large B-Cell Lymphoma (Germinal Center B-Cell-Like)

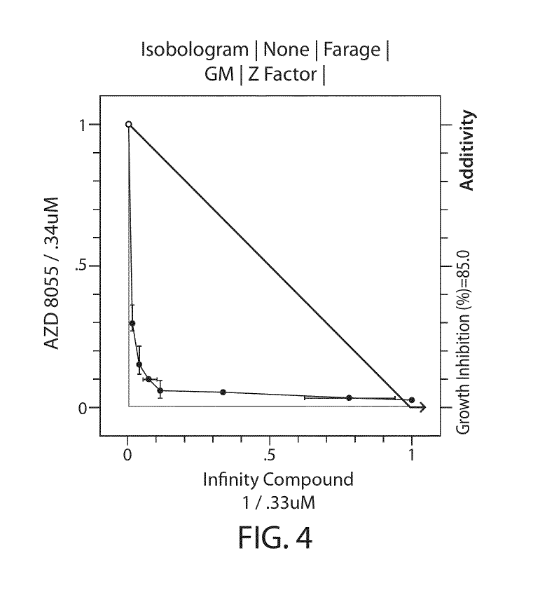

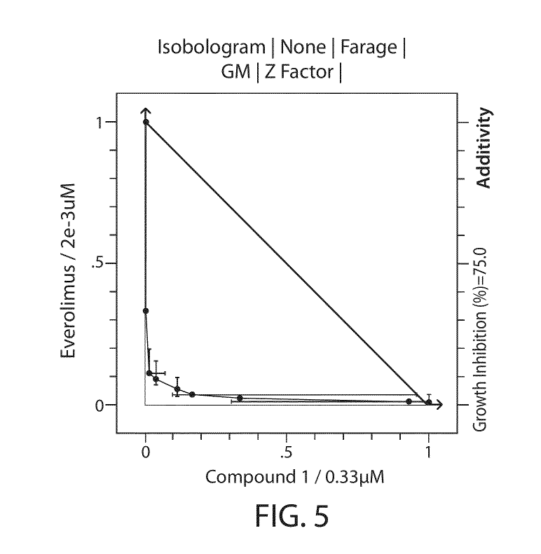

Cell lines related to DBCL germinal center B-cell-like (GCB) were exposed to a combination of Compound 1 and a therapeutic agent. These cell lines include DOHH-2, Farage, OCI-Ly7, SU-DHL-10-epst, and SU-DHL-4-epst. The results are shown in Table 2 below. An isobologram depicting the synergistic effect of the combination of Compound 1 and AZD8055 in Farage DLBCL cell line is provided in FIG. 4. An isobologram depicting the synergistic effect of the combination of Compound 1 and everolimus in Farage DLBCL cell line is provided in FIG. 5.

TABLE 2

| | | Synergy | | | |
|---|---|---|---|---|---|
| | | Score | CI₅₀ | Synergy | |
| | | growth | growth | Score | CI₅₀ |
| therapeutic | | inhibi- | inhibi- | inhibi- | inhibi- |
| agent | Cell Line | tion | tion | tion | tion |
| AZD 8055 | OCI-Ly7 | A3 | S | A2 | T |
| AZD 8055 | SU-DHL-4-epst | A3 | T | A3 | S |
| AZD 8055 | DOHH-2 | A3 | S | A3 | S |
| AZD 8055 | Farage | A3 | S | A3 | S |
| AZD 8055 | SU-DHL-10-epst | A3 | S | A3 | S |
| Bortezomib | SU-DHL-10-epst | A1 | U | A1 | U |
| Bortezomib | DOHH-2 | A2 | U | A1 | U |
| Bortezomib | OCI-Ly7 | A2 | U | A1 | U |
| Bortezomib | SU-DHL-4-epst | A3 | U | A1 | T |
| Bortezomib | Farage | A3 | U | A1 | U |
| Carfilzomib | OCI-Ly7 | A2 | W | A1 | W |
| Carfilzomib | DOHH-2 | A3 | U | A1 | U |
| Carfilzomib | Farage | A3 | U | A1 | U |
| Carfilzomib | SU-DHL-10-epst | A3 | U | A2 | U |
| Carfilzomib | SU-DHL-4-epst | A3 | U | A2 | U |
| Everolimus | OCI-Ly7 | A2 | S | A2 | W |
| Everolimus | DOHH-2 | A3 | S | A3 | S |
| Everolimus | Farage | A3 | T | A3 | S |
| Everolimus | SU-DHL-4-epst | A3 | S | A3 | S |
| Everolimus | SU-DHL-10-epst | A3 | S | A3 | S |
| MK-2206 | OCI-Ly7 | A1 | U | A1 | W |
| MK-2206 | SU-DHL-4-epst | A3 | S | A3 | S |
| MK-2206 | DOHH-2 | A3 | S | A3 | S |
| MK-2206 | Farage | A3 | S | A3 | S |
| MK-2206 | SU-DHL-10-epst | A3 | S | A3 | S |
| PD0325901 | OCI-Ly7 | A1 | | A1 | |
| PD0325901 | DOHH-2 | A1 | W | A1 | W |
| PD0325901 | Farage | A2 | W | A1 | W |
| PD0325901 | SU-DHL-4-epst | A2 | S | A2 | T |
| PD0325901 | SU-DHL-10-epst | A3 | S | A2 | S |
| Perifosine | OCI-Ly7 | A2 | U | A1 | W |
| Perifosine | SU-DHL-4-epst | A2 | S | A1 | U |
| Perifosine | DOHH-2 | A3 | T | A2 | U |
| Perifosine | Farage | A3 | S | A2 | T |
| Perifosine | SU-DHL-10-epst | A2 | T | A2 | U |
| Trametinib | OCI-Ly7 | A1 | | A1 | |
| Trametinib | DOHH-2 | A3 | U | A2 | W |
| Trametinib | Farage | A2 | W | A2 | U |
| Trametinib | SU-DHL-4-epst | A3 | S | A2 | S |
| Trametinib | SU-DHL-10-epst | A3 | S | A3 | S |
| Lenalidomide | DOHH-2 | A3 | S | A2 | S |
| Lenalidomide | SU-DHL-10-epst | A2 | T | A2 | T |
| Lenalidomide | SU-DHL-4-epst | A2 | W | A1 | T |
| Lenalidomide | Farage | A2 | W | A1 | W |
| Lenalidomide | OCI-Ly7 | A1 | | A1 | |
| Dexamethasone | DOHH-2 | A3 | S | A3 | T |
| Dexamethasone | OCI-Ly7 | A3 | S | A3 | S |
| Dexamethasone | SU-DHL-10-epst | A3 | S | A3 | S |
| Dexamethasone | Farage | A3 | U | A3 | T |
| Dexamethasone | SU-DHL-4-epst | A3 | T | A3 | T |
| Romidepsin | OCI-Ly7 | A3 | T | A2 | U |
| Romidepsin | SU-DHL-4-epst | A3 | U | A2 | U |
| Romidepsin | DOHH-2 | A3 | U | A1 | U |
| Romidepsin | Farage | A3 | T | A2 | T |
| Romidepsin | SU-DHL-10-epst | A3 | T | A2 | U |
| Tubastatin A | OCI-Ly7 | A3 | S | A2 | U |
| hydrochloride | | | | | |
| Tubastatin A | SU-DHL-4-epst | A3 | T | A2 | S |
| hydrochloride | | | | | |
| Tubastatin A | DOHH-2 | A3 | T | A2 | T |
| hydrochloride | | | | | |
| Tubastatin A | Farage | A3 | S | A2 | T |
| hydrochloride | | | | | |
| Tubastatin A | SU-DHL-10-epst | A3 | T | A2 | T |
| hydrochloride | | | | | |
| (+)-JQ1 | OCI-Ly7 | A2 | T | A2 | T |
| (+)-JQ1 | SU-DHL-4-epst | A3 | T | A2 | T |
| (+)-JQ1 | DOHH-2 | A3 | U | A2 | U |
| (+)-JQ1 | Farage | A3 | T | A3 | S |
| (+)-JQ1 | SU-DHL-10-epst | A3 | T | A2 | T |
| Azacitidine | OCI-Ly7 | A3 | T | A2 | T |
| Azacitidine | SU-DHL-4-epst | A3 | S | A3 | S |
| Azacitidine | DOHH-2 | A3 | U | A2 | U |
| Azacitidine | Farage | A3 | S | A2 | S |
| Azacitidine | SU-DHL-10-epst | A3 | s | A3 | S |
| Doxorubicin | OCI-Ly7 | A3 | S | A2 | S |
| HCl | | | | | |
| Doxorubicin | SU-DHL-4-epst | A3 | U | A2 | W |
| HCl | | | | | |
| Doxorubicin | DOHH-2 | A3 | T | A2 | T |
| HCl | | | | | |
| Doxorubicin | Farage | A3 | U | A1 | T |
| HCl | | | | | |
| Doxorubicin | SU-DHL-10-epst | A3 | T | A2 | T |
| HCl | | | | | |
| GDC-0941 | OCI-Ly7 | A1 | U | A1 | W |
| GDC-0941 | SU-DHL-4-epst | A3 | T | A2 | T |
| GDC-0941 | DOHH-2 | A3 | T | A2 | T |
| GDC-0941 | Farage | A3 | T | A2 | S |
| GDC-0941 | SU-DHL-10-epst | A3 | S | A2 | T |
| SCH772984 | OCI-Ly7 | A1 | | A1 | |
| SCH772984 | SU-DHL-4-epst | A1 | W | A1 | |
| SCH772984 | DOHH-2 | A1 | W | A1 | W |
| SCH772984 | Farage | A1 | T | A1 | W |
| SCH772984 | SU-DHL-10-epst | A2 | T | A2 | W |

The combination of Compound 1 with dexamethasone was also tested in the SUDHL6 cell line, and significant synergy was observed (data not shown).

### Follicular Lymphoma

Cell lines related to follicular lymphoma were exposed to a combination of Compound 1 and a therapeutic agent. These cell lines include Karpas-422, RL, and WSU-NHL. The results are shown in Table 3 below.

TABLE 3

| | | Synergy | | | |
|---|---|---|---|---|---|
| | | Score | CI₅₀ | Synergy | |
| | | growth | growth | Score | CI₅₀ |
| therapeutic | | inhibi- | inhibi- | inhibi- | inhibi- |
| agent | Cell Line | tion | tion | tion | tion |
| AZD 8055 | RL | A2 | U | A2 | U |
| AZD 8055 | KARPAS-422 | A2 | S | A2 | S |
| AZD 8055 | WSU-NHL | A3 | T | A3 | S |
| Bortezomib | RL | A1 | U | A1 | W |
| Bortezomib | WSU-NHL | A1 | U | A1 | U |
| Bortezomib | KARPAS-422 | A2 | W | A1 | W |
| Carfilzomib | RL | A1 | W | A1 | W |
| Carfilzomib | WSU-NHL | A2 | U | A1 | U |
| Carfilzomib | KARPAS-422 | A3 | T | A1 | U |
| Everolimus | KARPAS-422 | A2 | W | A2 | S |
| Everolimus | RL | A2 | T | A2 | S |
| Everolimus | WSU-NHL | A3 | T | A3 | S |
| MK-2206 | KARPAS-422 | A2 | T | A2 | S |
| MK-2206 | RL | A3 | S | A3 | S |
| MK-2206 | WSU-NHL | A3 | S | A3 | S |
| PD0325901 | RL | A1 | | A1 | |
| PD0325901 | KARPAS-422 | A2 | S | A2 | T |
| PD0325901 | WSU-NHL | A3 | S | A3 | S |
| Perifosine | RL | A1 | W | A1 | W |
| Perifosine | KARPAS-422 | A1 | W | A1 | U |
| Perifosine | WSU-NHL | A3 | U | A2 | T |
| Trametinib | RL | A1 | | A1 | |
| Trametinib | KARPAS-422 | A2 | S | A2 | S |
| Trametinib | WSU-NHL | A3 | T | A3 | S |
| Lenalidomide | WSU-NHL | A3 | T | A2 | S |
| Lenalidomide | KARPAS-422 | A2 | S | A2 | S |
| Lenalidomide | RL | A1 | | A1 | |
| Dexamethasone | RL | A3 | S | A3 | S |
| Dexamethasone | WSU-NHL | A3 | | A3 | T |
| Dexamethasone | KARPAS-422 | A3 | U | A3 | U |
| Romidepsin | RL | A2 | U | A1 | U |
| Romidepsin | KARPAS-422 | A1 | U | A1 | U |
| Romidepsin | WSU-NHL | A3 | T | A2 | T |
| Tubastatin A | RL | A2 | W | A2 | W |
| hydrochloride | | | | | |
| Tubastatin A | KARPAS-422 | A1 | | A1 | |
| hydrochloride | | | | | |
| Tubastatin A | WSU-NHL | A3 | T | A2 | T |
| hydrochloride | | | | | |
| (+)-JQ1 | RL | A3 | U | A2 | U |
| (+)-JQ1 | KARPAS-422 | A3 | U | A2 | T |
| (+)-JQ1 | WSU-NHL | A3 | U | A2 | T |
| Azacitidine | RL | A2 | T | A2 | U |
| Azacitidine | KARPAS-422 | A2 | T | A2 | U |
| Azacitidine | WSU-NHL | A3 | T | A2 | S |
| Doxorubicin | RL | A2 | U | A1 | W |
| HCl | | | | | |
| Doxorubicin | KARPAS-422 | A1 | W | A1 | W |
| HCl | | | | | |
| Doxorubicin | WSU-NHL | A3 | T | A2 | S |
| HCl | | | | | |
| GDC-0941 | RL | A2 | U | A2 | U |
| GDC-0941 | KARPAS-422 | A1 | T | A1 | U |
| GDC-0941 | WSU-NHL | A3 | U | A2 | T |
| SCH772984 | RL | A1 | | A1 | |
| SCH772984 | KARPAS-422 | A1 | T | A1 | W |
| SCH772984 | WSU-NHL | A1 | U | A1 | U |

### T-Cell Lymphoma

Cell lines related to T-cell lymphoma were exposed to a combination of Compound 1 and a therapeutic agent. The cell line includes HH and Karpas-299. The results are shown in Table 4 below.

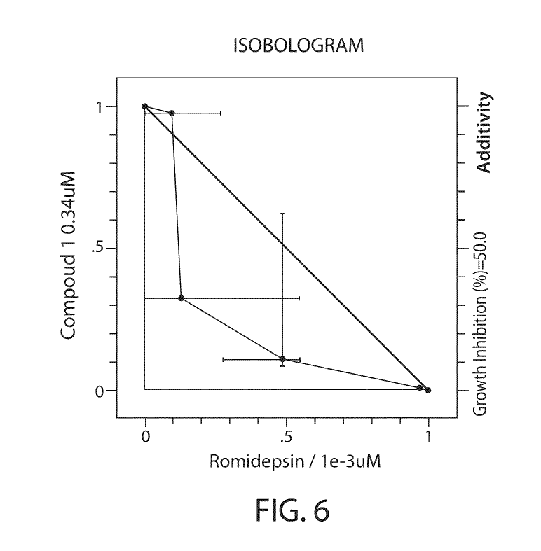

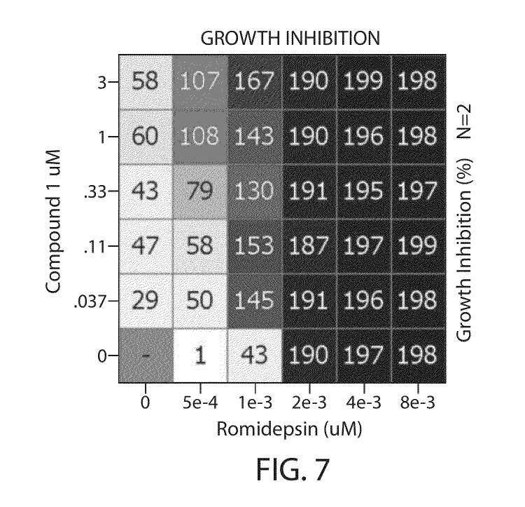

The experiments disclosed herein support a rationale for combining Compound 1 with one or more standard of care agents, such an HDAC inhibitor, e.g., romidepsin, for treatment of cancer, e.g., T-cell lymphoma. For example, the combination of Compound 1 and romidepsin shows synergistic effects in T-cell lymphoma. See FIGS. 6 and 7, which depict an isobologram and a matrix plot, respectively, demonstrating the synergistic effect of the combination of Compound 1 and romidepsin in HH cutaneous T-cell cell line.

TABLE 4

| | | Synergy | | | |
|---|---|---|---|---|---|
| | | Score | CI₅₀ | Synergy | |
| | | growth | growth | Score | CI₅₀ |
| therapeutic | | inhibi- | inhibi- | inhibi- | inhibi- |
| agent | Cell Line | tion | tion | tion | tion |
| AZD 8055 | KARPAS-299 | A1 | W | A1 | U |
| AZD 8055 | HH | A3 | S | A2 | S |
| Bortezomib | KARPAS-299 | A1 | U | A1 | U |
| Bortezomib | HH | A3 | U | A2 | U |
| Carfilzomib | KARPAS-299 | A1 | W | A1 | W |
| Carfilzomib | HH | A3 | T | A1 | T |
| Everolimus | KARPAS-299 | A1 | U | A1 | W |
| Everolimus | HH | A3 | S | A2 | S |
| MK-2206 | KARPAS-299 | A1 | | A1 | |
| MK-2206 | HH | A3 | S | A2 | S |
| PD0325901 | HH | A2 | W | A1 | U |
| PD0325901 | KARPAS-299 | A2 | S | A1 | |
| Perifosine | KARPAS-299 | A1 | W | A1 | W |
| Perifosine | HH | A3 | W | A2 | S |
| Trametinib | KARPAS-299 | A1 | | A1 | |
| Trametinib | HH | A3 | S | A2 | S |
| Lenalidomide | HH | A2 | W | A1 | T |
| Lenalidomide | KARPAS-299 | A1 | | A1 | |
| Dexamethasone | HH | A3 | S | A3 | S |
| Dexamethasone | KARPAS-299 | A1 | | A1 | |
| Romidepsin | KARPAS-299 | A1 | U | A1 | U |
| Romidepsin | HH | A3 | T | A2 | S |
| Tubastatin A | KARPAS-299 | A1 | | A1 | |
| hydrochloride | | | | | |
| Tubastatin A | HH | A3 | S | A2 | T |
| hydrochloride | | | | | |
| (+)-JQ1 | KARPAS-299 | A1 | W | A1 | W |
| (+)-JQ1 | HH | A3 | S | A3 | S |
| Azacitidine | KARPAS-299 | A1 | W | A1 | W |
| Azacitidine | HH | A3 | S | A2 | S |
| Doxorubicin | KARPAS-299 | A2 | U | A1 | U |
| HCl | | | | | |
| Doxorubicin | HH | A3 | W | A2 | T |
| HCl | | | | | |
| GDC-0941 | KARPAS-299 | A1 | W | A1 | |
| GDC-0941 | HH | A1 | W | A1 | W |
| SCH772984 | KARPAS-299 | A1 | | A1 | |
| SCH772984 | HH | A1 | T | A1 | S |

### Mantle Cell Lymphoma

Cell lines related to mantle cell lymphoma were exposed to a combination of Compound 1 and a therapeutic agent. These cell lines include GRANTA-519, Jeko-1 and Mino. The results are shown in Table 5 below.

TABLE 5

| | | Synergy | | | |
|---|---|---|---|---|---|
| | | Score | CI₅₀ | Synergy | |
| | | growth | growth | Score | CI₅₀ |
| therapeutic | | inhibi- | inhibi- | inhibi- | inhibi- |
| agent | Cell Line | tion | tion | tion | tion |
| AZD 8055 | GRANTA-519 | A2 | T | A1 | U |
| AZD 8055 | Mino | A2 | U | A1 | S |
| AZD 8055 | Jeko-1 | A3 | S | A2 | T |
| Bortezomib | Mino | A2 | U | A1 | U |
| Bortezomib | Jeko-1 | A1 | U | A1 | U |
| Bortezomib | GRANTA-519 | A2 | T | A1 | U |
| Carfilzomib | Jeko-1 | A1 | U | A1 | U |
| Carfilzomib | GRANTA-519 | A2 | T | A1 | W |
| Carfilzomib | Mino | A2 | U | A1 | U |
| Everolimus | GRANTA-519 | A2 | S | A1 | |
| Everolimus | Jeko-1 | A2 | T | A2 | U |
| Everolimus | Mino | A2 | T | A2 | U |
| MK-2206 | Mino | A1 | W | A1 | U |
| MK-2206 | GRANTA-519 | A1 | S | A1 | |
| MK-2206 | Jeko-1 | A2 | S | A2 | S |
| PD0325901 | Jeko-1 | A1 | U | A1 | |
| PD0325901 | GRANTA-519 | A3 | S | A2 | |
| PD0325901 | Mino | A3 | S | A3 | S |
| Perifosine | GRANTA-519 | A2 | S | A1 | W |
| Perifosine | Mino | A2 | U | A1 | W |
| Perifosine | Jeko-1 | A1 | U | A1 | W |
| Trametinib | GRANTA-519 | A2 | S | A1 | |
| Trametinib | Jeko-1 | A1 | U | A1 | W |
| Trametinib | Mino | A3 | S | A3 | S |
| Lenalidomide | Jeko-1 | A2 | S | A2 | T |
| Lenalidomide | Mino | A2 | T | A1 | W |
| Lenalidomide | GRANTA-519 | A1 | | A1 | |
| Dexamethasone | Jeko-1 | A3 | U | A3 | S |
| Dexamethasone | Mino | A3 | S | A2 | S |
| Dexamethasone | GRANTA-519 | A3 | S | A1 | |
| Romidepsin | GRANTA-519 | A2 | U | A1 | U |
| Romidepsin | Mino | A2 | U | A1 | U |
| Romidepsin | Jeko-1 | A2 | U | A1 | U |
| Tubastatin A | GRANTA-519 | A2 | S | A1 | W |
| hydrochloride | | | | | |
| Tubastatin A | Mino | A2 | U | A1 | U |
| hydrochloride | | | | | |
| Tubastatin A | Jeko-1 | A1 | W | A1 | W |
| hydrochloride | | | | | |
| (+)-JQ1 | GRANTA-519 | A3 | T | A2 | U |
| (+)-JQ1 | Mino | A3 | S | A3 | T |
| (+)-JQ1 | Jeko-1 | A3 | T | A2 | T |
| Azacitidine | GRANTA-519 | A1 | U | A1 | |
| Azacitidine | Mino | A3 | S | A2 | S |
| Azacitidine | Jeko-1 | A3 | S | A2 | T |
| Doxorubicin | GRANTA-519 | A3 | S | A1 | W |
| HCl | | | | | |
| Doxorubicin | Mino | A3 | T | A2 | T |
| HCl | | | | | |
| Doxorubicin | Jeko-1 | A3 | T | A1 | U |
| HCl | | | | | |
| GDC-0941 | GRANTA-519 | A2 | S | A1 | U |
| GDC-0941 | Mino | A2 | S | A2 | T |
| GDC-0941 | Jeko-1 | A1 | S | A1 | S |
| SCH772984 | GRANTA-519 | A1 | | A1 | |
| SCH772984 | Mino | A1 | W | A1 | |
| SCH772984 | Jeko-1 | A1 | W | A1 | |

### Multiple Myeloma

Cell lines related to multiple myeloma were exposed to a combination of Compound 1 and a therapeutic agent. These cell lines include NCI-H929, OMP-2, and RPMI-8226. The results are shown in Table 6 below.

TABLE 6

| | | Synergy | | | |
|---|---|---|---|---|---|
| | | Score | CI₅₀ | Synergy | |
| | | growth | growth | Score | CI₅₀ |
| therapeutic | | inhibi- | inhibi- | inhibi- | inhibi- |
| agent | Cell Line | tion | tion | tion | tion |
| AZD 8055 | OPM-2 | A2 | U | A1 | W |
| AZD 8055 | RPMI-8226 | A2 | U | A1 | W |
| AZD 8055 | NCI-H929 | A3 | W | A2 | T |
| Bortezomib | RPMI-8226 | A1 | U | A1 | U |
| Bortezomib | OPM-2 | A1 | W | A1 | W |
| Bortezomib | NCI-H929 | A1 | U | A1 | W |
| Carfilzomib | RPMI-8226 | A3 | U | A1 | W |
| Carfilzomib | OPM-2 | A2 | U | A1 | U |
| Carfilzomib | NCI-H929 | A3 | U | A1 | W |
| Everolimus | RPMI-8226 | A2 | U | A1 | W |
| Everolimus | OPM-2 | A2 | S | A2 | W |
| Everolimus | NCI-H929 | A3 | S | A2 | T |
| MK-2206 | RPMI-8226 | A2 | U | A1 | U |
| MK-2206 | NCI-H929 | A3 | T | A2 | U |
| MK-2206 | OPM-2 | A3 | S | A2 | U |
| PD0325901 | RPMI-8226 | A2 | U | A2 | |
| PD0325901 | OPM-2 | A3 | U | A2 | W |
| PD0325901 | NCI-H929 | A3 | S | A2 | U |
| Perifosine | NCI-H929 | A2 | U | A1 | W |
| Perifosine | RPMI-8226 | A3 | T | A1 | U |
| Perifosine | OPM-2 | A2 | U | A1 | W |
| Trametinib | RPMI-8226 | A2 | U | A1 | |
| Trametinib | OPM-2 | A2 | T | A2 | W |
| Trametinib | NCI-H929 | A3 | T | A2 | S |
| Lenalidomide | NCI-H929 | A3 | T | A3 | S |
| Lenalidomide | OPM-2 | A2 | S | A1 | |
| Lenalidomide | RPMI-8226 | A1 | | A1 | |
| Dexamethasone | NCI-H929 | A3 | S | A2 | U |
| Dexamethasone | OPM-2 | A2 | S | A1 | U |
| Dexamethasone | RPMI-8226 | A3 | S | A2 | U |
| Romidepsin | NCI-H929 | A2 | U | A1 | U |
| Romidepsin | OPM-2 | A2 | T | A1 | W |
| Romidepsin | RPMI-8226 | A3 | U | A1 | U |
| Tubastatin A | NCI-H929 | A3 | U | A2 | T |
| hydrochloride | | | | | |
| Tubastatin A | OPM-2 | A1 | W | A1 | |
| hydrochloride | | | | | |
| Tubastatin A | RPMI-8226 | A2 | U | A1 | U |
| hydrochloride | | | | | |
| (+)-JQ1 | NCI-H929 | A3 | T | A2 | T |
| (+)-JQ1 | OPM-2 | A3 | U | A1 | U |
| (+)-JQ1 | RPMI-8226 | A2 | U | A1 | U |
| Azacitidine | NCI-H929 | A3 | U | A1 | U |
| Azacitidine | OPM-2 | A3 | S | A1 | W |
| Azacitidine | RPMI-8226 | A3 | T | A1 | W |
| Doxorubicin | NCI-H929 | A3 | U | A1 | U |
| HCl | | | | | |
| Doxorubicin | OPM-2 | A2 | W | A1 | W |
| HCl | | | | | |
| Doxorubicin | RPMI-8226 | A3 | U | A2 | W |
| HCl | | | | | |
| GDC-0941 | NCI-H929 | A3 | T | A2 | T |
| GDC-0941 | OPM-2 | A3 | U | A1 | W |
| GDC-0941 | RPMI-8226 | A2 | U | A1 | W |
| SCH772984 | NCI-H929 | A1 | W | A1 | W |
| SCH772984 | OPM-2 | A1 | | A1 | |
| SCH772984 | RPMI-8226 | A1 | | A1 | |

### Example 2

### Combination Therapies of Compound 1 or CAL-101 and a Second Therapeutic Agent

A combination study of using Compound 1 or CAL-101 and a second therapeutic agent (e.g., dexamethasone, PCI-32765, LEE011, and PD-033299) was also carried out using procedures similar to those in Example 1 and the data are included below. The CI₅₀ values for growth inhibition and inhibition in Tables 7-9 are categorized as follows: S=0.01 to <0.5, T=0.5 to <0.7, U=0.7 to <1, and W=≧1. The synergy score values for growth inhibition and inhibition are categorized as follows: A1=0.0001 to <1, A2=1 to <3, and A3=>3.

TABLE 7
ABC DLBCL cell lines

| | | | Synergy Score | CI₅₀ | Synergy | |
|---|---|---|---|---|---|---|
| | therapeutic | | growth | growth | Score | CI₅₀ |
| Cmpd | agent | Cell Line | inhibition | inhibition | inhibition | inhibition |
| CAL- | Dexamethasone | HBL-1 | A2 | T | A2 | S |
| 101 | | | | | | |
| CAL- | LEE011 | HBL-1 | A1 | W | A1 | |
| 101 | | | | | | |
| CAL- | PCI-32765 | HBL-1 | A2 | S | A1 | |
| 101 | | | | | | |
| CAL- | PD-0332991 | HBL-1 | A1 | | A1 | |
| 101 | | | | | | |
| CAL- | Dexamethasone | OCI-Ly3 | A1 | | A1 | |
| 101 | | | | | | |
| CAL- | LEE011 | OCI-Ly3 | A2 | S | A1 | U |
| 101 | | | | | | |
| CAL- | PCI-32765 | OCI-Ly3 | A1 | | A1 | |
| 101 | | | | | | |
| CAL- | PD-0332991 | OCI-Ly3 | A3 | S | A2 | S |
| 101 | | | | | | |
| CAL- | Dexamethasone | TMD8 | A3 | S | A3 | S |
| 101 | | | | | | |
| CAL- | LEE011 | TMD8 | A3 | S | A2 | S |
| 101 | | | | | | |
| CAL- | PCI-32765 | TMD8 | A3 | S | A3 | S |
| 101 | | | | | | |
| CAL- | PD-0332991 | TMD8 | A3 | S | A2 | S |
| 101 | | | | | | |
| CAL- | Dexamethasone | U-2932 | A2 | S | A2 | S |
| 101 | | | | | | |
| CAL- | LEE011 | U-2932 | A1 | | A1 | |
| 101 | | | | | | |
| CAL- | PCI-32765 | U-2932 | A2 | T | A2 | U |
| 101 | | | | | | |
| CAL- | PD-0332991 | U-2932 | A1 | | A1 | |
| 101 | | | | | | |
| Cmpd 1 | Dexamethasone | HBL-1 | A2 | T | A2 | U |
| Cmpd 1 | LEE011 | HBL-1 | A1 | W | A1 | |
| Cmpd 1 | PCI-32765 | HBL-1 | A2 | S | A1 | |
| Cmpd 1 | PD-0332991 | HBL-1 | A1 | | A1 | |
| Cmpd 1 | Dexamethasone | OCI-Ly3 | A1 | W | A1 | |
| Cmpd 1 | LEE011 | OCI-Ly3 | A2 | S | A2 | U |
| Cmpd 1 | PCI-32765 | OCI-Ly3 | A1 | S | A1 | |
| Cmpd 1 | PD-0332991 | OCI-Ly3 | A2 | T | A1 | U |
| Cmpd 1 | Dexamethasone | TMD8 | A3 | T | A3 | S |
| Cmpd 1 | LEE011 | TMD8 | A3 | S | A2 | S |
| Cmpd 1 | PCI-32765 | TMD8 | A3 | U | A3 | T |
| Cmpd 1 | PD-0332991 | TMD8 | A3 | S | A2 | S |
| Cmpd 1 | Dexamethasone | U-2932 | A3 | S | A2 | S |
| Cmpd 1 | LEE011 | U-2932 | A1 | | A1 | |
| Cmpd 1 | PCI-32765 | U-2932 | A2 | U | A2 | U |
| Cmpd 1 | PD-0332991 | U-2932 | A1 | | A1 | |

TABLE 8
GCB DLBCL cell lines

| | | | Synergy Score | CI₅₀ | Synergy | |
|---|---|---|---|---|---|---|
| | therapeutic | | growth | growth | Score | CI₅₀ |
| Cmpd | agent | Cell Line | inhibition | inhibition | inhibition | inhibition |
| CAL- | Dexamethasone | DOHH-2 | A3 | T | A2 | U |
| 101 | | | | | | |
| CAL- | LEE011 | DOHH-2 | A3 | T | A2 | T |
| 101 | | | | | | |
| CAL- | PCI-32765 | DOHH-2 | A3 | S | A3 | S |
| 101 | | | | | | |
| CAL- | PD-0332991 | DOHH-2 | A3 | T | A2 | T |
| 101 | | | | | | |
| CAL- | Dexamethasone | Farage | A3 | U | A3 | S |
| 101 | | | | | | |
| CAL- | LEE011 | Farage | A1 | W | A1 | W |
| 101 | | | | | | |
| CAL- | PCI-32765 | Farage | A3 | S | A3 | S |
| 101 | | | | | | |
| CAL- | PD-0332991 | Farage | A2 | T | A1 | T |
| 101 | | | | | | |
| CAL- | Dexamethasone | OCI-Ly7 | A3 | S | A2 | T |
| 101 | | | | | | |
| CAL- | LEE011 | OCI-Ly7 | A1 | W | A1 | |
| 101 | | | | | | |
| CAL- | PCI-32765 | OCI-Ly7 | A1 | W | A1 | |
| 101 | | | | | | |
| CAL- | PD-0332991 | OCI-Ly7 | A2 | S | A2 | T |
| 101 | | | | | | |
| CAL- | Dexamethasone | SU-DHL- | A3 | S | A2 | S |
| 101 | | 10-epst | | | | |
| CAL- | LEE011 | SU-DHL- | A3 | T | A2 | T |
| 101 | | 10-epst | | | | |
| CAL- | PCI-32765 | SU-DHL- | A2 | U | A2 | U |
| 101 | | 10-epst | | | | |
| CAL- | PD-0332991 | SU-DHL- | A3 | S | A3 | S |
| 101 | | 10-epst | | | | |
| CAL- | Dexamethasone | SU-DHL- | A3 | S | A3 | S |
| 101 | | 4-epst | | | | |
| CAL- | LEE011 | SU-DHL- | A2 | S | A2 | T |
| 101 | | 4-epst | | | | |
| CAL- | PCI-32765 | SU-DHL- | A3 | S | A2 | S |
| 101 | | 4-epst | | | | |
| CAL- | PD-0332991 | SU-DHL- | A3 | S | A2 | S |
| 101 | | 4-epst | | | | |
| CAL- | Dexamethasone | SU-DHL- | A2 | T | A2 | S |
| 101 | | 6-epst | | | | |
| CAL- | LEE011 | SU-DHL- | A2 | S | A2 | S |
| 101 | | 6-epst | | | | |
| CAL- | PCI-32765 | SU-DHL- | A2 | T | A2 | T |
| 101 | | 6-epst | | | | |
| CAL- | PD-0332991 | SU-DHL- | A2 | T | A2 | T |
| 101 | | 6-epst | | | | |
| Cmpd 1 | Dexamethasone | DOHH-2 | A3 | S | A3 | S |
| Cmpd 1 | LEE011 | DOHH-2 | A3 | T | A2 | U |
| Cmpd 1 | PCI-32765 | DOHH-2 | A3 | S | A3 | S |
| Cmpd 1 | PD-0332991 | DOHH-2 | A3 | U | A2 | U |
| Cmpd 1 | Dexamethasone | Farage | A3 | U | A3 | S |
| Cmpd 1 | LEE011 | Farage | A1 | W | A1 | W |
| Cmpd 1 | PCI-32765 | Farage | A3 | S | A3 | S |
| Cmpd 1 | PD-0332991 | Farage | A2 | T | A2 | S |
| Cmpd 1 | Dexamethasone | OCI-Ly7 | A3 | S | A2 | S |
| Cmpd 1 | LEE011 | OCI-Ly7 | A2 | U | A1 | W |
| Cmpd 1 | PCI-32765 | OCI-Ly7 | A2 | W | A1 | |
| Cmpd 1 | PD-0332991 | OCI-Ly7 | A2 | T | A2 | S |
| Cmpd 1 | Dexamethasone | SU-DHL- | A3 | S | A3 | S |
| | | 10-epst | | | | |
| Cmpd 1 | LEE011 | SU-DHL- | A3 | S | A3 | S |
| | | 10-epst | | | | |
| Cmpd 1 | PCI-32765 | SU-DHL- | A3 | S | A3 | S |
| | | 10-epst | | | | |
| Cmpd 1 | PD-0332991 | SU-DHL- | A3 | S | A3 | S |
| | | 10-epst | | | | |
| Cmpd 1 | Dexamethasone | SU-DHL- | A3 | S | A3 | S |
| | | 4-epst | | | | |
| Cmpd 1 | LEE011 | SU-DHL- | A2 | S | A2 | S |
| | | 4-epst | | | | |
| Cmpd 1 | PCI-32765 | SU-DHL- | A3 | S | A3 | S |
| | | 4-epst | | | | |
| Cmpd 1 | PD-0332991 | SU-DHL- | A3 | S | A2 | S |
| | | 4-epst | | | | |
| Cmpd 1 | Dexamethasone | SU-DHL- | A3 | T | A2 | T |
| | | 6-epst | | | | |
| Cmpd 1 | LEE011 | SU-DHL- | A2 | S | A1 | |
| | | 6-epst | | | | |
| Cmpd 1 | PCI-32765 | SU-DHL- | A3 | S | A2 | S |
| | | 6-epst | | | | |
| Cmpd 1 | PD-0332991 | SU-DHL- | A2 | S | A2 | S |
| | | 6-epst | | | | |

TABLE 9
FL cell lines

| | | | Synergy | CI₅₀ | Synergy | |
|---|---|---|---|---|---|---|
| | therapeutic | | Score growth | growth | Score | CI₅₀ |
| Cmpd | agent | Cell Line | inhibition | inhibition | inhibition | inhibition |
| CAL- | Dexamethasone | KARPAS- | A3 | U | A2 | T |
| 101 | | 422 | | | | |
| CAL- | LEE011 | KARPAS- | A2 | T | A2 | T |
| 101 | | 422 | | | | |
| CAL- | PCI-32765 | KARPAS- | A3 | S | A2 | S |
| 101 | | 422 | | | | |
| CAL- | PD-0332991 | KARPAS- | A3 | S | A2 | S |
| 101 | | 422 | | | | |
| CAL- | Dexamethasone | RL | A3 | T | A2 | T |
| 101 | | | | | | |
| CAL- | LEE011 | RL | A2 | T | A1 | U |
| 101 | | | | | | |
| CAL- | PCI-32765 | RL | A2 | T | A2 | S |
| 101 | | | | | | |
| CAL- | PD-0332991 | RL | A2 | S | A2 | S |
| 101 | | | | | | |
| CAL- | Dexamethasone | WSU- | A3 | T | A3 | T |
| 101 | | NHL | | | | |
| CAL- | LEE011 | WSU- | A2 | T | A2 | S |
| 101 | | NHL | | | | |
| CAL- | PCI-32765 | WSU- | A3 | S | A3 | S |
| 101 | | NHL | | | | |
| CAL- | PD-0332991 | WSU- | A2 | S | A2 | S |
| 101 | | NHL | | | | |
| Cmpd 1 | Dexamethasone | KARPAS- | A3 | U | A2 | T |
| | | 422 | | | | |
| Cmpd 1 | LEE011 | KARPAS- | A2 | S | A2 | S |
| | | 422 | | | | |
| Cmpd 1 | PCI-32765 | KARPAS- | A3 | S | A2 | S |
| | | 422 | | | | |
| Cmpd 1 | PD-0332991 | KARPAS- | A2 | S | A2 | S |
| | | 422 | | | | |
| Cmpd 1 | Dexamethasone | RL | A3 | T | A3 | T |
| Cmpd 1 | LEE011 | RL | A2 | T | A1 | U |
| Cmpd 1 | PCI-32765 | RL | A2 | S | A2 | S |
| Cmpd 1 | PD-0332991 | RL | A2 | S | A2 | S |
| Cmpd 1 | Dexamethasone | WSU- | A3 | S | A3 | S |
| | | NHL | | | | |
| Cmpd 1 | LEE011 | WSU- | A2 | S | A2 | S |
| | | NHL | | | | |
| Cmpd 1 | PCI-32765 | WSU- | A3 | S | A3 | S |
| | | NHL | | | | |
| Cmpd 1 | PD-0332991 | WSU- | A3 | S | A2 | S |
| | | NHL | | | | |
| Cmpd 1 | PD-0332991 | WSU- | A2 | S | A3 | S |
| | | NHL | | | | |

### Example 3

### Combination Therapies of a PI3K Inhibitor and Dexamethasone

The effects of Compound 1 with dexamethasone were examined in a panel of cell lines. Cells were cultured and assayed as follows. Compound 1 was serially diluted with cell culture medium and various DMSO concentrations. The top concentration of Compound 1 exposed to cells was 3 uM for cell lines other than WSU-NHL and DOHH2. For WSU-NHL and DOHH2 cell lines, the top concentration of Compound 1 exposed to the cells was 0.3 uM. Dexamethasone was serially diluted in culture media and phosphate buffered saline (PBS). The top concentration of dexamethasone used on cells was 3 uM. Cells were at least 73% viable and were counted and diluted for plating at a density of about 92,000 cell/mL, 130 uL cells per well in 96 well plates for SUDHL6, Karpas 422, SUDHL4, WSU-NHL, RL, and DOHH2 cell lines. The SUDHL10 cell line was plated at a density of about 46,000 cells/mL, 130 uL cells per well in 96 well plates. Table 10 below provides the details of the count and dilute cells.

TABLE 10

| | | % | Desired | | mL | mL |
|---|---|---|---|---|---|---|
| Cell line | Count | viable | density | Fold | cells + | medium |
| SUDHL6 | 7.7 × 10 to 5 | 79 | 92300/mL | 8.3 | 3.6 | 26.4 |
| Karpas | 6.2 × 10 to 5 | 86 | 92300/mL | 6.7 | 4.5 | 25.5 |
| 422 | | | | | | |
| SUDHL4 | 7.1 × 10 to 5 | 77 | 92300/mL | 7.7 | 3.9 | 26.1 |
| WSU- | 2.4 × 10 to 6 | 94 | 92300/mL | 26 | 1.2 | 28.8 |
| NHL | | | | | | |
| SUDHL10 | 8.4 × 10 to 5 | 73 | 46150/mL | 18.2 | 1.7 | 28.3 |
| RL | 3.3 × 10 to 5 | 82 | 92300/mL | 3.6 | 8.3 | 21.7 |
| DOHH2 | 1.8 × 10 to 6 | 81 | 92300/mL | 19.5 | 1.5 | 28.5 |

Various concentrations of Compound 1 and various combinations of dexamethasone were added to various wells of cells. Single agents (Compound 1 or dexamethasone alone) were also added to some wells of cells. Cells were incubated with compounds for 72 hours. To assay for effects of the compounds on cell viability, a CellTiter-Glo® luminescent cell viability assay (commercially available) was used. To each plate of cells, 100 uL CellTiter-Glo reagent was added, incubated, and luminescence quantified using a spectrophotometer.

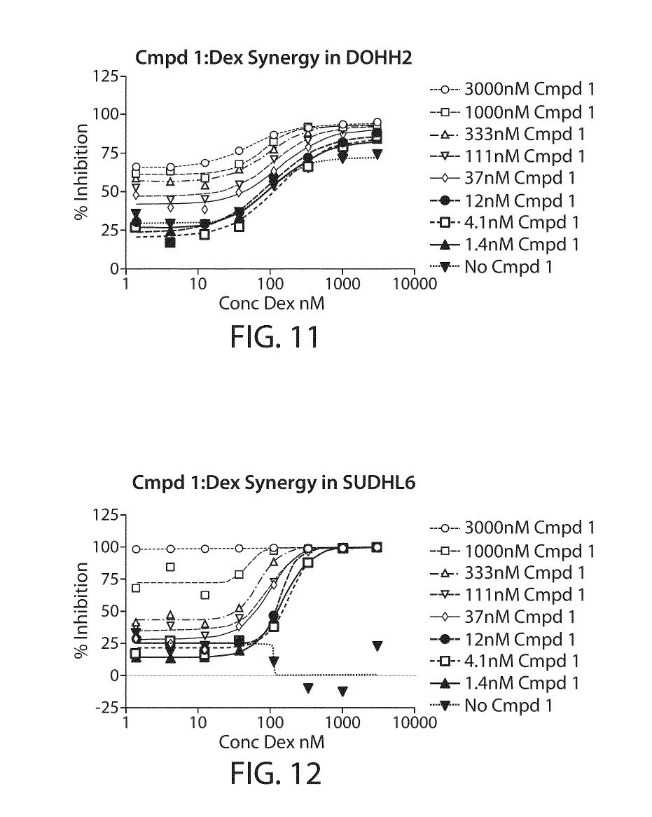

The results for cell line DOHH2 are depicted in FIG. 11 and Table 11. As shown in FIG. 11 and Table 11, synergy was observed in DOHH2 cells. Table 11 depicts combination index (CI) values for different combinations of dexamethasone/Compound 1 concentrations.

TABLE 11

| | | Dexamethasone (nM) | | | | | | | |
|---|---|---|---|---|---|---|---|---|---|
| | | 3000 | 1000 | 333.3333 | 111.1111 | 37.03704 | 12.34568 | 4.115226 | 1.371742 |
| Compound 1 | 300 | A | A | A | A | A | A | B | B |
| (nM) | 100 | A | A | A | A | A | C | A | B |
| | 33.33333 | A | A | A | A | A | D | B | B |
| | 11.11111 | A | A | A | A | C | E | E | B |
| | 3.703704 | A | A | A | A | D | E | E | B |
| | 1.234568 | A | A | A | B | E | E | E | E |
| | 0.411523 | A | A | B | C | E | E | E | E |
| | 0.137174 | A | A | A | C | E | E | E | E |

The CI₅₀ values for growth inhibition and inhibition categorized as follows: A = 0.0001 to <0.3, B = 0.3 to <0.5, C = 0.5 to <0.7, D = 0.7 to <1, and E = ≧1.

The result for cell line SUDHL6 is depicted in FIG. 12 and in Table 12. As shown in FIG. 12 and Table 12, synergy was observed in SUDHL6 cells. Table 12 depicts combination index (CI) values for different combinations of dexamethasone/Compound 1 concentrations.

TABLE 12

| | | Dexamethasone (nM) | | | | | | | |
|---|---|---|---|---|---|---|---|---|---|
| | | 3000 | 1000 | 333.3 | 111.1 | 37 | 12.3 | 4.1 | 1.4 |
| Compound 1 | 3000 | A | A | A | A | A | A | A | A |
| (nM) | 1000 | A | A | A | A | E | E | B | E |
| | 333.3 | A | A | A | A | E | E | E | E |
| | 111.1 | A | A | A | A | E | E | E | E |
| | 37 | A | A | A | A | E | E | E | E |
| | 12.3 | A | A | A | A | E | E | E | E |
| | 4.1 | A | A | A | A | C | E | B | E |
| | 1.4 | A | A | A | A | E | E | E | E |

The CI₅₀ values for growth inhibition and inhibition categorized as follows: A = 0.0001 to <0.3, B = 0.3 to <0.5, C = 0.5 to <0.7, D = 0.7 to <1, and E = ≧1.

### Example 4

### In Vivo Study of a Combination of Compound 1 with Dexamethasone

The effects of a combination of Compound 1 and dexamethasone were assessed in vivo in non-tumor bearing mice. Dexamethasone is an inducer of CYP2B6 and CYP3A4 and thus might be expected to decrease exposure of Compound 1. Tolerability of the combination was assessed in non-tumor bearing CD17. SCID female mice. Treatment was carried out for 14 days using the following treatment groups: 1) Vehicle 1 (5% NMP+95% PEG400)+Vehicle 2 (saline); 2) Compound 1 (50 mg/kg, QD, PO)+Vehicle 2; 3) Vehicle 1+Dex 1 (5 mg/kg, Q3D, IP); 4) Compound 1+Dex 1; 5) Vehicle 1+Dex 2 (1 mg/kg, Q3D, IP); 6) Compound 1+Dex 2. PEG400 refers to polyethylene glycol-400. NMP refers to N-methyl-2-pyrrolidone. Dex refers to dexamethasone. Q3D refers to administration every third day. PO refers to oral administration. IP refers to intraperitoneal administration. Plasma samples were collected on day 14 at the trough, 1, 2, 4, and 6 h post dose for pharmacokinetic analysis.

There was no significant weight loss observed with treatment in any of the six treatment groups. Also, a decrease in Compound 1 plasma exposure was observed when 50 mg/kg Compound 1 PO was dosed in combination with dexamethasone (5 mg/kg, Q3D, PO). The mean plasma concentration of Compound 1 was relatively similar between groups that were treated with 50 mg/kg Compound 1 alone and groups treated with 50 mg/kg Compound 1 plus 1 mg/kg dexamethasone (Q3D).

The effects of a combination of Compound 1 and dexamethasone were also assessed in vivo in tumor-bearing mice. In particular, tolerability of the combination treatment was assessed in DoHH2 tumor bearing CB17. SCID female mice (a follicular lymphoma subcutaneous model). Mice were treated for 14 days in the following groups: 1) Vehicle 1 (5% NMP+95% PEG400, QD PO)+Vehicle 2 (saline, Q3D, IP); 2) Compound 1 (50 mg/kg, QD, PO)+Vehicle 2; 3) Vehicle 1+Dex 1 (5 mg/kg, Q3D, IP); 4) Compound 1+Dex 1. Plasma samples were collected on day 7 (trough, 1, 2, 4, and 6 h post dose) and on day 14 (2 h post final dose).

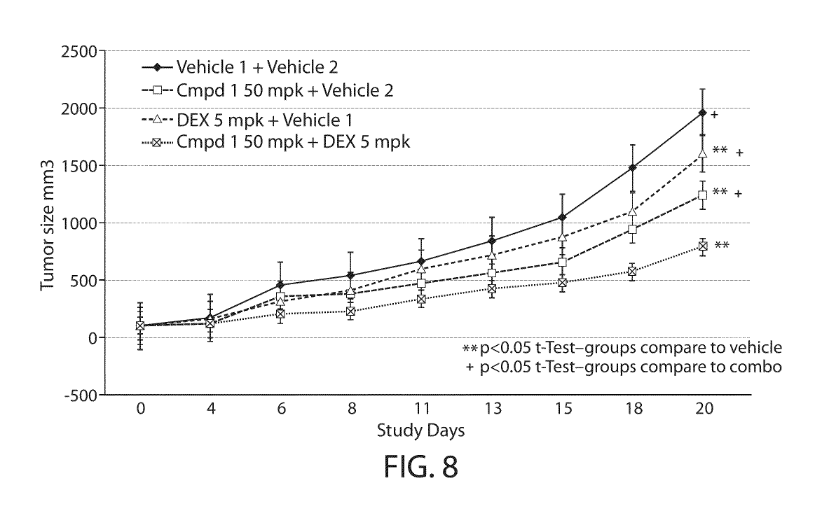

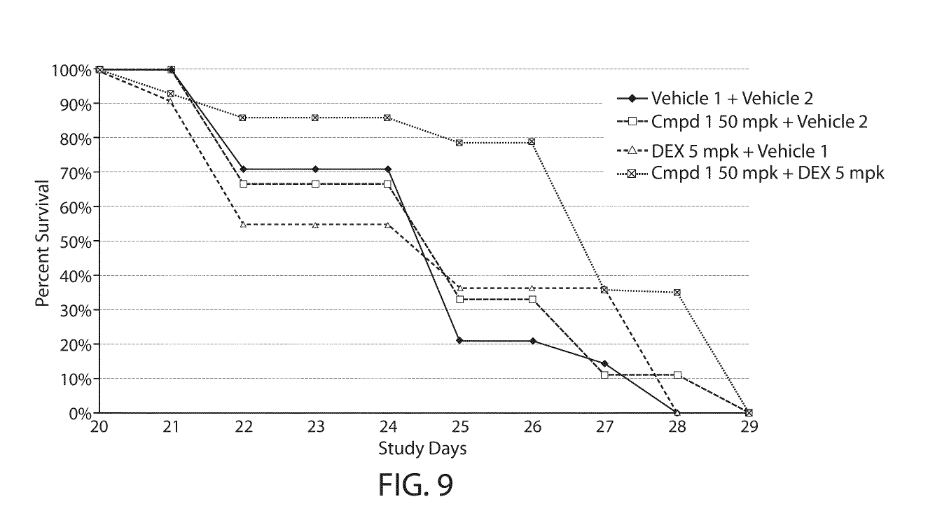

In mice treated with Compound 1 plus dexamethasone, tumor volume was lower after 12 days of treatment compared to mice treated with vehicle alone, Compound 1 alone, or dexamethasone alone. See FIG. 8. No significant weight loss was observed in any of the treatment groups after 12 days of treatment. FIG. 9 is a graph showing the effects of Compound 1 in combination with dexamethasone (DEX) on percent survival versus time for tumors to reach 3000 mm³ in the DoHH2 Follicular B cell lymphoma subcutaneous model.

These results show that Compound 1 (administered at 50 mg/kg QD in mice) in combination with dexamethasone (administered at 5 mg/kg, Q3D, IP in mice) exhibit greater tumor growth inhibition compared to either monotherapy (i.e., Compound 1 monotherapy or dexamethasone monotherapy). Also, the lack of significant changes in body weight upon co-administration of dexamethasone and Compound 1 suggest that the combination is tolerable. With respect to pharmacokinetic parameters, the degree of Compound 1 exposure was similar after administration as a single agent or in combination with dexamethasone (e.g., 1 mg/kg dexamethasone). When Compound 1 was co-administered with higher doses of dexamethasone, e.g., 5 mg/kg, Compound 1 plasma exposure decreased by about 30%. Thus, a higher dose of dexamethasone was capable of decreasing the plasma exposure of Compound 1. This result assists in the selection of a suitable dose of Compound 1 in combination with dexamethasone.

### Example 5

### Studies in Drug-Resistant DLBCL Cell Lines

Experiments were performed to examine the pathway and gene expression alterations in a cell line resistant to a PI3K inhibitor or BTK inhibitor. SU-DHL-4 is a DLBCL cell line. Gene expression analysis were performed between the resistant cell lines versus control to determine molecular signatures of resistance.

The cell line media is RMPI 10% FBS/1% Pen/strep. Doses were selected based on CTG assay IC50˜1 uM. The IC50 and 5× above the IC50 were selected for treatment.

1 uM or 5 uM from Compound 1 20 mM or ibrutinib 10 mM (Selleck#52680 Lot#7).

On Day 0, Viable SU-DHL-4 cells were plated at 2.5×10̂5 c/mL in a total on 10 mL in a 20 mm Petri dish (Corning#353003). The DMSO stock for each compound was diluted to 5 mM or 1 mM with DMSO. A 1:1000 dilution was performed into the plated cells (10 uL) for a final of 1 uM or 5 uM (final DMSO 0.01%). Cells were counted 2 times per week and cell densities were adjusted back to 2.5×10̂5 c/mL as needed. Media/Compound was replenished as needed and Media/Compound was replaced 1× per week. Compound treatment lasted for 28 days. Compound was washed out for 1 week and cells were subsequently used in assays to determine resistance after 4 weeks of treatment. At this time, cells were frozen down for each condition (Parental, DMSO, Compound 1 or ibrutinib treated) at each of the following days:

21 (on treatment)
24 (on treatment)
31 (3 days off treatment)
41 (12 days off treatment)

Once resistance was confirmed, cells were thawed from the Day 31 (3 days off treatment) under the presence of either Compound 1 at 5 uM or ibrutinib at 5 uM. The cell line pool was treated with compound for an additional 28 days prior to subcloning. Altogether, the cell line pool was under selective pressure for a total of 8 weeks.

Cell lines were subcloned at 3, 1, and 0.3 cells per well (cpw). 1:10 of conditioned medium (CM) was included in medium in addition to corresponding DMSO control or compound. During subcloning optimization experiments, the SU-DHL-4 cell line could only be subcloned in the presence of 1:10 conditioned medium. Conditioned medium (CM) was collected 4 days after splitting the parent cell line and when cells were approximately 90% confluent. Cells were spun down and the supernatant (CM) was collected down. The CM was filtered (0.22 uM) prior to adding to cells or complete medium.

All cell lines subcloned grew out. A total of 12 subclones per cell line were picked to grow and expand in 24 well plates. Parent, DMSO and ibrutinib clones were added to 400 uL of fresh media, while the Compound 1-resistant (also referred to as Compound 1-R) were added to 200 uL of fresh media. Once expanded in a 24 well plate, the 12 clones per cell line were evaluated for viability by flow using the Dead_Live kit (Invitrogen#L23101).

Clones for parent, DMSO and Compound 1-R cell lines were selected based on their P1 vs P2 distribution determined by FSCH vs SSCH scatter plot. The following clones were selected to determine Compound 1 and ibrutinib IC50's by CTG:

### Parent: 2C.3, 5F.3, 6D.3, 8C.3, 11F.3

### DMSO: 3D.3, 5G.3, 7B.3, 9C.3, 11B.3

### Compound 1-R: 2B.3, 3E.3, 7C.3, 9D.3, 11C.3

All 12 ibrutinib resistant (also referred to as ibrutinib-R or IBR-R) clones were selected to determine Compound 1 and IBR IC50's by CTG: 2B.3, 2G.3, 5C.3, 5F.3, 6B.3, 6D.3, 7B.3, 8C.3, 9G.3, 10B.3, 11D.3, 11G.3.

Once the CTG results were obtained for all 12 ibrutinib resistant clones, the following 5 ibrutinib clones were selected that showed no cross-resistance to Compound 1: 2B.3, 5C.3, 10B.3, 11D.3, 11G.3.

Samples from the following clones for the DMSO (3D.3, 5G.3, 7B.3, 9C.3, 11B.3), Compound 1-R (2B.3, 3E.3, 7C.3, 9D.3, 11C.3) and ibrutinib-R (2B.3, 5C.3, 10B.3, 11D.3, 11G.3) lines were selected for RNA extraction using the RNeasy Mini kit (Qiagen kit#74104). RNA quantification was determined using Nanodrop. 500 ng of intact RNA was submitted for subsequent gene expression analysis using RNA-seq techniques.

The raw sequence reads were aligned to Hg19 using OmicSoft ArrayStudio. The exported FPKM values were normalized relative to the housekeeping gene CFL1, which was selected using Normfinder (Anderson, 2004). Genes showing overall expression levels very close to the limit of detection were removed. Filters were applied to obtain a list of genes for each group (Compound 1-resistant and ibrutinib-resistant) which showed statistically significant adjusted p-values (<0.05) and expression level changes >1.5-fold relative to the DMSO controls. Finally, a knowledge-based filter was applied to select for genes from well-characterized cell-signaling and cancer pathways, compiled from KEGG Pathways, GO ontologies, WikiPathways and recent literature (4093 genes). See Andersen C. L. et al., Cancer Res 2004; 64 5245-5250.

The results of the RNASeq analysis showed 378 genes having at least a 1.5-fold change in mRNA levels and were significantly regulated (p<0.05), in the clones where resistance to a PI3K inhibitor such as compound 1 was generated. Out of the 378 genes, 217 were up-regulated and 161 were down-regulated as compared to the reference sample e.g. control.

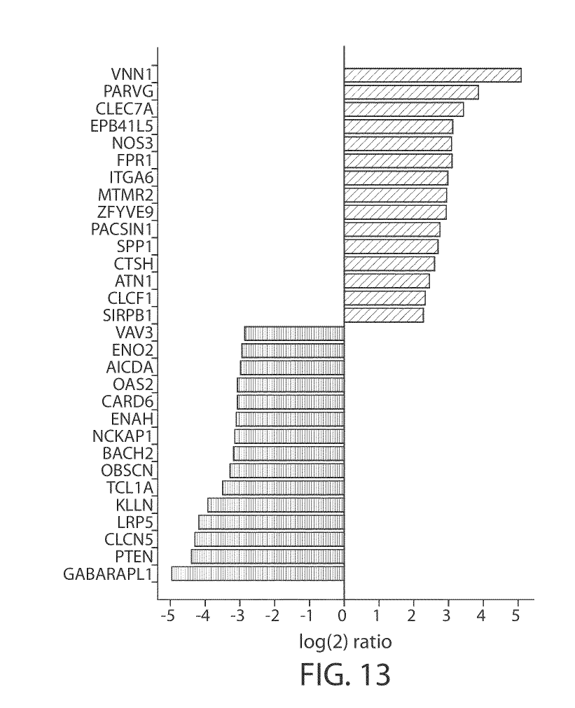

The top 15 upregulated or downregulated genes (having at least a 2-fold change in RNA levels) in the Compound 1-resistant cells are shown in FIG. 13. These genes are therefore useful as biomarkers indicating resistance to a PI3K inhibitor such as Compound 1. Specifically, upregulation of one or more of VNN1, PARVG, CLEC7A, EPB41L5, NOS3, FPR1, ITGA5, MTMR2, ZFYVE9, PACSIN1, SPP1, CTSH, ATN1, CLCF1, and SIRPB1 is indicative of resistance to a PI3K inhibitor such as Compound 1. In addition, downregulation of one or more of VAV3, ENO2, AICDA, CARD6, DNAH, NCKAP1, BACH2, OSBCN, TCL1A, KLLN, LRP5, CLCN5, PTEN, and GABARAPL1 SIRPB1 is indicative of resistance to a PI3K inhibitor such as Compound 1.

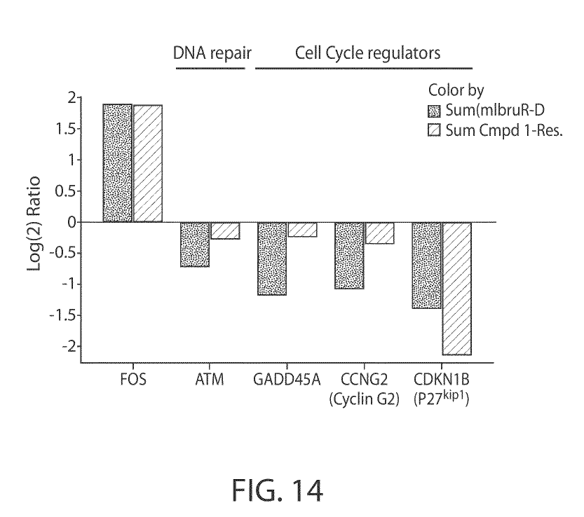

Also, the fold change in expression level of several genes involved in DNA repair or cell cycle regulation/cell proliferation was analyzed and shown in FIG. 14. FOS, a gene that promotes cell proliferation, is elevated in cells resistant to Compound 1 or ibrutinib. ATM, a DNA repair gene, is down-regulated in the resistant cells. The cell cycle checkpoint regulators GADD45A, CCNG2, and CDKN1B, are down-regulated in resistant cells as well. Dysregulation of cell proliferation, DNA repair, and cell cycle checkpoint markers indicate an unchecked increased proliferation in resistant clones. This result indicates that cells resistant to Compound 1 are chemosensitized which can allow the use of less toxic doses of chemotherapy in combination with Compound 1 or ibrutinib.

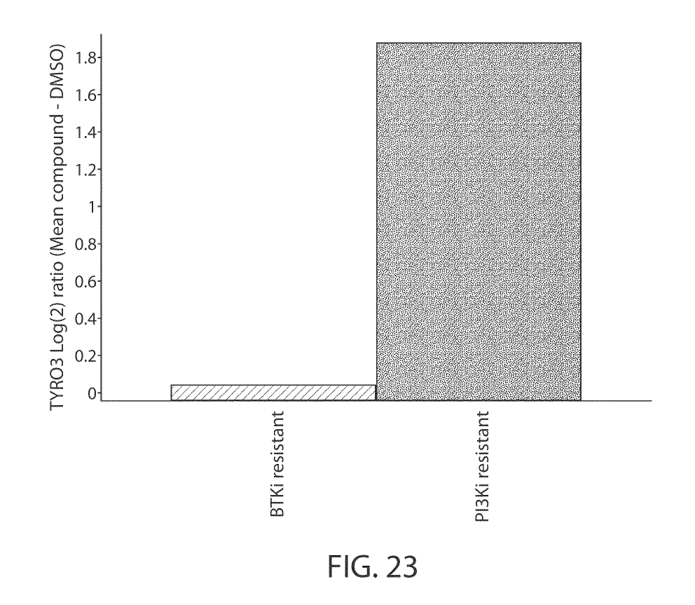

In addition, FIG. 23 is a bar chart showing the log (2) fold change of TYRO3 in Compound 1 resistant and ibrutinib resistant clones. TYRO3 is significantly up-regulated (3.5 fold) in the Compound 1 DHL-4 resistant clones as compared to control (i.e., DMSO). This indicates that Compound 1 can be combined with a TAM inhibitor, e.g., a TYRO3 inhibitor, in the treatment of cancer.

Further, in Compound 1-resistant cells, the greatest differential regulation was found in the following nine pathways: apoptotic signaling pathway, cellular response to cytokine stimulus pathway, cytokine mediated signaling pathway, endocytosis pathway, innate immune response signaling pathway, MAPK pathway, neurotrophin TRK receptor signaling pathway, PI3K pathway, and TLR pathway. The particular genes within each pathway that were differentially regulated in the Compound 1-resistant cells are given below.

Regulated genes in the apoptotic signaling pathway included VAV3, AIM2, MAPK8, SGPL1, KLLN, PTEN, TCTN3, SMNDC1, PDCD4, BNIP3, APBB1, HIPK3, PAK1, NR4A1, DDIT3, CCNB1IP1, TRAF3, PACS2, LITAF, MAPK3, BCL7C, CSNK2A2, ALDOC, KANK2, DNASE2, DMPK, EIF2AK3, ELMO2, TIAM1, ITGB2, PRAME, BIK, TSPO, GRAMD4, SATB1, ARHGEF3, IFT57, MFSD10, MZB1, PIM1, FOXO3, TRAF3IP2, TIAM2, TNFRSF10B, TNFRSF10D, SCRIB, PLEC, FGD3, GSN, AIFM1, and ARHGEF6.

Regulated genes in the cellular response to cytokine stimulus pathway included ISG15, PTAFR, AIM2, IFITM2, UBE2L6, SELPLG, OAS1, OAS2, RPS6KA5, TRAF3, NEDD4, BBS4, PML, MAPK3, MT1X, FASN, IFI30, NUMBL, RPL13A, STAT1, PTPN1, HYAL2, WNT5A, FLNB, MME, IRF2, KPNA5, HSPA5, and IRAK1.

Regulated genes in the cytokine mediated signaling pathway included ISG15, PTAFR, AIM2, IFITM2, UBE2L6, OAS1, OAS2, RPS6KA5, TRAF3, NEDD4, BBS4, PML, MAPK3, IFI30, NUMBL, STAT1, PTPN1, FLNB, IRF2, KPNA5, and IRAK1.

Regulated genes in the endocytosis pathway included LDLRAP1, NR1H3, LRP5, CORO1C, TYRO3, NEDD4, MAPK3, EPN2, RARA, ABCA7, TRIP10, ELMO2, CLTCL1, MAPKAPK3, TNK2, MYO6, and SCRIB.

Regulated genes in the innate immune response signaling pathway included ISG15, FGR, VAV3, TXNIP, IFI16, AIM2, CD55, CD46, MAPK8, PTEN, CHUK, UBE2L6, DAK, PAK1, CDKN1B, NR4A1, FRS2, RPS6KA5, TRAF3, MAPK3, MYO1C, DUSP3, TBKBP1, PRKCA, ELMO2, ITGB2, MAPK11, PRKAR2A, MAPKAPK3, TLR9, TREM1, FOXO3, FYN, TAB2, ULBP2, ULBP3, SRPK2, ZC3HAV1, LY96, TRIM32, C5, CYBB, and IRAK1.

Regulated genes in the MAPK pathway (defined by KEGG criteria) included MAPK8, CHUK, DUSP5, PAK1, NR4A1, DDIT3, FGF14, RPS6KA5, MAPK8IP3, MAPK3, DUSP3, PRKCA, DUSP2, ZAK, ATF4, MAPK12, MAPK11, MAPKAPK3, FLNB, TAB2, GNAl2, and FLNA. Regulated genes in the MAPK pathway (defined by Wild criteria) included PRKCZ, MAPK8, DUSP5, PAK1, ACVR1B, NR4A1, DDIT3, RPS6KA5, MAPK8IP3, MAPK3, ZAK, ATF4, MAPK12, TAB2, GNAl2, HSPA5, and FLNA.

Regulated genes in the neurotrophin TRK receptor signaling pathway included VAV3, MAPK8, DDIT4, PTEN, CHUK, CDKN1B, NR4A1, FRS2, RPS6KA5, MAPK3, DUSP3, PRKCA, TIAM1, MAPK12, MAPK11, PRKAR2A, MAPKAPK3, ARHGEF3, FOXO3, FYN, TIAM2, FGD3, ARHGEF6, and IRAK1.

Regulated genes in the PI3K pathway included PRKCZ, PIK3R3, LAMC1, DDIT4, PTEN, CHUK, GNB3, CDKN1B, NR4A1, EIF4B, FGF14, TCL1A, MAPK3, GNGT2, PRKCA, INSR, LPAR2, ATF4, SPP1, PRL, FOXO3, MYB, and CREB5.

Regulated genes in the TLR pathway included PIK3R3, MAPK8, CHUK, TRAF3, MAPK3, SARM1, STAT1, RBCK1, MAPK12, MAPK11, TLR9, SPP1, TREM1, TAB2, LY96, and IRAK1.

Overall, regulated genes in the Compound 1-resistant cells included ISG15, PRKCZ, ZBTB17, PINK1, LDLRAP1, FGR, PTAFR, PLK3, PIK3R3, ZFYVE9, JUN, CTH, VAV3, SORT1, NOTCH2, TXNIP, HIST2H4A, MLLT11, S100A13, IFI16, AIM2, SLAMF7, FCGR2B, LAMC1, PIK3C2B, PFKFB2, CD55, CD46, PROX1, ENAH, OBSCN, EGLN1, CAMK1D, COMMD3-BMI1, MAPK8, SRGN, SGPL1, DDIT4, KLLN, PTEN, LIPA, HHEX, HELLS, TCTN3, ENTPD1, BLNK, FRAT1, FRAT2, AVPI1, CHUK, BTRC, LDB1, NT5C2, SMNDC1, DUSP5, SMC3, PDCD4, SHOC2, CASP7, BAGS, BNIP3, IFITM2, SMPD1, APBB1, HIPK3, CD59, RAG1, LRP4, NR1H3, PTPRJ, UBE2L6, DTX4, DAK, FERMT3, PPP2R5B, CD248, CLCF1, LRP5, PAK1, GAB2, MTMR2, TRPC6, IL10RA, AMICA1, CD3E, THY1, CCND2, GNB3, ENO2, ATN1, AICDA, CLEC7A, GABARAPL1, CDKN1B, PRICKLE1, RAPGEF3, WNT10B, GPD1, ACVR1B, NR4A1, EIF4B, MAP3K12, LRP1, DDIT3, FRS2, E2F7, SELPLG, CORO1C, OAST, OAS2, HRK, PXN, HNF1A, TSC22D1, FGF14, CCNB1IP1, ZNF219, ARHGAP5, PRKCH, ESR2, DPF3, MLH3, FOS, RPS6KA5, TCL6, TCL1A, TRAF3, TNFAIP2, JAG2, BRF1, PACS2, SLC12A6, SPRED1, PLCB2, TYRO3, SHF, MYO5A, RAB27A, NEDD4, BBS4, PML, CTSH, IL16, ADAMTSL3, NMB, IGF1R, ALDH1A3, PIGQ, MAPK8IP3, LITAF, MYH11, DCUN1D3, LAT, MAPK3, BCL7C, MYLK3, MT1X, NLRC5, CSNK2A2, CKLF, NQO1, CBFA2T3, MYO1C, P2RX1, NLRP1, TNFSF13, EPN2, VTN, SARM1, ALDOC, CDK5R1, CCL5, RARA, DUSP3, TBKBP1, HOXB3, GNGT2, TMEM100, PECAM1, PRKCA, UNC13D, ASPSCR1, FASN, SLC16A3, SETBP1, SMAD7, ABCA7, TRIP10, INSR, FCER2, KANK2, DNASE2, NOTCH3, IFI30, HOMER3, MEF2B, LPAR2, PLEKHF1, NFKBID, SPRED3, MAP3K10, LTBP4, NUMBL, ERCC1, GIPR, DMPK, SPHK2, RPL13A, FPR1, TP53I3, SLC8A1, SPRED2, MEIS1, RTKN, EIF2AK3, DUSP2, INPP4A, EPB41L5, CCNT2, ITGA6, ZAK, TTN, NCKAP1, STAT1, IKZF2, STK36, DNER, RBCK1, SIRPB1, JAG1, ADA, ELMO2, PTPN1, BMP7, PMEPA1, MYT1, JAM2, TIAM1, ETS2, ITGB2, ADARB1, CLTCL1, PRAME, BCR, CBY1, ATF4, BIK, TSPO, PARVG, GRAMD4, MAPK12, MAPK11, MAPK8IP2, OXTR, SATB1, PRKAR2A, MST1R, HYAL2, MAPKAPK3, TLR9, ITIH4, WNT5A, ARHGEF3, FLNB, MITF, NFKBIZ, IFT57, MYLK, MGLL, PLXND1, CHST2, MME, HEST, TNK2, DGKQ, FGFRL1, SH3BP2, MFSD10, RHOH, TEC, ARHGAP24, SPP1, PKD2, PLA2G12A, IRF2, C1QTNF3, CARD6, IL6ST, PDE4D, ERBB2IP, OCLN, NAIP, FCHO2, SEMA6A, CAMLG, MZB1, TMEM173, HBEGF, CCNG1, TFAP2A, CD83, PRL, HIST1H1C, BTN3A2, PACSIN1, PPARD, CDKN1A, PIM1, TREM1, CRIP3, SUPT3H, TNFRSF21, MYO6, BACH2, FOXO3, TRAF3IP2, FYN, KPNA5, VNN1, MYB, CITED2, TAB2, ULBP2, ULBP3, TIAM2, FNDC1, PLG, THBS2, GNAl2, HOXA5, HOXA13, CREB5, PDE1C, SAMD9, SRPK2, BCAP29, ZC3HAV1, NOS3, PRKAG2, CLDN23, TNFRSF10B, TNFRSF10D, GPR124, LY96, E2F5, RRM2B, SCRIB, PLEC, PLGRKT, IL11RA, SHB, PIP5K1B, TJP2, FGD3, TNFSF15, TRIM32, C5, GSN, HSPA5, PBX3, CACFD1, CYBB, CLCN5, OCRL, BCORL1, ELF4, AIFM1, GPC4, PHF6, ARHGEF6, MTM1, MTMR1, IRAK1, FLNA, RPL10, F8, MTCP1, and CD24.

In ibrutinib-resistant cells, the greatest differential regulation was found in the following nine pathways: apoptotic signaling pathway, cellular response to cytokine stimulus pathway, FOXO pathway, innate immune response pathway, MAPK pathway, neurotrophin TRK receptor signaling pathway, PI3K pathway, positive regulation of apoptosis pathway, and T cell activation pathway. The particular genes within each pathway that were differentially regulated in the ibrutinib-resistant cells are given below.

Regulated genes in the apoptotic signaling pathway included ITGB3BP, GADD45A, SH3GLB1, PKN2, VAV3, DRAM2, MDM4, MAPK8, PDCD4, RTN3, BIRC2, ATM, ARHGEF12, ING4, FGD4, CSRNP2, DDIT3, ITM2B, CCNB1IP1, SOS2, SGPP1, GPR65, BCL2A1, RHOT2, AKTIP, GABARAP, RFFL, MAP2K6, PSMG2, PSMA8, PMAIP1, BCL2, MAP1S, BRE, EIF2AK3, RALB, CXCR4, DAPL1, CSRNP3, TIAM1, ITGB2, CHEK2, SATB1, IFT57, MFSD10, RNF144B, PIM1, TIAM2, PPP3CC, BNIP3L, RIPK2, TP53INP1, RAD21, PLEC, TRAF1, MAGEH1, ARHGEF9, OGT, AIFM1, and ARHGEF6.

Regulated genes in the cellular response to cytokine stimulus pathway included KRAS, IRAK4, NEDD4, BBS4, CIITA, MT2A, MT1X, FASN, UBE2E1, WNT5A, MME, IRF2, IRF1, NFIL3, HSPA5, and RBMX.

Regulated genes in the FOXO signaling pathway included GADD45A, MAPK8, ATM, CDKN1B, KRAS, PRKAG1, PCK2, SOS2, GABARAP, NLK, SMAD2, SMAD4, PIK3CA, CCNG2, BRAF, PRKAG2, and FBXO25.

Regulated genes in the innate immune response pathway included VAV3, MAPK8, UNC93B1, BIRC2, KLRG1, CDKN1B, KRAS, IRAK4, MAP2K6, MALT1, BCL2, CEBPG, PELI1, TANK, ITGB2, TLR9, PIK3CA, RICTOR, MAP3K1, AKIRIN2, FYN, ZC3HAV1, RIPK2, C5, TUBB4B, and TAB3.

Regulated genes in the MAPK signaling pathway (Kegg criteria) included GADD45A, MAPK8, DUSP5, DUSP16, KRAS, DDIT3, FGF14, SOS2, PPM1A, NLK, NF1, MAP2K6, DUSP2, ATF4, RAPGEF2, MAP3K1, RASA1, MAP3K4, BRAF, and PPP3CC.

Regulated genes in the MAPK pathway (Wild criteria) included GADD45A, MAPK8, DUSP5, KRAS, DDIT3, SOS2, PPM1A, NLK, NF1, ATF4, MAP3K1, RASA1, MAP3K4, BRAF, PPP3CC, and HSPA5.

Regulated genes in the neurotrophin TRK receptor signaling pathway included ITGB3BP, VAV3, MAPK8, DDIT4, ARHGEF12, CDKN1B, KRAS, FGD4, SOS2, MAG, RALB, TIAM1, PRKCI, PIK3CA, RICTOR, FYN, TIAM2, BRAF, RIPK2, ARHGEF9, and ARHGEF6.

Regulated genes in the PI3K pathway included PKN2, CREB3L4, ITGB1, DDIT4, PDGFD, CDKN1B, KRAS, FGF14, PCK2, SOS2, PPP2R5C, BCL2, COL4A4, ATF4, PIK3CA, CREB5, and TSC1.

Regulated genes in the positive regulation of apoptosis pathway included ITGB1, ATM, ING4, LRP6, WNT10B, DDIT3, NFATC4, SAV1, NF1, MAP2K6, PMAIP1, CSRNP3, CAPN10, ATF4, WNT5A, PRKCI, FNIP1, GPLD1, CNR1, HOXA5, BNIP3L, RIPK2, and NOTCH1.

Regulated genes in the T cell activation pathway included CD48, BATF, RAB27A, NEDD4, MALT1, BCL2, CXCR4, ICOSLG, ITGB2, SATB1, CBLB, IRF1, FYN, RIPK2, ATP7A, and ELF4.

### Example 6

### STK11 Copy Number Loss in Patient with CLL

A patient diagnosed with CLL was treated by a monotherapy of Compound 1 (25 mg bid) in a clinical trial. Serum samples of the patient were collected at various points in the treatment. The copy number of STK11 in the serum samples was determined by CytoScan (Affymetrix). The results are described below:
At C1D1 (cycle 1, day 1), Absolute lymphocyte count (ALC)=257, wildtype STK11 was detected;
At C3D1 (cycle 3, day 1), patient achieved partial response;
At C5D1 (cycle 5, day 1), ALC=134, STK11 copy loss was detected;
After C7 (cycle 7), patient progressed.

The result indicates that STK11 copy number loss can be acquired and can be a contributing factor in acquired resistance to the treatment of Compound 1.

### Example 7

### Genomic Profiling Protocol

Genomic DNA can be profiled by one or more of CytoScan microarray analysis, targeted NexGen Sequencing and Sanger Sequencing. The protocols for these methods are described herein. CytoScan microarray analysis on genomic DNA can be used to determine copy number alterations (CNAs), such as copy number loss or gain. NexGen Sequencing on genomic DNA can be used to determine gene mutations. Sanger sequencing on genomic DNA can be used to determine IgHV mutation status. Results from genomic DNA profiling were used to assess whether genomic alterations in individuals treated with Compound 1 predict responsiveness or resistance to treatment with Compound 1 and whether genomic alterations occur with acquired resistance.

Preparation of DNA Sample

Peripheral whole blood samples were collected from CLL patients being treated with Compound 1. Genomic DNA was extracted from Cycle 1 Day 1 blood samples of 43 CLL patients, using QIAamp DNA Blood Midi kit (Qiagen, cat #51185) according to the manufacturer's protocol.

CytoScan Array Data Analysis

CytoScan array analysis allows for genome-wide identification of copy number changes. The CytoScan HD array has 750,000 SNP probes and 1.9 million non-polymorphic probes, providing even copy number coverage across the genome. The CytoScan HD array also has intragenic coverage of 36,000 RefSeq genes.

Genomic DNA samples were applied for hybridization to Affymetrix CytoScan HD arrays according to the manufacturer's manual. CEL files were analyzed using Affymetrix software for initial quality control, followed by the use of Nexus 7.5 software (BioDiscovery, Inc.) for copy number and allelic analysis. Following the profiling of copy number variations (CNVs) in each sample, Nexus 7.5 software was used to identify the CNVs that are significantly different between patients who responded to treatment with Compound 1 and patients who did not (differential frequency>25%; p<0.05). Copy number variances were initially assessed with Nexus default setting (500 kb minimum LOH) for the first set of 43 samples. In order to efficiently utilize allelle information, the segmentation window was changed to minimum LOH at 2 kb. Furthermore, gains that are not covered by an allelic event were filtered out. The cancer-related genes were annotated based on the Cancer Gene Census database. Association between CNVs and clinical features were assessed by Fisher's exact test.

Targeted NexGen Sequencing and Data Analysis

Protocols for NexGen sequencing and hybrid capture are described in Gnirke et al. (Nat Biotechnol. 27(2): 182-189, 2009). In these experiments, hybrid capture approach was used with the OncoGxOne leukemia/lymphoma panel (GeneWiz) containing 374 genes, including all 4 PI3K isoforms, BTK, and PLCγ. Illumina HiSeq sequencing was used.

Agilent SureSelect solutions were used for the targeted DNA capture of a panel of genes. According to the manufacturer's protocol, DNA-Seq libraries were constructed and sequenced on Illumina HiSeq 2500 using 100 bp paired-end reads. FASTQ files were aligned by the OSA algorithm in Omicsoft Array Studio to generate BAM files with default parameter setting. Non-synonymous mutations including single-nucleotide variations (SNVs), insertions/deletions (InDels) and stop codon gain/loss were detected by Array Studio's mutation calling algorithm with the mutational allelic frequency (MAF) threshold set to be above 0.1. Detected SNVs were annotated with RefSeq gene model along with the Single Nucleotide Polymorphism Database (dbSNP), Catalogue Of Somatic Mutations In Cancer (COSMIC), and ClinVar databases to highlight the known germline polymorphisms and the clinically relevant somatic mutations. The putative somatic mutations were determined by eliminating the SNVs that are known human single-nucleotide polymorphisms (SNPs) archived in dbSNP and ClinVar and that were detected in normal control samples. KEGG and MetoCore Pathway Database was used to define the signaling pathways that are significantly enriched with the genes that have somatic mutations as detected in the CLL patients of this study (p<0.05). Association between mutations and clinical features were assessed by Fisher's exact test.

### Example 8

### Baseline Mutation Frequency in CLL

Using the targeted NexGen sequencing method described in Example 7, the baseline mutation frequency of CLL patients in the Compound 1-treated patient population was determined, prior to treatment of the patients with Compound 1 (Table 13). The patients were treated as part of a clinical trial (identifier NCT01476657) which is a phase 1 study in patients with advanced hematologic malignancies. Many genes that were previously described in the literature as being commonly mutated in CLL were found to be mutated in the Compound 1-treated population, suggesting that the Compound 1-treated population is similar to what has been described for CLL (Landau et al. Cell 152, 714, 2013). The TP53 mutation rate was twice what has been previously reported. This suggests that the Compound 1-treated population has more aggressive disease than previously published cohorts.

TABLE 13
Comparison of Compound 1-treated baseline
mutation frequency with literature.

| | Landau et al. (%) | Compound 1-treated (%) | |
|---|---|---|---|
| Gene | N = 160 | N = 55 | |
| SF3B1 | 14 | 9 | (5/55) |
| TP53 | 13 | 24 | (13/55) |
| NOTCH1 | 10 | 20 | (11/55) |
| MYD88 | 8 | 5 | (3/55) |
| ATM | 8 | 11 | (6/55) |
| XPO1 | 4 | 9 | (5/55) |
| POT1 | 3 | 0 | |
| NRAS | 3 | 0 | |
| BCOR | 3 | 0 | |
| KRAS | 2 | 0 | |
| MED12 | 2 | 5 | (3/55) |
| DDX3X | 2 | 0 | |
| FBXW7 | 3 | 2 | (1/55) |

In addition, it was found that the average number of baseline mutations per patient was relatively similar among patients who show a complete or partial response to Compound 1 treatment, compared to non-responders (e.g., patients with stable disease or progressive disease). The average number of baseline mutations per patient was also relatively similar among R/R and Tx-naïve patients. Thus, the difference between a mutation profile predictive of response and a mutation profile predictive of non-response seems not to be the total number of mutations, but the identity of the mutations.

### Example 9

### Baseline Copy Number Changes in CLL

Using the CytoScan array analysis, a genome-wide scan for baseline copy number changes in the Compound 1-treated patient population was performed, prior to treatment of the patients with Compound 1. Copy number losses were in association with del(11q), del(13q), and del(17p). In particular, genetic changes observed at baseline included del(13q14), and del(11q22-23), del(17p13). Del(8p) was also observed in the R/R population (6.5%) but not the Tx-naïve population. Also, copy number gain was observed in association with trisomy 12. In summary, a copy gain at chromosome 12, trisomy 12; a copy loss at chromosome 11q22-23, del(11q22-23); a copy loss at chromosome 13q14, del(13q14); and a copy loss at chromosome 17p, del(17p) were observed.

### Example 10

### Copy Number Alterations in CLL

Using the CytoScan microarray analysis for genome wide as described in Example 7, copy number alterations and losses of heterozygosity were compared between responders and nonresponders to treatment with Compound 1. This analysis was performed in the same CLL patient population as was assessed at baseline in Examples 8 and 9. Tumor response to drug is defined by SD/PD (Stable Disease/Progressive Disease, i.e., non-responders) and CR/PR (Complete Remission/Partial Remission, i.e., responders). Also included in the responder group were PR patients with lymphocytosis. See Brown, J. R. (2014) Blood, 123(22):3390-3397 and Chesson, B. D. et al. Journal of Clinical Oncology, 30(23):2820-2822 for additional information regarding classifications of patient responsiveness.

The genes for which differences between groups were significant included BRAF, CTNNB1, FHIT, IRF4, MITF, MN1, NF2, RET, STK11, TSC2, RB1, RANBP17, FGFR3, GMPS, and WHSC1. Summaries of genetic alterations (500 kb minimum LOH) that were high in the SD/PD group or low in the SD/PD group are provided in Tables 14 and 15 respectively.

TABLE 14
Summary of Changes High in SD/PD group

| High in SD/PD | Count of Region |
|---|---|
| Allelic Imbalance | 2 |
| CN Gain | 30 |
| CN Loss | 37 |
| LOH | 66 |
| Total | 135 |

TABLE 15
Summary of Changes Low in SD/PD groups

| Low in SD/PD | Count of Region |
|---|---|
| CN Gain | 6 |
| CN Loss | 6 |
| LOH | 56 |
| Total | 68 |

Table 16 shows copy number alterations for cancer genes with a higher frequency in SD/PD (i.e., non-responder) patients compared with CR/PR (i.e., responder) patients. BRAF, CTNNB1, FHIT, IRF4, MITF, MN1, and NF2 had increased frequency of copy number gain in SD/PD patients relative to CR/PR patients. NF2, RET, STK11, and TSC2 had increased frequency of copy number loss in SD/PD patients relative to CR/PR patients. RB1 showed a higher frequency of loss of heterozygosity in SD/PD patients relative to CR/PR patients.

The results presented in Table 16 indicate that copy number gain in each of BRAF, CTNNB1, FHIT, IRF4, MITF, MN1, and NF2 is associated with or predictive of nonresponsiveness or resistance (e.g., acquired resistance) of a cancer (e.g., a CLL) to a PI3K inhibitor (e.g., Compound 1). The results presented in Table 16 also indicate that copy number loss in each of NF2, RET, STK11, and TSC2 is associated with or predictive of nonresponsiveness or resistance (e.g., acquired resistance) of a cancer (e.g., a CLL) to a PI3K inhibitor (e.g., Compound 1). The results presented in Table 16 further suggest loss of heterozygosity in RB1 is associated with or predictive of nonresponsiveness or resistance (e.g., acquired resistance) of a cancer (e.g., a CLL) to a PI3K inhibitor (e.g., Compound 1).

TABLE 16
Cancer genes with higher frequency in SD/PD

| CN gain | CN loss | LOH |
|---|---|---|
| BRAF | NF2 | RB1 |
| CTNNB1 | RET | |
| FHIT | STK11 | |
| IRF4 | TSC2 | |
| MITF | | |
| MN1 | | |
| NF2 | | |

Table 17 shows copy number alterations for cancer genes with a lower frequency in SD/PD patients compared with CR/PR patients. Copy number gain in RANBP17 had a lower frequency in SD/PD (i.e., nonresponder) patients compared with CR/PR (i.e., responder) patients. Also, loss of heterozygosity in FGFR3, GMPS, and WHSC1 had a lower frequency in SD/PD (i.e., nonresponder) patients compared with CR/PR (i.e., responder) patients.

These results presented in Table 17 indicate that copy number gain in RANBP17 is associated with responsiveness or lack of resistance (e.g., acquired resistance) of a cancer (e.g., a CLL) to a PI3K inhibitor (e.g., Compound 1). These results presented in Table 17 also indicate that loss of heterozygosity in each of FGFR3, GMPS, and WHSC1 is associated with or predictive of responsiveness or lack of resistance (e.g., acquired resistance) of a cancer (e.g., a CLL) to a PI3K inhibitor (e.g., Compound 1).

TABLE 17
Cancer genes w/Lower frequency in SD/PD

| CN gain | CN loss | LOH |
|---|---|---|
| RANBP17 | | FGFR3 |
| | | GMPS |
| | | WHSC1 |

In order to get more specific LOH calls and increase confidence of copy number calling, the CNV data was analyzed with a different segmentation window (minimum LOH is 2 kb).

Table 18 shows copy number alterations for cancer genes with a higher frequency of loss in SD/PD patients compared with CR/PR patients (>25% frequency difference, p<0.05). Loss of CBFA2T3, YWHAE, TP53, PER1 and GAS7 are accompanied with an allelic event (allele imbalance or loss of heterozygosity); while only copy number loss was found in STK11, FSTL3 and USP6. Among all patients, loss of YWHAE, STK11, TP53, FSTL3 and USP6 are significantly more frequent in SD/PD patients compared with CR/PR patients. Within the refractory/relapsed cohort (R/R), loss of STK11, TP53, PER1, GAS7 and FSTL3 occur more significantly in SD/PD patients compared to CR/PR patients.

The results presented in Table 18 indicate loss of YWHAE, STK11, TP53, FSTL3 and USP6 are associated with or predictive of nonresponsiveness or resistance (e.g., acquired resistance) of a cancer (e.g., a CLL) for all patients to a PI3K inhibitor (e.g., Compound 1). The results presented in Table 18 further suggest loss of STK11, TP53, PER1, GAS7 and FSTL3 is associated with or predictive of nonresponsiveness or resistance (e.g., acquired resistance) of a cancer (e.g., a CLL) among refractory or relapsed patients to a PI3K inhibitor (e.g., Compound 1).

Table 19 shows copy number alterations for cancer genes with a differential frequency of loss in nodal responders compared to nodal nonresponders (>25% frequency difference, p<0.05). For these three cancer genes, copy number loss was identified without coverage of an allelic event. TSC1 and NF2 are more frequently loss in nodal nonresponders compared to nodal responders, whereas EGFR loss is found significantly frequently lost in nodal responders.

The results presented in Table 19 indicate that loss of EGFR is associated with or predictive of responsiveness or lack of resistance (e.g., acquired resistance) of a cancer (e.g., a CLL) for all patients to a PI3K inhibitor (e.g., Compound 1).

TABLE 18
Cancer genes w/higher frequency in SD/PD

| | | | Fisher's | Fisher's |
|---|---|---|---|---|
| | | | exact | exact |
| | | Allelic | (all patients | (R/R only |
| Loss | Chr | Event | n = 56) | n = 46) |
| CBFA2T3 | 16q24 | Yes | 0.174 | 0.1378 |
| YWHAE | 17p13 | Yes | 0.0459* | 0.0626 |
| STK11 | 19p13 | | 0.0459* | 0.0042** |
| TP53 | 17p13 | Yes | 0.0371* | 0.0274* |
| PER1 | 17p13 | Yes | 0.0696 | 0.0274* |
| GAS7 | 17p13 | Yes | 0.0696 | 0.0274* |
| FSTL3 | 19p13 | | 0.006** | 0.0022** |
| USP6 | 17p13 | | 0.0459* | 0.0626 |
| MAP2K4 | 17p12 | | 0.0696 | 0.0274* |

TABLE 19
Cancer genes w/differential frequency between
nodal responders and nodal nonresponders

| Loss | Higher Freqency | Fisher's exact |
|---|---|---|
| TSC1 | Noresponder | 0.09 |
| NF2 | Nonresponder | 0.057 |
| EGFR | Responder | 0.035* |

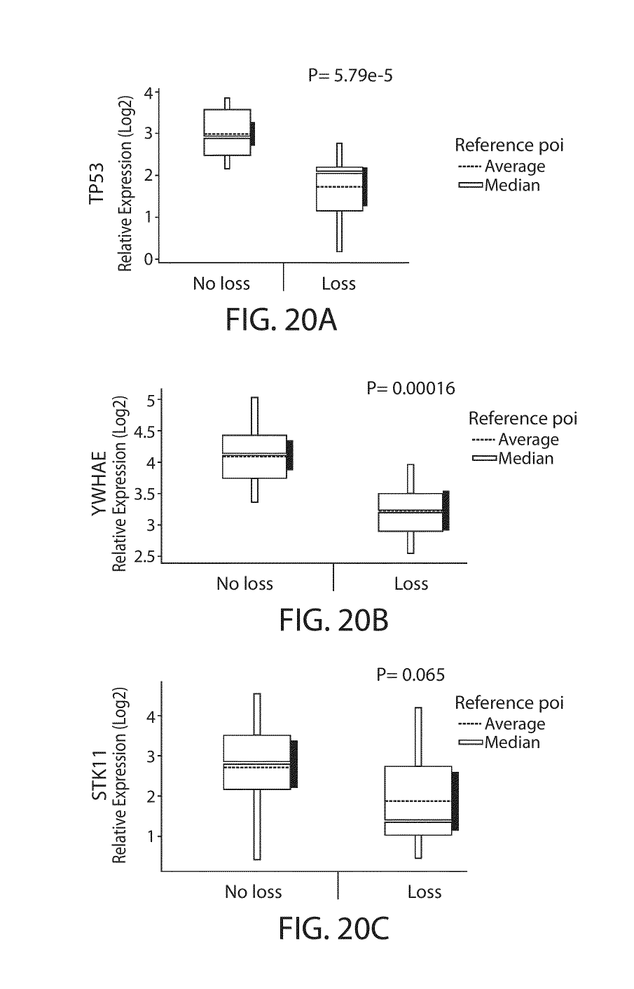

Validation of copy number losses of several genes was performed by RNAseq, e.g., as described in Wong et al. Nature Reviews Genetics 10.1(2009):57-63, incorporated herein by reference. The relative expression levels of TP53, YWHAE, and STK11 are reduced in patients having a loss in copy number, compared to patients with no loss in copy number, as shown in FIGS. 20A, 20B, and 20C.

### Example 11

### Relationship Between Mutational and Copy Number Variation Frequencies and Responses

The relationship between certain genetic alterations (e.g., exonic deletions) and patient responsiveness to Compound 1 was analyzed.

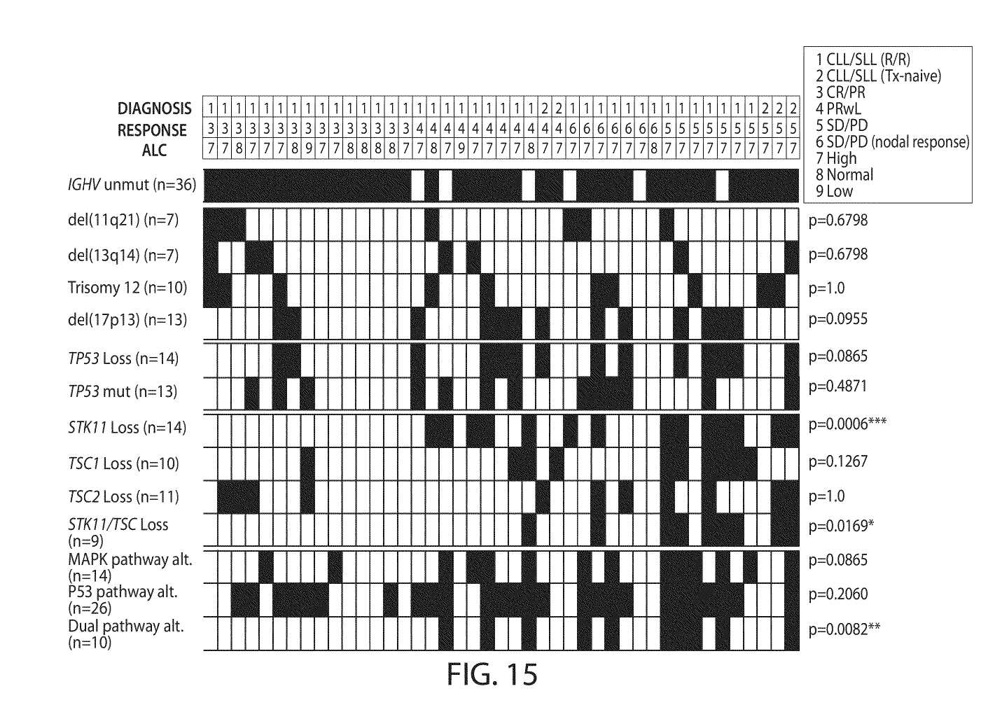

The results are shown in FIG. 15. The genes that belonged to the MAPK pathway and the p53 pathway were determined based on pathway identities from KEGG.

The results indicate that STK11 copy number loss is associated with or predictive of nonresponsiveness or resistance (e.g., acquired resistance) of a cancer (e.g., a CLL) to a PI3K inhibitor (e.g., Compound 1).

In addition, the results indicate that a dual pathway alteration (a mutation in both MAPK and p53 pathways) is associated with or predictive of nonresponsiveness or resistance (e.g., acquired resistance) of a cancer (e.g., a CLL) to a PI3K inhibitor (e.g., Compound 1). Genes in the MAPK and p53 pathways that were frequently mutated are indicated in Tables 23 and 25 below.

Furthermore, the results indicate that copy number loss of STK11 combined with copy number loss of TSC1, TSC2, or both (shown as “STK11/TSC loss” in FIG. 15) is associated with or predictive of nonresponsiveness or resistance (e.g., acquired resistance) of a cancer (e.g., a CLL) to a PI3K inhibitor (e.g., Compound 1).

Mutations in TP53 were further characterized, by determining the frequency of TP35 mutations in responders versus non-responders. Specifically, Table 20 below shows that TP53 alterations, including loss of TP53 and TP53 mutations, were more common in non-responders than responders. Thus, loss of TP53 correlated with a poorer prognosis.

TABLE 20

| | CR/PR | SD/PD | P value |
|---|---|---|---|
| Genetic alterations | (n = 32) | (n = 23) | (Fisher's exact) |
| Loss of TP53 | 6 | 11 | 0.0368* |
| TP53 mutation | 6 | 7 | 0.34 |
| Both | 3 | 5 | 0.2573 |
| Any TP53 alterations | 9 | 13 | 0.0511 |

### Example 12

### Relationship Between Mutational and Copy Number Variation Frequencies and Responses

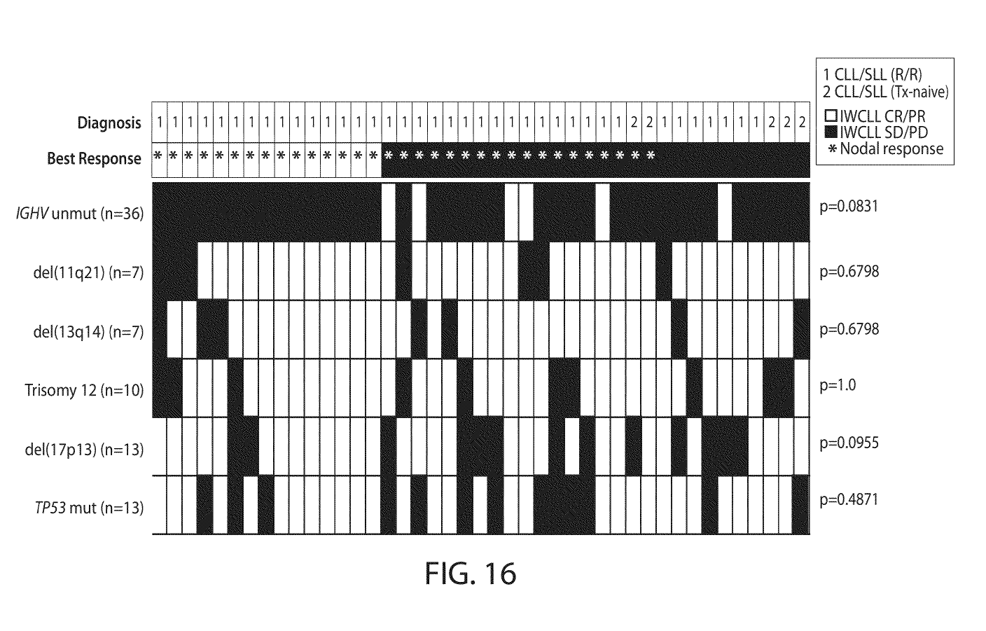

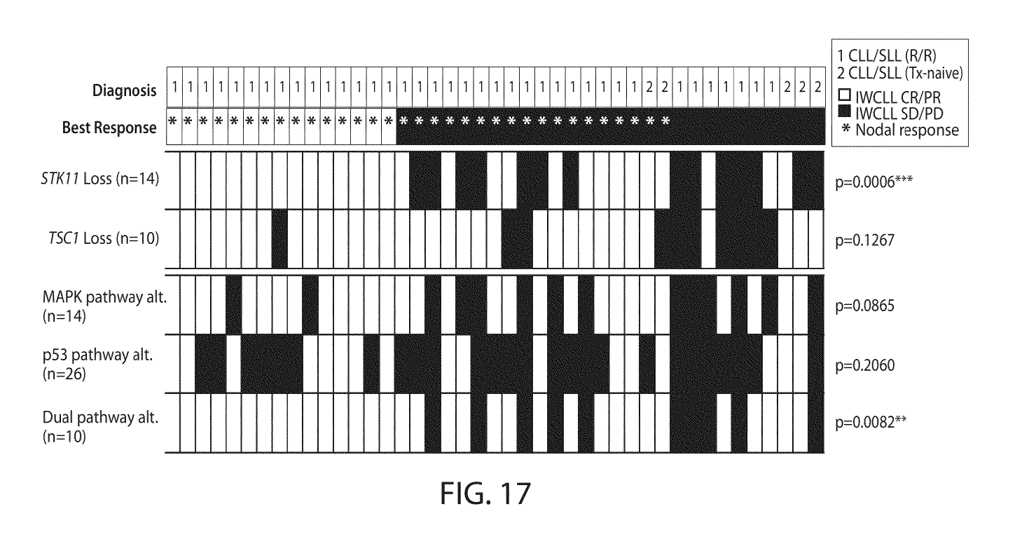

FIGS. 16 and 17 show the results of a re-analysis of the same data that were used in the analysis presented in Example 11, except that PR patients with lymphocytosis were classified as non-responders, whereas such patients were classified as responders in Example 11.

The results of the re-analysis confirmed that STK11 copy number loss is associated with or predictive of nonresponsiveness or resistance (e.g., acquired resistance) of a cancer (e.g., a CLL) to a PI3K inhibitor (e.g., Compound 1). Furthermore, the results confirmed that a dual pathway alteration (a mutation in both MAPK and p53 pathways) and mutation of BCR pathway is associated with or predictive of nonresponsiveness or resistance (e.g., acquired resistance) of a cancer (e.g., a CLL) to a PI3K inhibitor (e.g., Compound 1).

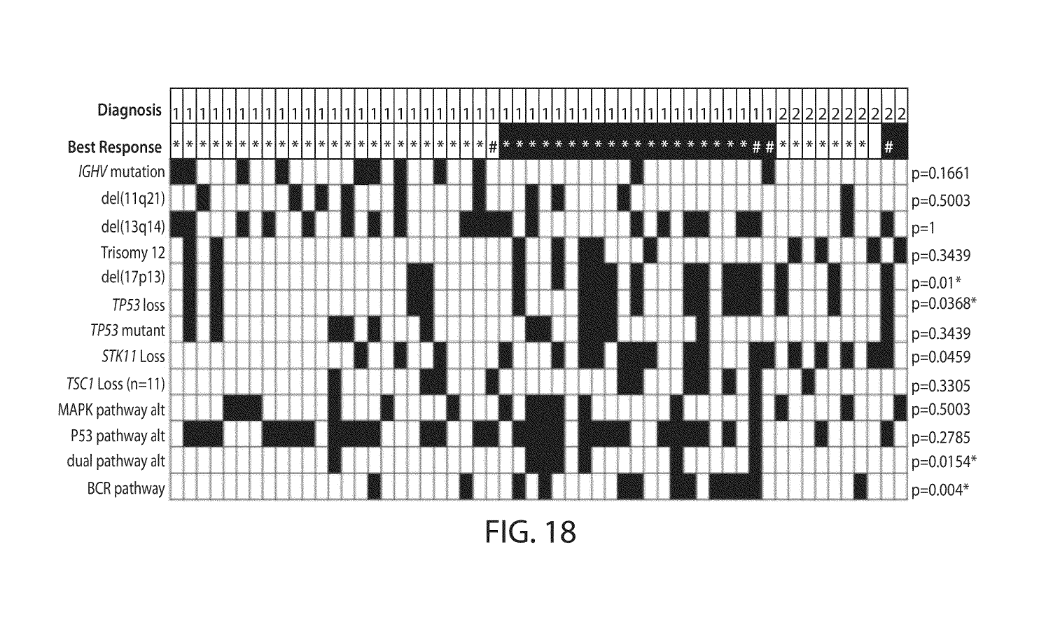

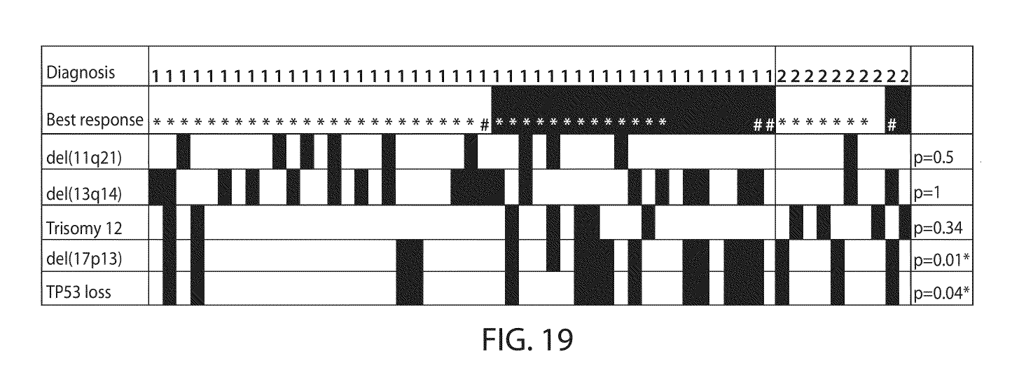

FIG. 18 shows additional results of an analysis of relationships between mutations and copy number variations and responses. Correlations between CLL common CNVs and response to Compound 1 are shown in FIG. 19.

### Example 13

### Additional Data Regarding CNVs and Mutations in CLL Patients

Using the methods described in the Examples above, CNVs that are more frequently present in non-responders versus responders to Compound 1 were determined. The results are shown in Table 21.

TABLE 21
CNVs that are more frequently present
in Compound 1 non-responders.

| | Chromosome | | |
|---|---|---|---|
| Region | location | Gene | Event |
| chr19: 1,205,798-1,228,434 | 19p13.3 | STK11 | Copy number |
| | | | loss |
| chr9: 135,766,735-135,820,020 | 9q34.13 | TSC1 | Copy number |
| | | | loss |
| chr16: 2,097,990-2,138,713 | 16p13.3 | TSC2 | Copy number |
| | | | loss |

In total 140 genes were detected with baseline mutations in Compound 1-treated CLL patients (Table 22).

TABLE 22
List of genes that have mutations detected
in Compound 1-treated CLL patients

| GeneName | Refseq ID |
|---|---|
| ABCA13 | NM_152701 |
| ABCA7 | NM_019112 |
| ADAMTSL3 | NM_207517 |
| AKAP8 | NM_005858 |
| ALK | NM_004304 |
| ARID1A | NM_006015 |
| ARID1B | NM_020732 |
| ASXL1 | NM_015338 |
| ATM | NM_000051 |
| ATR | NM_001184 |
| ATRX | NM_000489 |
| BCL11A | NM_022893 |
| BCL2 | NM_000633 |
| BCR | NM_004327 |
| BIRC3 | NM_001165 |
| BRAF | NM_004333 |
| BTG1 | NM_001731 |
| BTK | NM_001287344 |
| CARD11 | NM_032415 |
| CBFA2T3 | NM_005187 |
| CBL | NM_005188 |
| CCND3 | NM_001287427 |
| CCT6B | NM_006584 |
| CD36 | NM_001001548 |
| CDC73 | NM_024529 |
| CDH1 | NM_004360 |
| CDH11 | NM_001797 |
| CIC | NM_015125 |
| CIITA | NM_001286402 |
| COL4A2 | NM_001846 |
| CREBBP | NM_004380 |
| CSMD1 | NM_033225 |
| CSMD3 | NM_198123 |
| DAXX | NM_001141969 |
| DCHS1 | NM_003737 |
| DEK | NM_003472 |
| DIS3 | NM_014953 |
| DNM2 | NM_001005361 |
| DNMT1 | NM_001130823 |
| DPYD | NM_000110 |
| DST | NM_001144769 |
| EP300 | NM_001429 |
| EPHB1 | NM_004441 |
| EPHB2 | NM_004442 |
| ERBB4 | NM_005235 |
| ETV6 | NM_001987 |
| FAT2 | NM_001447 |
| FAT4 | NM_024582 |
| FBXO11 | NM_001190274 |
| FBXW7 | NM_033632 |
| FGFR1 | NM_001174064 |
| FGFR2 | NM_022970 |
| FGFR4 | NM_213647 |
| FLT3 | NM_004119 |
| FOXO1 | NM_002015 |
| FTCD | NM_006657 |
| FUBP1 | NM_003902 |
| FUS | NM_004960 |
| GNAQ | NM_002072 |
| GNAS | NM_001077490 |
| GRM8 | NM_001127323 |
| H3F3A | NM_002107 |
| HLF | NM_002126 |
| HNF1A | NM_000545 |
| HOXC13 | NM_017410 |
| HRAS | NM_176795 |
| IDH1 | NM_001282387 |
| JAK3 | NM_000215 |
| KIT | NM_000222 |
| LPHN3 | NM_015236 |
| LRP1B | NM_018557 |
| LRRK2 | NM_198578 |
| MAF | NM_001031804 |
| MAGI1 | NM_015520 |
| MALT1 | NM_006785 |
| MAP2K1 | NM_002755 |
| MAP3K1 | NM_005921 |
| MDM2 | NM_002392 |
| MED12 | NM_005120 |
| MEF2B | NM_001145785 |
| MKL1 | NM_001282662 |
| MSH2 | NM_000251 |
| MSH6 | NM_000179 |
| MTOR | NM_004958 |
| MYC | NM_002467 |
| MYD88 | NM_001172567 |
| NCOA2 | NM_006540 |
| NCOR1 | NM_006311 |
| NF1 | NM_001042492 |
| NIN | NM_020921 |
| NKX2-1 | NM_003317 |
| NOTCH1 | NM_017617 |
| NOTCH2 | NM_024408 |
| NSD1 | NM_022455 |
| NTRK1 | NM_002529 |
| NTRK3 | NM_001007156 |
| NUMA1 | NM_001286561 |
| NUP214 | NM_005085 |
| NUP98 | NM_016320 |
| OGT | NM_181672 |
| PCDH15 | NM_001142771 |
| PCLO | NM_033026 |
| PCM1 | NM_006197 |
| PCSK7 | NM_004716 |
| PDE4DIP | NM_014644 |
| PDGFRA | NM_006206 |
| PDGFRB | NM_002609 |
| PER1 | NM_002616 |
| PKHD1 | NM_138694 |
| PLCG2 | NM_002661 |
| PML | NM_033238 |
| PMS2 | NM_000535 |
| PRDM16 | NM_022114 |
| PRKDC | NM_006904 |
| PTCH1 | NM_001083602 |
| PTPRD | NM_002839 |
| PTPRT | NM_133170 |
| RALGDS | NM_006266 |
| RB1 | NM_000321 |
| RELN | NM_005045 |
| RNF213 | NM_001256071 |
| ROBO2 | NM_001290040 |
| RYR1 | NM_000540 |
| SETD2 | NM_014159 |
| SF3B1 | NM_012433 |
| SH2B3 | NM_005475 |
| SMARCA4 | NM_001128844 |
| STAT6 | NM_001178078 |
| SUZ12 | NM_015355 |
| SYNE1 | NM_182961 |
| TAL1 | NM_003189 |
| TCF3 | NM_003200 |
| TET1 | NM_030625 |
| TET2 | NM_001127208 |
| TLL2 | NM_012465 |
| TNFAIP3 | NM_001270508 |
| TP53 | NM_001276696 |
| TRIP11 | NM_004239 |
| XPO1 | NM_003400 |
| ZRSR2 | NM_005089 |

Frequently altered signaling pathways in Compound 1 non-responders and the involved genes and mutation sites are shown in Table 23, Table 24 and Table 25.

In the MAPK and ERBB signaling pathways, 16 genes were frequently mutated: BIRC3, BRAF,CBL, ERBB4, FGFR1, FGFR2, FGFR4, FLT3, HRAS, MAP2K1, MAP3K1, MTOR, MYC, NF1, NTRK1, PDGFRA and PDGFRB. See Table 23.

In the BCR pathway, 7 genes were frequently mutated: BCL2, BTK, CARD11, MALT1, MTOR, MYD88 AND PLCG2. See Table 24.

In the p53 signaling and cell cycle pathways 12 genes were frequently mutated: ATM, ATR, CCND3, MYC, CREBBP, EP300, FBXW7, MDM2, PRKDC, RB1, TP53 and XPO1. See Table 25.

In addition, mutations in the JAK/STAT, NFkB, and apoptosis pathways are enriched in IWCLL non-responders.

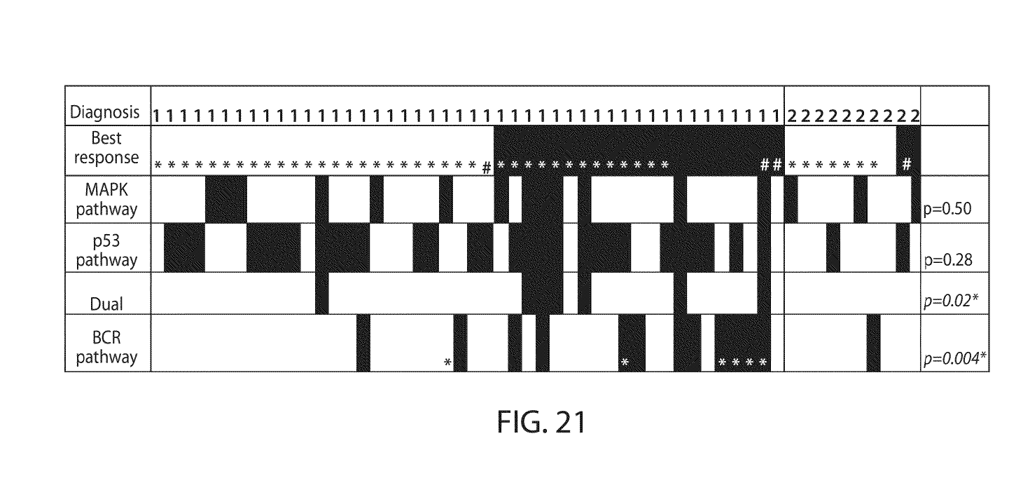

FIG. 21 shows relationships between response and alterations in genes of various pathways, including the MAPK pathway, p53 pathway, dual p53 and MAPK pathways, and BCR pathway.

TABLE 23
The frequently mutated MAPK pathway genes and mutation sites
in Compound 1 non-responders.

| | | | | | Reference | Mutation | |
|---|---|---|---|---|---|---|---|
| Pathway | GeneName | Refseq ID | Chromosome | Position | Allele | Allele | AAMutation |
| MAPK | BIRC3 | NM_001165 | 11 | 102201966 | G | G-AATC | E440DEL |
| | BRAF | NM_004333 | 7 | 140534536 | G | C | S126C |
| | CBL | NM_005188 | 11 | 119155775 | C | G | P510A |
| | ERBB4 | NM_005235 | 2 | 212295820 | C | A | M831I |
| | ERBB4 | NM_005235 | 2 | 212989562 | C | T | R50H |
| | ERBB4 | NM_005235 | 2 | 213403221 | T | A | S12C |
| | FGFR1 | NM_001174064 | 8 | 38272320 | C | T | D642N |
| | FGFR2 | NM_022970 | 10 | 123310807 | G | C | Y207* |
| | FGFR4 | NM_213647 | 5 | 176520277 | A | C | H399P |
| | FGFR4 | NM_213647 | 5 | 176517445 | T | G | L49R |
| | FLT3 | NM_004119 | 13 | 28623641 | T | G | N306H |
| | HRAS | NM_176795 | 11 | 533509 | C | T | D132N |
| | MAP2K1 | NM_002755 | 15 | 66727455 | G | T | K57N |
| | MAP2K1 | NM_002755 | 15 | 66782068 | C | G | N345K |
| | MAP3K1 | NM_005921 | 5 | 56168815 | G | T | A557S |
| | MTOR | NM_004958 | 1 | 11204742 | C | T | R1612Q |
| | MTOR | NM_004958 | 1 | 11301623 | C | T | A510T |
| | MYC | NM_002467 | 8 | 128750680 | A | C | T73P |
| | NF1 | NM_001042492 | 17 | 29557906 | A | C | N1054H |
| | NTRK1 | NM_002529 | 1 | 156836766 | G | A | E142K |
| | PDGFRA | NM_006206 | 4 | 55139810 | G | A | A491T |
| | PDGFRA | NM_006206 | 4 | 5512932 | G | A | E156K |
| | PDGFRB | NM_002609 | 5 | 149510109 | G | A | L454F |

TABLE 24
The frequently mutated BCR pathway genes and mutation sites
in Compound 1 non-responders.

| | | | | | Reference | Mutation | |
|---|---|---|---|---|---|---|---|
| Pathway | GeneName | Refseq ID | Chromosome | Position | Allele | Allele | AAMutation |
| BCR | BCL2 | NM_000633 | 18 | 60985508 | G | T | A131D |
| | BTK | NM_001287344 | X | 100611164 | C | A | C481F |
| | BTK | NM-001287344 | X | 100611164 | C | G | C481S |
| | CARD11 | NM_032415 | 7 | 2959106 | G | A | R804C |
| | MALT1 | NM_006785 | 18 | 56411677 | A | G | K621E |
| | MALT1 | NM_006785 | 18 | 56414750 | G | A | M717I |
| | MTOR | NM_004958 | 1 | 11204742 | C | T | R1612Q |
| | MTOR | NM_004958 | 1 | 11301623 | C | T | A510T |
| | MYC | NM_002467 | 8 | 128750680 | A | C | T73P |
| | MYD88 | NM_001172567 | 3 | 38182025 | G | T | V217F |
| | MYD88 | NM_001172567 | 3 | 38182337 | C | T | P266L |
| | PLCG2 | NM_002661 | 16 | 81973605 | T | G | M1141R |
| | PLCG2 | NM_002661 | 16 | 81953154 | C | T | S707F |

TABLE 25
The frequently mutated p53/cell cycle pathway genes and mutation sites in
Compound 1 non-responders.

| | Gene | | | | Reference | Mutation | |
|---|---|---|---|---|---|---|---|
| Pathway | Name | Refseq ID | Chromosome | Position | Allele | Allele | AAMutation |
| p53/Cell cycle | ATM | NM_000051 | 11 | 108196836 | G | A | G2287R |
| | ATM | NM_000051 | 11 | 108129788 | A | A- | I818DEL |
| | | | | | | TTTGTAAAAG | |
| | ATM | NM_000051 | 11 | 108200967 | T | A | L2445Q |
| | ATM | NM_000051 | 11 | 108164152 | G | T | R1575L |
| | ATM | NM_000051 | 11 | 108186596 | T | C | L2018S |
| | ATM | NM_000051 | 111 | 108115724 | A | G | H291R |
| | ATR | NM_001184 | 3 | 142274725 | T | A | K779* |
| | ATR | NM_001184 | 3 | 142274853 | C | A | G736V |
| | CCND3 | NM_001287427 | 6 | 41904413 | G | A | P149S |
| | CCND3 | NM_001287427 | 6 | 41903707 | G | A | P234S |
| | CREBBP | NM_004380 | 16 | 3795324 | T | A | M1290L |
| | EP300 | NM_001429 | 22 | 41574510 | T | T-CAG | L2265DEL |
| | EP300 | NM_001429 | 22 | 41513811 | C | G | P239A |
| | FBXW7 | NM_033632 | 4 | 153253763 | T | A | K324* |
| | MDM2 | NM_002392 | 12 | 69233526 | T | G | L464R |
| | MDM2 | NM_002392 | 12 | 69233160 | A | G | K342R |
| | MDM2 | NM_002392 | 12 | 69233130 | G | A | R332H |
| | MDM2 | NM_002392 | 12 | 69233252 | G | A | V373M |
| | PRKDC | NM_006904 | 8 | 48855869 | T | C | N289S |
| | PRKDC | NM_006904 | 8 | 48761821 | C | G | V2391L |
| | PRKDC | NM_006904 | 8 | 48691647 | T | C | R3832G |
| | PRKDC | NM_006904 | 8 | 48767904 | G | A | R2213* |
| | RB1 | NM_000321 | 13 | 48878084 | C | C- | T12DEL |
| | | | | | | GCCGCCGCT | |
| | RB1 | NM_000321 | 13 | 49039396 | G | C | S794T |
| | TP53 | NM_001276696 | 17 | 7578199 | A | C | V178G |
| | TP53 | NM_001276696 | 17 | 7578196 | A | T | V179E |
| | TP53 | NM_001276696 | 17 | 7578211 | C | A | R174L |
| | TP53 | NM_001276696 | 17 | 7578437 | G | A | Q126* |
| | TP53 | NM_001276696 | 17 | 7578508 | C | T | C102Y |
| | TP53 | NM_001276696 | 17 | 7577114 | C | T | C236Y |
| | TP53 | NM_001276696 | 17 | 7578221 | T | T-TC | R209DEL |
| | TP53 | NM_001276696 | 17 | 7578394 | G | C | H140R |
| | TP53 | NM_001276696 | 17 | 7578272 | G | A | H154Y |
| | TP53 | NM_001276696 | 17 | 7578554 | A | C | Y87D |
| | TP53 | NM_001276696 | 17 | 7578394 | T | C | H140R |
| | TP53 | NM_001276696 | 17 | 7578484 | G | G-A | S110DEL |
| | TP53 | NM_001276696 | 17 | 7578263 | G | C | R157G |
| | TP53 | NM_001276696 | 17 | 7577144 | A | G | L226P |
| | TP53 | NM_001276696 | 17 | 7577123 | A | T | V233E |
| | XPO1 | NM_003400 | 2 | 61719472 | C | T | E571K |

### Example 14

### PTEN is a Biomarker for Compound 1 Resistance

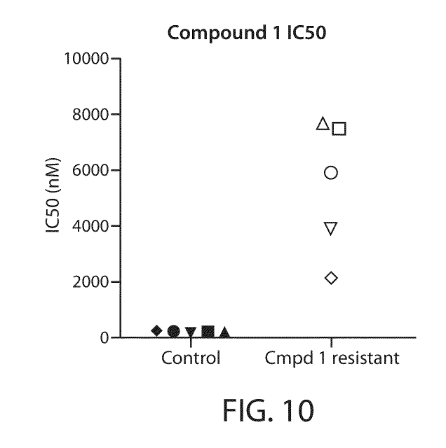

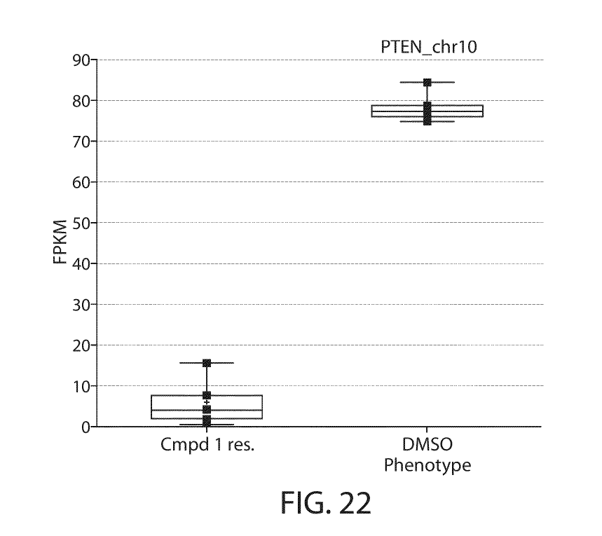

Experiments were performed to assess the expression of PTEN in cells that were resistant to Compound 1. Compound 1 resistant cells were generated by culturing cells in the presence of Compound 1 or DMSO as a control for 8 weeks. Cells were subcloned under selective pressure from the drug, seeding at densities of 3 cells per well, 1 cell per well, or 0.3 cell per well. Parental, DMSO-treated, and Compound 1-resistant clones were selected for expansion. Five clones from each group were expanded. Cells were harvested for various assays, including CTG (Cell Titer Glo, Promega, an assay that measures ATP levels as a surrogate for cell number in order to observe cell viability and changes in proliferation rate), PD (pharmacodynamic assay), RNA analysis, DNA analysis, and short tandem repeat (STR) fingerprinting. A CTG assay was performed to confirm that cells were resistant to Compound 1 at the time of sample collection. As shown in FIG. 10 and Table 26, the average 1050 for Compound 1 inhibition of the resistant cells was higher than the control cells. RNA-seq experiments were also performed on the samples from DMSO control and Compound 1 resistant cells. Five clones of each—DMSO-treated control cells that are not resistant to Compound 1, and Compound 1 resistant cells—were tested. As shown in FIG. 22, there was a substantial downregulation in PTEN expression in Compound 1 resistant cell clones, but not in the DMSO control-treated cell clones. This downregulation in PTEN expression was seen at the RNA level as well as the protein level. These results show that PTEN is a biomarker for Compound 1 resistance, where low PTEN levels correlate with resistance.

TABLE 26

| Clones | AVG Compound 1 IC50 (nM) |
|---|---|
| Control | 241 ± 17 |
| Compound 1 resistant | 5420 ± 1079 |

### EQUIVALENTS

While this invention has been disclosed with reference to specific aspects, it is apparent that other aspects and variations of this invention can be devised by others skilled in the art without departing from the true spirit and scope of the invention. The appended claims are intended to be construed to include all such aspects and equivalent variations.

1. (canceled)

2. A method of treating a cancer in a subject comprising administering to the subject a synergistic combination of a PI3K inhibitor or a pharmaceutically acceptable form thereof, wherein the PI3K inhibitor is (S)-3-(1-((9H-purin-6-yl)amino)ethyl)-8-chloro-2-phenylisoquinolin-1(2H)-one or (S)-2-(1-(9H-purin-6-ylamino)propyl)-5-fluoro-3-phenylquinazolin-4(3H)-one,
and a CDK 4/6 inhibitor or a pharmaceutically acceptable form thereof.

3. A composition comprising a synergistic combination of a PI3K inhibitor or a pharmaceutically acceptable form thereof, wherein the PI3K inhibitor is (S)-3-(1-((9H-purin-6-yl)amino)ethyl)-8-chloro-2-phenylisoquinolin-1(2H)-one or (S)-2-(1-(9H-purin-6-ylamino)propyl)-5-fluoro-3-phenylquinazolin-4(3H)-one,
and a second therapeutic agent or a pharmaceutically acceptable form thereof, wherein the second therapeutic agent is chosen from one or more of: 1) a CDK 4/6 inhibitor, 2) an HDAC inhibitor, 3) a MEK inhibitor, 4) a mTOR inhibitor, 5) an AKT inhibitor, 6) a proteasome inhibitor, 7) an immunomodulator, 8) a glucocorticosteroid, 9) a BET inhibitor, 10) an epigenetic inhibitor, 11) a PI3K alpha inhibitor, 12) a topoisomerase inhibitor, or 13) an ERK inhibitor.

4. The method of claim 2, wherein the combination is synergistic as indicated by a combination index value that is less than 1, less than 0.7, or less than 0.5 for the combination of the PI3K inhibitor and the second therapeutic agent.

5-6. (canceled)

7. The method of claim 4, wherein the combination index value is assessed at 50% inhibition.

8. The method of claim 4, wherein the combination index value is assessed at 50% growth inhibition.

9-10. (canceled)

11. The method of claim 2, wherein the PI3K inhibitor is Compound 1:

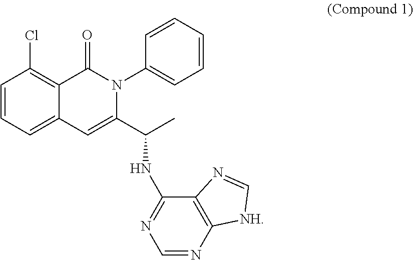 [SMILES: C[C@H](Nc1ncnc2[nH]cnc12)c1cc2cccc(Cl)c2c(=O)n1-c1ccccc1]

12. The method of claim 2, wherein the PI3K inhibitor is CAL-101 (GS1101):

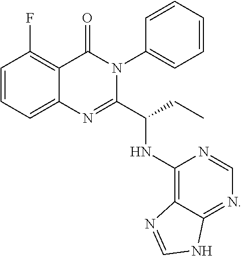 [SMILES: CC[C@H](Nc1ncnc2[nH]cnc12)c1nc2cccc(F)c2c(=O)n1-c1ccccc1]

13. (canceled)

14. The method of claim 121, wherein the MEK inhibitor is AZD8330, MEK162 (ARRY438162), PD-0325901, pimasertib (AS703026, MSC1935369), refametinib (BAY869766, RDEA119), RO5126766, selumetinib, TAK733, trametinib (GSK1120212), WX-554, RO4987655 (CH4987655), XL-518 (GDC-0973), PD184352 (CI-1040), AZD2644, or GDC0623, or a combination thereof.

15-16. (canceled)

17. The method of claim 121, wherein the mTOR inhibitor is AP23841, AZD8055, BEZ235, BGT226, deferolimus (AP23573/MK-8669), EM101/LY303511, everolimus (RAD001), EX2044, EX3855, EX7518, GDC0980, INK-128, KU-0063794, NV-128, OSI-027, PF-4691502, rapalogs, rapamycin, ridaforolimus, SAR543, SF1126, temsirolimus (CCI-779), WYE-125132, XL765, zotarolimus (ABT578), torin 1, GSK2126458, AZD2014, GDC-0349, or XL388, or a combination thereof.

18-19. (canceled)

20. The method of claim 121, wherein the AKT inhibitor is AZD5363, miltefosine, perifosine, VQD-002, MK-2206, GSK690693, GDC-0068, triciribine, CCT128930, PHT-427, or honokiol, or a combination thereof.

21-22. (canceled)

23. The method of claim 121, wherein the proteasome inhibitor is bortezomib, carfilzomib, CEP-18770, disulfiram, epigallocatechin-3-gallate, epoxomicin, lactacystin, MG132, MLN9708, ONX 0912, or salinosporamide A, or a combination thereof.

24-25. (canceled)

26. The method of claim 121, wherein the immunomodulator is lenalidomide, pomalidomide, or thalidomide, or a combination thereof.

27-28. (canceled)

29. The method of claim 121, wherein the glucocorticosteroid is dexamethasone, aldosterone, beclomethasone, betamethasone, hydrocortisone, cortisone, deoxycorticosterone acetate (DOCA), fludrocortisone acetate, methylprednisolone, prednisolone, or prednisone, or a combination thereof.

30-31. (canceled)

32. The method of claim 2, wherein the CDK4/6 inhibitor is LEE011, PD0332991 (palbociclib), or LY2835219 (Abemaciclib), or a combination thereof.

33. (canceled)

34. The method of claim 121, wherein the HDAC inhibitor is vorinostat (SAHA), romidepsin (depsipeptide or FK-228), panobinostat, valproic acid, belinostat (PXD101), mocetinostat, abrexinostat, entinostat, SB939, resminostat, givinostat, CUDC-101, AR-42, CHR-2845, CHR-3996, 4SC-202, CG200745, LAQ824, ACY-1215, or kevetrin, or a combination thereof.

35-38. (canceled)

39. The method of claim 121, wherein the BET inhibitor is (+)-JQ1, GSK525762, I-BET151, PF-6405761, I-BET-762, RVX-208, OF-1, MS436, I-BET726, PFI-3, or CPI-203, or a combination thereof.

40-41. (canceled)

42. The method of claim 121, wherein the epigenetic inhibitor is azacitidine, decitabine, RG108, thioguanine, zebularine, procainamide HCl, SGI-1027, or lomeguatrib or a combination thereof.

43-44. (canceled)

45. The method of claim 121, wherein the PI3K alpha inhibitor is GDC-0941, GDC-0032, HS-173, A66, PIK-75, Alpelisib, Gedatolisib, CH5132799, or Copanlisib, or a combination thereof.

46-47. (canceled)

48. The method of claim 121, wherein the topoisomerase inhibitor is doxorubicin HCl, Podophyllotoxin, Etoposide, Oxolinic Acid, Sedanolide, Mitoxantrone Dihydrochloride, 9-Hydroxyellipticine, or Amrubicin, or a combination thereof.

49-50. (canceled)

51. The method of claim 121, wherein the ERK inhibitor is SCH772984, BVD-523, MEK162, hypothemycin, or VX-11e, or a combination thereof.

52-53. (canceled)

54. The method of claim 2, wherein the composition comprises the PI3K inhibitor, or pharmaceutically acceptable form thereof, at an amount of in the range of from about 0.01 mg to about 75 mg and the CDK4/6 inhibitor, or pharmaceutically acceptable form thereof, at an amount of in the range of from about 0.01 mg to about 1100 mg.

55. (canceled)

56. The method of claim 2, wherein the PI3K inhibitor or pharmaceutically acceptable form thereof, and the CDK4/6 inhibitor or pharmaceutically acceptable form thereof, are in a single dosage form or in separate dosage forms.

57-60. (canceled)

61. The method of claim 2, wherein the concentration of the PI3K inhibitor that is required to achieve inhibition is at least 20% lower when the PI3K inhibitor is administered in combination with the CDK4/6 inhibitor than when the PI3K inhibitor is administered alone.

62-64. (canceled)

65. The method claims of claim 2, wherein the anti-cancer effect provided by the composition is greater than the anti-cancer effect provided by a monotherapy with the same dose of the PI3K inhibitor or pharmaceutically acceptable form thereof as is included in the composition.

66. The method of claim 2, wherein the anti-cancer effect provided by the composition is at least 2 fold greater, at least 3 fold greater, at least 5 fold greater, or at least 10 fold greater than the anti-cancer effect provided by the monotherapy with the PI3K inhibitor or pharmaceutically acceptable form thereof.

67-69. (canceled)

70. The method of claim 2, wherein the PI3K inhibitor, or a pharmaceutically acceptable form, is administered concurrently with, subsequent to, or prior to the CDK4/6 inhibitor.

71-72. (canceled)

73. The method claims of claim 2, wherein the combination delays resistance of the cancer to the PI3K inhibitor or reduces the risk that the cancer becomes resistant to the PI3K inhibitor.

74. (canceled)

75. The method claims of claim 73, wherein the cancer does not become resistant to the PI3K inhibitor for at least 12 months.

76. The method claims of claim 2, wherein the combination prolongs remission in the subject.

77. The method of claim 2, wherein the subject experiences remission for at least 12, 18, or 24 months.

78. (canceled)

79. The method of claim 2, wherein the subject experiences complete remission.

80. The method of claim 2, which results in a reduction in the level of minimal residual disease (MRD).

81. The method of claim 2, wherein the subject has substantially no detectable MRD.

82-83. (canceled)

84. A method of delaying or decreasing resistance of a subject having a cancer, comprising administering to the subject a synergistic amount of a PI3K inhibitor, or a pharmaceutically acceptable form thereof, and a second therapeutic agent selected from 1) a CDK 4/6 inhibitor, 2) an HDAC inhibitor, 3) a MEK inhibitor, 4) a mTOR inhibitor, 5) an AKT inhibitor, 6) a proteasome inhibitor, 7) an immunomodulator, 8) a glucocorticosteroid, 9) a BET inhibitor, 10) an epigenetic inhibitor, 11) a PI3K alpha inhibitor, 12) a topoisomerase inhibitor, or 13) an ERK inhibitor, or a pharmaceutically acceptable form thereof, thereby delaying or decreasing resistance.

85-87. (canceled)

88. A method of reducing the level of minimal residual disease (MRD) compared to a reference value in a subject having a cancer, comprising administering to the subject a synergistic amount of a PI3K inhibitor, or a pharmaceutically acceptable form thereof, and a second therapeutic agent selected from from 1) a CDK 4/6 inhibitor, 2) an HDAC inhibitor, 3) a MEK inhibitor, 4) a mTOR inhibitor, 5) an AKT inhibitor, 6) a proteasome inhibitor, 7) an immunomodulator, 8) a glucocorticosteroid, 9) a BET inhibitor, 10) an epigenetic inhibitor, 11) a PI3K alpha inhibitor, 12) a topoisomerase inhibitor, or 13) an ERK inhibitor, or a pharmaceutically acceptable form thereof, thereby reducing the level of MRD in the subject.

89. (canceled)

90. A method of treating a cancer or tumor in a subject, comprising:
acquiring a value for one or more of: the presence, absence, amount or level of an alteration or biomarker chosen from one, two, three, four, five, six, seven, eight, nine, 10, 11, 12, 13, 14, 15, or all of: an STK11 copy number, TSC1 copy number, TSC2 copy number, TP53 copy number, PTEN copy number, CBFA2T3 copy number, YWHAE copy number, PER1 copy number, GAS7 copy number, FSTL3 copy number, USP6 copy number, MAP2K4 copy number, EGFR copy number, a BCR pathway mutation a p53 pathway mutation, or a MAPK pathway mutation, or any combination thereof, and
responsive to said value, administering to the subject a composition according to claim 3.

91-97. (canceled)

98. The method of claim 90, wherein one, two, three, four, five, six, seven, eight, nine, 10, 11, 12, 13, or all of the following is indicative of decreased responsiveness of the cancer or tumor, or the subject, to the treatment:
(i) a copy number loss of STK11;
(ii) a copy number loss of TSC1 or TSC2, or both;
(iii) a copy number loss of TP53;
(iv) a copy number loss of PTEN;
(v) a copy number loss of CBFAT2T3;
(vi) a copy number loss of YWHAE;
(vii) a copy number loss of PER1;
(viii) a copy number loss of GAS7;
(ix) a copy number loss of FSTL3;
(x) a copy number loss of USP6;
(xi) a copy number loss of MAP2K4;
(xii) a BCR pathway mutation;
(xiii) a p53 pathway mutation; or
(xiv) a MAPK pathway mutation.

99-102. (canceled)

103. The method of claim 2, wherein the cancer is of hematopoietic origin.

104. The method of claim 103, wherein the cancer is a lymphoma or leukemia.

105. The method of claim 104, wherein the cancer is B-cell lymphoma, mantle cell lymphoma, non-Hodgkin's B-cell lymphoma, non-Hodgkin's lymphoma T-cell lymphoma, cutaneous lymphoma, anaplastic large cell lymphoma, multiple myeloma, myeloma, plasmacytoma, a non-Hodgkin lymphoma, a diffuse large B-cell lymphoma, a diffuse large B-cell lymphoma activated B-cell like, a diffuse large B-cell lymphoma germinal center B-cell like, an indolent non-Hodgkin lymphoma, a follicular lymphoma, or a T-cell lymphoma.

106-114. (canceled)

115. The method of claim 2, wherein the subject is a human.

116-120. (canceled)

121. A method of treating a cancer in a subject comprising administering to the subject a synergistic combination of a PI3K inhibitor or a pharmaceutically acceptable form thereof, wherein the PI3K inhibitor is (S)-3-(1-((9H-purin-6-yl)amino)ethyl)-8-chloro-2-phenylisoquinolin-1(2H)-one or (S)-2-(1-(9H-purin-6-ylamino)propyl)-5-fluoro-3-phenylquinazolin-4(3H)-one,
and a second therapeutic agent, or a pharmaceutically acceptable form thereof, wherein the second agent is selected from one or more of 1) an HDAC inhibitor, 2) a MEK inhibitor, 3) a mTOR inhibitor, 4) an AKT inhibitor, 5) a proteasome inhibitor, 6) an immunomodulator, 7) a glucocorticosteroid, 8) a BET inhibitor, 9) an epigenetic inhibitor, 10) a PI3K alpha inhibitor, 11) a topoisomerase inhibitor, or 12) an ERK inhibitor or a combination thereof.


SEQUENCE LISTING (3 sequences)

SEQ ID NO: 1 - protein, 6 aa, Artificial Sequence
  Description of Artificial Sequence Synthetic peptide
MDGCEL

SEQ ID NO: 2 - DNA, 11 bp, Homo sapiens
atttgtaaaag

SEQ ID NO: 3 - DNA, 10 bp, Homo sapiens
cgccgccgct